SEQUENCE LISTING (86 sequences)

SEQ ID NO: 1 - protein, 925 aa, Artificial Sequence
  ENPP1 Amino Acid Sequence - Wild Type
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 1077 aa, Artificial Sequence
  Azurocidin-ENPP1-FC
  SIGNAL (19)..(20): Cleavage point of the signal sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 1468 aa, Artificial Sequence
  Azurocidin-ENPP1-Alb
  SIGNAL (19)..(20): Cleavage point of the signal sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 4 - protein, 1594 aa, Artificial Sequence
  Azurocidin-ENPP1
  SIGNAL (19)..(20): Cleavage point of the signal sequence
MTRLTVLALLAGLLASSRAAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 5 - protein, 888 aa, Artificial Sequence
  ENPP2 Amino Acid Sequence - Wild Type
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKFRGSRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 6 - protein, 827 aa, Artificial Sequence
  Extracellular Domain of ENPP3
EKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 7 - protein, 875 aa, Artificial Sequence [sequence omitted]

SEQ ID NO: 8 - protein, 1073 aa, Artificial Sequence
  Azurocidin-ENPP3-FC
  SIGNAL (19)..(20): Cleavage point of the signal sequence
  MISC_FEATURE (847)..(1074): Indicate Fc sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 1464 aa, Artificial Sequence
  Azurocidin-ENPP3-Albumin
  SIGNAL (19)..(20): Cleavage point of the signal sequence
  MISC_FEATURE (847)..(1465): Indicate albumin sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 10 - protein, 846 aa, Artificial Sequence
  Azurocidin-ENPP3
  SIGNAL (19)..(20): Cleavage point of the signal sequence
MTRLTVLALLAGLLASSRAAKQGSCRKKCFDASFRGLENCRCDVACKDRGDCCWDFEDTCVESTRIWMCNKFRCGETRLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMNLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTI

SEQ ID NO: 11 - protein, 453 aa, Artificial Sequence
  ENPP4 Amino Acid Sequence - Wild Type
MKLLVILLFSGLITGFRSDSSSSLPPKLLLVSFDGFRADYLKNYEFPHLQNFIKEGVLVEHVKNVFITKTFPNHYSIVTGLYEESHGIVANSMYDAVTKKHFSDSNDKDPFWWNEAVPIWVTNQLQENRSSAAAMWPGTDVPIHDTISSYFMNYNSSVSFEERLNNITMWLNNSNPPVTFATLYWEEPDASGHKYGPEDKENMSRVLKKIDDLIGDLVQRLKMLGLWENLNVIITSDHGMTQCSQDRLINLDSCIDHSYYTLIDLSPVAAILPKINRTEVYNKLKNCSPHMNVYLKEDIPNRFYYQHNDRIQPIILVADEGWTIVLNESSQKLGDHGYDNSLPSMHPFLAAHGPAFHKGYKHSTINIVDIYPMMCHILGLKPHPNNGTFGHTKCLLVDQWCINLPEAIAIVIGSLLVLTMLTCLIIIMQNRLSVPRPFSRLQLQEDDDDPLIG

SEQ ID NO: 12 - protein, 850 aa, Artificial Sequence
  ENPP51 Amino Acid Sequence
  SIGNAL (24)..(25): Cleavage position at the signal peptide sequence
MTSKFLLVSFILAALSLSTTFSLQPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQED

SEQ ID NO: 13 - protein, 1474 aa, Artificial Sequence
  ENPP51 - ALB Amino Acid Sequence
  SIGNAL (24)..(25): Cleavage position at the signal peptide sequence
  MISC_FEATURE (857)..(1474): Indicate albumin sequence
MTSKFLLVSFILAALSLSTTFSLQPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQEDGGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 14 - protein, 1074 aa, Artificial Sequence
  ENPP5-NPP3-Fc sequence
  SIGNAL (22)..(23): Cleavage position at the signal peptide sequence
  MISC_FEATURE (848)..(1074): Indicate albumin sequence
MTSKFLLVSFILAALSLSTTFSKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 1469 aa, Artificial Sequence
  ENPP5-NPP3-Albumin sequence
  SIGNAL (22)..(23): Cleavage position at the signal peptide sequence
  MISC_FEATURE (848)..(1469): Indicate albumin sequence
MTSKFLLVSFILAALSLSTTFSKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 16 - protein, 23 aa, Artificial Sequence
  ENPP5 Protein Export Signal Sequence
  MISC_FEATURE (23)..(23): X can be any amino acid
MTSKFLLVSFILAALSLSTTFSX

SEQ ID NO: 17 - protein, 1079 aa, Artificial Sequence
  ENPP51-Fc
  SIGNAL (22)..(23): Cleavage position at the signal peptide sequence
  MISC_FEATURE (853)..(1079): Indicate Fc sequence
MTSKFLLVSFILAALSLSTTFSGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 1082 aa, Artificial Sequence
  ENPP71-Fc Amino Acid Sequence
  SIGNAL (22)..(23): Cleavage position at the signal peptide sequence
  MISC_FEATURE (856)..(1082): Indicate Fc sequence
MRGPAVLLTVALATLLAPGAGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 849 aa, Artificial Sequence
  ENPP71 (lacking NPP1 N-Terminus GLK) Amino Acid Sequence
  SIGNAL (22)..(23): Cleavage position at the signal peptide sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 20 - protein, 1079 aa, Artificial Sequence
  ENPP71 (lacking NPP1 N-Terminus GLK) - Fc Amino Acid Sequence
  SIGNAL (22)..(23): Cleavage position at the signal peptide sequence
  MISC_FEATURE (854)..(1079): Indicate Fc sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 1474 aa, Artificial Sequence
  ENPP71 (lacking NPP1 N-Terminus GLK) - ALB Amino Acid Sequence
  SIGNAL (22)..(23): Cleavage position at the signal peptide sequence
  MISC_FEATURE (857)..(1474): Indicate albumin sequence
MRGPAVLLTVALATLLAPGAGAPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 22 - protein, 1072 aa, Artificial Sequence
  ENPP7-NPP3-Fc sequence
  SIGNAL (20)..(21): Cleavage position at the signal peptide sequence
  MISC_FEATURE (846)..(1072): Indicate Fc sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 1396 aa, Artificial Sequence
  ENPP71-Albumin
  SIGNAL (23)..(24): Cleavage position at the signal peptide sequence
  MISC_FEATURE (857)..(1396): Indicate Fc sequence
MRGPAVLLTVALATLLAPGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDGGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTL

SEQ ID NO: 24 - protein, 1467 aa, Artificial Sequence
  ENPP7-NPP3-Albumin
  SIGNAL (20)..(21): Cleavage position at the signal peptide sequence
  MISC_FEATURE (860)..(1467): Indicate albumin sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 25 - protein, 1507 aa, Artificial Sequence
  ENPP7-ENPP3-Albumin
  SIGNAL (20)..(21): Cleavage position at the signal peptide sequence
  MISC_FEATURE (900)..(1506): Indicate albumin sequence
MRGPAVLLTVALATLLAPGAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 26 - protein, 852 aa, Artificial Sequence
  ENPP71 Amino Acid Sequence
  SIGNAL (22)..(23): Cleavage position at the signal peptide sequence
MRGPAVLLTVALATLLAPGAGAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 27 - protein, 925 aa, Artificial Sequence
  ENPP121 Amino Acid Sequence
  SIGNAL (92)..(93): Cleavage position at the signal peptide sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 28 - protein, 1155 aa, Artificial Sequence
  ENPP121-Fc Amino Acid Sequence
  SIGNAL (92)..(93): Cleavage position at the signal peptide sequence
  MISC_FEATURE (929)..(1155): Indicate Fc sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 1550 aa, Artificial Sequence
  ENPP121-ALB Amino Acid Sequence
  SIGNAL (92)..(93): Cleavage position at the signal peptide sequence
  MISC_FEATURE (933)..(1550): Indicate albumin sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDRSGSGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 30 - protein, 1147 aa, Artificial Sequence
  ENPP121-NPP3-Fc sequence
  SIGNAL (95)..(96): Cleavage position at the signal peptide sequence
  MISC_FEATURE (921)..(1147): Indicate Fc sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 1542 aa, Artificial Sequence
  ENPP121-NPP3-Albumin sequence
  SIGNAL (95)..(96): Cleavage position at the signal peptide sequence
  MISC_FEATURE (935)..(1542): Indicate albumin sequence
MERDGCAGGGSRGGEGGRAPREGPAGNGRDRGRSHAAEAPGDPQAAASLLAPMDVGEEPLEKAARARTAKDPNTYKIISLFTFAVGVNICLGFTAKQGSCRKKCFDASFRGLENCRCDVACKDRGDCCWDFEDTCVESTRIWMCNKFRCGERLEASLCSCSDDCLQRKDCCADYKSVCQGETSWLEENCDTAQQSQCPEGFDLPPVILFSMDGFRAEYLYTWDTLMPNINKLKTCGIHSKYMRAMYPTKTFPNHYTIVTGLYPESHGIIDNNMYDVNLNKNFSLSSKEQNNPAWWHGQPMWLTAMYQGLKAATYFWPGSEVAINGSFPSIYMPYNGSVPFEERISTLLKWLDLPKAERPRFYTMYFEEPDSSGHAGGPVSARVIKALQVVDHAFGMLMEGLKQRNLHNCVNIILLADHGMDQTYCNKMEYMTDYFPRINFFYMYEGPAPRIRAHNIPHDFFSFNSEEIVRNLSCRKPDQHFKPYLTPDLPKRLHYAKNVRIDKVHLFVDQQWLAVRSKSNTNCGGGNHGYNNEFRSMEAIFLAHGPSFKEKTEVEPFENIEVYNLMCDLLRIQPAPNNGTHGSLNHLLKVPFYEPSHAEEVSKFSVCGFANPLPTESLDCFCPHLQNSTQLEQVNQMLNLTQEEITATVKVNLPFGRPRVLQKNVDHCLLYHREYVSGFGKAMRMPMWSSYTVPQLGDTSPLPPTVPDCLRADVRVPPSESQKCSFYLADKNITHGFLYPPASNRTSDSQYDALITSNLVPMYEEFRKMWDYFHSVLLIKHATERNGVNVVSGPIFDYNYDGHFDAPDEITKHLANTDVPIPTHYFVVLTSCKNKSHTPENCPGWLDVLPFIIPHRPTNVESCPEGKPEALWVEERFTAHIARVRDVELLTGLDFYQDKVQPVSEILQLKTYLPTFETTIGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 32 - protein, 92 aa, Artificial Sequence
  ENPP121GLK Protein Export Signal Sequence
MERDGCAGGGSRGGEGGRAPREGPAGNGRDRGRSHAAEAPGDPQAAASLLAPMDVGEEPLEKAARARTAKDPNTYKIISLFTFAVGVNICLG

SEQ ID NO: 33 - protein, 623 aa, Artificial Sequence
  Albumin Sequence
GGGGSGGGGSGGGGSMKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 34 - protein, 227 aa, Artificial Sequence
  Human IgG Fc domain, Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 618 aa, Artificial Sequence
  Albumin Sequence
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALARSWSHPQFEK

SEQ ID NO: 36 - protein, 16 aa, Artificial Sequence
  ENPP2 Signal Peptide
LFTFAVGVNICLGFTA

SEQ ID NO: 37 - protein, 20 aa, Artificial Sequence
  Signal Sequence ENPP7
MRGPAVLLTVALATLLAPGA

SEQ ID NO: 38 - protein, 22 aa, Artificial Sequence
  Signal sequence ENPP7
MRGPAVLLTVALATLLAPGAGA

SEQ ID NO: 39 - protein, 95 aa, Artificial Sequence
  Signal Sequence ENPP1-2-1
MERDGCAGGGSRGGEGGRAPREGPAGNGRDRGRSHAAEAPGDPQAAASLLAPMDVGEEPLEKAARARTAKDPNTYKIISLFTFAVGVNICLGFTA

SEQ ID NO: 40 - protein, 17 aa, Artificial Sequence
  exENPP3
LLVIMSLGLGLGLGLRK

SEQ ID NO: 41 - protein, 22 aa, Artificial Sequence
  Signal Sequence ENPP5
MTSKFLLVSFILAALSLSTTFS

SEQ ID NO: 42 - DNA, 3255 bp, Artificial Sequence
  Azurocidin-ENPP1-FC Nucleotide sequence
  misc_feature (72)..(73): Nucleotide sequence of signal peptide
ggtaccgccaccatgacaagactgacagtgctggctctgctggccggactgttggcctcttctagagctgctccttcctgcgccaaagaagtgaagtcctgcaagggcagatgcttcgagcggaccttcggcaactgtagatgtgacgccgcttgcgtggaactgggcaactgctgcctggactaccaagagacatgcatcgagcccgagcacatctggacctgcaacaagttcagatgcggcgagaagcggctgaccagatctctgtgcgcctgctctgacgactgcaaggacaagggcgactgctgcatcaactactcctctgtgtgccagggcgagaagtcctgggttgaagaaccctgcgagtccatcaacgagcctcagtgtcctgccggcttcgagacacctcctactctgctgttctccctggatggcttcagagccgagtacctgcatacttggggaggcctgctgccagtgatctccaagctgaagaagtgcggcacctacaccaagaacatgaggcctgtgtaccctaccaagacattccccaaccactactccatcgtgaccggcctgtatcctgagagccacggcatcatcgacaacaagatgtacgaccccaagatgaacgcctccttcagcctgaagtccaaagagaagttcaaccccgagtggtataagggcgagcctatctgggtcaccgctaagtaccagggactgaagtctggcaccttcttttggcctggctccgacgtggaaatcaacggcatcttccccgacatctataagatgtacaacggctccgtgcctttcgaggaacgcattctggctgttctgcagtggctgcagctgcctaaggatgagaggcctcacttctacaccctgtacctggaagaacctgactcctccggccactcttatggccctgtgtcctctgaagtgatcaaggccctgcagcgagtggacggaatggtcggaatgctgatggacggcctgaaagagctgaacctgcacagatgcctgaacctgatcctgatctccgaccacggcatggaacaggggagctgcaagaagtacatctacctgaacaagtacctgggcgacgtgaagaacatcaaagtgatctacggcccagccgccagactgaggccttctgatgtgcctgacaagtactactccttcaactacgagggaatcgcccggaacctgtcctgcagagagcctaaccagcacttcaagccctacctgaagcactttctgcctaagcggctgcacttcgccaagtctgacagaatcgagcccctgaccttctatctggaccctcagtggcagctggccctgaatcctagcgagagaaagtactgtggctccggcttccacggctccgacaacgtgttctctaatatgcaggccctgttcgtcggctacggccctggctttaaacacggcatcgaggccgacaccttcgagaacatcgaggtgtacaatctgatgtgtgacctgctgaatctgacccctgctcctaacaacggcacccacggatctctgaaccatctgctgaagaatcccgtgtacacccctaagcaccccaaagaggttcaccctctggtccagtgtcctttcaccagaaatcctcgggacaacctgggctgctcttgcaacccttctatcctgcctatcgaggactttcagacccagttcaacctgaccgtggccgaggaaaagatcatcaagcacgagacactgccctacggcagacctagagtgctgcagaaagagaacaccatctgcctgctgtcccagcaccagttcatgtccggctactcccaggacatcctgatgcctctgtggacctcctacaccgtggaccggaacgatagcttctccaccgaggacttcagcaactgcctgtaccaggatttcagaatccctctgagccccgtgcacaagtgcagcttctacaagaacaacaccaaggtgtcctacggcttcctgtctcctccacagctgaacaagaactccagcggcatctactctgaggccctgctgaccaccaacatcgtgcccatgtaccagtccttccaagtgatctggcggtacttccacgacaccctgctgaggaagtacgccgaagaaagaaacggcgtgaacgtggtgtctggccccgtgttcgacttcgactacgacggcagatgcgactctctggaaaacctgcggcagaaaagacgagtgatccggaatcaagagatcctgattcctacacacttctttatcgtgctgaccagctgcaaggatacctctcagacccctctgcactgcgagaatctggacaccctggccttcattctgcctcacagaaccgacaactccgagtcctgtgtgcacggcaagcacgactcctcttgggtcgaagaactgctgatgctgcaccgggccagaatcaccgatgtggaacacatcaccggcctgagcttctaccagcagcggaaagaacctgtgtccgatatcctgaagctgaaaacccatctgccaaccttcagccaagaggacctgatcaacgacaagacccacacctgtcctccatgtcctgctccagaactgctcggaggcccctctgtgttcctgtttccacctaagccaaaggacacactgatgatctctcggacccctgaagtgacctgcgtggtggtggatgtgtctcacgaagatcccgaagtcaagttcaattggtacgtggacggcgtggaagtgcacaacgccaagaccaagcctagagaggaacagtacaactccacctacagagtggtgtccgtgctgactgtgctgcaccaggattggctgaacggcaaagagtacaagtgcaaagtgtccaacaaggctctgcccgctcctatcgaaaagaccatctccaaggctaagggccagcctcgggaacctcaggtttacaccctgcctccatctcgggaagagatgaccaagaaccaggtgtccctgacctgcctggtcaagggcttctacccttccgatatcgccgtggaatgggagtccaatggccagcctgagaacaactacaagacaacccctcctgtgctggacagcgacggctcattcttcctgtactctaagctgacagtggacaagtcccggtggcagcaaggcaatgtgttttcctgctctgtgatgcacgaggccctccacaatcactacacccagaagtccctgtctctgtcccctggcaaatgatagctcgag

SEQ ID NO: 43 - DNA, 4404 bp, Artificial Sequence
  Azurocidin-ENPP1-Albumin Nucleotide sequence
atgacaagactgacagtgctggctctgctggccggactgttggcctcttctagagctgctccttcctgcgccaaagaagtgaagtcctgcaagggcagatgcttcgagcggaccttcggcaactgtagatgtgacgccgcttgcgtggaactgggcaactgctgcctggactaccaagagacatgcatcgagcccgagcacatctggacctgcaacaagttcagatgcggcgagaagcggctgaccagatctctgtgcgcctgctctgacgactgcaaggacaagggcgactgctgcatcaactactcctctgtgtgccagggcgagaagtcctgggttgaagaaccctgcgagtccatcaacgagcctcagtgtcctgccggcttcgagacacctcctactctgctgttctccctggatggcttcagagccgagtacctgcatacttggggaggcctgctgccagtgatctccaagctgaagaagtgcggcacctacaccaagaacatgaggcctgtgtaccctaccaagacattccccaaccactactccatcgtgaccggcctgtatcctgagagccacggcatcatcgacaacaagatgtacgaccccaagatgaacgcctccttcagcctgaagtccaaagagaagttcaaccccgagtggtataagggcgagcctatctgggtcaccgctaagtaccagggactgaagtctggcaccttcttttggcctggctccgacgtggaaatcaacggcatcttccccgacatctataagatgtacaacggctccgtgcctttcgaggaacgcattctggctgttctgcagtggctgcagctgcctaaggatgagaggcctcacttctacaccctgtacctggaagaacctgactcctccggccactcttatggccctgtgtcctctgaagtgatcaaggccctgcagcgagtggacggaatggtcggaatgctgatggacggcctgaaagagctgaacctgcacagatgcctgaacctgatcctgatctccgaccacggcatggaacaggggagctgcaagaagtacatctacctgaacaagtacctgggcgacgtgaagaacatcaaagtgatctacggcccagccgccagactgaggccttctgatgtgcctgacaagtactactccttcaactacgagggaatcgcccggaacctgtcctgcagagagcctaaccagcacttcaagccctacctgaagcactttctgcctaagcggctgcacttcgccaagtctgacagaatcgagcccctgaccttctatctggaccctcagtggcagctggccctgaatcctagcgagagaaagtactgtggctccggcttccacggctccgacaacgtgttctctaatatgcaggccctgttcgtcggctacggccctggctttaaacacggcatcgaggccgacaccttcgagaacatcgaggtgtacaatctgatgtgtgacctgctgaatctgacccctgctcctaacaacggcacccacggatctctgaaccatctgctgaagaatcccgtgtacacccctaagcaccccaaagaggttcaccctctggtccagtgtcctttcaccagaaatcctcgggacaacctgggctgctcttgcaacccttctatcctgcctatcgaggactttcagacccagttcaacctgaccgtggccgaggaaaagatcatcaagcacgagacactgccctacggcagacctagagtgctgcagaaagagaacaccatctgcctgctgtcccagcaccagttcatgtccggctactcccaggacatcctgatgcctctgtggacctcctacaccgtggaccggaacgatagcttctccaccgaggacttcagcaactgcctgtaccaggatttcagaatccctctgagccccgtgcacaagtgcagcttctacaagaacaacaccaaggtgtcctacggcttcctgtctcctccacagctgaacaagaactccagcggcatctactctgaggccctgctgaccaccaacatcgtgcccatgtaccagtccttccaagtgatctggcggtacttccacgacaccctgctgaggaagtacgccgaagaaagaaacggcgtgaacgtggtgtctggccccgtgttcgacttcgactacgacggcagatgcgactctctggaaaacctgcggcagaaaagacgagtgatccggaatcaagagatcctgattcctacacacttctttatcgtgctgaccagctgcaaggatacctctcagacccctctgcactgcgagaatctggacaccctggccttcattctgcctcacagaaccgacaactccgagtcctgtgtgcacggcaagcacgactcctcttgggtcgaagaactgctgatgctgcaccgggccagaatcaccgatgtggaacacatcaccggcctgagcttctaccagcagcggaaagaacctgtgtccgatatcctgaagctgaaaacccatctgccaaccttcagccaagaggacctgatcaacatgaagtgggtgaccttcctgctgctgctgttcgtgagcggcagcgccttcagcagaggcgtgttcagaagagaggcccacaagagcgagatcgcccacagatacaacgacctgggcgagcagcacttcaagggcctggtgctgatcgccttcagccagtacctgcagaagtgcagctacgacgagcacgccaagctggtgcaggaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaggagcccgagagaaacgagtgcttcctgcagcacaaggacgacaaccccagcctgccccccttcgagagacccgaggccgaggccatgtgcaccagcttcaaggagaaccccaccaccttcatgggccactacctgcacgaggtggccagaagacacccctacttctacgcccccgagctgctgtactacgccgagcagtacaacgagatcctgacccagtgctgcgccgaggccgacaaggagagctgcctgacccccaagctggacggcgtgaaggagaaggccctggtgagcagcgtgagacagagaatgaagtgcagcagcatgcagaagttcggcgagagagccttcaaggcctgggccgtggccagactgagccagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaggccaccatcagcagcaagctgcagacctgctgcgacaagcccctgctgaagaaggcccactgcctgagcgaggtggagcacgacaccatgcccgccgacctgcccgccatcgccgccgacttcgtggaggaccaggaggtgtgcaagaactacgccgaggccaaggacgtgttcctgggcaccttcctgtacgagtacagcagaagacaccccgactacagcgtgagcctgctgctgagactggccaagaagtacgaggccaccctggagaagtgctgcgccgaggccaacccccccgcctgctacggcaccgtgctggccgagttccagcccctggtggaggagcccaagaacctggtgaagaccaactgcgacctgtacgagaagctgggcgagtacggcttccagaacgccatcctggtgagatacacccagaaggccccccaggtgagcacccccaccctggtggaggccgccagaaacctgggcagagtgggcaccaagtgctgcaccctgcccgaggaccagagactgccctgcgtggaggactacctgagcgccatcctgaacagagtgtgcctgctgcacgagaagacccccgtgagcgagcacgtgaccaagtgctgcagcggcagcctggtggagagaagaccctgcttcagcgccctgaccgtggacgagacctacgtgcccaaggagttcaaggccgagaccttcaccttccacagcgacatctgcaccctgcccgagaaggagaagcagatcaagaagcagaccgccctggccgagctggtgaagcacaagcccaaggccaccgccgagcagctgaagaccgtgatggacgacttcgcccagttcctggacacctgctgcaaggccgccgacaaggacacctgcttcagcaccgagggccccaacctggtgaccagatgcaaggacgccctggccagaagctggagccacccccagttcgagaag

SEQ ID NO: 44 - DNA, 2541 bp, Artificial Sequence
  Azurocidin-ENPP1 Nucleotide sequence
atgacaagactgacagtgctggctctgctggccggactgttggcctcttctagagctgctccttcctgcgccaaagaagtgaagtcctgcaagggcagatgcttcgagcggaccttcggcaactgtagatgtgacgccgcttgcgtggaactgggcaactgctgcctggactaccaagagacatgcatcgagcccgagcacatctggacctgcaacaagttcagatgcggcgagaagcggctgaccagatctctgtgcgcctgctctgacgactgcaaggacaagggcgactgctgcatcaactactcctctgtgtgccagggcgagaagtcctgggttgaagaaccctgcgagtccatcaacgagcctcagtgtcctgccggcttcgagacacctcctactctgctgttctccctggatggcttcagagccgagtacctgcatacttggggaggcctgctgccagtgatctccaagctgaagaagtgcggcacctacaccaagaacatgaggcctgtgtaccctaccaagacattccccaaccactactccatcgtgaccggcctgtatcctgagagccacggcatcatcgacaacaagatgtacgaccccaagatgaacgcctccttcagcctgaagtccaaagagaagttcaaccccgagtggtataagggcgagcctatctgggtcaccgctaagtaccagggactgaagtctggcaccttcttttggcctggctccgacgtggaaatcaacggcatcttccccgacatctataagatgtacaacggctccgtgcctttcgaggaacgcattctggctgttctgcagtggctgcagctgcctaaggatgagaggcctcacttctacaccctgtacctggaagaacctgactcctccggccactcttatggccctgtgtcctctgaagtgatcaaggccctgcagcgagtggacggaatggtcggaatgctgatggacggcctgaaagagctgaacctgcacagatgcctgaacctgatcctgatctccgaccacggcatggaacaggggagctgcaagaagtacatctacctgaacaagtacctgggcgacgtgaagaacatcaaagtgatctacggcccagccgccagactgaggccttctgatgtgcctgacaagtactactccttcaactacgagggaatcgcccggaacctgtcctgcagagagcctaaccagcacttcaagccctacctgaagcactttctgcctaagcggctgcacttcgccaagtctgacagaatcgagcccctgaccttctatctggaccctcagtggcagctggccctgaatcctagcgagagaaagtactgtggctccggcttccacggctccgacaacgtgttctctaatatgcaggccctgttcgtcggctacggccctggctttaaacacggcatcgaggccgacaccttcgagaacatcgaggtgtacaatctgatgtgtgacctgctgaatctgacccctgctcctaacaacggcacccacggatctctgaaccatctgctgaagaatcccgtgtacacccctaagcaccccaaagaggttcaccctctggtccagtgtcctttcaccagaaatcctcgggacaacctgggctgctcttgcaacccttctatcctgcctatcgaggactttcagacccagttcaacctgaccgtggccgaggaaaagatcatcaagcacgagacactgccctacggcagacctagagtgctgcagaaagagaacaccatctgcctgctgtcccagcaccagttcatgtccggctactcccaggacatcctgatgcctctgtggacctcctacaccgtggaccggaacgatagcttctccaccgaggacttcagcaactgcctgtaccaggatttcagaatccctctgagccccgtgcacaagtgcagcttctacaagaacaacaccaaggtgtcctacggcttcctgtctcctccacagctgaacaagaactccagcggcatctactctgaggccctgctgaccaccaacatcgtgcccatgtaccagtccttccaagtgatctggcggtacttccacgacaccctgctgaggaagtacgccgaagaaagaaacggcgtgaacgtggtgtctggccccgtgttcgacttcgactacgacggcagatgcgactctctggaaaacctgcggcagaaaagacgagtgatccggaatcaagagatcctgattcctacacacttctttatcgtgctgaccagctgcaaggatacctctcagacccctctgcactgcgagaatctggacaccctggccttcattctgcctcacagaaccgacaactccgagtcctgtgtgcacggcaagcacgactcctcttgggtcgaagaactgctgatgctgcaccgggccagaatcaccgatgtggaacacatcaccggcctgagcttctaccagcagcggaaagaacctgtgtccgatatcctgaagctgaaaacccatctgccaaccttcagccaagaggac

SEQ ID NO: 45 - DNA, 3219 bp, Artificial Sequence
  Azurocidin-ENPP3-FC Nucleotide sequence
atgaccagactgaccgtgctggccctgctggccggcctgctggccagcagcagagccgccaagcagggcagctgcagaaagaagtgcttcgacgccagcttcagaggcctggagaactgcagatgcgacgtggcctgcaaggacagaggcgactgctgctgggacttcgaggacacctgcgtggagagcaccagaatctggatgtgcaacaagttcagatgcggcgagaccagactggaggccagcctgtgcagctgcagcgacgactgcctgcagagaaaggactgctgcgccgactacaagagcgtgtgccagggcgagaccagctggctggaggagaactgcgacaccgcccagcagagccagtgccccgagggcttcgacctgccccccgtgatcctgttcagcatggacggcttcagagccgagtacctgtacacctgggacaccctgatgcccaacatcaacaagctgaagacctgcggcatccacagcaagtacatgagagccatgtaccccaccaagaccttccccaaccactacaccatcgtgaccggcctgtaccccgagagccacggcatcatcgacaacaacatgtacgacgtgaacctgaacaagaacttcagcctgagcagcaaggagcagaacaaccccgcctggtggcacggccagcccatgaacctgaccgccatgtaccagggcctgaaggccgccacctacttctggcccggcagcgaggtggccatcaacggcagcttccccagcatctacatgccctacaacggcagcgtgcccttcgaggagagaatcagcaccctgctgaagtggctggacctgcccaaggccgagagacccagattctacaccatgtacttcgaggagcccgacagcagcggccacgccggcggccccgtgagcgccagagtgatcaaggccctgcaggtggtggaccacgccttcggcatgctgatggagggcctgaagcagagaaacctgcacaactgcgtgaacatcatcctgctggccgaccacggcatggaccagacctactgcaacaagatggagtacatgaccgactacttccccagaatcaacttcttctacatgtacgagggccccgcccccagaatcagagcccacaacatcccccacgacttcttcagcttcaacagcgaggagatcgtgagaaacctgagctgcagaaagcccgaccagcacttcaagccctacctgacccccgacctgcccaagagactgcactacgccaagaacgtgagaatcgacaaggtgcacctgttcgtggaccagcagtggctggccgtgagaagcaagagcaacaccaactgcggcggcggcaaccacggctacaacaacgagttcagaagcatggaggccatcttcctggcccacggccccagcttcaaggagaagaccgaggtggagcccttcgagaacatcgaggtgtacaacctgatgtgcgacctgctgagaatccagcccgcccccaacaacggcacccacggcagcctgaaccacctgctgaaggtgcccttctacgagcccagccacgccgaggaggtgagcaagttcagcgtgtgcggcttcgccaaccccctgcccaccgagagcctggactgcttctgcccccacctgcagaacagcacccagctggagcaggtgaaccagatgctgaacctgacccaggaggagatcaccgccaccgtgaaggtgaacctgcccttcggcagacccagagtgctgcagaagaacgtggaccactgcctgctgtaccacagagagtacgtgagcggcttcggcaaggccatgagaatgcccatgtggagcagctacaccgtgccccagctgggcgacaccagccccctgccccccaccgtgcccgactgcctgagagccgacgtgagagtgccccccagcgagagccagaagtgcagcttctacctggccgacaagaacatcacccacggcttcctgtacccccccgccagcaacagaaccagcgacagccagtacgacgccctgatcaccagcaacctggtgcccatgtacgaggagttcagaaagatgtgggactacttccacagcgtgctgctgatcaagcacgccaccgagagaaacggcgtgaacgtggtgagcggccccatcttcgactacaactacgacggccacttcgacgcccccgacgagatcaccaagcacctggccaacaccgacgtgcccatccccacccactacttcgtggtgctgaccagctgcaagaacaagagccacacccccgagaactgccccggctggctggacgtgctgcccttcatcatcccccacagacccaccaacgtggagagctgccccgagggcaagcccgaggccctgtgggtggaggagagattcaccgcccacatcgccagagtgagagacgtggagctgctgaccggcctggacttctaccaggacaaggtgcagcccgtgagcgagatcctgcagctgaagacctacctgcccaccttcgagaccaccatcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagaggagatgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgatgcacgaggccctgcacaaccactacacccagaagagcctgagcctgagccccggcaag

SEQ ID NO: 46 - DNA, 4392 bp, Artificial Sequence
  Azurocidin-ENPP3-Albumin Nucleotide sequence
atgaccagactgaccgtgctggccctgctggccggcctgctggccagcagcagagccgccaagcagggcagctgcagaaagaagtgcttcgacgccagcttcagaggcctggagaactgcagatgcgacgtggcctgcaaggacagaggcgactgctgctgggacttcgaggacacctgcgtggagagcaccagaatctggatgtgcaacaagttcagatgcggcgagaccagactggaggccagcctgtgcagctgcagcgacgactgcctgcagagaaaggactgctgcgccgactacaagagcgtgtgccagggcgagaccagctggctggaggagaactgcgacaccgcccagcagagccagtgccccgagggcttcgacctgccccccgtgatcctgttcagcatggacggcttcagagccgagtacctgtacacctgggacaccctgatgcccaacatcaacaagctgaagacctgcggcatccacagcaagtacatgagagccatgtaccccaccaagaccttccccaaccactacaccatcgtgaccggcctgtaccccgagagccacggcatcatcgacaacaacatgtacgacgtgaacctgaacaagaacttcagcctgagcagcaaggagcagaacaaccccgcctggtggcacggccagcccatgaacctgaccgccatgtaccagggcctgaaggccgccacctacttctggcccggcagcgaggtggccatcaacggcagcttccccagcatctacatgccctacaacggcagcgtgcccttcgaggagagaatcagcaccctgctgaagtggctggacctgcccaaggccgagagacccagattctacaccatgtacttcgaggagcccgacagcagcggccacgccggcggccccgtgagcgccagagtgatcaaggccctgcaggtggtggaccacgccttcggcatgctgatggagggcctgaagcagagaaacctgcacaactgcgtgaacatcatcctgctggccgaccacggcatggaccagacctactgcaacaagatggagtacatgaccgactacttccccagaatcaacttcttctacatgtacgagggccccgcccccagaatcagagcccacaacatcccccacgacttcttcagcttcaacagcgaggagatcgtgagaaacctgagctgcagaaagcccgaccagcacttcaagccctacctgacccccgacctgcccaagagactgcactacgccaagaacgtgagaatcgacaaggtgcacctgttcgtggaccagcagtggctggccgtgagaagcaagagcaacaccaactgcggcggcggcaaccacggctacaacaacgagttcagaagcatggaggccatcttcctggcccacggccccagcttcaaggagaagaccgaggtggagcccttcgagaacatcgaggtgtacaacctgatgtgcgacctgctgagaatccagcccgcccccaacaacggcacccacggcagcctgaaccacctgctgaaggtgcccttctacgagcccagccacgccgaggaggtgagcaagttcagcgtgtgcggcttcgccaaccccctgcccaccgagagcctggactgcttctgcccccacctgcagaacagcacccagctggagcaggtgaaccagatgctgaacctgacccaggaggagatcaccgccaccgtgaaggtgaacctgcccttcggcagacccagagtgctgcagaagaacgtggaccactgcctgctgtaccacagagagtacgtgagcggcttcggcaaggccatgagaatgcccatgtggagcagctacaccgtgccccagctgggcgacaccagccccctgccccccaccgtgcccgactgcctgagagccgacgtgagagtgccccccagcgagagccagaagtgcagcttctacctggccgacaagaacatcacccacggcttcctgtacccccccgccagcaacagaaccagcgacagccagtacgacgccctgatcaccagcaacctggtgcccatgtacgaggagttcagaaagatgtgggactacttccacagcgtgctgctgatcaagcacgccaccgagagaaacggcgtgaacgtggtgagcggccccatcttcgactacaactacgacggccacttcgacgcccccgacgagatcaccaagcacctggccaacaccgacgtgcccatccccacccactacttcgtggtgctgaccagctgcaagaacaagagccacacccccgagaactgccccggctggctggacgtgctgcccttcatcatcccccacagacccaccaacgtggagagctgccccgagggcaagcccgaggccctgtgggtggaggagagattcaccgcccacatcgccagagtgagagacgtggagctgctgaccggcctggacttctaccaggacaaggtgcagcccgtgagcgagatcctgcagctgaagacctacctgcccaccttcgagaccaccatcatgaagtgggtgaccttcctgctgctgctgttcgtgagcggcagcgccttcagcagaggcgtgttcagaagagaggcccacaagagcgagatcgcccacagatacaacgacctgggcgagcagcacttcaagggcctggtgctgatcgccttcagccagtacctgcagaagtgcagctacgacgagcacgccaagctggtgcaggaggtgaccgacttcgccaagacctgcgtggccgacgagagcgccgccaactgcgacaagagcctgcacaccctgttcggcgacaagctgtgcgccatccccaacctgagagagaactacggcgagctggccgactgctgcaccaagcaggagcccgagagaaacgagtgcttcctgcagcacaaggacgacaaccccagcctgccccccttcgagagacccgaggccgaggccatgtgcaccagcttcaaggagaaccccaccaccttcatgggccactacctgcacgaggtggccagaagacacccctacttctacgcccccgagctgctgtactacgccgagcagtacaacgagatcctgacccagtgctgcgccgaggccgacaaggagagctgcctgacccccaagctggacggcgtgaaggagaaggccctggtgagcagcgtgagacagagaatgaagtgcagcagcatgcagaagttcggcgagagagccttcaaggcctgggccgtggccagactgagccagaccttccccaacgccgacttcgccgagatcaccaagctggccaccgacctgaccaaggtgaacaaggagtgctgccacggcgacctgctggagtgcgccgacgacagagccgagctggccaagtacatgtgcgagaaccaggccaccatcagcagcaagctgcagacctgctgcgacaagcccctgctgaagaaggcccactgcctgagcgaggtggagcacgacaccatgcccgccgacctgcccgccatcgccgccgacttcgtggaggaccaggaggtgtgcaagaactacgccgaggccaaggacgtgttcctgggcaccttcctgtacgagtacagcagaagacaccccgactacagcgtgagcctgctgctgagactggccaagaagtacgaggccaccctggagaagtgctgcgccgaggccaacccccccgcctgctacggcaccgtgctggccgagttccagcccctggtggaggagcccaagaacctggtgaagaccaactgcgacctgtacgagaagctgggcgagtacggcttccagaacgccatcctggtgagatacacccagaaggccccccaggtgagcacccccaccctggtggaggccgccagaaacctgggcagagtgggcaccaagtgctgcaccctgcccgaggaccagagactgccctgcgtggaggactacctgagcgccatcctgaacagagtgtgcctgctgcacgagaagacccccgtgagcgagcacgtgaccaagtgctgcagcggcagcctggtggagagaagaccctgcttcagcgccctgaccgtggacgagacctacgtgcccaaggagttcaaggccgagaccttcaccttccacagcgacatctgcaccctgcccgagaaggagaagcagatcaagaagcagaccgccctggccgagctggtgaagcacaagcccaaggccaccgccgagcagctgaagaccgtgatggacgacttcgcccagttcctggacacctgctgcaaggccgccgacaaggacacctgcttcagcaccgagggccccaacctggtgaccagatgcaaggacgccctggccagaagctggagccacccccagttcgagaag

SEQ ID NO: 47 - DNA, 2538 bp, Artificial Sequence
  Azurocidin-ENPP3-Nucleotide sequence
atgaccagactgaccgtgctggccctgctggccggcctgctggccagcagcagagccgccaagcagggcagctgcagaaagaagtgcttcgacgccagcttcagaggcctggagaactgcagatgcgacgtggcctgcaaggacagaggcgactgctgctgggacttcgaggacacctgcgtggagagcaccagaatctggatgtgcaacaagttcagatgcggcgagaccagactggaggccagcctgtgcagctgcagcgacgactgcctgcagagaaaggactgctgcgccgactacaagagcgtgtgccagggcgagaccagctggctggaggagaactgcgacaccgcccagcagagccagtgccccgagggcttcgacctgccccccgtgatcctgttcagcatggacggcttcagagccgagtacctgtacacctgggacaccctgatgcccaacatcaacaagctgaagacctgcggcatccacagcaagtacatgagagccatgtaccccaccaagaccttccccaaccactacaccatcgtgaccggcctgtaccccgagagccacggcatcatcgacaacaacatgtacgacgtgaacctgaacaagaacttcagcctgagcagcaaggagcagaacaaccccgcctggtggcacggccagcccatgaacctgaccgccatgtaccagggcctgaaggccgccacctacttctggcccggcagcgaggtggccatcaacggcagcttccccagcatctacatgccctacaacggcagcgtgcccttcgaggagagaatcagcaccctgctgaagtggctggacctgcccaaggccgagagacccagattctacaccatgtacttcgaggagcccgacagcagcggccacgccggcggccccgtgagcgccagagtgatcaaggccctgcaggtggtggaccacgccttcggcatgctgatggagggcctgaagcagagaaacctgcacaactgcgtgaacatcatcctgctggccgaccacggcatggaccagacctactgcaacaagatggagtacatgaccgactacttccccagaatcaacttcttctacatgtacgagggccccgcccccagaatcagagcccacaacatcccccacgacttcttcagcttcaacagcgaggagatcgtgagaaacctgagctgcagaaagcccgaccagcacttcaagccctacctgacccccgacctgcccaagagactgcactacgccaagaacgtgagaatcgacaaggtgcacctgttcgtggaccagcagtggctggccgtgagaagcaagagcaacaccaactgcggcggcggcaaccacggctacaacaacgagttcagaagcatggaggccatcttcctggcccacggccccagcttcaaggagaagaccgaggtggagcccttcgagaacatcgaggtgtacaacctgatgtgcgacctgctgagaatccagcccgcccccaacaacggcacccacggcagcctgaaccacctgctgaaggtgcccttctacgagcccagccacgccgaggaggtgagcaagttcagcgtgtgcggcttcgccaaccccctgcccaccgagagcctggactgcttctgcccccacctgcagaacagcacccagctggagcaggtgaaccagatgctgaacctgacccaggaggagatcaccgccaccgtgaaggtgaacctgcccttcggcagacccagagtgctgcagaagaacgtggaccactgcctgctgtaccacagagagtacgtgagcggcttcggcaaggccatgagaatgcccatgtggagcagctacaccgtgccccagctgggcgacaccagccccctgccccccaccgtgcccgactgcctgagagccgacgtgagagtgccccccagcgagagccagaagtgcagcttctacctggccgacaagaacatcacccacggcttcctgtacccccccgccagcaacagaaccagcgacagccagtacgacgccctgatcaccagcaacctggtgcccatgtacgaggagttcagaaagatgtgggactacttccacagcgtgctgctgatcaagcacgccaccgagagaaacggcgtgaacgtggtgagcggccccatcttcgactacaactacgacggccacttcgacgcccccgacgagatcaccaagcacctggccaacaccgacgtgcccatccccacccactacttcgtggtgctgaccagctgcaagaacaagagccacacccccgagaactgccccggctggctggacgtgctgcccttcatcatcccccacagacccaccaacgtggagagctgccccgagggcaagcccgaggccctgtgggtggaggagagattcaccgcccacatcgccagagtgagagacgtggagctgctgaccggcctggacttctaccaggacaaggtgcagcccgtgagcgagatcctgcagctgaagacctacctgcccaccttcgagaccaccatc

SEQ ID NO: 48 - DNA, 3228 bp, Artificial Sequence
  ENPP7-1-Fc Nucleotide sequence
atgagaggacctgccgtcctgctgaccgtcgccctggctaccttgctggcccctggtgctggtgcacccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagatgacaagacccacacttgccccccctgcccagctcctgaactgctgggaggaccctctgtgttcctgttccccccaaagcccaaggacaccctgatgatctctaggacccccgaagtcacttgcgtcgtcgtcgacgtgtcccacgaggaccctgaagtcaagttcaactggtacgtcgacggtgtcgaagtccacaacgccaagaccaagcccagggaagaacagtacaactctacctaccgcgtcgtcagcgtcctgaccgtcctgcaccaggactggctgaacggaaaggaatacaagtgcaaggtgtccaacaaggccctgcctgcccccatcgaaaagaccatctctaaggccaagggacagccccgcgaaccccaggtctacaccctgccaccctctagggaagaaatgaccaagaaccaggtgtccctgacctgcctggtcaagggattctacccctctgacatcgccgtcgaatgggaatctaacggacagcccgaaaacaactacaagaccaccccccctgtcctggactctgacggatcattcttcctgtactctaagctgactgtcgacaagtctaggtggcagcagggaaacgtgttctcttgctctgtcatgcacgaagccctgcacaaccactacacccagaagtctctgtctctgtcccccggaaag

SEQ ID NO: 49 - DNA, 4434 bp, Artificial Sequence
  ENPP7-NPP1 Albumin Nucleotide sequence
atgagaggacctgccgtcctgctgaccgtcgccctggctaccttgctggcccctggtgctggtgcacccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagatggtggaggaggctctggtggaggcggtagcggaggcggagggtcgggaggttctggatcaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 50 - DNA, 3447 bp, Artificial Sequence
  Nucleotide sequence of NPP121-NPP3-Fc
atggaaagggacggatgcgccggtggtggatctcgcggaggcgaaggtggaagggcccctagggaaggacctgccggaaacggaagggacaggggacgctctcacgccgctgaagctccaggcgaccctcaggccgctgcctctctgctggctcctatggacgtcggagaagaacccctggaaaaggccgccagggccaggactgccaaggaccccaacacctacaagatcatctccctcttcactttcgccgtcggagtcaacatctgcctgggattcaccgccgaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactattgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 51 - DNA, 4638 bp, Artificial Sequence
  Nucleotide sequence of NPP121-NPP3-Fc
atggaaagggacggatgcgccggtggtggatctcgcggaggcgaaggtggaagggcccctagggaaggacctgccggaaacggaagggacaggggacgctctcacgccgctgaagctccaggcgaccctcaggccgctgcctctctgctggctcctatggacgtcggagaagaacccctggaaaaggccgccagggccaggactgccaaggaccccaacacctacaagatcatctccctcttcactttcgccgtcggagtcaacatctgcctgggattcaccgccgaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactattggtggaggaggctctggtggaggcggtagcggaggcggagggtcgatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 52 - DNA, 8852 bp, Artificial Sequence
  Nucleotide sequence of hNPP3-hFc-pcDNA3
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttatggaaagggacggatgcgccggtggtggatctcgcggaggcgaaggtggaagggcccctagggaaggacctgccggaaacggaagggacaggggacgctctcacgccgctgaagctccaggcgaccctcaggccgctgcctctctgctggctcctatggacgtcggagaagaacccctggaaaaggccgccagggccaggactgccaaggaccccaacacctacaagatcatctccctcttcactttcgccgtcggagtcaacatctgcctgggattcaccgccgaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactattgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgaaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 53 - DNA, 3456 bp, Artificial Sequence
  Nucleotide sequence of hNPP3-hFc-pcDNA3
atggaaagggacggatgcgccggtggtggatctcgcggaggcgaaggtggaagggcccctagggaaggacctgccggaaacggaagggacaggggacgctctcacgccgctgaagctccaggcgaccctcaggccgctgcctctctgctggctcctatggacgtcggagaagaacccctggaaaaggccgccagggccaggactgccaaggaccccaacacctacaagatcatctccctcttcactttcgccgtcggagtcaacatctgcctgggattcaccgccggactgaagcccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagatgacaagacccacacttgccccccctgcccagctcctgaactgctgggaggaccctctgtgttcctgttccccccaaagcccaaggacaccctgatgatctctaggacccccgaagtcacttgcgtcgtcgtcgacgtgtcccacgaggaccctgaagtcaagttcaactggtacgtcgacggtgtcgaagtccacaacgccaagaccaagcccagggaagaacagtacaactctacctaccgcgtcgtcagcgtcctgaccgtcctgcaccaggactggctgaacggaaaggaatacaagtgcaaggtgtccaacaaggccctgcctgcccccatcgaaaagaccatctctaaggccaagggacagccccgcgaaccccaggtctacaccctgccaccctctagggaagaaatgaccaagaaccaggtgtccctgacctgcctggtcaagggattctacccctctgacatcgccgtcgaatgggaatctaacggacagcccgaaaacaactacaagaccaccccccctgtcctggactctgacggatcattcttcctgtactctaagctgactgtcgacaagtctaggtggcagcagggaaacgtgttctcttgctctgtcatgcacgaagccctgcacaaccactacacccagaagtctctgtctctgtcccccggaaag

SEQ ID NO: 54 - DNA, 4662 bp, Artificial Sequence
  ENPP121- Albumin Nucleotide sequence
atggaaagggacggatgcgccggtggtggatctcgcggaggcgaaggtggaagggcccctagggaaggacctgccggaaacggaagggacaggggacgctctcacgccgctgaagctccaggcgaccctcaggccgctgcctctctgctggctcctatggacgtcggagaagaacccctggaaaaggccgccagggccaggactgccaaggaccccaacacctacaagatcatctccctcttcactttcgccgtcggagtcaacatctgcctgggattcaccgccggactgaagcccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagatggtggaggaggctctggtggaggcggtagcggaggcggagggtcgggaggttctggatcaatgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 55 - DNA, 2625 bp, Artificial Sequence
  ENPP3 Nucleotide sequence
atggaatctacgttgactttagcaacggaacaacctgttaagaagaacactcttaagaaatataaaatagcttgcattgttcttcttgctttgctggtgatcatgtcacttggattaggcctggggcttggactcaggaaactggaaaagcaaggcagctgcaggaagaagtgctttgatgcatcatttagaggactggagaactgccggtgtgatgtggcatgtaaagaccgaggtgattgctgctgggattttgaagacacctgtgtggaatcaactcgaatatggatgtgcaataaatttcgttgtggagagaccagattagaggccagcctttgctcttgttcagatgactgtttgcagaggaaagattgctgtgctgactataagagtgtttgccaaggagaaacctcatggctggaagaaaactgtgacacagcccagcagtctcagtgcccagaagggtttgacctgccaccagttatcttgttttctatggatggatttagagctgaatatttatacacatgggatactttaatgccaaatatcaataaactgaaaacatgtggaattcattcaaaatacatgagagctatgtatcctaccaaaaccttcccaaatcattacaccattgtcacgggcttgtatccagagtcacatggcatcattgacaataatatgtatgatgtaaatctcaacaagaatttttcactttcttcaaaggaacaaaataatccagcctggtggcatgggcaaccaatgtggctgacagcaatgtatcaaggtttaaaagccgctacctacttttggcccggatcagaagtggctataaatggctcctttccttccatatacatgccttacaacggaagtgtcccatttgaagagaggatttctacactgttaaaatggctggacctgcccaaagctgaaagacccaggttttataccatgtattttgaagaacctgattcctctggacatgcaggtggaccagtcagtgccagagtaattaaagccttacaggtagtagatcatgcttttgggatgttgatggaaggcctgaagcagcggaatttgcacaactgtgtcaatatcatccttctggctgaccatggaatggaccagacttattgtaacaagatggaatacatgactgattattttcccagaataaacttcttctacatgtacgaagggcctgccccccgcatccgagctcataatatacctcatgacttttttagttttaattctgaggaaattgttagaaacctcagttgccgaaaacctgatcagcatttcaagccctatttgactcctgatttgccaaagcgactgcactatgccaagaacgtcagaatcgacaaagttcatctctttgtggatcaacagtggctggctgttaggagtaaatcaaatacaaattgtggaggaggcaaccatggttataacaatgagtttaggagcatggaggctatctttctggcacatggacccagttttaaagagaagactgaagttgaaccatttgaaaatattgaagtctataacctaatgtgtgatcttctacgcattcaaccagcaccaaacaatggaacccatggtagtttaaaccatcttctgaaggtgcctttttatgagccatcccatgcagaggaggtgtcaaagttttctgtttgtggctttgctaatccattgcccacagagtctcttgactgtttctgccctcacctacaaaatagtactcagctggaacaagtgaatcagatgctaaatctcacccaagaagaaataacagcaacagtgaaagtaaatttgccatttgggaggcctagggtactgcagaagaacgtggaccactgtctcctttaccacagggaatatgtcagtggatttggaaaagctatgaggatgcccatgtggagttcatacacagtcccccagttgggagacacatcgcctctgcctcccactgtcccagactgtctgcgggctgatgtcagggttcctccttctgagagccaaaaatgttccttctatttagcagacaagaatatcacccacggcttcctctatcctcctgccagcaatagaacatcagatagccaatatgatgctttaattactagcaatttggtacctatgtatgaagaattcagaaaaatgtgggactacttccacagtgttcttcttataaaacatgccacagaaagaaatggagtaaatgtggttagtggaccaatatttgattataattatgatggccattttgatgctccagatgaaattaccaaacatttagccaacactgatgttcccatcccaacacactactttgtggtgctgaccagttgtaaaaacaagagccacacaccggaaaactgccctgggtggctggatgtcctaccctttatcatccctcaccgacctaccaacgtggagagctgtcctgaaggtaaaccagaagctctttgggttgaagaaagatttacagctcacattgcccgggtccgtgatgtagaacttctcactgggcttgacttctatcaggataaagtgcagcctgtctctgaaattttgcaactaaagacatatttaccaacatttgaaaccactatt

SEQ ID NO: 56 - DNA, 2775 bp, Artificial Sequence
  ENPP1 Nucleotide sequence
atggaacgggacggctgtgccggcggaggatcaagaggcggagaaggcggcagagcccctagagaaggacctgccggcaacggcagagacagaggcagatctcatgccgccgaagcccctggcgatcctcaggctgctgcttctctgctggcccccatggatgtgggcgaggaacctctggaaaaggccgccagagccagaaccgccaaggaccccaacacctacaaggtgctgagcctggtgctgtccgtgtgcgtgctgaccaccatcctgggctgcatcttcggcctgaagcccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagat

SEQ ID NO: 57 - protein, 15 aa, Artificial Sequence
  Linker
  MISC_FEATURE (0)..(15): The sequence can be anywhere from 0 to 15 amino acids
RRRRRRRRRRRRRRR

SEQ ID NO: 58 - protein, 16 aa, Artificial Sequence
  Linker
DSSSEEKFLRRIGRFG

SEQ ID NO: 59 - protein, 12 aa, Artificial Sequence
  Linker
EEEEEEEPRGDT

SEQ ID NO: 60 - protein, 12 aa, Artificial Sequence
  Linker
APWHLSSQYSRT

SEQ ID NO: 61 - protein, 12 aa, Artificial Sequence
  Linker
STLPIPHEFSRE

SEQ ID NO: 62 - protein, 12 aa, Artificial Sequence
  Linker
VTKHLNQISQSY

SEQ ID NO: 63 - protein, 15 aa, Artificial Sequence
  Linker
  MISC_FEATURE (1)..(15): The sequence can be anywhere from 1 to 15 amino acids
EEEEEEEEEEEEEEE

SEQ ID NO: 64 - protein, 6 aa, Artificial Sequence
  Linker
GGSGGS

SEQ ID NO: 65 - protein, 7 aa, Artificial Sequence
  Linker
RSGSGGS

SEQ ID NO: 66 - protein, 15 aa, Artificial Sequence
  Linker
  MISC_FEATURE (1)..(15): The sequence can be anywhere from 1 to 15 amino acids
DDDDDDDDDDDDDDD

SEQ ID NO: 67 - protein, 16 aa, Artificial Sequence
  Linker
LVIMSLGLGLGLGLRK

SEQ ID NO: 68 - protein, 15 aa, Artificial Sequence
  Linker
VIMSLGLGLGLGLRK

SEQ ID NO: 69 - protein, 14 aa, Artificial Sequence
  Linker
IMSLGLGLGLGLRK

SEQ ID NO: 70 - protein, 13 aa, Artificial Sequence
  Linker
MSLGLGLGLGLRK

SEQ ID NO: 71 - protein, 12 aa, Artificial Sequence
  Linker
SLGLGLGLGLRK

SEQ ID NO: 72 - protein, 11 aa, Artificial Sequence
  Linker
LGLGLGLGLRK

SEQ ID NO: 73 - protein, 10 aa, Artificial Sequence
  Linker
GLGLGLGLRK

SEQ ID NO: 74 - protein, 9 aa, Artificial Sequence
  Linker
LGLGLGLRK

SEQ ID NO: 75 - protein, 8 aa, Artificial Sequence
  Linker
GLGLGLRK

SEQ ID NO: 76 - protein, 7 aa, Artificial Sequence
  Linker
LGLGLRK

SEQ ID NO: 77 - protein, 6 aa, Artificial Sequence
  Linker
GLGLRK

SEQ ID NO: 78 - protein, 5 aa, Artificial Sequence
  Linker
LGLRK

SEQ ID NO: 79 - protein, 4 aa, Artificial Sequence
  Linker
GLRK

SEQ ID NO: 80 - protein, 15 aa, Artificial Sequence
  Linker
  MISC_FEATURE (1)..(15): The sequence can be anywhere from 1 to 15 amino acids
KKKKKKKKKKKKKKK

SEQ ID NO: 81 - protein, 15 aa, Artificial Sequence
  Linker
  MISC_FEATURE (1)..(15): The sequence can be anywhere from 1 to 15 amino acids
DDDDDDDDDDDDDDD

SEQ ID NO: 82 - protein, 1063 aa, Artificial Sequence
  Soluble NPP1-Fc fuion protein sequence
  MISC_FEATURE (837)..(1063): Indicate Fc sequence
FTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - DNA, 3180 bp, Artificial Sequence
  Nucleotide sequence of solube NPP1-Fc
ttcaccgccggactgaagcccagctgcgccaaagaagtgaagtcctgcaagggccggtgcttcgagcggaccttcggcaactgcagatgcgacgccgcctgtgtggaactgggcaactgctgcctggactaccaggaaacctgcatcgagcccgagcacatctggacctgcaacaagttcagatgcggcgagaagcggctgaccagatccctgtgtgcctgcagcgacgactgcaaggacaagggcgactgctgcatcaactacagcagcgtgtgccagggcgagaagtcctgggtggaagaaccctgcgagagcatcaacgagccccagtgccctgccggcttcgagacacctcctaccctgctgttcagcctggacggctttcgggccgagtacctgcacacatggggaggcctgctgcccgtgatcagcaagctgaagaagtgcggcacctacaccaagaacatgcggcccgtgtaccccaccaagaccttccccaaccactactccatcgtgaccggcctgtaccccgagagccacggcatcatcgacaacaagatgtacgaccccaagatgaacgccagcttcagcctgaagtccaaagagaagttcaaccccgagtggtataagggcgagcccatctgggtcaccgccaagtaccagggcctgaaaagcggcacattcttttggcccggcagcgacgtggaaatcaacggcatcttccccgacatctataagatgtacaacggcagcgtgcccttcgaggaacggatcctggctgtgctgcagtggctgcagctgcccaaggatgagcggccccacttctacaccctgtacctggaagaacctgacagcagcggccacagctacggccctgtgtccagcgaagtgatcaaggccctgcagcgggtggacggcatggtgggaatgctgatggacggcctgaaagagctgaacctgcacagatgcctgaacctgatcctgatcagcgaccacggcatggaacagggatcctgcaagaagtacatctacctgaacaagtacctgggcgacgtgaagaacatcaaagtgatctacggcccagccgccagactgaggcctagcgacgtgcccgacaagtactacagcttcaactacgagggaatcgcccggaacctgagctgcagagagcccaaccagcacttcaagccctacctgaagcacttcctgcccaagcggctgcacttcgccaagagcgacagaatcgagcccctgaccttctacctggacccccagtggcagctggccctgaatcccagcgagagaaagtactgcggcagcggcttccacggctccgacaacgtgttcagcaacatgcaggccctgttcgtgggctacggacccggctttaagcacggcatcgaggccgacaccttcgagaacatcgaggtgtacaatctgatgtgcgacctgctgaatctgacccctgcccccaacaatggcacccacggcagcctgaaccatctgctgaagaaccccgtgtacacccctaagcaccccaaagaggtgcaccccctggtgcagtgccccttcaccagaaaccccagagacaacctgggctgtagctgcaaccccagcatcctgcccatcgaggacttccagacccagttcaacctgaccgtggccgaggaaaagatcatcaagcacgagacactgccctacggcagaccccgggtgctgcagaaagagaacaccatctgcctgctgagccagcaccagttcatgagcggctactcccaggacatcctgatgcccctgtggaccagctacaccgtggaccggaacgacagcttctccaccgaggatttcagcaactgcctgtaccaggatttccggatccccctgagccccgtgcacaagtgcagcttctacaagaacaacaccaaggtgtcctacggcttcctgagccctccccagctgaacaagaacagctccggcatctacagcgaggccctgctgactaccaacatcgtgcccatgtaccagagcttccaagtgatctggcggtacttccacgacaccctgctgcggaagtacgccgaagaacggaacggcgtgaacgtggtgtccggcccagtgttcgacttcgactacgacggcagatgtgacagcctggaaaatctgcggcagaaaagaagagtgatccggaaccaggaaattctgatccctacccacttctttatcgtgctgacaagctgcaaggataccagccagacccccctgcactgcgagaacctggataccctggccttcatcctgcctcaccggaccgacaacagcgagagctgtgtgcacggcaagcacgacagctcttgggtggaagaactgctgatgctgcaccgggccagaatcaccgatgtggaacacatcaccggcctgagcttttaccagcagcggaaagaacccgtgtccgatatcctgaagctgaaaacccatctgcccaccttcagccaggaagatgacaagacccacacttgccccccctgcccagctcctgaactgctgggaggaccctctgtgttcctgttccccccaaagcccaaggacaccctgatgatctctaggacccccgaagtcacttgcgtcgtcgtcgacgtgtcccacgaggaccctgaagtcaagttcaactggtacgtcgacggtgtcgaagtccacaacgccaagaccaagcccagggaagaacagtacaactctacctaccgcgtcgtcagcgtcctgaccgtcctgcaccaggactggctgaacggaaaggaatacaagtgcaaggtgtccaacaaggccctgcctgcccccatcgaaaagaccatctctaaggccaagggacagccccgcgaaccccaggtctacaccctgccaccctctagggaagaaatgaccaagaaccaggtgtccctgacctgcctggtcaagggattctacccctctgacatcgccgtcgaatgggaatctaacggacagcccgaaaacaactacaagaccaccccccctgtcctggactctgacggatcattcttcctgtactctaagctgactgtcgacaagtctaggtggcagcagggaaacgtgttctcttgctctgtcatgcacgaagccctgcacaaccactacacccagaagtctctgtctctgtcccccggaaag

SEQ ID NO: 84 - protein, 1060 aa, Artificial Sequence
  Soluble NPP1-(GLK)-Fc fusion protein sequence
  MISC_FEATURE (4)..(830): beginning and end of NPP1
  MISC_FEATURE (834)..(1060): Fc sequence
GLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 1057 aa, Artificial Sequence
  Soluble NPP1-Fc fusion protein sequence
  MISC_FEATURE (1)..(827): beginning and end of NPP1
  MISC_FEATURE (831)..(1057): Fc sequence
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 1058 aa, Artificial Sequence
  Soluble NPP1-Fc fusion protein sequence
  MISC_FEATURE (2)..(828): beginning and end of NPP1
  MISC_FEATURE (832)..(1058): fc sequence
APSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2021/0187067 A1
(43) Pub. Date: Jun. 24, 2021

(54) TREATMENT OF DISEASES INVOLVING DEFICIENCY OF ENPP1 OR ENPP3

(71) Applicant: Inozyme Pharma, Inc., Boston, MA (US)

(72) Inventors: Steven Jungles, Key West, FL (US); Demetrios Braddock, Guilford, CT (US)

(21) Appl. No.: 17/178,127

(22) Filed: Feb. 17, 2021

Related U.S. Application Data
(63) Continuation of application No. PCT/US2020/014296, filed on Jan. 20, 2020.
(60) Provisional application No. 62/877,044, filed on Jul. 22, 2019.
(60) Provisional application No. 62/821,692, filed on Mar. 21, 2019.
(60) Provisional application No. 62/794,450, filed on Jan. 18, 2019.

(51) Int. Cl. A61K 38/17; C12N 15/86; C12N 7/00; A61P 13/12

(52) U.S. Cl. CPC A61K 38/177; C12N 15/86; C12N 2750/14171; A61P 13/12; C12N 2750/14143; C12N 7/00

(57) ABSTRACT

The present disclosure provides, among other things, vectors for expression of ENPP1 or ENPP3 in vivo and methods for the treatment of diseases of calcification and ossification in a subject.

### CROSS REFERENCE

This application is a continuation application of International Application No. PCT/US2020/014296, which claims priority to U.S. Application No. 62/794,450 filed on Jan. 18, 2019, U.S. Application No. 62/821,692 filed on Mar. 21, 2019, and U.S. Application No. 62/877,044 filed on Jul. 22, 2019, the contents of each of which are herein incorporated by reference in their entirety.

### SEQUENCE LISTING

This application contains a Sequence Listing which has been submitted electronically in ASCII format and is hereby incorporated by reference in its entirety. Said ASCII copy, created on Feb. 17, 2021, is named 4427-10006_sequence_ST25.txt and is 446.707 bytes in size.

### FIELD

The invention generally relates to the treatment of diseases involving a deficiency of ENPP1 or ENPP3 by providing nucleic acid encoding ENPP1 or ENPP3 to a mammal.

### BACKGROUND

ENPP1 (also known as PC-1) is a type 2 extracellular membrane-bound glycoprotein located on the mineral-depositing matrix vesicles of osteoblasts and chondrocytes and hydrolyzes extracellular nucleotides (principally ATP) into adenosine monophosphate (AMP) and inorganic pyrophosphate (PPi). PPi functions as a potent inhibitor of ectopic tissue mineralization by binding to nascent hydroxyapatite (HA) crystals, thereby preventing the future growth of these crystals. ENPP1 generates PPi via hydrolysis of nucleotide triphosphates (NTPs), Progressive Ankylosis Protein (ANK) transports intracellular PPi into the extracellular space, and Tissue Non-specific Alkaline Phosphatase (TNAP) removes PPi via direct hydrolysis of PPi into Pi. WO 2011/113027—Quinn et al., WO 2012/125182—Quinn et al, WO 2016/100803—Quinn et al and WO 2017/218786—Yan et al. describe NPP1.

ENPP3 like ENPP1 also belongs to the phosphodiesterase I/nucleotide pyrophosphatase enzyme family. These enzymes are type II transmembrane proteins that catalyze the cleavage of phosphodiester and phosphosulfate bonds of a variety of molecules, including deoxynucleotides, NAD, and nucleotide sugars. ENPP1 been shown to be effective in treating certain diseases of ectopic tissue calcification, such as reducing generalized arterial calcifications in a mouse model for GACI (generalized arterial calcification of infants), which is a severe disease occurring in infants and involving extensive arterial calcification (Albright, et al., 2015, Nature Comm. 10006).

### SUMMARY OF THE INVENTION

In one aspect, the disclosure provides a recombinant polynucleotide encoding a recombinant polypeptide comprising ectonucleotide pyrophosphatase/phosphodiesterase-1 (ENPP1) or ectonucleotide pyrophosphatase/phosphodiesterase-3 (ENPP3).

In another aspect, the disclosure provides a viral vector comprising any of the recombinant polynucleotides described herein

In some embodiments, the recombinant polynucleotide encodes a human ENPP1 or a human ENPP3 polypeptide. Thus, the disclosure also provides a viral vector comprising a recombinant polynucleotide encoding a recombinant polypeptide comprising ectonucleotide pyrophosphatase/phosphodiesterase-1 (ENPP1) or ectonucleotide pyrophosphatase/phosphodiesterase-3 (ENPP3).

In some embodiments of any of the polynucleotides or viral vectors described herein, the recombinant polypeptide is an ENPP1 fusion polypeptide.

In some embodiments of any of the polynucleotides or viral vectors described herein, the recombinant polypeptide is an ENPP3 fusion polypeptide.

In some embodiments of any of the polynucleotides or viral vectors described herein, the ENPP1 fusion polypeptide is an ENPP1-Fc fusion polypeptide or ENPP1-Albumin fusion polypeptide.

In some embodiments of any of the polynucleotides or viral vectors described herein, the ENPP3 fusion polypeptide is an ENPP3-Fc fusion polypeptide or ENPP3-Albumin fusion polypeptide.

In some embodiments of any of the polynucleotides or viral vectors described herein, the recombinant polypeptide comprises a signal peptide fused to ENPP1 or ENPP3.

In some embodiments of any of the polynucleotides or viral vectors described herein, the signal peptide is Azurocidin signal peptide or NPP2 signal peptide or NPP7 signal peptide.

In some embodiments of any of the polynucleotides or viral vectors described herein, the viral vector is Adeno-Associated Viral Vector, or Herpes Simplex Vector, or Alphaviral Vector, or Lentiviral Vectors. In one aspect of the invention, the serotype of Adeno-Associated viral vector (AAV) is AAV1, or AAV2, or AAV3, or AAV4, or AAV5, or AAV6, or AAV7, or AAV8, or AAV9, or AAV-rh74.

In yet another aspect, the disclosure provides an Adeno-Associated viral vector comprising a recombinant polypeptide encoding an ENPP1-Fc fusion polypeptide.

In yet another aspect, the disclosure provides an Adeno-Associated viral vector comprising a recombinant polypeptide encoding a recombinant polypeptide comprising an Azurocidin signal peptide fused to ENPP1-Fc fusion polypeptide.

In some embodiments, the viral vector is not an insect viral vector, such as a baculoviral vector.

In some embodiments, the viral vector is capable of infecting mammalian cells such as human cells (e.g human liver cells or HEK cells, HeLa or A549 or Hepatocytes). In some embodiments the viral vector is capable of infecting, entering, and/or fusing with mammalian cells, such as human cells. In some embodiments, all or a functional part (e.g., that capable of expressing a polypeptide described herein) of the polynucleotide of the viral vector integrates or is integrated into the genome of the cell contacted by a viral vector described herein. In some embodiments, all or a functional part of the polynucleotide of the viral vector is capable of persisting in an extrachromosomal state without integrating into the genome of the mammaliancell contacted with a viral vector described herein.

In some embodiments, the recombinant polynucleotide comprises a vector or a plasmid that encodes viral proteins and/or a human ENPP1. In some embodiments, the recombinant polynucleotide comprises a vector or a plasmid that encodes viral proteins and/or a human ENPP3. In some embodiments, the vector or said plasmid is capable of expressing the encoded polypeptide comprising an Azurocidin signal peptide fused to ectonucleotide pyrophosphatase/phosphodiesterase-1 (ENPP1) or to ectonucleotide pyrophosphatase/phosphodiesterase-3 (ENPP3).

In some embodiments, the encoded polypeptide comprises an Azurocidin signal peptide fused to ectonucleotide pyrophosphatase/phosphodiesterase-1 (ENPP1) comprises a transmembrane domain, a somatomedin domain, catalytic domain and a nuclease domain.

In some embodiments, the encoded polypeptide comprises an Azurocidin signal peptide fused to ectonucleotide pyrophosphatase/phosphodiesterase-1 (ENPP1) is secreted into the cytosol.

In some embodiments, the recombinant polynucleotide encoding polypeptide comprises a transmembrane domain fused to ectonucleotide pyrophosphatase/phosphodiesterase-1 (ENPP1) is not secreted and is membrane bound.

In some embodiments, the disclosure provides a recombinant polynucleotide encoding a polypeptide comprising ectonucleotide pyrophosphatase/phosphodiesterase-1 (ENPP1) In some embodiments the polypeptide comprising ectonucleotide pyrophosphatase/phosphodiesterase-1 (ENPP1) comprises amino acid residues of SEQ ID NO: 1.

In some embodiments, the encoded polypeptide comprises an Azurocidin signal peptide fused to ectonucleotide pyrophosphatase/phosphodiesterase-1 (ENPP1)

In some embodiments, the encoded polypeptide comprising an Azurocidin signal peptide fused to ectonucleotide pyrophosphatase/phosphodiesterase-1 (ENPP1) lacks polyaspartic domain or negatively charged bone targeting domain.

In some embodiments, the vector is a viral vector. In some embodiments the viral vector is an Adeno-associated viral (AAV) vector. In some embodiments, any of the polynucleotides described herein encodes the Azurocidin signal peptide fused to the ENPP1 or Azurocidin signal peptide fused to the ENPP3 and the ENPP1 or the ENPP3 fused to an Fc polypeptide to form in amino to carboxy terminal order Azurocidin signal peptide-ENPP1-Fc or Azurocidin signal peptide-ENPP3-Fc, respectively.

In some embodiments, the recombinant polynucleotide encodes the Azurocidin signal peptide fused to ENPP1 or the Azurocidin signal peptide fused to ENPP3 and the ENPP1 or the ENPP3 fused to human serum albumin to form in amino to carboxy terminal order Azurocidin signal peptide-ENPP1-albumin or Azurocidin signal peptide-ENPP3-albumin, respectively.

In some embodiments, the Fc or albumin sequence is fused directly to the C terminus of the ENPP1 or ENPP3 protein. In some embodiments, the Fc or albumin sequence is fused through a linker, such as a flexible linker to the C terminus of the ENPP1 or ENPP3 protein. In some embodiments, the linker is selected from SEQ ID No: 57-88.

In some embodiments, the viral vector comprising and capable of expressing a nucleic acid sequence encoding a signal peptide fused to the N-terminus of ENPP1 or ENPP3. In some embodiments of the viral vector, the vector comprises a promoter. In some embodiments of the viral vector, the promoter is a liver specific promoter.

In some embodiments of the viral vector, the liver specific promoter is selected from the group consisting of: albumin promoter, phosphoenol pyruvate carboxykinase (PEPCK) promoter and alpha-1-antitrypsin promoter. In some embodiments of the viral vector, the vector comprises a sequence encoding a polyadenylation signal.

In some embodiments of the viral vector, the signal peptide is an Azurocidin signal peptide. In some embodiments of the viral vector, the viral vector is an Adeno-associated viral (AAV) vector. In some embodiments of the viral vector, the AAV vector having a serotype is selected from the group consisting of: AAV1, AAV2, AAV3, AAV4, AAVS, AAV6, AAV7, AAV8, AAV9, and AAV-rh74.

In some embodiments of the viral vector, the polynucleotide of the invention encodes Azurocidin signal peptide fused to ENPP1 or Azurocidin signal peptide fused to ENPP3, and the ENPP1 or the ENPP3 fused to an Fc polypeptide to form in amino to carboxy terminal order Azurocidin signal peptide-ENPP1-Fc or Azurocidin signal peptide-ENPP3-Fc, respectively.

In some embodiments of the viral vector, the polynucleotide encodes Azurocidin signal peptide fused to ENPP1 or Azurocidin signal peptide fused to ENPP3, and the ENPP1 or the ENPP3 fused to human serum albumin to form in amino to carboxy terminal order Azurocidin signal peptide-ENPP1-albumin or Azurocidin signal peptide-ENPP3-albumin, respectively.

In yet another aspect, the disclosure provides a cell (e.g., a mammalian cell, such as a rodent cell, a non-human primate cell, or a human cell) comprising any of the polynucleotides described herein.

In some embodiments, the invention also provides a method of obtaining a recombinant viral vector comprising the steps of:
i. providing a cell comprising a polynucleotide of the invention,
ii. maintaining the cell under conditions adequate for assembly of the virus, and
iii. purifying the viral vector produced by the cell.

In another aspect, the disclosure provides a method of producing a recombinant viral vector. The method comprises:
i. providing a cell or population of cells comprising a polynucleotide described herein, wherein the cell expresses viral proteins essential for packaging or assembly of the polynucleotide into a recombinant viral vector; and
ii. maintaining the cell or population of cells under conditions adequate for the assembly of packaging of said recombinant viral vector.

In some embodiments, the method comprises purifying the viral vector from the cell or population of cells, or from the media in which the cell or population of cells were maintained.

In some embodiments, the cell is a mammalian cell, such as a rodent cell (e.g., rat cell, mouse cell, hamster cell), non-human primate cell, or a human cell (e.g., HEK293, HeLa or A549).

In some embodiments, the method further comprises introducing into the cell or population of cells a recombinant nucleic acid encoding one or more viral proteins (such as those that are essential for packaging or assembly of a viral vector), e.g., infecting the cell or population of cells with a helper virus containing such recombinant nucleic acid, transfection or the cell or population of cells with a helper plasmids comprising such recombinant nucleic acid, and the like.

In some embodiments, the viral vector is capable of expressing one or more polypeptides described herein upon infection in a target cell.

In some embodiments, the disclosure provides a pharmaceutical composition comprising the purified viral vector as described herein. In some embodiments, the disclosure provides a sterile pharmaceutical composition comprising the strerile/endotoxin free purified viral vector as described herein.

In another aspect, the disclosure provides a viral vector obtained and purified by the any of the methods described herein.

In another aspect, the disclosure provides a pharmaceutical composition comprising any of the viral vectors obtained and purified by any of the methods described herein.

In certain embodiments, the invention provides a method of providing ENPP1 or ENPP3 to a mammal, the method comprising administering to the mammal a viral vector of the invention.

In certain embodiments, the disclosure provides a method of expressing ENPP1 or ENPP3 in a mammal (e.g., a human, such as a human in need of such expression), the method comprising administering to the mammal any of the viral vectors described herein. Prior to, at the same time as, and/or following administration of the viral vector to the mammal, the method can further include detecting and/or measuring in a biological sample obtained from the mammal one or more of the following parameters: expression of ENPP1 and/or ENPP3, levels of activity of ENPP1 and/or ENPP3, and/or pyrophosphate levels or concentration. In some embodiments, the one or more parameters are detected or measured within a week, 1-2 weeks, and/or within a month, following administration of the viral vector to the mammal. In some embodiments, the mammal (e.g., a human) is one with an ENPP1 or ABCC6 deficiency.

In another aspect, the disclosure provides a pharmaceutical composition comprising any of the viral vectors as described herein and a physiologically compatible carrier.

In some embodiments, the disclosure provides a method of preventing or reducing the progression of a condition or disease in a mammal in need thereof, the method comprising administering to said mammal a therapeutically effective amount of a composition according to the invention, wherein the condition or disease includes, without limitation, one or more of the following: a deficiency of NPP1, a low level of PPi, a progressive disorder characterized by accumulation of deposits of calcium and other minerals in arterial and/or connective tissues, ectopic calcification of soft tissue, arterial or venous calcification, calcification of heart tissue, such as aorta tissue and coronary tissue, Pseudoxanthoma elasticum (PXE), X-linked hypophosphatemia (XLH), Chronic kidney disease (CKD), Mineral bone disorders (MBD), vascular calcification, pathological calcification of soft tissue, pathological ossification of soft tissue, Generalized arterial calcification of infants (GACI), and Ossification of posterior longitudinal ligament (OPLL), whereby said disease in said mammal is prevented or its progress reduced.

In another aspect, the disclosure provides a method of treating, preventing, and/or ameliorating a disease or disorder of pathological calcification or pathological ossification in a subject in need thereof, the method comprising administering a therapeutically effective amount of any of the viral vectors described herein, thereby treating, preventing, or ameliorating said disease or disorder. In some embodiments, the viral vector comprises a polynucleotide encoding a human ENPP1 or a human ENPP3 polypeptide.

In another aspect, the disclosure provides a method of treating a subject having an ENPP1 protein deficiency, the method comprising administering a therapeutically effective amount of a viral vector which encodes a recombinant ENPP1 or ENPP3 polypeptide to a subject, thereby treating the subject. In one aspect of the invention, the viral vector encodes a human ENPP1 or a human ENPP3 polypeptide.

In another aspect, the subject has a disease or disorder or an ENPP1 protein deficiency that is associated with a loss of function mutation in an NPP1 gene of the subject or a loss of function mutation in an ABCC6 gene of the subject.

In some embodiments of any of the methods described herein, the viral vector is an AAV vector encoding ENPP1-Fc fusion polypeptide, and the vector is administered to a subject at a dosage of 1×10¹² to 1×10¹⁵ vg/kg , preferably 1×10¹³ to 1×10¹⁴ vg/kg.

In some embodiments of any of the methods described herein, the viral vector is an AAV vector encoding ENPP1-Fc fusion polypeptide, and the vector is administered to a subject at a dosage of 5×10¹¹-5×10¹⁵ vg/kg.

In some embodiments of any of the methods described herein, the viral vector is an AAV vector encoding ENPP1-Fc fusion polypeptide, and approximately 1×10¹²-1×10¹⁵ vg/kg per subject is administered for delivering and expressing an ENPP1-Fc polypeptide.

In some embodiments of any of the methods described herein, the viral vector is an AAV vector encoding ENPP3-Fc fusion polypeptide, and the vector is administered to a subject at a dosage of 1×10¹² to 1×10¹⁵ vg/kg , preferably 1×10¹³ to 1×10¹⁴ vg/kg.

In some embodiments of any of the methods described herein, the viral vector is an AAV vector encoding ENPP3-Fc fusion polypeptide, and the vector is administered to a subject at a dosage of 5×10¹¹-5×10¹⁵ vg/kg.

In some embodiments of any of the methods described herein, the viral vector is an AAV vector encoding ENPP3-Fc fusion polypeptide, and approximately 1×10¹²-1×10¹⁵ vg/kg per subject is administered for delivering and expressing an ENPP3-Fc polypeptide.

In some embodiments of any of the methods described herein, administration of AAV vectors encoding an ENPP1-Fc polypeptide to a subject produces a dose dependent increase in plasma pyrophosphate (PPi) and a dose dependent increase in plasma ENPP1 concentration in said subject.

Prior to, at the same time as, and/or following administration of the viral vector to the mammal, any of the methods described herein can further include detecting and/or measuring in a biological sample obtained from the mammal one or more of the following parameters: expression of ENPP1 and/or ENPP3, levels of activity of ENPP1 and/or ENPP3, and/or pyrophosphate levels or concentration. In some embodiments, the one or more parameters are detected or measured within a week, 1-2 weeks, and/or within a month, following administration of the viral vector to the mammal.

In yet another aspect, the disclosure provides a method of treating or preventing a disease or disorder of pathological calcification or pathological ossification in a subject in need thereof, comprising administering a therapeutically effective amount of a viral vector which encodes a recombinant ENPP1 or ENPP3 polypeptide to said subject, thereby treating or preventing said disease or disorder.

In another aspect, the disclosure provides a method of of treating a subject having an ENPP1 protein deficiency, comprising administering a therapeutically effective amount of a viral vector which encodes a recombinant ENPP1 or ENPP3 polypeptide to said subject, thereby treating said subject.

In some embodiments of any of the methods described herein, said disease or disorder or said ENPP1 protein deficiency is associated with a loss of function mutation in an NPP1 gene or a loss of function mutation in an ABCC6 gene in said subject.

In some embodiments of any of the methods described herein, said viral vector encodes recombinant ENPP1 polypeptide.

In some embodiments of any of the methods described herein, said viral vector encodes recombinant ENPP3 polypeptide.

In some embodiments of any of the methods described herein, said viral vector encodes a recombinant ENPP1-Fc fusion polypeptide or a recombinant ENPP1-albumin fusion polypeptide.

In some embodiments of any of the methods described herein, said viral vector encodes a recombinant ENPP3-Fc fusion polypeptide or a recombinant ENPP3-albumin fusion polypeptide.

In some embodiments of any of the methods described herein, said viral vector encodes a recombinant polypeptide comprising a signal peptide fused to ENPP1 or ENPP3.

In some embodiments of any of the methods described herein, said vector encodes ENPP1-Fc or ENPP1-albumin.

In some embodiments of any of the methods described herein, said signal peptide is an azurocidin signal peptide, an NPP2 signal peptide, or an NPP7 signal peptide.

In some embodiments of any of the methods described herein, the viral vector is Adeno-Associated Viral Vector, or Herpes Simplex Vector, or Alphaviral Vector, or Lentiviral Vectors.

In some embodiments of any of the methods described herein, the serotype of Adeno-Associated viral vector (AAV) is AAV1, or AAV2, or AAV3, or AAV4, or AAV5, or AAV6, or AAV7, or AAV8, or AAV9, or AAV-rh74.

In some embodiments of any of the methods described herein, the viral vector is an Adeno-Associated viral (AAV) vector encoding a recombinant polypeptide comprising an Azurocidin signal peptide fused to ENPP1-Fc fusion polypeptide.

In some embodiments of any of the methods described herein, said AAV vector encoding said ENPP1-Fc fusion polypeptide is administered to subjects at a dosage of 1×10¹² to 1×10¹⁵ vg/kg.

In some embodiments of any of the methods described herein, said dosage is 1×10¹³ to 1×10¹⁴ vg/kg.

In some embodiments of any of the methods described herein, said AAV vector is administered to a subject at a dosage of 5×10¹¹-5×10¹⁵ vg/kg.

In some embodiments of any of the methods described herein, said vector is an AAV vector encoding ENPP1-Fc and is administered to a subject at dosage of 1×10¹²-1×10¹⁵ vg/kg. In some embodiments of any of the aforesaid methods, wherein administration of said AAV vector encoding ENPP1-Fc polypeptide to a subject produces a dose dependent increase in plasma pyrophosphate (PPi) and a dose dependent increase in plasma ENPP1 concentration in said subject.

In another aspect, the disclosure features a viral vector comprising a polynucleotide sequence encoding a polypeptide comprising the catalytic domain of an ENPP1 or an ENPP3 protein.

In some embodiments of any of the viral vectors described herein, polypeptide sequence comprises the extracellular domain of an ENPP1 or ENPP3 protein.

In some embodiments of any of the viral vectors described herein, the polypeptide comprises the transmembrane domain of an ENPP1 or ENPP3 protein.

In some embodiments of any of the viral vectors described herein, the polypeptide comprises the nuclease domain of an ENPP1 or ENPP3 protein.

In some embodiments of any of the viral vectors described herein, the polypeptide comprises residues 99-925(Pro Ser Cys to Gln Glu Asp) of SEQ ID NO: 1.

In some embodiments of any of the viral vectors described herein, the polypeptide comprises residues 31-875 (Leu Leu Val to Thr Thr Ile) of SEQ ID NO: 7.

In some embodiments of any of the viral vectors described herein, the polypeptide comprises residues 191-591 (Val Glu Glu to Gly Ser Leu) of SEQ ID NO: 1.

In some embodiments of any of the viral vectors described herein, the polypeptide comprises residues 140-510 (Leu Glu Glu to Glu Val Glu) of SEQ ID NO: 7.

In some embodiments of any of the viral vectors described herein, the polypeptide comprises residues 1-827 (Pro Ser Cys to Gln Glu Asp) of SEQ ID NO: 92.

In some embodiments of any of the viral vectors described herein, the polypeptide comprises residues 1-833 (Phe Thr Ala to Gln Glu Asp) of SEQ ID NO: 89 or residues 1-830 (Gly Leu Lys to Gln Glu Asp) of SEQ ID NO: 91

In some embodiments of any of the viral vectors described herein, the viral vector is not an insect viral vector.

In some embodiments of any of the viral vectors described herein, the viral vector infects or is capable of infecting mammalian cells.

In some embodiments of any of the viral vectors described herein, the polynucleotide sequence encodes a promoter sequence.

In some embodiments of any of the viral vectors described herein, said promoter is a liver specific promoter.

In some embodiments of any of the viral vectors described herein, the liver specific promoter is selected from the group consisting of: albumin promoter, phosphoenol pyruvate carboxykinase (PEPCK) promoter, and alpha-1-antitrypsin promoter.

In some embodiments of any of the viral vectors described herein, the polynucleotide sequence comprises a nucleotide sequence encoding a polyadenylation signal.

In some embodiments of any of the viral vectors described herein, the polynucleotide encodes a signal peptide amino-terminal to nucleotide sequence encoding the ENPP1 or ENPP3 protein.

In some embodiments of any of the viral vectors described herein, the signal peptide is an Azurocidin signal peptide.

In some embodiments of any of the viral vectors described herein, the viral vector is an Adeno-associated viral (AAV) vector.

In some embodiments of any of the viral vectors described herein, said AAV vector has a serotype selected from the group consisting of: AAV1, AAV2, AAV3, AAV4, AAV5, AAV6, AAV7, AAV8, AAV9, and AAV-rh74.

In some embodiments of any of the viral vectors described herein, said polynucleotide sequence encodes said Azurocidin signal peptide fused to said ENPP1 or said Azurocidin signal peptide fused to said ENPP3, and said ENPP1 or said ENPP3 fused to an Fc polypeptide to form in amino to carboxy terminal order Azurocidin signal peptide-ENPP1-Fc or Azurocidin signal peptide-ENPP3-Fc, respectively.

In some embodiments of any of the viral vectors described herein, said polynucleotide sequence encodes said Azurocidin signal peptide fused to said ENPP1 or said Azurocidin signal peptide fused to said ENPP3, and said ENPP1 or said ENPP3 fused to human serum albumin to form in amino to carboxy terminal order Azurocidin signal peptide-ENPP1-albumin or Azurocidin signal peptide-ENPP3-albumin, respectively.

In some embodiments of any of the viral vectors described herein, the polypeptide is a fusion protein comprising: (i) an ENPP1 protein or an ENPP3 protein and (ii) a half-life extending domain.

In some embodiments of any of the viral vectors described herein, the half-life extending domain is an IgG Fc domain or a functional fragment of the IgG Fc domain capable of extending the half-life of the polypeptide in a mammal, relative to the half-life of the polypeptide in the absence of the IgG Fc domain or functional fragment thereof.

In some embodiments of any of the viral vectors described herein, the half-life extending domain is an albumin domain or a functional fragment of the albumin domain capable of extending the half-life of the polypeptide in a mammal, relative to the half-life of the polypeptide in the absence of the albumin domain or functional fragment thereof.

In some embodiments of any of the viral vectors described herein, the half-life extending domain is carboxyterminal to the ENPP1 or ENPP3 protein in the fusion protein.

In some embodiments of any of the viral vectors described herein, the IgG Fc domain comprises the amino acid sequence as shown in SEQ ID NO: 34

In some embodiments of any of the viral vectors described herein, the albumin domain comprises the amino acid sequence as shown in SEQ ID NO: 35

In some embodiments of any of the viral vectors described herein, the polynucleotide encodes a linker sequence.

In some embodiments of any of the viral vectors described herein, the linker sequence is selected from the group consisting of SINs: 57 to 88.

In some embodiments of any of the viral vectors described herein, the linker sequence joins the ENPP1 or ENPP3 protein and the half-life extending domain of the fusion protein.

In some embodiments of any of the viral vectors described herein, the polypeptide comprises the amino acid sequence depicted in SEQ ID NO: 89, 91, 92 and 93.

In another aspect, the disclosure provides a method for producing a recombinant viral vector, the method comprising:
i. providing a cell or population of cells comprising a polynucleotide encoding a polypeptide comprising the catalytic domain of an ENPP1 or an ENPP3 protein, wherein the cell expresses viral proteins essential for packaging and/or assembly of the polynucleotide into a recombinant viral vector; and
ii. maintaining the cell or population of cells under conditions adequate for the assembly of packaging of said recombinant viral vector comprising the polynucleotide.

In some embodiments of any of the methods described herein, the mammalian cell is a rodent cell or a human cell.

In some embodiments of any of the methods described herein, the viral vector is any one of the viral vectors described herein.

In some embodiments, any of the methods described herein can further comprise purifying the recombinant viral vector from the cell or population of cells, or from the media in which the cell or population of cells were maintained.

In another aspect, the disclosure features the recombinant viral vector purified from the methods for producing and/or purifying a recombinant viral vector described herein.

In another aspect, the disclosure provides a pharmaceutical composition comprising any one of the viral vectors or recombinant viral vectors described herein and a pharmaceutically acceptable carrier.

In yet another aspect, the disclosure provides a method of preventing or reducing the progression of a disease in a mammal in need thereof, the method comprising: administering to said mammal a therapeutically effective amount of any one of the pharmaceutical compositions described herein to thereby prevent or reduce the progression of the disease or disorder.

In some embodiments of any of the methods described herein, the mammal is a human. In some embodiments of any of the methods described herein, the disease is selected from the group consisting of: X-linked hypophosphatemia (XLH), Chronic kidney disease (CKD), Mineral bone disorders (MBD), vascular calcification, pathological calcification of soft tissue, pathological ossification of soft tissue, PXE, Generalized arterial calcification of infants (GACI), and Ossification of posterior longitudinal ligament (OPLL).

In another aspect, the disclosure provides a method of treating or preventing a disease or disorder of pathological calcification or pathological ossification in a subject in need thereof, the method comprising administering to the subject a therapeutically effective amount of any one of the viral vectors or pharmaceutical compositions described herein, thereby treating or preventing said disease or disorder.

In another aspect, the disclosure features a method of treating a subject having an ENPP1 protein deficiency, the method comprising administering to the subject a therapeutically effective amount of any one of the viral vectors or pharmaceutical compositions described herein, thereby treating said subject.

In some embodiments of any of the methods described herein, said disease or disorder or said ENPP1 protein deficiency is associated with a loss of function mutation in an NPP1 gene or a loss of function mutation in an ABCC6 gene in said subject.

In some embodiments of any of the methods described herein, the viral vector or pharmaceutical composition is administered at a dosage of 1×10¹² to 1×10¹⁴ vg/kg of the subject or mammal.

In some embodiments of any of the methods described herein, the viral vector or pharmaceutical composition is administered at a dosage of 1×10¹³ to 1×10¹⁴ vg/kg of the subject or mammal.

In some embodiments of any of the methods described herein, the viral vector or pharmaceutical composition is administered at a dosage of 5×10¹²-5×10¹⁵ vg/kg of the subject or mammal.

In some embodiments of any of the methods described herein, the viral vector or pharmaceutical composition is administered at a dosage of 1×10¹²-1×10¹⁵ vg/kg of the subject or mammal.

In some embodiments of any of the methods described herein, administration of said viral vector or pharmaceutical composition to the subject or mammal increases plasma pyrophosphate (PPi) and/or plasma ENPP1 or ENPP3 concentration in said subject or mammal.

In some embodiments, any of the aforesaid methods can further comprise detecting or measuring in a biological sample obtained from the subject or mammal one or more of the following parameters: (i) the concentration of pyrophosphate, (ii) the expression level of ENPP1 or ENPP3, and (iii) the enzymatic activity of ENPP1 or ENPP3.

In some embodiments of any of the methods described herein, the detecting or measuring occurs before administering the viral vector or pharmaceutical composition.

### BRIEF DESCRIPTION OF THE FIGURES

FIG. 1—Schematic showing AAV construct

FIG. 2—Figure showing increased amount of expression of ENPP1when using Azurocidin signal sequence as compared with NPP2 and NPP7 signal sequences.

FIG. 3—Plasmid map of vector expressing ENPP1-Fc fusion

FIG. 4—Schematic view showing the administration of viral particles comprising ENPP1 constructs to model mice.

FIG. 5—Figure showing dose dependent increase in ENPP1 activity in blood plasma samples obtained from control, low dose and high dose mice cohorts collected at 7 days, 28 days and 56 days post administration of viral vector.

FIG. 6—Figure showing dose dependent increase in ENPP1 concentration in blood plasma samples obtained from control, low dose and high dose mice cohorts collected at 7 days, 28 days and 56 days post administration of viral vector.

FIG. 7—Figure showing dose dependent increase in Plasma PPi concentration in blood plasma samples obtained from control, low dose and high dose mice cohorts collected at 7 days, 28 days and 56 days post administration of viral vector.

FIG. 8—Figure showing persistent expression of Enpp1 for up to 112 days post viral vector administration.

FIG. 9—Figure showing dose dependent increase in ENPP1 activity in blood plasma samples obtained from control, low dose and high dose mice cohorts collected at 7 days, 28 days, 56 days and 112 days post administration of viral vector.

### DETAILED DESCRIPTION ACCORDING TO THE INVENTION

The invention pertains to delivery of nucleic acid encoding mammal ENPP1 or mammal ENPP3 to a mammal having a deficiency in ENPP1 or ENPP3.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, illustrative methods and materials are described. As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The following notation conventions are applied to the present disclosure for the sake of clarity. In any case, any teaching herein that does not follow this convention is still part of the present disclosure and can be fully understood in view of the context in which the teaching is disclosed. Protein symbols are disclosed in non-italicized capital letters. As non-limiting examples, ‘ENPP1’ refer to the protein. In certain embodiments, if the protein is a human protein, an ‘h’ is used before the protein symbol. In other embodiments, if the protein is a mouse protein, an ‘m’ is used before the symbol. Human ENPP1 is referred to as ‘hENPP1’, and mouse ENPP1 is referred to as ‘mENPP1’. Human gene symbols are disclosed in italicized capital letters. As a non-limiting example, the human gene corresponding to the protein hENPP1 is ENPP1. Mouse gene symbols are disclosed with the first letter in upper case and the remaining letters in lower case; further, the mouse gene symbol is italicized. As a non-limiting example, the mouse gene that makes the protein mEnpp1 is Enpp1. Notations about gene mutations are shown as uppercase text.

“Human ENPP1”: Human NPP1 (NCBI accession NP_006199/Uniprot-Swissprot P22413)

“Soluble human ENPP_1”: residues 96 to 925 of NCBI accession NP_006199

“Human ENPP3”: Human NPP3 (UniProtKB/Swiss-Prot: O14638.2)

“Soluble human ENPP3”: residues 49-875 of UniProtKB/Swiss-Prot: O14638.2

“Reduction of calcification”: As used herein, reduction of calcification is observed by using non-invasive methods like X-rays, micro CT and MM. Reduction of calcification is also inferred by using radio imaging with ^{99m}Tc-pyrophosphate (^{99m}PYP) uptake. The presence of calcifications in mice are evaluated via post-mortem by micro-computed tomography (CT) scans and histologic sections taken from the heart, aorta and kidneys with the use of dyes such as Hematoxylin and Eosin (H&E) and Alizarin red by following protocols established by Braddock et al. (Nature Communications volume 6, Article number: 10006 (2015))

“Enzymatically active” with respect to ENPP1 or ENPP3: is defined as possessing ATP hydrolytic activity into AMP and PPi and/or AP3a hydrolysis to ATP. possessing substrate binding activity.

ATP hydrolytic activity may be determined as follows.

ATP Hydrolytic Activity of NPP1

NPP1 readily hydrolyzes ATP into AMP and PPi. The steady-state Michaelis-Menten enzymatic constants of NPP1 are determined using ATP as a substrate. NPP1 can be demonstrated to cleave ATP by HPLC analysis of the enzymatic reaction, and the identity of the substrates and products of the reaction are confirmed by using ATP, AMP, and ADP standards. The ATP substrate degrades over time in the presence of NPP1, with the accumulation of the enzymatic product AMP. Using varying concentrations of ATP substrate, the initial rate velocities for NPP1 are derived in the presence of ATP, and the data is fit to a curve to derive the enzymatic rate constants. At physiologic pH, the kinetic rate constants of NPP1 are Km=144 and kcatt=7.8 s⁻¹.

ATP Hydrolytic Activity of NPP3

The enzymatic activity of NPP3 was measured with pNP-TMP or ATP as substrates. The NPP3 protein was incubated at 37° C. in the presence of 100 mM Tris-HCl at pH 8.9 and either 5 mM pNP-TMP or 50 μM [γ-32P] ATP. The hydrolysis of pNP-TMP was stopped by a 10-fold dilution in 3% (w/v) trichloroacetic acid. Subsequently, the reaction mixture was neutralized with 60 μl 5 N NaOH and the formed p-nitrophenol (pNP) was quantified colorimetrically at 405 nm. The hydrolysis of ATP was arrested by the addition of 100 mM EDTA. One μl of the reaction mixture was analyzed by thin-layer chromatography on polyethyleneimine cellulose plates (Merck). Nucleotides and degradation products were separated by ascending chromatography in 750 mM KH2PO4 at pH 3.0. Radioactive spots were visualized by autoradiography. The nucleotidylated intermediate, formed during the hydrolysis of 50 μM [α-32P] ATP, was trapped according to Blytt et al. (H. J. Blytt, J. E. Brotherton, L. Butler Anal. Biochem. 147 (1985), pp. 517-520), with slight modifications (R. Gijsbers, H. Ceulemans, W. Stalmans, M. Bollen J. Biol. Chem., 276 (2001), pp. 1361-1368). Following SDS-PAGE, the trapped intermediate was visualized by autoradiography. Bis-pNPP and pNPP were also tested as substrates for NPP3. The NPP3 isoforms were incubated in 100 mM Tris-HCl at pH 8.9 and either 5 mM bis-pNPP or pNPP for 2.5 h at 37° C. Subsequently, the formed pNP was quantified colorimetrically at 405 nm. (Gijsbers R I, Aoki J, Arai H, Bollen M, FEBS Lett. 2003 Mar. 13; 538(1-3):60-4.) At physiologic pH, NPP3 has a kcat value of about 2.59(±0.04) s⁻¹ and Km (<8 μM) values similar to ENPP1. (WO 2017/087936)

HPLC Protocol

The HPLC protocol used to measure ATP cleavage by NPP1, and for product identification, is modified from the literature (Stocchi et al., 1985, Anal. Biochem. 146:118-124). The reactions containing varying concentrations of ATP in 50 mM Tris pH 8.0, 140 mM NaCl, 5 mM KCl, 1 mM MgCl₂ and 1 mM CaCl₂ buffer are started by addition of 0.2-1 μM NPP1 and quenched at various time points by equal volume of 3M formic acid, or 0.5N KOH and re-acidified by glacial acetic acid to pH 6. The quenched reaction solution is diluted systematically, loaded onto a HPLC system (Waters, Milford Mass.), and substrates and products are monitored by UV absorbance at 254 or 259 nm. Substrates and products are separated on a C18, Sum 250×4.6 mm HPLC column (Higgins Analytical, Mountain View, Calif.), using 15 mM ammonium acetate pH 6.0 solution, with a 0% to 10% (or 20%) methanol gradient. The products and substrate are quantified according to the integration of their correspondent peaks and the formula:

$$[ product / substrate ] = \frac{\begin{matrix}{Area}_{product / substrate} / \\ {Σ}_{product / substrate}\end{matrix}}{\begin{matrix}{Area}_{product} / {Σ}_{product} + \\ {Area}_{substrate} / {Σ}_{substrate}\end{matrix}} [ substrate ]$$

where [substrate] is the initial substrate concentration. The extinction coefficients of AMP, ADP and ATP used in the formula were 15.4 mM⁻¹ cm⁻¹. If monitoring at 254 nm, substrate and product standards run on the same day as the reactions were used to convert integrated product/substrate peak areas to concentrations.

“pathological calcification”: As used herein, the term refers to the abnormal deposition of calcium salts in soft tissues, secretory and excretory passages of the body causing it to harden. There are two types, dystrophic calcification which occurs in dying and dead tissue and metastatic calcification which elevated extracellular levels of calcium (hypercalcemia), exceeding the homeostatic capacity of cells and tissues. Calcification can involve cells as well as extracellular matrix components such as collagen in basement membranes and elastic fibers in arterial walls. Some examples of tissues prone to calcification include: Gastric mucosa—the inner epithelial lining of the stomach, Kidneys and lungs, Cornea, Systemic arteries and Pulmonary veins.

“pathological ossification”: As used herein, the term refers to a pathological condition in which bone arises in tissues not in the osseous system and in connective tissues usually not manifesting osteogenic properties. Ossification is classified into three types depending on the nature of the tissue or organ being affected, endochondral ossification is ossification that occurs in and replaces cartilage. Intramembranous ossification is ossification of bone that occurs in and replaces connective tissue. Metaplastic ossification the development of bony substance in normally soft body structures; called also heterotrophic ossification.

A “deficiency” of NPP1 refers to a condition in which the subject has less than or equal to 5%-10% of normal levels of NPP1 in blood plasma. Normal levels of NPP1 in healthy human subjects is approximately between 10 to 30 ng/ml. (Am J Pathol. 2001 February; 158(2): 543-554.)

A “low” level of PPi refers to a condition in which the subject has less than or equal to 2%-5% of normal levels of plasma pyrophosphate (PPi). Normal levels of Plasma PPi in healthy human subjects is approximately 1.8 to 2.6 μM. (Arthritis and Rheumatism, Vol. 22, No. 8 (August 1979))

“Ectopic calcification” refers to a condition characterized by a pathologic deposition of calcium salts in tissues or bone growth in soft tissues.

“Ectopic calcification of soft tissue” refers to inappropriate biomineralization, typically composed of calcium phosphate, hydroxyapatite, calcium oxalates and ocatacalcium phosphates occurring in soft tissues leading to loss of hardening of soft tissues. “Arterial calcification” refers to ectopic calcification that occurs in arteries and heart valves leading to hardening and or narrowing of arteries. Calcification in arteries is correlated with atherosclerotic plaque burden and increased risk of myocardial infarction, increased ischemic episodes in peripheral vascular disease, and increased risk of dissection following angioplasty.

“Venous calcification” refers to ectopic calcification that occurs in veins that reduces the elasticity of the veins and restricts blood flow which can then lead to increase in blood pressure and coronary defects

“Vascular calcification” refers to the pathological deposition of mineral in the vascular system. It has a variety of forms, including intimal calcification and medial calcification, but can also be found in the valves of the heart. Vascular calcification is associated with atherosclerosis, diabetes, certain heredity conditions, and kidney disease, especially CKD. Patients with vascular calcification are at higher risk for adverse cardiovascular events. Vascular calcification affects a wide variety of patients. Idiopathic infantile arterial calcification is a rare form of vascular calcification where the arteries of neonates calcify.

“Brain calcification” (BC) refers to a nonspecific neuropathology wherein deposition of calcium and other mineral in blood vessel walls and tissue parenchyma occurs leading to neuronal death and gliosis. Brain calcification is” often associated with various chronic and acute brain disorders including Down's syndrome, Lewy body disease, Alzheimer's disease, Parkinson's disease, vascular dementia, brain tumors, and various endocrinologic conditions

Calcification of heart tissue refers to accumulation of deposits of calcium (possibly including other minerals) in tissues of the heart, such as aorta tissue and coronary tissue.

“Chronic kidney disease (CKD)” As used herein, the term refers to abnormalities of kidney structure or function that persist for more than three months with implications for health. Generally excretory, endocrine and metabolic functions decline together in most chronic kidney diseases. Cardiovascular disease is the most common cause of death in patients with chronic kidney disease (CKD) and vascular calcification is one of the strongest predictors of cardiovascular risk. With decreasing kidney function, the prevalence of vascular calcification increases and calcification occurs years earlier in CKD patients than in the general population. Preventing, reducing and/or reversing vascular calcification may result in increased survival in patients with CKD.

Clinical symptoms of chronic kidney diseases include itching, muscle cramps, nausea, lack of appetite, swelling of feet and ankles, sleeplessness and labored breathing. Chronic kidney disease if left untreated tends to progress into End stage renal disease (ESRD). Common symptoms of ESRD include an inability to urinate, fatigue, malaise, weight loss, bone pain, changes in skin color, a frequent formation of bruises, and edema of outer extremities like fingers, toes, hands and legs. Calciphylaxis or calcific uremic arteriolopathy (CUA) is a condition that causes calcium to build up inside the blood vessels of the fat and skin. A subpopulation of patients suffering from ESRD can also develop Calciphylaxis. Common symptoms of Calciphylaxis include large purple net-like patterns on skin, deep and painful lumps that ulcerate creating open sores with black-brown crust that fails to heal, skin lesions on the lower limbs or areas with higher fat content, such as thighs, breasts, buttocks, and abdomen. A person with calciphylaxis may have higher than normal levels of calcium (hypercalcemia) and phosphate (hyperphosphatemia) in the blood. They may also have symptoms of hyperparathyroidism. Hyperparathyroidism occurs when the parathyroid glands make excess parathyroid hormone (PTH). Reduced plasma pyrophosphate (PPi) levels are also present in vascular calcification associated with end stage renal disease (ESRD).

Vascular calcifications associated with ESRD contributes to poor outcomes by increasing pulse pressure, causing or exacerbating hypertension, and inducing or intensifying myocardial infarctions and strokes. Most patients with ESRD do not die of renal failure, but from the cardiovascular complications of ESRD, and it is important to note that many very young patients with ESRD on dialysis possess coronary artery calcifications. The histologic subtype of vascular calcification associated with CKD is known as Monckeburg's sclerosis, which is a form of vessel hardening in which calcium deposits are found in the muscular layers of the medial vascular wall. This form of calcification is histologically distinct from intimal or neo-intimal vascular wall calcification commonly observed in atherosclerosis but identical to the vascular calcifications observed in human CKD patients, and in the rodent models of the disease described herein.

“Generalized arterial calcification of infants (GACI)” (also known as IACI)”, as used herein, refers to a disorder affecting the circulatory system that becomes apparent before birth or within the first few months of life. It is characterized by abnormal accumulation of the mineral calcium (calcification) in the walls of the blood vessels that carry blood from the heart to the rest of the body (the arteries). Calcification often occurs along with thickening of the lining of the arterial walls (the intima). These changes lead to narrowing (stenosis) and stiffness of the arteries, which forces the heart to work harder to pump blood. As a result, heart failure may develop in affected individuals, with signs and symptoms including difficulty breathing, accumulation of fluid (edema) in the extremities, a bluish appearance of the skin or lips (cyanosis), severe high blood pressure (hypertension), and an enlarged heart (cardiomegaly). People with GACI may also have calcification in other organs and tissues, particularly around the joints. In addition, they may have hearing loss or softening and weakening of the bones referred to as rickets.

General arterial calcification (GACI) or Idiopathic Infantile Arterial Calcification (IIAC) characterized by abnormal accumulation of the mineral calcium (calcification) in the walls of the blood vessels that carry blood from the heart to the rest of the body (the arteries). The calcification often occurs along with thickening of the lining of the arterial walls (the intima). These changes lead to narrowing (stenosis) and stiffness of the arteries, which forces the heart to work harder to pump blood. As a result, heart failure may develop in affected individuals, with signs and symptoms including difficulty breathing, accumulation of fluid (edema) in the extremities, a bluish appearance of the skin or lips (cyanosis), severe high blood pressure (hypertension), and an enlarged heart (cardiomegaly).

“Arterial calcification” or “Vascular calcification” or “hardening of arteries”, As used herein, the term refers to a process characterized by thickening and loss of elasticity of muscular arteries walls. The thickening and loss of elasticity occurs in two distinct sites, the intimal and medial layers of the vasculatures (Medial vascular calcification). Intimal calcification is associated with atherosclerotic plaques and medial calcification is characterized by vascular stiffening and arteriosclerosis. This results in a reduction of arterial elasticity and an increased propensity for morbidity and mortality due to the impairment of the cardiovascular system's hemodynamics.

“Mineral bone disorders (MBD)”, as used herein, the term refers to a disorder characterized by abnormal hormone levels cause calcium and phosphorus levels in a person's blood to be out of balance. Mineral and bone disorder commonly occurs in people with CKD and affects most people with kidney failure receiving dialysis.

Osteopenia is a bone condition characterized by decreased bone density, which leads to bone weakening and an increased risk of bone fracture. Osteomalacia is a bone disorder characterized by decreased mineralization of newly formed bone. Osteomalacia is caused by severe vitamin D deficiency (which can be nutritional or caused by a hereditary syndrome) and by conditions that cause very low blood phosphate levels. Both osteomalacia and osteopenia increase the risk of breaking a bone. Symptoms of osteomalacia include bone pain and muscle weakness, bone tenderness, difficulty walking, and muscle spasms.

“Age related osteopenia”, as used herein refers to a condition in which bone mineral density is lower than normal. Generally, patients with osteopenia have a bone mineral density T-score of between −1.0 and −2.5. Osteopenia if left untreated progresses into Osetoporosis where bones become brittle and are extremely prone to fracture.

“Ossification of posterior longitudinal ligament (OPLL)”, as used herein, the term refers to a hyperostotic (excessive bone growth) condition that results in ectopic calcification of the posterior longitudinal ligament. The posterior longitudinal ligament connects and stabilizes the bones of the spinal column. The thickened or calcified ligament may compress the spinal cord, producing myelopathy. Symptoms of myelopathy include difficulty walking and difficulty with bowel and bladder control. OPLL may also cause radiculopathy, or compression of a nerve root. Symptoms of cervical radiculopathy include pain, tingling, or numbness in the neck, shoulder, arm, or hand.

Clinical symptoms and signs caused by OPLL are categorized as: (1) myelopathy, or a spinal cord lesion with motor and sensory disturbance of the upper and lower limbs, spasticity, and bladder dysfunction; (2) cervical radiculopathy, with pain and sensory disturbance of the upper limbs; and (3) axial discomfort, with pain and stiffness around the neck. The most common symptoms in the early stages of OPLL include dysesthesia and tingling sensation in hands, and clumsiness. With the progression of neurologic deficits, lower extremity symptoms, such as gait disturbance may appear. OPLL is detected on lateral plain radiographs, and the diagnosis and morphological details of cervical OPLL have been clearly demonstrated by magnetic resonance imaging (MRI) and computed tomography (CT).

“Pseudoxanthoma elasticum (PXE)”, as used herein, the term refers a progressive disorder that is characterized by the accumulation of deposits of calcium and other minerals (mineralization) in elastic fibers. Elastic fibers are a component of connective tissue, which provides strength and flexibility to structures throughout the body. In PXE, mineralization can affect elastic fibers in the skin, eyes, and blood vessels, and less frequently in other areas such as the digestive tract. People with PXE may have yellowish bumps called papules on their necks, underarms, and other areas of skin that touch when a joint bends. Mineralization of the blood vessels that carry blood from the heart to the rest of the body (arteries) may cause other signs and symptoms of PXE. For example, people with this condition can develop narrowing of the arteries (arteriosclerosis) or a condition called claudication that is characterized by cramping and pain during exercise due to decreased blood flow to the arms and legs.

Pseudoxanthoma elasticum (PXE), also known as Grönblad-Strandberg syndrome, is a genetic disease that causes fragmentation and mineralization of elastic fibers in some tissues. The most common problems arise in the skin and eyes, and later in blood vessels in the form of premature atherosclerosis. PXE is caused by autosomal recessive mutations in the ABCC6 gene on the short arm of chromosome 16 (16p13.1). In some cases, a portion of infants survive GACI and end up developing Pseudoxanthoma elasticum (PXE) when they grow into adults. PXE is characterized by the accumulation of calcium and other minerals (mineralization) in elastic fibers, which are a component of connective tissue. Connective tissue provides strength and flexibility to structures throughout the body. Features characteristic of PXE that also occur in GACI include yellowish bumps called papules on the underarms and other areas of skin that touch when a joint bends (flexor areas); arterial stenosis, and abnormalities called angioid streaks affecting tissue at the back of the eye (retinal hemorrhage), which is detected during an eye examination.

“End stage renal disease (ESRD): as used herein, the term refers to an advanced stage of chronic kidney disease where kidneys of the patient are no longer functional. Common symptoms include fatigue associated with anemia (low blood iron), decreased appetite, nausea, vomiting, abnormal lab values including elevated potassium, abnormalities in hormones related to bone health, elevated phosphorus and/or decreased calcium, high blood pressure (hypertension), swelling in hands/legs/eyes/lower back (sacrum) and shortness of breath.

“Calcific uremic arteriolopathy (CUA)” or “Calciphylaxis”, as used herein refers to a condition with high morbidity and mortality seen in patients with kidney disease, especially in those with end stage renal disease (ESRD). It is characterized by calcification of the small blood vessels located within the fatty tissue and deeper layers of the skin leading to blood clots, and the death of skin cells due to reduced blood flow caused by excessive calcification.

“Hypophosphatemic rickets”, as used herein refers to a disorder in which the bones become soft and bend easily, due to low levels of phosphate in the blood. Symptoms usually begin in early childhood and can range in severity from bowing of the legs, bone deformities; bone pain; joint pain; poor bone growth; and short stature.

“Hereditary Hypophosphatemic Rickets” as used herein refers to a disorder related to low levels of phosphate in the blood (hypophosphatemia). Phosphate is a mineral that is essential for the normal formation of bones and teeth. Most commonly, it is caused by a mutation in the PHEX gene. Other genes that can be responsible for the condition include the CLCN5, DMP1, ENPP1, FGF23, and SLC34A3 genes. Other signs and symptoms of hereditary hypophosphatemic rickets can include premature fusion of the skull bones (craniosynostosis) and dental abnormalities. The disorder may also cause abnormal bone growth where ligaments and tendons attach to joints (enthesopathy). In adults, hypophosphatemia is characterized by a softening of the bones known as osteomalacia. Another rare type of the disorder is known as hereditary hypophosphatemic rickets with hypercalciuria (HHRH) wherein in addition to hypophosphatemia, this condition is characterized by the excretion of high levels of calcium in the urine (hypercalciuria).

“X-linked hypophosphatemia (XH)”, as used herein, the term X-linked hypophosphatemia (XLH), also called X-linked dominant hypophosphatemic rickets, or X-linked Vitamin D-resistant rickets, is an X-linked dominant form of rickets (or osteomalacia) that differs from most cases of rickets in that vitamin D supplementation does not cure it. It can cause bone deformity including short stature and genu varum (bow leggedness). It is associated with a mutation in the PHEX gene sequence (Xp.22) and subsequent inactivity of the PHEX protein.

“Autosomal Recessive Hypophosphatemia Rickets type 2 (ARHR2)”, as used herein, the term refers to a hereditary renal phosphate-wasting disorder characterized by hypophosphatemia, rickets and/or osteomalacia and slow growth. Autosomal recessive hypophosphatemic rickets type 2 (ARHR2) is caused by homozygous loss-of-function mutation in the ENPP1 gene.

“Autosomal Dominant Hypophosphatemic Rickets (ADHR)”, as used herein refers to a rare hereditary disease in which excessive loss of phosphate in the urine leads to poorly formed bones (rickets), bone pain, and tooth abscesses. ADHR is caused by a mutation in the fibroblast growth factor 23 (FGF23). ADHR is characterized by impaired mineralization of bone, rickets and/or osteomalacia, suppressed levels of calcitriol (1, 25-dihydroxyvitamin D3), renal phosphate wasting, and low serum phosphate. Mutations in FGF23 render the protein more stable and uncleavable by proteases resulting in enhanced bioactivity of FGF23. The enhanced activity of FGF23 mutants reduce expression of sodium-phosphate co-transporters, NPT2a and NPT2c, on the apical surface of proximal renal tubule cells, resulting in renal phosphate wasting.

Hypophosphatemic rickets (previously called vitamin D-resistant rickets) is a disorder in which the bones become painfully soft and bend easily, due to low levels of phosphate in the blood. Symptoms may include bowing of the legs and other bone deformities; bone pain; joint pain; poor bone growth; and short stature. In some affected babies, the space between the skull bones closes too soon leading to craniosynostosis. Most patients display Abnormality of calcium-phosphate metabolism, Abnormality of dental enamel, Delayed eruption of teeth and long, narrow head (Dolichocephaly).

The terms “adeno-associated viral vector”,“AAV vector”,“adeno-associated virus”, “AAV virus”,“AAV virion”, “AAV viral particle” and “AAV particle”, as used interchangeably herein, refer to a viral particle composed of at least one AAV capsid protein (preferably by all of the capsid proteins of a particular AAV serotype) and an encapsidated recombinant viral genome. The particle comprises a recombinant viral genome having a heterologous polynucleotide comprising a sequence encoding human ENPP1 or human ENPP3 or a functionally equivalent variant thereof,) and a transcriptional regulatory region that at least comprises a promoter flanked by the AAV inverted terminal repeats. The particle is typically referred to as an “AAV vector particle” or “AAV vector”.

As used herein, the term “vector” means a nucleic acid molecule capable of transporting another nucleic acid to which it has been linked. In some embodiments, the vector is a plasmid, i.e., a circular double stranded DNA loop into which additional DNA segments may be ligated. In some embodiments, the vector is a viral vector, wherein additional nucleotide sequences may be ligated into the viral genome. In some embodiments, the vectors are capable of autonomous replication in a host cell into which they are introduced (e.g., bacterial vectors having a bacterial origin of replication and episomal mammalian vectors). In other embodiments, the vectors (e.g., non-episomal mammalian vectors) is integrated into the genome of a host cell upon introduction into the host cell, and thereby are replicated along with the host genome. Moreover, certain vectors (expression vectors) are capable of directing the expression of genes to which they are operatively linked.

As used herein, the term “recombinant host cell” (or simply “host cell”), as used herein, means a cell into which an exogenous nucleic acid and/or recombinant vector has been introduced. It should be understood that “recombinant host cell” and “host cell” mean not only the particular subject cell but also the progeny of such a cell. Because certain modifications may occur in succeeding generations due to either mutation or environmental influences, such progeny may not, in fact, be identical to the parent cell, but are still included within the scope of the term “host cell” as used herein.

The term “recombinant viral genome”, as used herein, refers to an AAV genome in which at least one extraneous expression cassette polynucleotide is inserted into the naturally occurring AAV genome. The genome of the AAV according to the invention typically comprises the cis-acting 5′ and 3′ inverted terminal repeat sequences (ITRs) and an expression cassette.

The term “expression cassette”, as used herein, refers to a nucleic acid construct, generated recombinantly or synthetically, with a series of specified nucleic acid elements, which permit transcription of a particular nucleic acid in a target cell. The expression cassette of the recombinant viral genome of the AAV vector according to the invention comprises a transcriptional regulatory region operatively linked to a nucleotide sequence encoding ENPP1 or ENPP3 or a functionally equivalent variant thereof.

The term “transcriptional regulatory region”, as used herein, refers to a nucleic acid fragment capable of regulating the expression of one or more genes. The transcriptional regulatory region according to the invention includes a promoter and, optionally, an enhancer.

The term “promoter”, as used herein, refers to a nucleic acid fragment that functions to control the transcription of one or more polynucleotides, located upstream the polynucleotide sequence(s), and which is structurally identified by the presence of a binding site for DNA-dependent RNA polymerase, transcription initiation sites, and any other DNA sequences including, but not limited to, transcription factor binding sites, repressor, and activator protein binding sites, and any other sequences of nucleotides known in the art to act directly or indirectly to regulate the amount of transcription from the promoter. Any kind of promoters may be used in the invention including inducible promoters, constitutive promoters and tissue-specific promoters.

The term “enhancer”, as used herein, refers to a DNA sequence element to which transcription factors bind to increase gene transcription. Examples of enhancers may be, without limitation, RSV enhancer, CMV enhancer, HCR enhancer, etc. In another embodiment, the enhancer is a liver-specific enhancer, more preferably a hepatic control region enhancer (HCR).

The term “operatively linked”, as used herein, refers to the functional relation and location of a promoter sequence with respect to a polynucleotide of interest (e.g. a promoter or enhancer is operably linked to a coding sequence if it affects the transcription of the sequence). Generally, a promoter operatively linked is contiguous to the sequence of interest. However, an enhancer does not have to be contiguous to the sequence of interest to control its expression. In another embodiment, the promoter and the nucleotide sequence encoding ENPP1 or ENPP3 or a functionally equivalent variant thereof.

The term “therapeutically effective amount” refers to a nontoxic but sufficient amount of a viral vector encoding ENPP1 or ENPP3 to provide the desired biological result. That result may be reduction and/or alleviation of the signs, symptoms, or causes of a disease, or any other desired alteration of a biological system. For example, a therapeutically effective amount of an AAV vector according to the invention is an amount sufficient to produce

The term “Cap protein”, as used herein, refers to a polypeptide having at least one functional activity of a native AAV Cap protein (e.g. VP1, VP2, VP3). Examples of functional activities of Cap proteins include the ability to induce formation of a capsid, facilitate accumulation of single-stranded DNA, facilitate AAV DNA packaging into capsids (i.e. encapsidation), bind to cellular receptors, and facilitate entry of the virion into host cells. In principle, any Cap protein can be used in the context of the present invention.

The term “capsid”, as used herein, refers to the structure in which the viral genome is packaged. A capsid consists of several oligomeric structural subunits made of proteins. For instance, AAV have an icosahedral capsid formed by the interaction of three capsid proteins: VP1, VP2 and VP3.

The term “Rep protein”, as used herein, refers to a polypeptide having at least one functional activity of a native AAV Rep protein (e.g. Rep 40, 52, 68, 78). A “functional activity” of a Rep protein is any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome. In a particular embodiment, AAV rep genes derive from the serotypes AAV1, AAV2, AAV4, AAVS, AAV6, AAV7, AAV8, AAV9, AAV10 or AAVrh10; more preferably from an AAV serotype selected from the group consisting of AAV2, AAV5, AAV7, AAV8, AAV9, AAV10 and AAVrh10.

The expression “viral proteins upon which AAV is dependent for replication”, as used herein, refers to polypeptides which perform functions upon which AAV is dependent for replication (i.e. “helper functions”). The helper functions include those functions required for AAV replication including, without limitation, those moieties involved in activation of AAV gene transcription, stage specific AAV mRNA splicing, AAV DNA replication, synthesis of cap expression products, and AAV capsid assembly. Viral-based accessory functions are derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, ULB, UL52, and UL29, and herpesvirus polymerase. In another embodiment, the proteins upon which AAV is dependent for replication are derived from adenovirus.

The term “adeno-associated virus ITRs” or “AAV ITRs”, as used herein, refers to the inverted terminal repeats present at both ends of the DNA strand of the genome of an adeno-associated virus. The ITR sequences are required for efficient multiplication of the AAV genome. Another property of these sequences is their ability to form a hairpin. This characteristic contributes to its self-priming which allows the primase-independent synthesis of the second DNA strand. Procedures for modifying these ITR sequences are known in the art (Brown T, “Gene Cloning”, Chapman & Hall, London, GB, 1995; Watson R, et al., “Recombinant DNA”, 2^{nd} Ed. Scientific American Books, New York, N.Y., US, 1992; Alberts B, et al., “Molecular Biology of the Cell”, Garland Publishing Inc., New York, N.Y., US, 2008; Innis M, et al., Eds., “PCR Protocols. A Guide to Methods and Applications”, Academic Press Inc., San Diego, Calif., US, 1990; and Schleef M, Ed., “Plasmid for Therapy and Vaccination”, Wiley-VCH Verlag GmbH, Weinheim, Del., 2001).

The term “tissue-specific” promoter is only active in specific types of differentiated cells or tissues. Typically, the downstream gene in a tissue-specific promoter is one which is active to a much higher degree in the tissue(s) for which it is specific than in any other. In this case there may be little or substantially no activity of the promoter in any tissue other than the one(s) for which it is specific.

The term “skeletal muscle-specific promoter”, as used herein, refers to a nucleic acid sequence that serves as a promoter (i.e. regulates expression of a selected nucleic acid sequence operably linked to the promoter), and which promotes expression of a selected nucleic acid sequence in specific tissue cells of skeletal muscle. Examples of skeletal muscle-specific promoters include, without limitation, myosin light chain promoter (MLC) and the muscle creatine kinase promoter (MCK).

The term “liver specific promoter”, as used herein, refers to a nucleic acid sequence that serves as a promoter (i.e. regulates expression of a selected nucleic acid sequence operably linked to the promoter), and which promotes expression of a selected nucleic acid sequence in hepatocytes. Typically, a liver-specific promoter is more active in liver as compared to its activity in any other tissue in the body. The liver-specific promoter can be constitutive or inducible. Suitable liver-specific promoters include, without limitation, an [alpha] 1-anti-trypsin (AAT) promoter, a thyroid hormone-binding globulin promoter, an alpha fetoprotein promoter, an alcohol dehydrogenase promoter, the factor VIII (FVIII) promoter, a HBV basic core promoter (BCP) and PreS2 promoter, an albumin promoter, a −460 to 73 bp phosphoenol pyruvate carboxykinase (PEPCK) promoter, a thyroxin-binding globulin (TBG) promoter, an Hepatic Control Region (HCR)-ApoCII hybrid promoter, an HCR-hAAT hybrid promoter, an AAT promoter combined with the mouse albumin gene enhancer (Ea1b) element, an apolipoprotein E promoter, a low density lipoprotein promoter, a pyruvate kinase promoter, a lecithin-cholesterol acyl transferase (LCAT) promoter, an apolipoprotein H (ApoH) promoter, the transferrin promoter, a transthyretin promoter, an alpha-fibrinogen and beta-fibrinogen promoters, an alpha 1-antichymotrypsin promoter, an alpha 2-HS glycoprotein promoter, an haptoglobin promoter, a ceruloplasmin promoter, a plasminogen promoter, promoters of the complement proteins (CIq, CIr, C2, C3, C4, C5, C6, C8, C9, complement Factor I and Factor H), C3 complement activator and the [alpha]-acid glycoprotein promoter. Additional tissue-specific promoters may be found in the Tissue-Specific Promoter Database, TiProD (Nucleic Acids Research, J4:D104-D107 (2006)). In another embodiment, the liver-specific promoter is selected from the group consisting of albumin promoter, phosphoenol pyruvate carboxykinase (PEPCK) promoter and alpha 1-antitrypsin promoter; more preferably alpha 1-antitrypsin promoter; even more preferably human alpha 1-antitrypsin promoter.

The term “inducible promoter”, as used herein, refers to a promoter that is physiologically or developmentally regulated, e.g. by the application of a chemical inducer. For example, it can be a tetracycline-inducible promoter, a mifepristone (RU-486)-inducible promoter and the like.

The term “constitutive promoter”, as used herein, refers to a promoter whose activity is maintained at a relatively constant level in all cells of an organism, or during most developmental stages, with little or no regard to cell environmental conditions. In another embodiment, the transcriptional regulatory region allows constitutive expression of ENPP1. Examples of constitutive promoters include, without limitation, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1a promoter (Boshart M, et al., Cell 1985; 41:521-530). Preferably, the constitutive promoter is suitable for expression of ENPP1 in liver and include, without limitation, a promoter of hypoxanthine phosphoribosyl transferase (HPTR), a promoter of the adenosine deaminase, a promoter of the pyruvate kinase, a promoter of β-actin, an elongation factor 1 alpha (EF1) promoter, a phosphoglycerate kinase (PGK) promoter, a ubiquitin (Ubc) promoter, an albumin promoter, and other constitutive promoters. Exemplary viral promoters which function constitutively in cells include, for example, the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), or the herpes thymidine kinase promoter (Wagner et al., 1981, Proc. Natl. Acad. Sci. U.S.A. 78:1441-1445).

The term “polyadenylation signal”, as used herein, relates to a nucleic acid sequence that mediates the attachment of a polyadenine stretch to the 3′ terminus of the mRNA. Suitable polyadenylation signals include, without limitation, the SV40 early polyadenylation signal, the SV40 late polyadenylation signal, the HSV thymidine kinase polyadenylation signal, the protamine gene polyadenylation signal, the adenovirus 5 EIb polyadenylation signal, the bovine growth hormone polyadenylation signal, the human variant growth hormone polyadenylation signal and the like.

The term “nucleotide or nucleic acid sequence”, is used herein interchangeably with “polynucleotide”, and relates to any polymeric form of nucleotides of any length. Said nucleotide sequence encodes signal peptide and ENPP1 protein or a functionally equivalent variant thereof.

The term “signal peptide”, as used herein, refers to a sequence of amino acid residues (ranging in length from 10-30 residues) bound at the amino terminus of a nascent protein of interest during protein translation. The signal peptide is recognized by the signal recognition particle (SRP) and cleaved by the signal peptidase following transport at the endoplasmic reticulum. (Lodish et al., 2000, Molecular Cell Biology, 4th edition).

The term “subject”, as used herein, refers to an individualmammal, such as a human, a non-human primate (e.g. chimpanzees and other apes and monkey species), a farm animal (e.g. birds, fish, cattle, sheep, pigs, goats, and horses), a domestic mammal (e.g. dogs and cats), or a laboratory animal (e.g. rodents, such as mice, rats and guinea pigs). The term includes a subject of any age or sex. In another embodiment the subject is a mammal, preferably a human.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “immune response” or “immune reaction” refers to the host's immune system to antigen in an invading (infecting) pathogenic organism, or to introduction or expression of foreign protein. The immune response is generally humoral and local; antibodies produced by B cells combine with antigen in an antigen-antibody complex to inactivate or neutralize antigen. Immune response is often observed when human proteins are injected into mouse model systems. Generally, the mouse model system is made immune tolerant by injecting immune suppressors prior to the introduction of a foreign antigen to ensure better viability.

As used herein, the term “immunesuppression” is a deliberate reduction of the activation or efficacy of the host immune system using immunesuppresant drugs to facilitate immune tolerance towards foreign antigens such as foreign proteins, organ transplants, bone marrow and tissue transplantation. Non limiting examples of immunosuppressant drugs include anti-CD4(GK1.5) antibody, Cyclophosphamide, Azathioprine (Imuran), Mycophenolate mofetil (Cellcept), Cyclosporine (Neoral, Sandimmune, Gengraf), Methotrexate (Rheumatrex), Leflunomide (Arava), Cyclophosphamide (Cytoxan) and Chlorambucil (Leukeran).

As used herein, the term “ENPP” or “NPP” refers to ectonucleotide pyrophosphatase/phosphodiesterase.

As used herein, the term “ENPP1 protein” or “ENPP1 polypeptide” refers to ectonucleotide pyrophosphatase/phosphodiesterase-1 protein encoded by the ENPP1 gene. The encoded protein is a type II transmembrane glycoprotein and cleaves a variety of substrates, including phosphodiester bonds of nucleotides and nucleotide sugars and pyrophosphate bonds of nucleotides and nucleotide sugars. ENPP1 protein has a transmembrane domain and soluble extracellular domain. The extracellular domain is further subdivided into somatomedin B domain, catalytic domain and the nuclease domain. The sequence and structure of wild-type ENPP1 is described in detail in PCT Application Publication No. WO 2014/126965 to Braddock, et al., which is incorporated herein in its entirety by reference.

Mammal ENPP1 and ENPP3 polypeptides, mutants, or mutant fragments thereof, have been previously disclosed in International PCT Application Publications No. WO/2014/126965—Braddock et al., WO/2016/187408—Braddock et al., WO/2017/087936—Braddock et al., and WO2018/027024—Braddock et al., all of which are incorporated by reference in their entireties herein.

As used herein, the term “ENPP3 protein” or “ENPP3 polypeptide” refers to ectonucleotide pyrophosphatase/phosphodiesterase-3 protein encoded by the ENPP3 gene. The encoded protein is a type II transmembrane glycoprotein and cleaves a variety of substrates, including phosphodiester bonds of nucleotides and nucleotide sugars and pyrophosphate bonds of nucleotides and nucleotide sugars. ENPP3 protein has a transmembrane domain and soluble extracellular domain. The sequence and structure of wild-type ENPP3 is described in detail in PCT Application Publication No. WO/2017/087936 to Braddock, et al., which is incorporated herein in its entirety by reference.

As used herein, the term “ENPP1 precursor protein” refers to ENPP1 with its signal peptide sequence at the ENPP1 N-terminus. Upon proteolysis, the signal sequence is cleaved from ENPP1 to provide the ENPP1 protein. Signal peptide sequences useful within the invention include, but are not limited to, Albumin signal sequence, Azurocidin signal sequence, ENPP1 signal peptide sequence, ENPP2 signal peptide sequence, ENPP7 signal peptide sequence, and/or ENPP5 signal peptide sequence.

As used herein, the term “ENPP3 precursor protein” refers to ENPP3 with its signal peptide sequence at the ENPP3 N-terminus. Upon proteolysis, the signal sequence is cleaved from ENPP3 to provide the ENPP3 protein. Signal peptide sequences useful within the invention include, but are not limited to, Albumin signal peptide sequence, Azurocidin signal peptide sequence, ENPP1 signal peptide sequence, ENPP2 signal peptide sequence, ENPP7 signal peptide sequence, and/or ENPP5 signal peptide sequence.

As used herein, the term “Azurocidin signal peptide sequence” refers to the signal peptide derived from human azurocidin. Azurocidin, also known as cationic antimicrobial protein CAP37 or heparin-binding protein (HBP), is a protein that in humans is encoded by the AZU1 gene. The nucleotide sequence encoding Azurocin signal peptide (MTRLTVLALLAGLLASSRA) is fused with the nucleotide sequence of NPP1 or NPP3 gene which when encoded generates ENPP1 precursor protein or ENPP3 precursor protein. (Optimized signal peptides for the development of high expressing CHO cell lines, Kober et al., Biotechnol Bioeng. 2013 April; 110(4): 1164-73)

As used herein, the term “ENPP1-Fc construct” refers to ENPP1 recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “ENPP3-Fc construct” refers to ENPP3 recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “Fc” refers to a human IgG (immunoglobulin) Fc domain. Subtypes of IgG such as IgG1, IgG2, IgG3, and IgG4 are contemplated for use as Fc domains.

As used herein, the “Fc region or Fc polypeptide” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231 -447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

An “oligonucleotide” or “polynucleotide” is a nucleic acid ranging from at least 2, in certain embodiments at least 8, 15 or 25 nucleotides in length, but may be up to 50, 100, 1000, or 5000 nucleotides long or a compound that specifically hybridizes to a polynucleotide.

As used herein, the term “patient,” “individual” or “subject” refers to a human.

As used herein, the term “pharmaceutical composition” or “composition” refers to a mixture of at least one compound useful within the invention with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained; for example, phosphate-buffered saline (PBS)

As used herein the term “plasma pyrophosphate (PPi) levels” refers to the amount of pyrophosphate present in plasma of animals. In certain embodiments, animals include rat, mouse, cat, dog, human, cow and horse. It is necessary to measure PPi in plasma rather than serum because of release from platelets. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications. Typically, normal PPi levels in healthy subjects range from about 1 μm to about 3 μM, in some cases between 1-2 μm. Subjects who have defective ENPP1 expression tend to exhibit low ppi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with GACI, the ppi levels are found to be less than 1 μm and in some cases are below the level of detection. In patients afflicted with PXE, the ppi levels are below 0.5 μm. (Arterioscler Thromb Vasc Biol. 2014 September; 34(9): 1985-9; Braddock et al., Nat Commun. 2015; 6: 10006.)

As used herein, the term “polypeptide” refers to a polymer composed of amino acid residues, related naturally occurring structural variants, and synthetic non-naturally occurring analogs thereof linked via peptide bonds.

As used herein, the term “PPi” refers to pyrophosphate.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

As used herein, the term “treatment” or “treating” is defined as the application or administration of a therapeutic agent, i.e., a compound useful within the invention (alone or in combination with another pharmaceutical agent), to a patient, or application or administration of a therapeutic agent to an isolated tissue or cell line from a patient (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder, or the potential to develop the disease or disorder. Such treatments may be specifically tailored or modified, based on knowledge obtained from the field of pharmacogenomics.

The terms “prevent,” “preventing,” and “prevention”, as used herein, refer to inhibiting the inception or decreasing the occurrence of a disease in a subject. Prevention may be complete (e.g. the total absence of pathological cells in a subject) or partial. Prevention also refers to a reduced susceptibility to a clinical condition.

As used herein, the term “wild-type” refers to a gene or gene product isolated from a naturally occurring source. A wild-type gene is most frequently observed in a population and is thus arbitrarily designed the “normal” or “wild-type” form of the human NPP1 or NPP3 genes. In contrast, the term “functionally equivalent” refers to a NPP1 or NPP3 gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

The term “functional equivalent variant”, as used herein, relates to a polypeptide substantially homologous to the sequences of ENPP1 or ENPP3 (defined above) and that preserves the enzymatic and biological activities of ENPP1 or ENPP3, respectively. Methods for determining whether a variant preserves the biological activity of the native ENPP1 or ENPP3 are widely known to the skilled person and include any of the assays used in the experimental part of said application. Particularly, functionally equivalent variants of ENPP1 or ENPP3 delivered by viral vectors is encompassed by the present invention.

The functionally equivalent variants of ENPP1 or ENPP3 are polypeptides substantially homologous to the native ENPP1 or ENPP3 respectively. The expression “substantially homologous”, relates to a protein sequence when said protein sequence has a degree of identity with respect to the ENPP1 or ENPP3 sequences described above of at least 80%, at least 85%, at least 90%, at least 91%, at least 92%, at least 93%, at least 94%, at least 95%, at least 96%, at least 97%, at least 98% or at least 99% respectively.

The degree of identity between two polypeptides is determined using computer algorithms and methods that are widely known for the persons skilled in the art. The identity between two amino acid sequences is preferably determined by using the BLASTP algorithm (BLAST Manual, Altschul, S., et al., NCBI NLM NIH Bethesda, Md. 20894, Altschul, S., et al., J. Mol. Biol. 215: 403-410 (1990)), though other similar algorithms can also be used. BLAST and BLAST 2.0 are used, with the parameters described herein, to determine percent sequence identity. Software for performing BLAST analyses is publicly available through the National Center for Biotechnology Information.

“Functionally equivalent variants” of ENPP1 or ENPP3 may be obtained by replacing nucleotides within the polynucleotide accounting for codon preference in the host cell that is to be used to produce the ENPP1 or ENPP3 respectively. Such “codon optimization” can be determined via computer algorithms which incorporate codon frequency tables such as “Human high.cod” for codon preference as provided by the University of Wisconsin Package Version 9.0, Genetics Computer Group, Madison, Wis.

“About” as used herein when referring to a measurable value such as an amount, a temporal duration, and the like, is meant to encompass variations of ±20% or ±10%, in certain embodiments ±5%, in certain embodiments ±1%, in certain embodiments ±0.1% from the specified value, as such variations are appropriate to perform the disclosed methods.

The disclosure provides a representative example of protein sequence and nucleic acid sequences of the invention. The protein sequences described can be converted into nucleic acid sequences by performing revere translation and codon optimization. There are several tools available in art such as Expasy (https://www.expasy.org/)and bioinformatics servers (http://www.bioinformatics.org)that enable such conversions

Ranges: throughout this disclosure, various aspects according to the invention can be presented in a range format. It should be understood that the description in range format is merely for convenience and brevity and should not be construed as an inflexible limitation on the scope according to the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Viral Vectors for In Vivo Expression of ENPP1 and ENPP3

Genetic material such as a polynucleotide comprising an NPP1 or an NPP3 sequence can be introduced to a mammal in order to compensate for a deficiency in ENPP1 or ENPP3 polypeptide

Certain modified viruses are often used as vectors to carry a coding sequence because after administration to a mammal, a virus infects a cell and expresses the encoded protein. Modified viruses useful according to the invention are derived from viruses which include, for example: parvovirus, picornavirus, pseudorabies virus, hepatitis virus A, B or C, papillomavirus, papovavirus (such as polyoma and SV40) or herpes virus (such as Epstein-Barr Virus, Varicella Zoster Virus, Cytomegalovirus, Herpes Zoster and Herpes Simplex Virus types 1 and 2), an RNA virus or a retrovirus, such as the Moloney murine leukemia virus or a lentivirus (i.e. derived from Human Immunodeficiency Virus, Feline Immunodeficiency Virus, equine infectious anemia virus, etc.). Among DNA viruses useful according to the invention are: Adeno-associated viruses adenoviruses, Alphaviruses, and Lentiviruses.

A viral vector is generally administered by injection, most often intravenously (by IV) directly into the body, or directly into a specific tissue, where it is taken up by individual cells. Alternately, a viral vector may be administered by contacting the viral vector ex vivo with a sample of the patient's cells, thereby allowing the viral vector to infect the cells, and cells containing the vector are then returned to the patient. Once the viral vector is delivered, the coding sequence expressed and results in a functioning protein. Generally, the infection and transduction of cells by viral vectors occur by a series of sequential events as follows: interaction of the viral capsid with receptors on the surface of the target cell, internalization by endocytosis, intracellular trafficking through the endocytic/proteasomal compartment, endosomal escape, nuclear import, virion uncoating, and viral DNA double-strand conversion that leads to the transcription and expression of the recombinant coding sequence interest. (Colella et al., Mol Ther Methods Clin Dev. 2017 Dec. 1; 8:87-104.).

Adeno-Associated Viral Vectors According to the Invention

AAV refers to viruses belonging to the genus Dependovirus of the Parvoviridae family. The AAV genome is approximately 4.7 kilobases long and is composed of linear single-stranded deoxyribonucleic acid (ssDNA) which may be either positive- or negative-sensed. The genome comprises inverted terminal repeats (ITRs) at both ends of the DNA strand, and two open reading frames (ORFs): rep and cap. The rep frame is made of four overlapping genes encoding non-structural replication (Rep) proteins required for the AAV life cycle. The cap frame contains overlapping nucleotide sequences of structural VP capsid proteins: VP1, VP2 and VP3, which interact together to form a capsid of an icosahedral symmetry.

The terminal 145 nucleotides are self-complementary and are organized so that an energetically stable intramolecular duplex forming a T-shaped hairpin may be formed. These hairpin structures function as an origin for viral DNA replication, serving as primers for the cellular DNA polymerase complex. Following wild type AAV infection in mammalian cells the rep genes (i.e. Rep78 and Rep52) are expressed from the P5 promoter and the P19 promoter, respectively, and both Rep proteins have a function in the replication of the viral genome. A splicing event in the rep ORF results in the expression of actually four Rep proteins (i.e. Rep78, Rep68, Rep52 and Rep40). However, it has been shown that the unspliced mRNA, encoding Rep78 and Rep52 proteins, in mammalian cells are sufficient for AAV vector production. Also in insect cells the Rep78 and Rep52 proteins suffice for AAV vector production.

The AAV vector typically lacks rep and cap frames. Such AAV vectors can be replicated and packaged into infectious viral particles when present in a host cell that has been transfected with a vector encoding and expressing rep and cap gene products (i.e. AAV Rep and Cap proteins), and wherein the host cell has been transfected with a vector which encodes and expresses a protein from the adenovirus open reading frame E4orf6.

In one embodiment, the invention relates to an adeno-associated viral (AAV) expression vector comprising a sequence encoding mammal ENPP1 or mammal ENPP3, and upon administration to a mammal the vector expresses an ENPP1 or ENPP3 precursor in a cell, the precursor including an Azurocidin signal peptide fused at its carboxy terminus to the amino terminus of ENPP1 or ENPP3. The ENPP1 or ENPP3 precursor may include a stabilizing domain, such as an IgG Fc region or human albumin. Upon secretion of the precursor from the cell, the signal peptide is cleaved off and enzymatically active soluble mammal ENPP1 or ENPP3 is provided extracellularly.

An AAV expression vector may include an expression cassette comprising a transcriptional regulatory region operatively linked to a nucleotide sequence comprising a transcriptional regulatory region operatively linked to a recombinant nucleic acid sequence encoding a polypeptide comprising a Azurocidin signal peptide sequence and an ectonucleotide pyrophosphatase/phosphodiesterase (ENPP1) polypeptide sequence.

In some embodiments, the expression cassette comprises a promoter and enhancer, the Kozak sequence GCCACCATGG, a nucleotide sequence encoding mammal NPP1 protein or a nucleotide sequence encoding mammal NPP3 protein, other suitable regulatory elements and a polyadenylation signal.

In some embodiments, the AAV recombinant genome of the AAV vector according to the invention lacks the rep open reading frame and/or the cap open reading frame.

The AAV vector according to the invention comprises a capsid from any serotype. In general, the AAV serotypes have genomic sequences of significant homology at the amino acid and the nucleic acid levels, provide an identical set of genetic functions, and replicate and assemble through practically identical mechanisms. In particular, the AAV of the present invention may belong to the serotype 1 of AAV (AAV1), AAV2, AAV3 (including types 3A and 3B), AAV4, AAV5, AAV6, AAV7, AAV8, AAV9, AAV10, AAVrh10, AAV11, avian AAV, bovine AAV, canine AAV, equine AAV, or ovine AAV.

Examples of the sequences of the genome of the different AAV serotypes may be found in the literature or in public databases such as GenBank. For example, GenBank accession numbers NC_001401.2 (AAV2), NC_001829.1 (AAV4), NC_006152.1 (AAV5), AF028704.1 (AAV6), NC_006260.1 (AAV7), NC_006261.1 (AAV8), AX753250.1 (AAV9) and AX753362.1 (AAV10).

In some embodiments, the adeno-associated viral vector according to the invention comprises a capsid derived from a serotype selected from the group consisting of the AAV2, AAV5, AAV7, AAV8, AAV9, AAV10 and AAVrh10 serotypes. In another embodiment, the serotype of the AAV is AAV8. If the viral vector comprises sequences encoding the capsid proteins, these may be modified so as to comprise an exogenous sequence to direct the AAV to a particular cell type or types, or to increase the efficiency of delivery of the targeted vector to a cell, or to facilitate purification or detection of the AAV, or to reduce the host response.

The published application, US 2017/0290926—Smith et al., the contents of which are incorporated by reference in their entirety herein, describes in detail the process by which AAV vectors are generated, delivered and administered.

Adeno Viral Vectors Useful According to the Invention

Adenovirus can be manipulated such that it encodes and expresses the desired gene product, (e.g., ENPP1 or ENPP3), and at the same time is inactivated in terms of its ability to replicate in a normal lytic viral life cycle. In addition, adenovirus has a natural tropism for airway epithelial. The viruses are able to infect quiescent cells as are found in the airways, offering a major advantage over retroviruses. Adenovirus expression is achieved without integration of the viral DNA into the host cell chromosome, thereby alleviating concerns about insertional mutagenesis. Furthermore, adenoviruses have been used as live enteric vaccines for many years with an excellent safety profile (Schwartz, A. R. et al. (1974) Am. Rev. Respir. Dis. 109:233-238). Finally, adenovirus mediated gene transfer has been demonstrated in a number of instances including transfer of alpha-1-antitrypsin and CFTR to the lungs of cotton rats (Rosenfeld, M. A. et al. (1991) Science 252:431-434; Rosenfeld et al., (1992) Cell 68:143-155). Furthermore, extensive studies to attempt to establish adenovirus as a causative agent in human cancer were uniformly negative (Green, M. et al. (1979) Proc. Natl. Acad. Sci. USA 76:6606).

Pseudo-Adenovirus Vectors (PAV)—PAVs contain adenovirus inverted terminal repeats and the minimal adenovirus 5′ sequences required for helper virus dependent replication and packaging of the vector. These vectors contain no potentially harmful viral genes, have a theoretical capacity for foreign material of nearly 36 kb, may be produced in reasonably high titers and maintain the tropism of the parent virus for dividing and non-dividing human target cell types. The PAV vector can be maintained as either a plasmid-borne construct or as an infectious viral particle. As a plasmid construct, PAV is composed of the minimal sequences from wild type adenovirus type 2 necessary for efficient replication and packaging of these sequences and any desired additional exogenous genetic material, by either a wild-type or defective helper virus.

The US patent publication, U.S. Pat. No. 7,318,919—Gregory et al., describes in detail the process by which adenoviral vectors are generated, delivered and their corresponding use for treatment of diseases, the contents of which are incorporated by reference in their entirety herein. The present invention contemplates the use of Adenoviral vectors to deliver nucleotides encoding ENPP1 or ENPP3 to a subject in need thereof and the methods of treatment using the same.

Herpes Simplex Vectors Useful According to the Invention

A Herpes Simplex Vector (HSV based viral vector) is suitable for use as a vector to introduce a nucleic acid sequence into numerous cell types. The mature HSV virion consists of an enveloped icosahedral capsid with a viral genome consisting of a linear double-stranded DNA molecule that is 152 kb. In another embodiment, the HSV based viral vector is deficient in at least one essential HSV gene. In some embodiments, the HSV based viral vector that is deficient in at least one essential HSV gene is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb.

HSV-based vectors are described in, for example, U.S. Pat. No. 5,837,532—Preston et al., U.S. Pat. No. 5,846,782—Wickham et al., and U.S. Pat. No. 5,804,413—Deluca et al., and International Patent Applications WO 91/02788—Preston et al., WO 96/04394—Preston et al., WO 98/15637—Deluca et al., and WO 99/06583—Glorioso et al., which are incorporated herein by reference. The HSV vector can be deficient in replication-essential gene functions of only the early regions of the HSV genome, only the immediate-early regions of the HSV genome, only the late regions of the HSV genome, or both the early and late regions of the HSV genome. The production of HSV vectors involves using standard molecular biological techniques well known in the art.

Replication deficient HSV vectors are typically produced in complementing cell lines that provide gene functions not present in the replication deficient HSV vectors, but required for viral propagation, at appropriate levels in order to generate high titers of viral vector stock. The expression of the nucleic acid sequence encoding the protein is controlled by a suitable expression control sequence operably linked to the nucleic acid sequence. An “expression control sequence” is any nucleic acid sequence that promotes, enhances, or controls expression (typically and preferably transcription) of another nucleic acid sequence.

Suitable expression control sequences include constitutive promoters, inducible promoters, repressible promoters, and enhancers. The nucleic acid sequence encoding the protein in the vector can be regulated by its endogenous promoter or, preferably, by a non-native promoter sequence. Examples of suitable non-native promoters include the human cytomegalovirus (HCMV) promoters, such as the HCMV immediate-early promoter (HCMV IEp), promoters derived from human immunodeficiency virus (HIV), such as the HIV long terminal repeat promoter, the phosphoglycerate kinase (PGK) promoter, Rous sarcoma virus (RSV) promoters, such as the RSV long terminal repeat, mouse mammary tumor virus (MMTV) promoters, the Lap2 promoter, or the herpes thymidine kinase promoter (Wagner et al., Proc. Natl. Acad. Sci., 78, 1444-1445 (1981)), promoters derived from SV40 or Epstein Barr virus, and the like. In another embodiment, the promoter is HCMV IEp.

The promoter can also be an inducible promoter, i.e., a promoter that is up- and/or down-regulated in response to an appropriate signal. For example, an expression control sequence up-regulated by a pharmaceutical agent is particularly useful in pain management applications. For example, the promoter can be a pharmaceutically-inducible promoter (e.g., responsive to tetracycline).The promoter can be introduced into the genome of the vector by methods known in the art, for example, by the introduction of a unique restriction site at a given region of the genome.

The US patent publication, U.S. Pat. No. 7,531,167—Glorioso et al., describes in detail the process by which Herpes Simplex vectors are generated, delivered and their corresponding use for treatment of diseases, the contents of which are incorporated by reference in their entirety herein. The present invention contemplates the use of Herpes Simplex vectors to deliver nucleotides encoding ENPP1 or ENPP3 to a subject in need thereof and the methods of treatment using the same.

Alphaviral Vectors Useful According to the Invention

Alphaviral expression vectors have been developed from different types of alphavirus, including Sindbis virus (SIN), Semliki Forest Virus (SFV) and Venezuelan equine encephalitis (VEE) virus. The alphavirus replicon contains at its 5′ end an open reading frame encoding viral replicase (Rep) which is translated when viral RNA is transfected into cells. Rep is expressed as a polyprotein which is subsequently processed into four subunits (nsps 1 to 4). Unprocessed Rep can copy the RNA vector into negative-strand RNA, a process that only takes place during the first 3 to 4 hours after transfection or infection. Once processed, the Rep will use the negative-strand RNA as a template for synthesizing more replicon molecules. Processed Rep can also recognize an internal sequence in the negative-strand RNA, or subgenomic promoter, from which it will synthesize a subgenomic positive-strand RNA corresponding to the 3′ end of the replicon. This subgenomic RNA will be translated to produce the heterologous protein in large amounts.

A non-cytopathic mutant isolated from SIN containing a single amino acid change (P for L) in position 726 in nsp2 (SIN P726L vector in nsp2) showed Rep hyper processing (Frolov et al., 1999, J. Virol. 73: 3854-65). This mutant was capable of efficiently establishing continuous replication in BHK cells. This non-cytopathic SIN vector has been widely used in vitro as it is capable of providing long-lasting transgene expression with good stability levels and expression levels that were about 4% of those obtained with the original SIN vector (Agapov et al., 1998, Proc. Natl. Acad. Sci. USA. 95: 12989-94). Likewise, the Patent application WO2008065225—Smerdou et al., describes a non-cytopathic SFV vector has mutations R649H/P718T in the replicase nsp2 subunit. The aforesaid vector allows obtaining cell lines capable of constitutively and stably expressing the gene of interest by means of culturing in the presence of an antibiotic the resistance gene of which is incorporated in the alphaviral vector (Casales et al. 2008. Virology. 376:242-51).

The invention contemplates designing a vector comprising a DNA sequence complementary to an alphavirus replicon in which a sequence of a gene of interest such as NPP1 or NPP3 has been incorporated along with recognition sequences for site-specific recombination. By means of said vector, it is possible to obtain and select cells in which the alphaviral replicon, including the sequence of the gene of interest, has been integrated in the cell genome, such that the cells stably express ENPP1 or ENPP3 polypeptide. The invention also contemplates generating an expression vector in which the alphaviral replicon is under the control of an inducible promoter. Said vector when incorporated to cells which have additionally been modified by means of incorporating an expression cassette encoding a transcriptional activator which, in the presence of a given ligand, is capable of positively regulating the activity of the promoter which regulates alphavirus replicon transcription.

The US patent publication, U.S. Pat. No. 10,011,847—Aranda et al., describes in detail the process by which Alphaviral vectors are generated, delivered and their corresponding use for treatment of diseases, the contents of which are incorporated by reference in their entirety herein. The present invention contemplates the use of Alphaviral vectors to deliver nucleotides encoding ENPP1 or ENPP3 to a subject in need thereof and methods of treatment using the same.

Lentiviral Vectors Useful According to the Invention

Lentiviruses belong to a genus of viruses of the Retroviridae family and are characterized by a long incubation period. Lentiviruses can deliver a significant amount of viral RNA into the DNA of the host cell and have the unique ability among retroviruses of being able to infect non-dividing cells. Lentiviral vectors, especially those derived from HIV-1, are widely studied and frequently used vectors. The evolution of the lentiviral vectors backbone and the ability of viruses to deliver recombinant DNA molecules (transgenes) into target cells have led to their use in restoration of functional genes in genetic therapy and in vitro recombinant protein production.

The invention contemplates a lentiviral vector comprising a suitable promoter and a transgene to express protein of interest such as ENPP1 or ENPP3. Typically, the backbone of the vector is from a simian immunodeficiency virus (SIV), such as SIV1 or African green monkey SIV (SIV-AGM). In one embodiment, the promoter is preferably a hybrid human CMV enhancer/EF1a (hCEF) promoter. The present invention encompasses methods of manufacturing Lentiviral vectors, compositions comprising Lentiviral vectors expressing genes of interest, and use in gene therapy to express ENPP1 or ENPP3 protein in order to treat diseases of calcification or ossification. The lentiviral vectors according to the invention can also be used in methods of gene therapy to promote secretion of therapeutic proteins. By way of further example, the invention provides secretion of therapeutic proteins into the lumen of the respiratory tract or the circulatory system. Thus, administration of a vector according to the invention and its uptake by airway cells may enable the use of the lungs (or nose or airways) as a “factory” to produce a therapeutic protein that is then secreted and enters the general circulation at therapeutic levels, where it can travel to cells/tissues of interest to elicit a therapeutic effect. In contrast to intracellular or membrane proteins, the production of such secreted proteins does not rely on specific disease target cells being transduced, which is a significant advantage and achieves high levels of protein expression. Thus, other diseases which are not respiratory tract diseases, such as cardiovascular diseases and blood disorders can also be treated by the Lentiviral vectors. Lentiviral vectors, such as those according to the invention, can integrate into the genome of transduced cells and lead to long-lasting expression, making them suitable for transduction of stem/progenitor cells.

The US patent application publication, US 2017/0096684—Alton et al., describes in detail the process by which Lentiviral vectors are generated, delivered and their corresponding use for treatment of diseases, the contents of which are incorporated by reference in their entirety herein. The present invention contemplates the use of Lentiviral vectors to deliver nucleotides encoding ENPP1 or ENPP3 to a subject in need thereof and the methods of treatment using the same.

| Sequences | |
|---|---|
| ENPP1 Amino Acid Sequence - Wild Type | |
| SEQ ID NO: 1 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Val Leu Ser Leu | |
| 65 70 75 80 | |
| Val Leu Ser Val Cys Val Leu Thr Thr Ile Leu Gly Cys Ile Phe Gly | |
| 85 90 95 | |
| Leu Lys Pro Ser Cys Ala Lys Glu Val Lys Ser Cys Lys Gly Arg Cys | |
| 100 105 110 | |
| Phe Glu Arg Thr Phe Gly Asn Cys Arg Cys Asp Ala Ala Cys Val Glu | |
| 115 120 125 | |
| Leu Gly Asn Cys Cys Leu Asp Tyr Gln Glu Thr Cys Ile Glu Pro Glu | |
| 130 135 140 | |
| His Ile Trp Thr Cys Asn Lys Phe Arg Cys Gly Glu Lys Arg Leu Thr | |
| 145 150 155 160 | |
| Arg Ser Leu Cys Ala Cys Ser Asp Asp Cys Lys Asp Lys Gly Asp Cys | |
| 165 170 175 | |
| Cys Ile Asn Tyr Ser Ser Val Cys Gln Gly Glu Lys Ser Trp Val Glu | |
| 180 185 190 | |
| Glu Pro Cys Glu Ser Ile Asn Glu Pro Gln Cys Pro Ala Gly Phe Glu | |
| 195 200 205 | |
| Thr Pro Pro Thr Leu Leu Phe Ser Leu Asp Gly Phe Arg Ala Glu Tyr | |
| 210 215 220 | |
| Leu His Thr Trp Gly Gly Leu Leu Pro Val Ile Ser Lys Leu Lys Lys | |
| 225 230 235 240 | |
| Cys Gly Thr Tyr Thr Lys Asn Met Arg Pro Val Tyr Pro Thr Lys Thr | |
| 245 250 255 | |
| Phe Pro Asn His Tyr Ser Ile Val Thr Gly Leu Tyr Pro Glu Ser His | |
| 260 265 270 | |
| Gly Ile Ile Asp Asn Lys Met Tyr Asp Pro Lys Met Asn Ala Ser Phe | |
| 275 280 285 | |
| Ser Leu Lys Ser Lys Glu Lys Phe Asn Pro Glu Trp Tyr Lys Gly Glu | |
| 290 295 300 | |
| Pro Ile Trp Val Thr Ala Lys Tyr Gln Gly Leu Lys Ser Gly Thr Phe | |
| 305 310 315 320 | |
| Phe Trp Pro Gly Ser Asp Val Glu Ile Asn Gly Ile Phe Pro Asp Ile | |
| 325 330 335 | |
| Tyr Lys Met Tyr Asn Gly Ser Val Pro Phe Glu Glu Arg Ile Leu Ala | |
| 340 345 350 | |
| Val Leu Gln Trp Leu Gln Leu Pro Lys Asp Glu Arg Pro His Phe Tyr | |
| 355 360 365 | |
| Thr Leu Tyr Leu Glu Glu Pro Asp Ser Ser Gly His Ser Tyr Gly Pro | |
| 370 375 380 | |
| Val Ser Ser Glu Val Ile Lys Ala Leu Gln Arg Val Asp Gly Met Val | |
| 385 390 395 400 | |
| Gly Met Leu Met Asp Gly Leu Lys Glu Leu Asn Leu His Arg Cys Leu | |
| 405 410 415 | |
| Asn Leu Ile Leu Ile Ser Asp His Gly Met Glu Gln Gly Ser Cys Lys | |
| 420 425 430 | |
| Lys Tyr Ile Tyr Leu Asn Lys Tyr Leu Gly Asp Val Lys Asn Ile Lys | |
| 435 440 445 | |
| Val Ile Tyr Gly Pro Ala Ala Arg Leu Arg Pro Ser Asp Val Pro Asp | |
| 450 455 460 | |
| Lys Tyr Tyr Ser Phe Asn Tyr Glu Gly Ile Ala Arg Asn Leu Ser Cys | |
| 465 470 475 480 | |
| Arg Glu Pro Asn Gln His Phe Lys Pro Tyr Leu Lys His Phe Leu Pro | |
| 485 490 495 | |
| Lys Arg Leu His Phe Ala Lys Ser Asp Arg Ile Glu Pro Leu Thr Phe | |
| 500 505 510 | |
| Tyr Leu Asp Pro Gln Trp Gln Leu Ala Leu Asn Pro Ser Glu Arg Lys | |
| 515 520 525 | |
| Tyr Cys Gly Ser Gly Phe His Gly Ser Asp Asn Val Phe Ser Asn Met | |
| 530 535 540 | |
| Gln Ala Leu Phe Val Gly Tyr Gly Pro Gly Phe Lys His Gly Ile Glu | |
| 545 550 555 560 | |
| Ala Asp Thr Phe Glu Asn Ile Glu Val Tyr Asn Leu Met Cys Asp Leu | |
| 565 570 575 | |
| Leu Asn Leu Thr Pro Ala Pro Asn Asn Gly Thr His Gly Ser Leu Asn | |
| 580 585 590 | |
| His Leu Leu Lys Asn Pro Val Tyr Thr Pro Lys His Pro Lys Glu Val | |
| 595 600 605 | |
| His Pro Leu Val Gln Cys Pro Phe Thr Arg Asn Pro Arg Asp Asn Leu | |
| 610 615 620 | |
| Gly Cys Ser Cys Asn Pro Ser Ile Leu Pro Ile Glu Asp Phe Gln Thr | |
| 625 630 635 640 | |
| Gln Phe Asn Leu Thr Val Ala Glu Glu Lys Ile Ile Lys His Glu Thr | |
| 645 650 655 | |
| Leu Pro Tyr Gly Arg Pro Arg Val Leu Gln Lys Glu Asn Thr Ile Cys | |
| 660 665 670 | |
| Leu Leu Ser Gln His Gln Phe Met Ser Gly Tyr Ser Gln Asp Ile Leu | |
| 675 680 685 | |
| Met Pro Leu Trp Thr Ser Tyr Thr Val Asp Arg Asn Asp Ser Phe Ser | |
| 690 695 700 | |
| Thr Glu Asp Phe Ser Asn Cys Leu Tyr Gln Asp Phe Arg Ile Pro Leu | |
| 705 710 715 720 | |
| Ser Pro Val His Lys Cys Ser Phe Tyr Lys Asn Asn Thr Lys Val Ser | |
| 725 730 735 | |
| Tyr Gly Phe Leu Ser Pro Pro Gln Leu Asn Lys Asn Ser Ser Gly Ile | |
| 740 745 750 | |
| Tyr Ser Glu Ala Leu Leu Thr Thr Asn Ile Val Pro Met Tyr Gln Ser | |
| 755 760 765 | |
| Phe Gln Val Ile Trp Arg Tyr Phe His Asp Thr Leu Leu Arg Lys Tyr | |
| 770 775 780 | |
| Ala Glu Glu Arg Asn Gly Val Asn Val Val Ser Gly Pro Val Phe Asp | |
| 785 790 795 800 | |
| Phe Asp Tyr Asp Gly Arg Cys Asp Ser Leu Glu Asn Leu Arg Gln Lys | |
| 805 810 815 | |
| Arg Arg Val Ile Arg Asn Gln Glu Ile Leu Ile Pro Thr His Phe Phe | |
| 820 825 830 | |
| Ile Val Leu Thr Ser Cys Lys Asp Thr Ser Gln Thr Pro Leu His Cys | |
| 835 840 845 | |
| Glu Asn Leu Asp Thr Leu Ala Phe Ile Leu Pro His Arg Thr Asp Asn | |
| 850 855 860 | |
| Ser Glu Ser Cys Val His Gly Lys His Asp Ser Ser Trp Val Glu Glu | |
| 865 870 875 880 | |
| Leu Leu Met Leu His Arg Ala Arg Ile Thr Asp Val Glu His Ile Thr | |
| 885 890 895 | |
| Gly Leu Ser Phe Tyr Gln Gln Arg Lys Glu Pro Val Ser Asp Ile Leu | |
| 900 905 910 | |
| Lys Leu Lys Thr His Leu Pro Thr Phe Ser Gln Glu Asp | |
| 915 920 925 | |
| NPP1 amino acid sequence shown above comprises cytoplasmic domain, | |
| transmembrane domain, SMB1 domain, SMB2 domain, phosphodiesterase/ | |
| catalytic domain, linker domain and nuclease domain. The SMB1 domain, | |
| SMB2 domain, catalytic domain, linker domain and the nuclease domain | |
| are jointly referred to as the extracellular domain. Residues 1-76 | |
| (Met Glu Arg to Thr Tyr Lys) correspond to the cytoplasmic domain. | |
| Residues 77-97 (Val Leu Ser to Phe Gly Leu) correspond to the trans- | |
| membrane domain. Residues 99-925 (Pro Ser Cys to Gln Glu Asp) | |
| correspond to the extracellular domain. Residues 104-144 (Glu Val | |
| Lys to Glu Pro Glu) correspond to SMB1 domain and residues 145-189 | |
| (His Ile Trp to Glu Lys Ser) correspond to SMB2 domain. Residues | |
| 597-647 correspond to linker domain that connects catalytic and | |
| nuclease domains. Residues 191-591 (Val Glu Glu to Gly Ser Leu) | |
| correspond to the catalytic/phosphodiesterase domain. Residues | |
| 654-925 (His Glu Thr to Gln Glu Asp) correspond to the nuclease | |
| domain. The residue numbering and domain classification are based on | |
| human NPP1 sequence (NCBI accession NP_006199/Uniprot-Swissprot | |
| P22413) | |
| Azurocidin-ENPP1-FC | |
| SEQ ID NO: 2 | |
| MTRLTVLALLAGLLASSRA**APSCAKEVKSCKGRCFERTEGNCRCDAACVELGNCCLDYQETCIEPEHI | |
| WTCNKFRCGEKRLTRSLCACSDDCKDKGDCCINYSSVCQGEKSWVEEPCESINEPQCPAGFETPPTLLFS | |
| LDGFRAEYLHIWGGLLPVISKLKKCGTYTKNMRPVYPIKTFPNHYSIVTGLYPESHGIIDNKMYDPKMNA | |
| SFSLKSKEKFNPEWYKGEPIWVTAKYQGLKSGTFFWPGSDVEINGIFPDIYKMYNGSVPFEERILAVLQW | |
| LQLPKDERPHFYTLYLEEPDSSGHSYGPVSSEVIKALQRVDGMVGMLMDGLKELNLHRCLNLILISDHGM | |
| EQGSCKKYTYLNKYLGDVKNIKVIYGPAARLRPSDVPDKYYSFNYEGIARNLSCREPNQHFKPYLKHFLP | |
| KRLHFAKSDRIEPLTFYLDPQWQLALNPSERKYCGSGFHGSDNVFSNMQALFVGYGPGFKHGIEADTFEN | |
| IEVYNLMCDLLNLTPAPNNGTHGSLNHLLKNPVYTPKHPKEVHPLVQCPFTRNPRDNLGCSCNPSILPIE | |
| DFQTQFNLTVAEEKIIKHETLPYGRPRVLQKENTICLLSQHQFMSGYSQDILMPLWTSYTVDRNDSFSTE | |
| DFSNCLYQDFRIPLSPVHKCSFYKNNTKVSYGFLSPPQLNKNSSGIYSEALLTTNIVPMYQSFQVIWRYF | |
| HDTLLRKYAEERNGVNVVSGPVFDFDYDGRCDSLENLRQKRRVIRNQEILIPTHFFIVLTSCKDTSQTPL | |
| HCENLDTLAFILPHRTDNSESCVHGKHDSSWVEELLMLHRARITDVEHITGLSFYQQRKEPVSDILKLKT | |
| HLPTFSQEDLINDKTHTCPPCPAPELLGGPSVFLEPPKPKDTLMISRTPEVTCVVVDVSHEDPEVKFNWY | |
| VDGVEVHNAKTKPREEQYNSTYRVVSVLTVLHQDWLNGKEYKCKVSNKALPAPIEKTISKAKGQPREPQV | |
| YTLPPSREEMTKNQVSLTCLVKGFYPSDIAVEWESNGQPENNYKTTPPVLDSDGSFFLYSKLTVDKSRWQ | |
| QGNVFSCSVMHEALHNHYTQKSLSLSPGK | |
| Single underline - Azurocidin signal sequence, Double underline - | |
| Beginning and end of ENPP1 sequence, Bold residues - Fc sequence, ** | |
| indicates the cleavage point of the signal sequence. | |
| Azurocidin-ENPP1-Alb | |
| SEQ ID NO: 3 | |
| MTRLTVLALLAGLLASSRA**APSCAKEVKSCKGRCFERTEGNCRCDAACVELGNCCLDYQETCIEPEHI | |
| WTCNKFRCGEKRLTRSLCACSDDCKDKGDCCINYSSVCQGEKSWVEEPCESINEPQCPAGFETPPTLLFS | |
| LDGFRAEYLHIWGGLLPVISKLKKCGTYTKNMRPVYPIKTFPNHYSIVTGLYPESHGIIDNKMYDPKMNA | |
| SFSLKSKEKFNPEWYKGEPIWVTAKYQGLKSGTFFWPGSDVEINGIFPDIYKMYNGSVPFEERILAVLQW | |
| LQLPKDERPHFYTLYLEEPDSSGHSYGPVSSEVIKALQRVDGMVGMLMDGLKELNLHRCLNLILISDHGM | |
| EQGSCKKYTYLNKYLGDVKNIKVIYGPAARLRPSDVPDKYYSFNYEGIARNLSCREPNQHFKPYLKHFLP | |
| KRLHFAKSDRIEPLTFYLDPQWQLALNPSERKYCGSGFHGSDNVFSNMQALFVGYGPGFKHGIEADTFEN | |
| IEVYNLMCDLLNLTPAPNNGTHGSLNHLLKNPVYTPKHPKEVHPLVQCPFTRNPRDNLGCSCNPSILPIE | |
| DFQTQFNLTVAEEKIIKHETLPYGRPRVLQKENTICLLSQHQFMSGYSQDILMPLWTSYTVDRNDSFSTE | |
| DFSNCLYQDFRIPLSPVHKCSFYKNNTKVSYGFLSPPQLNKNSSGIYSEALLTTNIVPMYQSFQVIWRYF | |
| HDTLLRKYAEERNGVNVVSGPVFDFDYDGRCDSLENLRQKRRVIRNQEILIPTHFFIVLTSCKDTSQTPL | |
| HCENLDTLAFILPHRTDNSESCVHGKHDSSWVEELLMLHRARITDVEHITGLSFYQQRKEPVSDILKLKT | |
| HLPTFSQEDLINMKWVTELLLLEVSGSAFSRGVERREAHKSEIAHRYNDLGEQHFKGLVLIAFSQYLQKC | |
| SYDEHAKLVQEVTDFAKTCVADESAANCDKSLHTLFGDKLCAIPNLRENYGELADCCTKQEPERNECFLQ | |
| HKDDNPSLPPFERPEAEAMCTSFKENPTTFMGHYLHEVARRHPYFYAPELLYYAEQYNEILTQCCAEADK | |
| ESCLTPKLDGVKEKALVSSVRQRMKCSSMQKFGERAFKAWAVARLSQTFPNADFAEITKLATDLTKVNKE | |
| CCHGDLLECADDRAELAKYMCENQATISSKLQTCCDKPLLKKAHCLSEVEHDTMPADLPAIAADFVEDQE | |
| VCKNYAEAEDVFLGTFLYEYSRRHPDYSVSLLLRLAKKYEATLEKCCAEANPPACYGTVLAEFQPLVEEP | |
| KNLVKTNCDLYEKLGEYGFQNAILVRYTQKAPQVSTPTLVEAARNLGRVGTKCCTLPEDQRLPCVEDYLS | |
| AILNRVCLLHEKTPVSEHVTKCCSGSLVERRPCFSALTVDETYVPKEFKAETFTEHSDICTLPEKEKQIK | |
| KQTALAELVKHKPKATAEQLKTVMDDFAQFLDTCCKAADKDTCFSTEGPNLVTRCKDALARSWSHPQFEK | |
| Single underline - Azurocidin signal sequence, Double underline - | |
| Beginning and end of ENPP1 sequence, Bold residues - Albumin sequence, | |
| ** indicates the cleavage point of the signal sequence. | |
| Azurocidin-ENPP1 | |
| SEQ ID NO: 4 | |
| MTRLTVLALLAGLLASSRA**APSCAKEVKSCKGRCFERTEGNCRCDAACVELGNCCLDYQETCIEPEHI | |
| WTCNKFRCGEKRLTRSLCACSDDCKDKGDCCINYSSVCQGEKSWVEEPCESINEPQCPAGFETPPTLLFS | |
| LDGFRAEYLHIWGGLLPVISKLKKCGTYTKNMRPVYPIKTFPNHYSIVTGLYPESHGIIDNKMYDPKMNA | |
| SFSLKSKEKFNPEWYKGEPIWVTAKYQGLKSGTFFWPGSDVEINGIFPDIYKMYNGSVPFEERILAVLQW | |
| LQLPKDERPHFYTLYLEEPDSSGHSYGPVSSEVIKALQRVDGMVGMLMDGLKELNLHRCLNLILISDHGM | |
| EQGSCKKYTYLNKYLGDVKNIKVIYGPAARLRPSDVPDKYYSFNYEGIARNLSCREPNQHFKPYLKHFLP | |
| KRLHFAKSDRIEPLTFYLDPQWQLALNPSERKYCGSGFHGSDNVFSNMQALFVGYGPGFKHGIEADTFEN | |
| IEVYNLMCDLLNLTPAPNNGTHGSLNHLLKNPVYTPKHPKEVHPLVQCPFTRNPRDNLGCSCNPSILPIE | |
| DFQTQFNLTVAEEKIIKHETLPYGRPRVLQKENTICLLSQHQFMSGYSQDILMPLWTSYTVDRNDSFSTE | |
| DFSNCLYQDFRIPLSPVHKCSFYKNNTKVSYGFLSPPQLNKNSSGIYSEALLTTNIVPMYQSFQVIWRYF | |
| HDTLLRKYAEERNGVNVVSGPVFDFDYDGRCDSLENLRQKRRVIRNQEILIPTHFFIVLTSCKDTSQTAP | |
| SCAKEVKSCKGRCFERTFGNCRCDAACVELGNCCLDYQETCIEPEHIWTCNKFRCGEKRLTRSLCACSDD | |
| CKDKGDCCINYSSVCQGEKSWVEEPCESINEPQCPAGFETPPTLLFSLDGFRAEYLHTWGGLLPVISKLK | |
| KCGTYTKNMRPVYPTKTFPNHYSIVTGLYPESHGIIDNKMYDPKMNASFSLKSKEKFNPEWYKGEPIWVT | |
| AKYQGLKSGTFFWPGSDVEINGIFPDIYKMYNGSVPFEERILAVLQWLQLPKDERPHFYTLYLEEPDSSG | |
| HSYGPVSSEVIKALQRVDGMVGMLMDGLKELNLHRCLNLILISDHGMEQGSCKKYTYLNKYLGDVKNIKV | |
| IYGPAARLRPSDVPDKYYSFNYEGIARNLSCREPNQHFKPYLKHFLPKRLHFAKSDRIEPLTFYLDPQWQ | |
| LALNPSERKYCGSGFHGSDNVFSNMQALFVGYGPGFKHGIEADTFENIEVYNLMCDLLNLTPAPNNGTHG | |
| SLNHLLKNPVYTPKHPKEVHPLVQCPFTRNPRDNLGCSCNPSILPIEDFQTQFNLTVAEEKIIKHETLPY | |
| GRPRVLQKENTICLLSQHQFMSGYSQDILMPLWTSYTVDRNDSFSTEDFSNCLYQDFRIPLSPVHKCSFY | |
| KNNTKVSYGELSPPQLNKNSSGIYSEALLTTNIVPMYQSFQVIWRYFHDTLLRKYAEERNGVNVVSGPVF | |
| DFDYDGRCDSLENLRQKRRVIRNQEILIPTHFFIVLTSCKDTSQTPLHCENLDTLAFILPHRTDNSESCV | |
| HGKHDSSWVEELLMLHRARITDVEHITGLSFYQQRKEPVSDILKLKTHLPTESQED | |
| Single underline - Azurocidin signal sequence, Double underline - | |
| Beginning and end of ENPP1 sequence, ** indicates the cleavage point | |
| of the signal sequence. | |
| ENPP2 Amino Acid Sequence - Wild Type | |
| SEQ ID NO: 5 | |
| Met Ala Arg Arg Ser Ser Phe Gln Ser Cys Gln Ile Ile Ser Leu Phe | |
| 1 5 10 15 | |
| Thr Phe Ala Val Gly Val Asn Ile Cys Leu Gly Phe Thr Ala His Arg | |
| 20 25 30 | |
| Ile Lys Arg Ala Glu Gly Trp Glu Glu Gly Pro Pro Thr Val Leu Ser | |
| 35 40 45 | |
| Asp Ser Pro Trp Thr Asn Ile Ser Gly Ser Cys Lys Gly Arg Cys Phe | |
| 50 55 60 | |
| Glu Leu Gln Glu Ala Gly Pro Pro Asp Cys Arg Cys Asp Asn Leu Cys | |
| 65 70 75 80 | |
| Lys Ser Tyr Thr Ser Cys Cys His Asp Phe Asp Glu Leu Cys Leu Lys | |
| 85 90 95 | |
| Thr Ala Arg Gly Trp Glu Cys Thr Lys Asp Arg Cys Gly Glu Val Arg | |
| 100 105 110 | |
| Asn Glu Glu Asn Ala Cys His Cys Ser Glu Asp Cys Leu Ala Arg Gly | |
| 115 120 125 | |
| Asp Cys Cys Thr Asn Tyr Gln Val Val Cys Lys Gly Glu Ser His Trp | |
| 130 135 140 | |
| Val Asp Asp Asp Cys Glu Glu Ile Lys Ala Ala Glu Cys Pro Ala Gly | |
| 145 150 155 160 | |
| Phe Val Arg Pro Pro Leu Ile Ile Phe Ser Val Asp Gly Phe Arg Ala | |
| 165 170 175 | |
| Ser Tyr Met Lys Lys Gly Ser Lys Val Met Pro Asn Ile Glu Lys Leu | |
| 180 185 190 | |
| Arg Ser Cys Gly Thr His Ser Pro Tyr Met Arg Pro Val Tyr Pro Thr | |
| 195 200 205 | |
| Lys Thr Phe Pro Asn Leu Tyr Thr Leu Ala Thr Gly Leu Tyr Pro Glu | |
| 210 215 220 | |
| Ser His Gly Ile Val Gly Asn Ser Met Tyr Asp Pro Val Phe Asp Ala | |
| 225 230 235 240 | |
| Thr Phe His Leu Arg Gly Arg Glu Lys Phe Asn His Arg Trp Trp Gly | |
| 245 250 255 | |
| Gly Gln Pro Leu Trp Ile Thr Ala Thr Lys Gln Gly Val Lys Ala Gly | |
| 260 265 270 | |
| Thr Phe Phe Trp Ser Val Val Ile Pro His Glu Arg Arg Ile Leu Thr | |
| 275 280 285 | |
| Ile Leu Gln Trp Leu Thr Leu Pro Asp His Glu Arg Pro Ser Val Tyr | |
| 290 295 300 | |
| Ala Phe Tyr Ser Glu Gln Pro Asp Phe Ser Gly His Lys Tyr Gly Pro | |
| 305 310 315 320 | |
| Phe Gly Pro Glu Met Thr Asn Pro Leu Arg Glu Ile Asp Lys Ile Val | |
| 325 330 335 | |
| Gly Gln Leu Met Asp Gly Leu Lys Gln Leu Lys Leu His Arg Cys Val | |
| 340 345 350 | |
| Asn Val Ile Phe Val Gly Asp His Gly Met Glu Asp Val Thr Cys Asp | |
| 355 360 365 | |
| Arg Thr Glu Phe Leu Ser Asn Tyr Leu Thr Asn Val Asp Asp Ile Thr | |
| 370 375 380 | |
| Leu Val Pro Gly Thr Leu Gly Arg Ile Arg Ser Lys Phe Ser Asn Asn | |
| 385 390 395 400 | |
| Ala Lys Tyr Asp Pro Lys Ala Ile Ile Ala Asn Leu Thr Cys Lys Lys | |
| 405 410 415 | |
| Pro Asp Gln His Phe Lys Pro Tyr Leu Lys Gln His Leu Pro Lys Arg | |
| 420 425 430 | |
| Leu His Tyr Ala Asn Asn Arg Arg Ile Glu Asp Ile His Leu Leu Val | |
| 435 440 445 | |
| Glu Arg Arg Trp His Val Ala Arg Lys Pro Leu Asp Val Tyr Lys Lys | |
| 450 455 460 | |
| Pro Ser Gly Lys Cys Phe Phe Gln Gly Asp His Gly Phe Asp Asn Lys | |
| 465 470 475 480 | |
| Val Asn Ser Met Gln Thr Val Phe Val Gly Tyr Gly Ser Thr Phe Lys | |
| 485 490 495 | |
| Tyr Lys Thr Lys Val Pro Pro Phe Glu Asn Ile Glu Leu Tyr Asn Val | |
| 500 505 510 | |
| Met Cys Asp Leu Leu Gly Leu Lys Pro Ala Pro Asn Asn Gly Thr His | |
| 515 520 525 | |
| Gly Ser Leu Asn His Leu Leu Arg Thr Asn Thr Phe Arg Pro Thr Met | |
| 530 535 540 | |
| Pro Glu Glu Val Thr Arg Pro Asn Tyr Pro Gly Ile Met Tyr Leu Gln | |
| 545 550 555 560 | |
| Ser Asp Phe Asp Leu Gly Cys Thr Cys Asp Asp Lys Val Glu Pro Lys | |
| 565 570 575 | |
| Asn Lys Leu Asp Glu Leu Asn Lys Arg Leu His Thr Lys Gly Ser Thr | |
| 580 585 590 | |
| Glu Ala Glu Thr Arg Lys Phe Arg Gly Ser Arg Asn Glu Asn Lys Glu | |
| 595 600 605 | |
| Asn Ile Asn Gly Asn Phe Glu Pro Arg Lys Glu Arg His Leu Leu Tyr | |
| 610 615 620 | |
| Gly Arg Pro Ala Val Leu Tyr Arg Thr Arg Tyr Asp Ile Leu Tyr His | |
| 625 630 635 640 | |
| Thr Asp Phe Glu Ser Gly Tyr Ser Glu Ile Phe Leu Met Pro Leu Trp | |
| 645 650 655 | |
| Thr Ser Tyr Thr Val Ser Lys Gln Ala Glu Val Ser Ser Val Pro Asp | |
| 660 665 670 | |
| His Leu Thr Ser Cys Val Arg Pro Asp Val Arg Val Ser Pro Ser Phe | |
| 675 680 685 | |
| Ser Gln Asn Cys Leu Ala Tyr Lys Asn Asp Lys Gln Met Ser Tyr Gly | |
| 690 695 700 | |
| Phe Leu Phe Pro Pro Tyr Leu Ser Ser Ser Pro Glu Ala Lys Tyr Asp | |
| 705 710 715 720 | |
| Ala Phe Leu Val Thr Asn Met Val Pro Met Tyr Pro Ala Phe Lys Arg | |
| 725 730 735 | |
| Val Trp Asn Tyr Phe Gln Arg Val Leu Val Lys Lys Tyr Ala Ser Glu | |
| 740 745 750 | |
| Arg Asn Gly Val Asn Val Ile Ser Gly Pro Ile Phe Asp Tyr Asp Tyr | |
| 755 760 765 | |
| Asp Gly Leu His Asp Thr Glu Asp Lys Ile Lys Gln Tyr Val Glu Gly | |
| 770 775 780 | |
| Ser Ser Ile Pro Val Pro Thr His Tyr Tyr Ser Ile Ile Thr Ser Cys | |
| 785 790 795 800 | |
| Leu Asp Phe Thr Gln Pro Ala Asp Lys Cys Asp Gly Pro Leu Ser Val | |
| 805 810 815 | |
| Ser Ser Phe Ile Leu Pro His Arg Pro Asp Asn Glu Glu Ser Cys Asn | |
| 820 825 830 | |
| Ser Ser Glu Asp Glu Ser Lys Trp Val Glu Glu Leu Met Lys Met His | |
| 835 840 845 | |
| Thr Ala Arg Val Arg Asp Ile Glu His Leu Thr Ser Leu Asp Phe Phe | |
| 850 855 860 | |
| Arg Lys Thr Ser Arg Ser Tyr Pro Glu Ile Leu Thr Leu Lys Thr Tyr | |
| 865 870 875 880 | |
| Leu His Thr Tyr Glu Ser Glu Ile | |
| 885 | |
| Extracellular Domain of ENPP3: | |
| SEQ. ID NO: 6 | |
| Glu Lys Gln Gly Ser Cys Arg Lys Lys Cys Phe Asp Ala Ser Phe Arg | |
| 1 5 10 15 | |
| Gly Leu Glu Asn Cys Arg Cys Asp Val Ala Cys Lys Asp Arg Gly Asp | |
| 20 25 30 | |
| Cys Cys Trp Asp Phe Glu Asp Thr Cys Val Glu Ser Thr Arg Ile Trp | |
| 35 40 45 | |
| Met Cys Asn Lys Phe Arg Cys Gly Glu Thr Arg Leu Glu Ala Ser Leu | |
| 50 55 60 | |
| Cys Ser Cys Ser Asp Asp Cys Leu Gln Arg Lys Asp Cys Cys Ala Asp | |
| 65 70 75 80 | |
| Tyr Lys Ser Val Cys Gln Gly Glu Thr Ser Trp Leu Glu Glu Asn Cys | |
| 85 90 95 | |
| Asp Thr Ala Gln Gln Ser Gln Cys Pro Glu Gly Phe Asp Leu Pro Pro | |
| 100 105 110 | |
| Val Ile Leu Phe Ser Met Asp Gly Phe Arg Ala Glu Tyr Leu Tyr Thr | |
| 115 120 125 | |
| Trp Asp Thr Leu Met Pro Asn Ile Asn Lys Leu Lys Thr Cys Gly Ile | |
| 130 135 140 | |
| His Ser Lys Tyr Met Arg Ala Met Tyr Pro Thr Lys Thr Phe Pro Asn | |
| 145 150 155 160 | |
| His Tyr Thr Ile Val Thr Gly Leu Tyr Pro Glu Ser His Gly Ile Ile | |
| 165 170 175 | |
| Asp Asn Asn Met Tyr Asp Val Asn Leu Asn Lys Asn Phe Ser Leu Ser | |
| 180 185 190 | |
| Ser Lys Glu Gln Asn Asn Pro Ala Trp Trp His Gly Gln Pro Met Trp | |
| 195 200 205 | |
| Leu Thr Ala Met Tyr Gln Gly Leu Lys Ala Ala Thr Tyr Phe Trp Pro | |
| 210 215 220 | |
| Gly Ser Glu Val Ala Ile Asn Gly Ser Phe Pro Ser Ile Tyr Met Pro | |
| 225 230 235 240 | |
| Tyr Asn Gly Ser Val Pro Phe Glu Glu Arg Ile Ser Thr Leu Leu Lys | |
| 245 250 255 | |
| Trp Leu Asp Leu Pro Lys Ala Glu Arg Pro Arg Phe Tyr Thr Met Tyr | |
| 260 265 270 | |
| Phe Glu Glu Pro Asp Ser Ser Gly His Ala Gly Gly Pro Val Ser Ala | |
| 275 280 285 | |
| Arg Val Ile Lys Ala Leu Gln Val Val Asp His Ala Phe Gly Met Leu | |
| 290 295 300 | |
| Met Glu Gly Leu Lys Gln Arg Asn Leu His Asn Cys Val Asn Ile Ile | |
| 305 310 315 320 | |
| Leu Leu Ala Asp His Gly Met Asp Gln Thr Tyr Cys Asn Lys Met Glu | |
| 325 330 335 | |
| Tyr Met Thr Asp Tyr Phe Pro Arg Ile Asn Phe Phe Tyr Met Tyr Glu | |
| 340 345 350 | |
| Gly Pro Ala Pro Arg Ile Arg Ala His Asn Ile Pro His Asp Phe Phe | |
| 355 360 365 | |
| Ser Phe Asn Ser Glu Glu Ile Val Arg Asn Leu Ser Cys Arg Lys Pro | |
| 370 375 380 | |
| Asp Gln His Phe Lys Pro Tyr Leu Thr Pro Asp Leu Pro Lys Arg Leu | |
| 385 390 395 400 | |
| His Tyr Ala Lys Asn Val Arg Ile Asp Lys Val His Leu Phe Val Asp | |
| 405 410 415 | |
| Gln Gln Trp Leu Ala Val Arg Ser Lys Ser Asn Thr Asn Cys Gly Gly | |
| 420 425 430 | |
| Gly Asn His Gly Tyr Asn Asn Glu Phe Arg Ser Met Glu Ala Ile Phe | |
| 435 440 445 | |
| Leu Ala His Gly Pro Ser Phe Lys Glu Lys Thr Glu Val Glu Pro Phe | |
| 450 455 460 | |
| Glu Asn Ile Glu Val Tyr Asn Leu Met Cys Asp Leu Leu Arg Ile Gln | |
| 465 470 475 480 | |
| Pro Ala Pro Asn Asn Gly Thr His Gly Ser Leu Asn His Leu Leu Lys | |
| 485 490 495 | |
| Val Pro Phe Tyr Glu Pro Ser His Ala Glu Glu Val Ser Lys Phe Ser | |
| 500 505 510 | |
| Val Cys Gly Phe Ala Asn Pro Leu Pro Thr Glu Ser Leu Asp Cys Phe | |
| 515 520 525 | |
| Cys Pro His Leu Gln Asn Ser Thr Gln Leu Glu Gln Val Asn Gln Met | |
| 530 535 540 | |
| Leu Asn Leu Thr Gln Glu Glu Ile Thr Ala Thr Val Lys Val Asn Leu | |
| 545 550 555 560 | |
| Pro Phe Gly Arg Pro Arg Val Leu Gln Lys Asn Val Asp His Cys Leu | |
| 565 570 575 | |
| Leu Tyr His Arg Glu Tyr Val Ser Gly Phe Gly Lys Ala Met Arg Met | |
| 580 585 590 | |
| Pro Met Trp Ser Ser Tyr Thr Val Pro Gln Leu Gly Asp Thr Ser Pro | |
| 595 600 605 | |
| Leu Pro Pro Thr Val Pro Asp Cys Leu Arg Ala Asp Val Arg Val Pro | |
| 610 615 620 | |
| Pro Ser Glu Ser Gln Lys Cys Ser Phe Tyr Leu Ala Asp Lys Asn Ile | |
| 625 630 635 640 | |
| Thr His Gly Phe Leu Tyr Pro Pro Ala Ser Asn Arg Thr Ser Asp Ser | |
| 645 650 655 | |
| Gln Tyr Asp Ala Leu Ile Thr Ser Asn Leu Val Pro Met Tyr Glu Glu | |
| 660 665 670 | |
| Phe Arg Lys Met Trp Asp Tyr Phe His Ser Val Leu Leu Ile Lys His | |
| 675 680 685 | |
| Ala Thr Glu Arg Asn Gly Val Asn Val Val Ser Gly Pro Ile Phe Asp | |
| 690 695 700 | |
| Tyr Asn Tyr Asp Gly His Phe Asp Ala Pro Asp Glu Ile Thr Lys His | |
| 705 710 715 720 | |
| Leu Ala Asn Thr Asp Val Pro Ile Pro Thr His Tyr Phe Val Val Leu | |
| 725 730 735 | |
| Thr Ser Cys Lys Asn Lys Ser His Thr Pro Glu Asn Cys Pro Gly Trp | |
| 740 745 750 | |
| Leu Asp Val Leu Pro Phe Ile Ile Pro His Arg Pro Thr Asn Val Glu | |
| 755 760 765 | |
| Ser Cys Pro Glu Gly Lys Pro Glu Ala Leu Trp Val Glu Glu Arg Phe | |
| 770 775 780 | |
| Thr Ala His Ile Ala Arg Val Arg Asp Val Glu Leu Leu Thr Gly Leu | |
| 785 790 795 800 | |
| Asp Phe Tyr Gln Asp Lys Val Gln Pro Val Ser Glu Ile Leu Gln Leu | |
| 805 810 815 | |
| Lys Thr Tyr Leu Pro Thr Phe Glu Thr Thr Ile | |
| 820 825 | |
| NPP3 Amino Acid Sequence: | |
| SEQ. ID NO: 7 | |
| Met Glu Ser Thr Leu Thr Leu Ala Thr Glu Gln Pro Val Lys Lys Asn | |
| 1 5 10 15 | |
| Thr Leu Lys Lys Tyr Lys Ile Ala Cys Ile Val Leu Leu Ala Leu Leu | |
| 20 25 30 | |
| Val Ile Met Ser Leu Gly Leu Gly Leu Gly Leu Gly Leu Arg Lys Leu | |
| 35 40 45 | |
| Glu Lys Gln Gly Ser Cys Arg Lys Lys Cys Phe Asp Ala Ser Phe Arg | |
| 50 55 60 | |
| Gly Leu Glu Asn Cys Arg Cys Asp Val Ala Cys Lys Asp Arg Gly Asp | |
| 65 70 75 80 | |
| Cys Cys Trp Asp Phe Glu Asp Thr Cys Val Glu Ser Thr Arg Ile Trp | |
| 85 90 95 | |
| Met Cys Asn Lys Phe Arg Cys Gly Glu Thr Arg Leu Glu Ala Ser Leu | |
| 100 105 110 | |
| Cys Ser Cys Ser Asp Asp Cys Leu Gln Arg Lys Asp Cys Cys Ala Asp | |
| 115 120 125 | |
| Tyr Lys Ser Val Cys Gln Gly Glu Thr Ser Trp Leu Glu Glu Asn Cys | |
| 130 135 140 | |
| Asp Thr Ala Gln Gln Ser Gln Cys Pro Glu Gly Phe Asp Leu Pro Pro | |
| 145 150 155 160 | |
| Val Ile Leu Phe Ser Met Asp Gly Phe Arg Ala Glu Tyr Leu Tyr Thr | |
| 165 170 175 | |
| Trp Asp Thr Leu Met Pro Asn Ile Asn Lys Leu Lys Thr Cys Gly Ile | |
| 180 185 190 | |
| His Ser Lys Tyr Met Arg Ala Met Tyr Pro Thr Lys Thr Phe Pro Asn | |
| 195 200 205 | |
| His Tyr Thr Ile Val Thr Gly Leu Tyr Pro Glu Ser His Gly Ile Ile | |
| 210 215 220 | |
| Asp Asn Asn Met Tyr Asp Val Asn Leu Asn Lys Asn Phe Ser Leu Ser | |
| 225 230 235 240 | |
| Ser Lys Glu Gln Asn Asn Pro Ala Trp Trp His Gly Gln Pro Met Trp | |
| 245 250 255 | |
| Leu Thr Ala Met Tyr Gln Gly Leu Lys Ala Ala Thr Tyr Phe Trp Pro | |
| 260 265 270 | |
| Gly Ser Glu Val Ala Ile Asn Gly Ser Phe Pro Ser Ile Tyr Met Pro | |
| 275 280 285 | |
| Tyr Asn Gly Ser Val Pro Phe Glu Glu Arg Ile Ser Thr Leu Leu Lys | |
| 290 295 300 | |
| Trp Leu Asp Leu Pro Lys Ala Glu Arg Pro Arg Phe Tyr Thr Met Tyr | |
| 305 310 315 320 | |
| Phe Glu Glu Pro Asp Ser Ser Gly His Ala Gly Gly Pro Val Ser Ala | |
| 325 330 335 | |
| Arg Val Ile Lys Ala Leu Gln Val Val Asp His Ala Phe Gly Met Leu | |
| 340 345 350 | |
| Met Glu Gly Leu Lys Gln Arg Asn Leu His Asn Cys Val Asn Ile Ile | |
| 355 360 365 | |
| Leu Leu Ala Asp His Gly Met Asp Gln Thr Tyr Cys Asn Lys Met Glu | |
| 370 375 380 | |
| Tyr Met Thr Asp Tyr Phe Pro Arg Ile Asn Phe Phe Tyr Met Tyr Glu | |
| 385 390 395 400 | |
| Gly Pro Ala Pro Arg Ile Arg Ala His Asn Ile Pro His Asp Phe Phe | |
| 405 410 415 | |
| Ser Phe Asn Ser Glu Glu Ile Val Arg Asn Leu Ser Cys Arg Lys Pro | |
| 420 425 430 | |
| Asp Gln His Phe Lys Pro Tyr Leu Thr Pro Asp Leu Pro Lys Arg Leu | |
| 435 440 445 | |
| His Tyr Ala Lys Asn Val Arg Ile Asp Lys Val His Leu Phe Val Asp | |
| 450 455 460 | |
| Gln Gln Trp Leu Ala Val Arg Ser Lys Ser Asn Thr Asn Cys Gly Gly | |
| 465 470 475 480 | |
| Gly Asn His Gly Tyr Asn Asn Glu Phe Arg Ser Met Glu Ala Ile Phe | |
| 485 490 495 | |
| Leu Ala His Gly Pro Ser Phe Lys Glu Lys Thr Glu Val Glu Pro Phe | |
| 500 505 510 | |
| Glu Asn Ile Glu Val Tyr Asn Leu Met Cys Asp Leu Leu Arg Ile Gln | |
| 515 520 525 | |
| Pro Ala Pro Asn Asn Gly Thr His Gly Ser Leu Asn His Leu Leu Lys | |
| 530 535 540 | |
| Val Pro Phe Tyr Glu Pro Ser His Ala Glu Glu Val Ser Lys Phe Ser | |
| 545 550 555 560 | |
| Val Cys Gly Phe Ala Asn Pro Leu Pro Thr Glu Ser Leu Asp Cys Phe | |
| 565 570 575 | |
| Cys Pro His Leu Gln Asn Ser Thr Gln Leu Glu Gln Val Asn Gln Met | |
| 580 585 590 | |
| Leu Asn Leu Thr Gln Glu Glu Ile Thr Ala Thr Val Lys Val Asn Leu | |
| 595 600 605 | |
| Pro Phe Gly Arg Pro Arg Val Leu Gln Lys Asn Val Asp His Cys Leu | |
| 610 615 620 | |
| Leu Tyr His Arg Glu Tyr Val Ser Gly Phe Gly Lys Ala Met Arg Met | |
| 625 630 635 640 | |
| Pro Met Trp Ser Ser Tyr Thr Val Pro Gln Leu Gly Asp Thr Ser Pro | |
| 645 650 655 | |
| Leu Pro Pro Thr Val Pro Asp Cys Leu Arg Ala Asp Val Arg Val Pro | |
| 660 665 670 | |
| Pro Ser Glu Ser Gln Lys Cys Ser Phe Tyr Leu Ala Asp Lys Asn Ile | |
| 675 680 685 | |
| Thr His Gly Phe Leu Tyr Pro Pro Ala Ser Asn Arg Thr Ser Asp Ser | |
| 690 695 700 | |
| Gln Tyr Asp Ala Leu Ile Thr Ser Asn Leu Val Pro Met Tyr Glu Glu | |
| 705 710 715 720 | |
| Phe Arg Lys Met Trp Asp Tyr Phe His Ser Val Leu Leu Ile Lys His | |
| 725 730 735 | |
| Ala Thr Glu Arg Asn Gly Val Asn Val Val Ser Gly Pro Ile Phe Asp | |
| 740 745 750 | |
| Tyr Asn Tyr Asp Gly His Phe Asp Ala Pro Asp Glu Ile Thr Lys His | |
| 755 760 765 | |
| Leu Ala Asn Thr Asp Val Pro Ile Pro Thr His Tyr Phe Val Val Leu | |
| 770 775 780 | |
| Thr Ser Cys Lys Asn Lys Ser His Thr Pro Glu Asn Cys Pro Gly Trp | |
| 785 790 795 800 | |
| Leu Asp Val Leu Pro Phe Ile Ile Pro His Arg Pro Thr Asn Val Glu | |
| 805 810 815 | |
| Ser Cys Pro Glu Gly Lys Pro Glu Ala Leu Trp Val Glu Glu Arg Phe | |
| 820 825 830 | |
| Thr Ala His Ile Ala Arg Val Arg Asp Val Glu Leu Leu Thr Gly Leu | |
| 835 840 845 | |
| Asp Phe Tyr Gln Asp Lys Val Gln Pro Val Ser Glu Ile Leu Gln Leu | |
| 850 855 860 | |
| Lys Thr Tyr Leu Pro Thr Phe Glu Thr Thr Ile | |
| 865 870 875 | |
| NPP3 amino acid sequence shown above comprises cytoplasmic domain, | |
| transmembrane domain, phosphodiesterase/catalytic domain and Nuclease | |
| domain. The catalytic domain and the nuclease domain are jointly | |
| referred to as the extracellular domain. Residues 1-11 (Met Glu Ser to | |
| Ala Thr Glu) correspond to the cytoplasmic domain. Residues 12-30 (Gln | |
| Pro Val to Leu Leu Ala) correspond to the transmembrane domain. | |
| Residues 31-875 (Leu Leu Val to Thr Thr Ile) correspond to the extra- | |
| cellular domain. Residues 140-510 (Leu Glu Glu to Glu Val Glu) | |
| correspond to the catalytic/phosphodiesterase domain. Residues 605 to | |
| 875 (Lys Val Asn to Thr Thr Ile) correspond to the nuclease domain. | |
| The residue numbering and domain classification are based on human | |
| NPP3 sequence (UniProtKB/Swiss-Prot: 014638.2) | |
| Azurocidin-ENPP3-FC | |
| SEQ ID NO: 8 | |
| MTRLTVLALLAGLLASSRA**AKQGSCRKKCFDASFRGLENCRCDVACKDRGDCCWDFEDICVESTRIWM | |
| CNKFRCGETRLEASLCSCSDDCLQRKDCCADYKSVCQGETSWLEENCDTAQQSQCPEGFDLPPVILFSMD | |
| GFRAEYLYTWDTLMPNINKLKTCGIHSKYMRAMYPTKTFPNHYTIVTGLYPESHGIIDNNMYDVNLNKNF | |
| SLSSKEQNNPAWWHGQPMNLTAMYQGLKAATYFWPGSEVAINGSFPSIYMPYNGSVPFEERISTLLKWLD | |
| LPKAERPRFYTMYFEEPDSSGHAGGPVSARVIKALQVVDHAFGMLMEGLKQRNLHNCVNIILLADHGMDQ | |
| TYCNKMEYMIDYFPRINFFYMYEGPAPRIRAHNIPHDFFSENSEEIVRNLSCRKPDQHFKPYLTPDLPKR | |
| LHYAKNVRIDKVHLFVDQQWLAVRSKSNTNCGGGNHGYNNEFRSMEAIFLAHGPSFKEKTEVEPFENIEV | |
| YNLMCDLLRIQPAPNNGTHGSLNHLLKVPFYEPSHAEEVSKFSVCGFANPLPTESLDCFCPHLQNSTQLE | |
| QVNQMLNLIQEEITATVKVNLPFGRPRVLQKNVDHCLLYHREYVSGEGKAMRMPMWSSYTVPQLGDTSPL | |
| PPTVPDCLRADVRVPPSESQKCSFYLADKNITHGFLYPPASNRTSDSQYDALITSNLVPMYEEFRKMWDY | |
| FHSVLLIKHATERNGVNVVSGPIFDYNYDGHFDAPDEITKHLANTDVPIPTHYFVVLTSCKNKSHTPENC | |
| PGWLDVLPFIIPHRPTNVESCPEGKPEALWVEERFTAHIARVRDVELLTGLDFYQDKVQPVSEILQLKTY | |
| LPTFETTIDKTETCPPCPAPELLGGPSVFLEPPKPKDILMISRTPEVICVVVDVSHEDPEVKFNWYVDGV | |
| EVENAKTKPREEQYNSTYRVVSVLTVLHQDWLNGKEYKCKVSNKALPAPIEKTISKAKGQPREPQVYTLP | |
| PSREEMTKNQVSLICLVKGFYPSDIAVEWESNGQPENNYKTIPPVLDSDGSFELYSKLTVDKSRWQQGNV | |
| ESCSVMHEALENHYTQKSLSLSPGK | |
| Single underline - Azurocidin signal sequence, Double underline - | |
| Beginning and end of ENPP3 sequence, Bold residues - Fc sequence, ** | |
| indicates the cleavage point of the signal sequence. | |
| Azurocidin-ENPP3-Albumin | |
| SEQ ID NO: 9 | |
| MTRLTVLALLAGLLASSRA**AKQGSCRKKCFDASFRGLENCRCDVACKDRGDCCWDFEDICVESTRIWM | |
| CNKFRCGETRLEASLCSCSDDCLQRKDCCADYKSVCQGETSWLEENCDTAQQSQCPEGFDLPPVILFSMD | |
| GFRAEYLYTWDTLMPNINKLKTCGIHSKYMRAMYPTKTFPNHYTIVTGLYPESHGIIDNNMYDVNLNKNF | |
| SLSSKEQNNPAWWHGQPMNLTAMYQGLKAATYFWPGSEVAINGSFPSIYMPYNGSVPFEERISTLLKWLD | |
| LPKAERPRFYTMYFEEPDSSGHAGGPVSARVIKALQVVDHAFGMLMEGLKQRNLHNCVNIILLADHGMDQ | |
| TYCNKMEYMIDYFPRINFFYMYEGPAPRIRAHNIPHDFFSENSEEIVRNLSCRKPDQHFKPYLTPDLPKR | |
| LHYAKNVRIDKVHLFVDQQWLAVRSKSNTNCGGGNHGYNNEFRSMEAIFLAHGPSFKEKTEVEPFENIEV | |
| YNLMCDLLRIQPAPNNGTHGSLNHLLKVPFYEPSHAEEVSKFSVCGFANPLPTESLDCFCPHLQNSTQLE | |
| QVNQMLNLIQEEITATVKVNLPFGRPRVLQKNVDHCLLYHREYVSGEGKAMRMPMWSSYTVPQLGDTSPL | |
| PPTVPDCLRADVRVPPSESQKCSFYLADKNITHGFLYPPASNRTSDSQYDALITSNLVPMYEEFRKMWDY | |
| FHSVLLIKHATERNGVNVVSGPIFDYNYDGHFDAPDEITKHLANTDVPIPTHYFVVLTSCKNKSHTPENC | |
| PGWLDVLPFIIPHRPTNVESCPEGKPEALWVEERFTAHIARVRDVELLTGLDFYQDKVQPVSEILQLKTY | |
| LPTFETTIMKWVTFLLLLFVSGSAFSRGVFRREAHKSEIAHRYNDLGEQHFKGLVLIAFSQYLQKCSYDE | |
| HAKLVQEVTDFAKTCVADESAANCDKSLHTLFGDKLCAIPNLRENYGELADCCTKQEPERNECFLQHKDD | |
| NPSLPPFERPEAEAMCTSFKENPTTFMGHYLHEVARRHPYFYAPELLYYAEQYNEILTQCCAEADKESCL | |
| TPKLDGVEEKALVSSVRQRMKCSSMQKFGERAFKAWAVARLSQTFPNADFAEITKLATDLTKVNKECCHG | |
| DLLECADDRAELAKYMCENQATISSKLQTCCDKPLLKKAHCLSEVEHDTMPADLPAIAADFVEDQEVCKN | |
| YAEAEDVFLGTFLYEYSRRHPDYSVSLLLRLAKKYEATLEKCCAEANPPACYGTVLAEFQPLVEEPKNLV | |
| KTNCDLYEKLGEYGFQNAILVRYTQKAPQVSTPTLVEAARNLGRVGTKCCTLPEDQRLPCVEDYLSAILN | |
| RVCLLHEKTPVSEHVTKCCSGSLVERRPCFSALTVDETYVPKEFKAETFTFHSDICTLPEKEKQIKKQTA | |
| LAELVKHKPKATAEQLKTVMDDFAQFLDTCCKAADKDTCFSTEGPNLVTRCKDALARSWSHPQFEK | |
| Single underline - Azurocidin signal sequence, Double underline - | |
| Beginning and end of ENPP3 sequence, Bold residues - Albumin sequence, | |
| ** indicates the cleavage point of the signal sequence. | |
| Azurocidin-ENPP3 | |
| SEQ ID NO: 10 | |
| MTRLTVLALLAGLLASSRA**AKQGSCRKKCFDASFRGLENCRCDVACKDRGDCCWDFEDICVESTRIWM | |
| CNKFRCGETRLEASLCSCSDDCLQRKDCCADYKSVCQGETSWLEENCDTAQQSQCPEGFDLPPVILFSMD | |
| GFRAEYLYTWDTLMPNINKLKTCGIHSKYMRAMYPTKTFPNHYTIVTGLYPESHGIIDNNMYDVNLNKNF | |
| SLSSKEQNNPAWWHGQPMNLTAMYQGLKAATYFWPGSEVAINGSFPSIYMPYNGSVPFEERISTLLKWLD | |
| LPKAERPRFYTMYFEEPDSSGHAGGPVSARVIKALQVVDHAFGMLMEGLKQRNLHNCVNIILLADHGMDQ | |
| TYCNKMEYMIDYFPRINFFYMYEGPAPRIRAHNIPHDFFSENSEEIVRNLSCRKPDQHFKPYLTPDLPKR | |
| LHYAKNVRIDKVHLFVDQQWLAVRSKSNTNCGGGNHGYNNEFRSMEAIFLAHGPSFKEKTEVEPFENIEV | |
| YNLMCDLLRIQPAPNNGTHGSLNHLLKVPFYEPSHAEEVSKFSVCGFANPLPTESLDCFCPHLQNSTQLE | |
| QVNQMLNLIQEEITATVKVNLPFGRPRVLQKNVDHCLLYHREYVSGEGKAMRMPMWSSYTVPQLGDTSPL | |
| PPTVPDCLRADVRVPPSESQKCSFYLADKNITHGFLYPPASNRTSDSQYDALITSNLVPMYEEFRKMWDY | |
| FHSVLLIKHATERNGVNVVSGPIFDYNYDGHFDAPDEITKHLANTDVPIPTHYFVVLTSCKNKSHTPENC | |
| PGWLDVLPFIIPHRPTNVESCPEGKPEALWVEERFTAHIARVRDVELLTGLDFYQDKVQPVSEILQLKTY | |
| LPTFETTI | |
| Single underline - Azurocidin signal sequence, Double underline - | |
| Beginning and end of ENPP3 sequence, ** indicates the cleavage point | |
| of the signal sequence. | |
| ENPP4 Amino Acid Sequence - Wild Type | |
| SEQ. ID NO: 11 | |
| Met Lys Leu Leu Val Ile Leu Leu Phe Ser Gly Leu Ile Thr Gly Phe | |
| 1 5 10 15 | |
| Arg Ser Asp Ser Ser Ser Ser Leu Pro Pro Lys Leu Leu Leu Val Ser | |
| 20 25 30 | |
| Phe Asp Gly Phe Arg Ala Asp Tyr Leu Lys Asn Tyr Glu Phe Pro His | |
| 35 40 45 | |
| Leu Gln Asn Phe Ile Lys Glu Gly Val Leu Val Glu His Val Lys Asn | |
| 50 55 60 | |
| Val Phe Ile Thr Lys Thr Phe Pro Asn His Tyr Ser Ile Val Thr Gly | |
| 65 70 75 80 | |
| Leu Tyr Glu Glu Ser His Gly Ile Val Ala Asn Ser Met Tyr Asp Ala | |
| 85 90 95 | |
| Val Thr Lys Lys His Phe Ser Asp Ser Asn Asp Lys Asp Pro Phe Trp | |
| 100 105 110 | |
| Trp Asn Glu Ala Val Pro Ile Trp Val Thr Asn Gln Leu Gln Glu Asn | |
| 115 120 125 | |
| Arg Ser Ser Ala Ala Ala Met Trp Pro Gly Thr Asp Val Pro Ile His | |
| 130 135 140 | |
| Asp Thr Ile Ser Ser Tyr Phe Met Asn Tyr Asn Ser Ser Val Ser Phe | |
| 145 150 155 160 | |
| Glu Glu Arg Leu Asn Asn Ile Thr Met Trp Leu Asn Asn Ser Asn Pro | |
| 165 170 175 | |
| Pro Val Thr Phe Ala Thr Leu Tyr Trp Glu Glu Pro Asp Ala Ser Gly | |
| 180 185 190 | |
| His Lys Tyr Gly Pro Glu Asp Lys Glu Asn Met Ser Arg Val Leu Lys | |
| 195 200 205 | |
| Lys Ile Asp Asp Leu Ile Gly Asp Leu Val Gln Arg Leu Lys Met Leu | |
| 210 215 220 | |
| Gly Leu Trp Glu Asn Leu Asn Val Ile Ile Thr Ser Asp His Gly Met | |
| 225 230 235 240 | |
| Thr Gln Cys Ser Gln Asp Arg Leu Ile Asn Leu Asp Ser Cys Ile Asp | |
| 245 250 255 | |
| His Ser Tyr Tyr Thr Leu Ile Asp Leu Ser Pro Val Ala Ala Ile Leu | |
| 260 265 270 | |
| Pro Lys Ile Asn Arg Thr Glu Val Tyr Asn Lys Leu Lys Asn Cys Ser | |
| 275 280 285 | |
| Pro His Met Asn Val Tyr Leu Lys Glu Asp Ile Pro Asn Arg Phe Tyr | |
| 290 295 300 | |
| Tyr Gln His Asn Asp Arg Ile Gln Pro Ile Ile Leu Val Ala Asp Glu | |
| 305 310 315 320 | |
| Gly Trp Thr Ile Val Leu Asn Glu Ser Ser Gln Lys Leu Gly Asp His | |
| 325 330 335 | |
| Gly Tyr Asp Asn Ser Leu Pro Ser Met His Pro Phe Leu Ala Ala His | |
| 340 345 350 | |
| Gly Pro Ala Phe His Lys Gly Tyr Lys His Ser Thr Ile Asn Ile Val | |
| 355 360 365 | |
| Asp Ile Tyr Pro Met Met Cys His Ile Leu Gly Leu Lys Pro His Pro | |
| 370 375 380 | |
| Asn Asn Gly Thr Phe Gly His Thr Lys Cys Leu Leu Val Asp Gln Trp | |
| 385 390 395 400 | |
| Cys Ile Asn Leu Pro Glu Ala Ile Ala Ile Val Ile Gly Ser Leu Leu | |
| 405 410 415 | |
| Val Leu Thr Met Leu Thr Cys Leu Ile Ile Ile Met Gln Asn Arg Leu | |
| 420 425 430 | |
| Ser Val Pro Arg Pro Phe Ser Arg Leu Gln Leu Gln Glu Asp Asp Asp | |
| 435 440 445 | |
| Asp Pro Leu Ile Gly | |
| 450 | |
| ENPP51 Amino Acid Sequence | |
| SEQ. ID NO: 12 | |
| Met Thr Ser Lys Phe Leu Leu Val Ser Phe Ile Leu Ala Ala Leu Ser | |
| 1 5 10 15 | |
| Leu Ser Thr Thr Phe Ser Leu Gln**Pro Ser Cys Ala Lys Glu Val Lys | |
| 20 25 30 | |
| Ser Cys Lys Gly Arg Cys Phe Glu Arg Thr Phe Ser Asn Cys Arg Cys | |
| 35 40 45 | |
| Asp Ala Ala Cys Val Ser Leu Gly Asn Cys Cys Leu Asp Phe Gln Glu | |
| 50 55 60 | |
| Thr Cys Val Glu Pro Thr His Ile Trp Thr Cys Asn Lys Phe Arg Cys | |
| 65 70 75 80 | |
| Gly Glu Lys Arg Leu Ser Arg Phe Val Cys Ser Cys Ala Asp Asp Cys | |
| 85 90 95 | |
| Lys Thr His Asn Asp Cys Cys Ile Asn Tyr Ser Ser Val Cys Gln Asp | |
| 100 105 110 | |
| Lys Lys Ser Trp Val Glu Glu Thr Cys Glu Ser Ile Asp Thr Pro Glu | |
| 115 120 125 | |
| Cys Pro Ala Glu Phe Glu Ser Pro Pro Thr Leu Leu Phe Ser Leu Asp | |
| 130 135 140 | |
| Gly Phe Arg Ala Glu Tyr Leu His Thr Trp Gly Gly Leu Leu Pro Val | |
| 145 150 155 160 | |
| Ile Ser Lys Leu Lys Asn Cys Gly Thr Tyr Thr Lys Asn Met Arg Pro | |
| 165 170 175 | |
| Met Tyr Pro Thr Lys Thr Phe Pro Asn His Tyr Ser Ile Val Thr Gly | |
| 180 185 190 | |
| Leu Tyr Pro Glu Ser His Gly Ile Ile Asp Asn Lys Met Tyr Asp Pro | |
| 195 200 205 | |
| Lys Met Asn Ala Ser Phe Ser Leu Lys Ser Lys Glu Lys Phe Asn Pro | |
| 210 215 220 | |
| Leu Trp Tyr Lys Gly Gln Pro Ile Trp Val Thr Ala Asn His Gln Glu | |
| 225 230 235 240 | |
| Val Lys Ser Gly Thr Tyr Phe Trp Pro Gly Ser Asp Val Glu Ile Asp | |
| 245 250 255 | |
| Gly Ile Leu Pro Asp Ile Tyr Lys Val Tyr Asn Gly Ser Val Pro Phe | |
| 260 265 270 | |
| Glu Glu Arg Ile Leu Ala Val Leu Glu Trp Leu Gln Leu Pro Ser His | |
| 275 280 285 | |
| Glu Arg Pro His Phe Tyr Thr Leu Tyr Leu Glu Glu Pro Asp Ser Ser | |
| 290 295 300 | |
| Gly His Ser His Gly Pro Val Ser Ser Glu Val Ile Lys Ala Leu Gln | |
| 305 310 315 320 | |
| Lys Val Asp Arg Leu Val Gly Met Leu Met Asp Gly Leu Lys Asp Leu | |
| 325 330 335 | |
| Gly Leu Asp Lys Cys Leu Asn Leu Ile Leu Ile Ser Asp His Gly Met | |
| 340 345 350 | |
| Glu Gln Gly Ser Cys Lys Lys Tyr Val Tyr Leu Asn Lys Tyr Leu Gly | |
| 355 360 365 | |
| Asp Val Asn Asn Val Lys Val Val Tyr Gly Pro Ala Ala Arg Leu Arg | |
| 370 375 380 | |
| Pro Thr Asp Val Pro Glu Thr Tyr Tyr Ser Phe Asn Tyr Glu Ala Leu | |
| 385 390 395 400 | |
| Ala Lys Asn Leu Ser Cys Arg Glu Pro Asn Gln His Phe Arg Pro Tyr | |
| 405 410 415 | |
| Leu Lys Pro Phe Leu Pro Lys Arg Leu His Phe Ala Lys Ser Asp Arg | |
| 420 425 430 | |
| Ile Glu Pro Leu Thr Phe Tyr Leu Asp Pro Gln Trp Gln Leu Ala Leu | |
| 435 440 445 | |
| Asn Pro Ser Glu Arg Lys Tyr Cys Gly Ser Gly Phe His Gly Ser Asp | |
| 450 455 460 | |
| Asn Leu Phe Ser Asn Met Gln Ala Leu Phe Ile Gly Tyr Gly Pro Ala | |
| 465 470 475 480 | |
| Phe Lys His Gly Ala Glu Val Asp Ser Phe Glu Asn Ile Glu Val Tyr | |
| 485 490 495 | |
| Asn Leu Met Cys Asp Leu Leu Gly Leu Ile Pro Ala Pro Asn Asn Gly | |
| 500 505 510 | |
| Ser His Gly Ser Leu Asn His Leu Leu Lys Lys Pro Ile Tyr Asn Pro | |
| 515 520 525 | |
| Ser His Pro Lys Glu Glu Gly Phe Leu Ser Gln Cys Pro Ile Lys Ser | |
| 530 535 540 | |
| Thr Ser Asn Asp Leu Gly Cys Thr Cys Asp Pro Trp Ile Val Pro Ile | |
| 545 550 555 560 | |
| Lys Asp Phe Glu Lys Gln Leu Asn Leu Thr Thr Glu Asp Val Asp Asp | |
| 565 570 575 | |
| Ile Tyr His Met Thr Val Pro Tyr Gly Arg Pro Arg Ile Leu Leu Lys | |
| 580 585 590 | |
| Gln His Arg Val Cys Leu Leu Gln Gln Gln Gln Phe Leu Thr Gly Tyr | |
| 595 600 605 | |
| Ser Leu Asp Leu Leu Met Pro Leu Trp Ala Ser Tyr Thr Phe Leu Ser | |
| 610 615 620 | |
| Asn Asp Gln Phe Ser Arg Asp Asp Phe Ser Asn Cys Leu Tyr Gln Asp | |
| 625 630 635 640 | |
| Leu Arg Ile Pro Leu Ser Pro Val His Lys Cys Ser Tyr Tyr Lys Ser | |
| 645 650 655 | |
| Asn Ser Lys Leu Ser Tyr Gly Phe Leu Thr Pro Pro Arg Leu Asn Arg | |
| 660 665 670 | |
| Val Ser Asn His Ile Tyr Ser Glu Ala Leu Leu Thr Ser Asn Ile Val | |
| 675 680 685 | |
| Pro Met Tyr Gln Ser Phe Gln Val Ile Trp His Tyr Leu His Asp Thr | |
| 690 695 700 | |
| Leu Leu Gln Arg Tyr Ala His Glu Arg Asn Gly Ile Asn Val Val Ser | |
| 705 710 715 720 | |
| Gly Pro Val Phe Asp Phe Asp Tyr Asp Gly Arg Tyr Asp Ser Leu Glu | |
| 725 730 735 | |
| Ile Leu Lys Gln Asn Ser Arg Val Ile Arg Ser Gln Glu Ile Leu Ile | |
| 740 745 750 | |
| Pro Thr His Phe Phe Ile Val Leu Thr Ser Cys Lys Gln Leu Ser Glu | |
| 755 760 765 | |
| Thr Pro Leu Glu Cys Ser Ala Leu Glu Ser Ser Ala Tyr Ile Leu Pro | |
| 770 775 780 | |
| His Arg Pro Asp Asn Ile Glu Ser Cys Thr His Gly Lys Arg Glu Ser | |
| 785 790 795 800 | |
| Ser Trp Val Glu Glu Leu Leu Thr Leu His Arg Ala Arg Val Thr Asp | |
| 805 810 815 | |
| Val Glu Leu Ile Thr Gly Leu Ser Phe Tyr Gln Asp Arg Gln Glu Ser | |
| 820 825 830 | |
| Val Ser Glu Leu Leu Arg Leu Lys Thr His Leu Pro Ile Phe Ser Gln | |
| 835 840 845 | |
| Glu Asp | |
| 850 | |
| Singly underlined: signal peptide sequence; double-underlined: | |
| beginning and end of NPP1; ** = cleavage position at the signal | |
| peptide sequence | |
| ENPP51 - ALB Amino Acid Sequence: | |
| SEQ. ID NO: 13 | |
| Met Thr Ser Lys Phe Leu Leu Val Ser Phe Ile Leu Ala Ala Leu Ser | |
| 1 5 10 15 | |
| Leu Ser Thr Thr Phe Ser Leu Gln**Pro Ser Cys Ala Lys Glu Val Lys | |
| 20 25 30 | |
| Ser Cys Lys Gly Arg Cys Phe Glu Arg Thr Phe Ser Asn Cys Arg Cys | |
| 35 40 45 | |
| Asp Ala Ala Cys Val Ser Leu Gly Asn Cys Cys Leu Asp Phe Gln Glu | |
| 50 55 60 | |
| Thr Cys Val Glu Pro Thr His Ile Trp Thr Cys Asn Lys Phe Arg Cys | |
| 65 70 75 80 | |
| Gly Glu Lys Arg Leu Ser Arg Phe Val Cys Ser Cys Ala Asp Asp Cys | |
| 85 90 95 | |
| Lys Thr His Asn Asp Cys Cys Ile Asn Tyr Ser Ser Val Cys Gln Asp | |
| 100 105 110 | |
| Lys Lys Ser Trp Val Glu Glu Thr Cys Glu Ser Ile Asp Thr Pro Glu | |
| 115 120 125 | |
| Cys Pro Ala Glu Phe Glu Ser Pro Pro Thr Leu Leu Phe Ser Leu Asp | |
| 130 135 140 | |
| Gly Phe Arg Ala Glu Tyr Leu His Thr Trp Gly Gly Leu Leu Pro Val | |
| 145 150 155 160 | |
| Ile Ser Lys Leu Lys Asn Cys Gly Thr Tyr Thr Lys Asn Met Arg Pro | |
| 165 170 175 | |
| Met Tyr Pro Thr Lys Thr Phe Pro Asn His Tyr Ser Ile Val Thr Gly | |
| 180 185 190 | |
| Leu Tyr Pro Glu Ser His Gly Ile Ile Asp Asn Lys Met Tyr Asp Pro | |
| 195 200 205 | |
| Lys Met Asn Ala Ser Phe Ser Leu Lys Ser Lys Glu Lys Phe Asn Pro | |
| 210 215 220 | |
| Leu Trp Tyr Lys Gly Gln Pro Ile Trp Val Thr Ala Asn His Gln Glu | |
| 225 230 235 240 | |
| Val Lys Ser Gly Thr Tyr Phe Trp Pro Gly Ser Asp Val Glu Ile Asp | |
| 245 250 255 | |
| Gly Ile Leu Pro Asp Ile Tyr Lys Val Tyr Asn Gly Ser Val Pro Phe | |
| 260 265 270 | |
| Glu Glu Arg Ile Leu Ala Val Leu Glu Trp Leu Gln Leu Pro Ser His | |
| 275 280 285 | |
| Glu Arg Pro His Phe Tyr Thr Leu Tyr Leu Glu Glu Pro Asp Ser Ser | |
| 290 295 300 | |
| Gly His Ser His Gly Pro Val Ser Ser Glu Val Ile Lys Ala Leu Gln | |
| 305 310 315 320 | |
| Lys Val Asp Arg Leu Val Gly Met Leu Met Asp Gly Leu Lys Asp Leu | |
| 325 330 335 | |
| Gly Leu Asp Lys Cys Leu Asn Leu Ile Leu Ile Ser Asp His Gly Met | |
| 340 345 350 | |
| Glu Gln Gly Ser Cys Lys Lys Tyr Val Tyr Leu Asn Lys Tyr Leu Gly | |
| 355 360 365 | |
| Asp Val Asn Asn Val Lys Val Val Tyr Gly Pro Ala Ala Arg Leu Arg | |
| 370 375 380 | |
| Pro Thr Asp Val Pro Glu Thr Tyr Tyr Ser Phe Asn Tyr Glu Ala Leu | |
| 385 390 395 400 | |
| Ala Lys Asn Leu Ser Cys Arg Glu Pro Asn Gln His Phe Arg Pro Tyr | |
| 405 410 415 | |
| Leu Lys Pro Phe Leu Pro Lys Arg Leu His Phe Ala Lys Ser Asp Arg | |
| 420 425 430 | |
| Ile Glu Pro Leu Thr Phe Tyr Leu Asp Pro Gln Trp Gln Leu Ala Leu | |
| 435 440 445 | |
| Asn Pro Ser Glu Arg Lys Tyr Cys Gly Ser Gly Phe His Gly Ser Asp | |
| 450 455 460 | |
| Asn Leu Phe Ser Asn Met Gln Ala Leu Phe Ile Gly Tyr Gly Pro Ala | |
| 465 470 475 480 | |
| Phe Lys His Gly Ala Glu Val Asp Ser Phe Glu Asn Ile Glu Val Tyr | |
| 485 490 495 | |
| Asn Leu Met Cys Asp Leu Leu Gly Leu Ile Pro Ala Pro Asn Asn Gly | |
| 500 505 510 | |
| Ser His Gly Ser Leu Asn His Leu Leu Lys Lys Pro Ile Tyr Asn Pro | |
| 515 520 525 | |
| Ser His Pro Lys Glu Glu Gly Phe Leu Ser Gln Cys Pro Ile Lys Ser | |
| 530 535 540 | |
| Thr Ser Asn Asp Leu Gly Cys Thr Cys Asp Pro Trp Ile Val Pro Ile | |
| 545 550 555 560 | |
| Lys Asp Phe Glu Lys Gln Leu Asn Leu Thr Thr Glu Asp Val Asp Asp | |
| 565 570 575 | |
| Ile Tyr His Met Thr Val Pro Tyr Gly Arg Pro Arg Ile Leu Leu Lys | |
| 580 585 590 | |
| Gln His Arg Val Cys Leu Leu Gln Gln Gln Gln Phe Leu Thr Gly Tyr | |
| 595 600 605 | |
| Ser Leu Asp Leu Leu Met Pro Leu Trp Ala Ser Tyr Thr Phe Leu Ser | |
| 610 615 620 | |
| Asn Asp Gln Phe Ser Arg Asp Asp Phe Ser Asn Cys Leu Tyr Gln Asp | |
| 625 630 635 640 | |
| Leu Arg Ile Pro Leu Ser Pro Val His Lys Cys Ser Tyr Tyr Lys Ser | |
| 645 650 655 | |
| Asn Ser Lys Leu Ser Tyr Gly Phe Leu Thr Pro Pro Arg Leu Asn Arg | |
| 660 665 670 | |
| Val Ser Asn His Ile Tyr Ser Glu Ala Leu Leu Thr Ser Asn Ile Val | |
| 675 680 685 | |
| Pro Met Tyr Gln Ser Phe Gln Val Ile Trp His Tyr Leu His Asp Thr | |
| 690 695 700 | |
| Leu Leu Gln Arg Tyr Ala His Glu Arg Asn Gly Ile Asn Val Val Ser | |
| 705 710 715 720 | |
| Gly Pro Val Phe Asp Phe Asp Tyr Asp Gly Arg Tyr Asp Ser Leu Glu | |
| 725 730 735 | |
| Ile Leu Lys Gln Asn Ser Arg Val Ile Arg Ser Gln Glu Ile Leu Ile | |
| 740 745 750 | |
| Pro Thr His Phe Phe Ile Val Leu Thr Ser Cys Lys Gln Leu Ser Glu | |
| 755 760 765 | |
| Thr Pro Leu Glu Cys Ser Ala Leu Glu Ser Ser Ala Tyr Ile Leu Pro | |
| 770 775 780 | |
| His Arg Pro Asp Asn Ile Glu Ser Cys Thr His Gly Lys Arg Glu Ser | |
| 785 790 795 800 | |
| Ser Trp Val Glu Glu Leu Leu Thr Leu His Arg Ala Arg Val Thr Asp | |
| 805 810 815 | |
| Val Glu Leu Ile Thr Gly Leu Ser Phe Tyr Gln Asp Arg Gln Glu Ser | |
| 820 825 830 | |
| Val Ser Glu Leu Leu Arg Leu Lys Thr His Leu Pro Ile Phe Ser Gln | |
| 835 840 845 | |
| Glu Asp Gly Gly Ser Gly Gly Ser Met Lys Trp Val Thr Phe Leu Leu | |
| 850 855 860 | |
| Leu Leu Phe Val Ser Gly Ser Ala Phe Ser Arg Gly Val Phe Arg Arg | |
| 865 870 875 880 | |
| Glu Ala His Lys Ser Glu Ile Ala His Arg Tyr Asn Asp Leu Gly Glu | |
| 885 890 895 | |
| Gln His Phe Lys Gly Leu Val Leu Ile Ala Phe Ser Gln Tyr Leu Gln | |
| 900 905 910 | |
| Lys Cys Ser Tyr Asp Glu His Ala Lys Leu Val Gln Glu Val Thr Asp | |
| 915 920 925 | |
| Phe Ala Lys Thr Cys Val Ala Asp Glu Ser Ala Ala Asn Cys Asp Lys | |
| 930 935 940 | |
| Ser Leu His Thr Leu Phe Gly Asp Lys Leu Cys Ala Ile Pro Asn Leu | |
| 945 950 955 960 | |
| Arg Glu Asn Tyr Gly Glu Leu Ala Asp Cys Cys Thr Lys Gln Glu Pro | |
| 965 970 975 | |
| Glu Arg Asn Glu Cys Phe Leu Gln His Lys Asp Asp Asn Pro Ser Leu | |
| 980 985 990 | |
| Pro Pro Phe Glu Arg Pro Glu Ala Glu Ala Met Cys Thr Ser Phe Lys | |
| 995 1000 1005 | |
| Glu Asn Pro Thr Thr Phe Met Gly His Tyr Leu His Glu Val Ala | |
| 1010 1015 1020 | |
| Arg Arg His Pro Tyr Phe Tyr Ala Pro Glu Leu Leu Tyr Tyr Ala | |
| 1025 1030 1035 | |
| Glu Gln Tyr Asn Glu Ile Leu Thr Gln Cys Cys Ala Glu Ala Asp | |
| 1040 1045 1050 | |
| Lys Glu Ser Cys Leu Thr Pro Lys Leu Asp Gly Val Lys Glu Lys | |
| 1055 1060 1065 | |
| Ala Leu Val Ser Ser Val Arg Gln Arg Met Lys Cys Ser Ser Met | |
| 1070 1075 1080 | |
| Gln Lys Phe Gly Glu Arg Ala Phe Lys Ala Trp Ala Val Ala Arg | |
| 1085 1090 1095 | |
| Leu Ser Gln Thr Phe Pro Asn Ala Asp Phe Ala Glu Ile Thr Lys | |
| 1100 1105 1110 | |
| Leu Ala Thr Asp Leu Thr Lys Val Asn Lys Glu Cys Cys His Gly | |
| 1115 1120 1125 | |
| Asp Leu Leu Glu Cys Ala Asp Asp Arg Ala Glu Leu Ala Lys Tyr | |
| 1130 1135 1140 | |
| Met Cys Glu Asn Gln Ala Thr Ile Ser Ser Lys Leu Gln Thr Cys | |
| 1145 1150 1155 | |
| Cys Asp Lys Pro Leu Leu Lys Lys Ala His Cys Leu Ser Glu Val | |
| 1160 1165 1170 | |
| Glu His Asp Thr Met Pro Ala Asp Leu Pro Ala Ile Ala Ala Asp | |
| 1175 1180 1185 | |
| Phe Val Glu Asp Gln Glu Val Cys Lys Asn Tyr Ala Glu Ala Lys | |
| 1190 1195 1200 | |
| Asp Val Phe Leu Gly Thr Phe Leu Tyr Glu Tyr Ser Arg Arg His | |
| 1205 1210 1215 | |
| Pro Asp Tyr Ser Val Ser Leu Leu Leu Arg Leu Ala Lys Lys Tyr | |
| 1220 1225 1230 | |
| Glu Ala Thr Leu Glu Lys Cys Cys Ala Glu Ala Asn Pro Pro Ala | |
| 1235 1240 1245 | |
| Cys Tyr Gly Thr Val Leu Ala Glu Phe Gln Pro Leu Val Glu Glu | |
| 1250 1255 1260 | |
| Pro Lys Asn Leu Val Lys Thr Asn Cys Asp Leu Tyr Glu Lys Leu | |
| 1265 1270 1275 | |
| Gly Glu Tyr Gly Phe Gln Asn Ala Ile Leu Val Arg Tyr Thr Gln | |
| 1280 1285 1290 | |
| Lys Ala Pro Gln Val Ser Thr Pro Thr Leu Val Glu Ala Ala Arg | |
| 1295 1300 1305 | |
| Asn Leu Gly Arg Val Gly Thr Lys Cys Cys Thr Leu Pro Glu Asp | |
| 1310 1315 1320 | |
| Gln Arg Leu Pro Cys Val Glu Asp Tyr Leu Ser Ala Ile Leu Asn | |
| 1325 1330 1335 | |
| Arg Val Cys Leu Leu His Glu Lys Thr Pro Val Ser Glu His Val | |
| 1340 1345 1350 | |
| Thr Lys Cys Cys Ser Gly Ser Leu Val Glu Arg Arg Pro Cys Phe | |
| 1355 | |
| Ser Ala Leu Thr Val Asp Glu Thr Tyr Val Pro Lys Glu Phe Lys | |
| 1370 1375 1380 | |
| Ala Glu Thr Phe Thr Phe His Ser Asp Ile Cys Thr Leu Pro Glu | |
| 1385 1390 1395 | |
| Lys Glu Lys Gln Ile Lys Lys Gln Thr Ala Leu Ala Glu Leu Val | |
| 1400 1405 1410 | |
| Lys His Lys Pro Lys Ala Thr Ala Glu Gln Leu Lys Thr Val Met | |
| 1415 1420 1425 | |
| Asp Asp Phe Ala Gln Phe Leu Asp Thr Cys Cys Lys Ala Ala Asp | |
| 1430 1435 1440 | |
| Lys Asp Thr Cys Phe Ser Thr Glu Gly Pro Asn Leu Val Thr Arg | |
| 1445 1450 1455 | |
| Cys Lys Asp Ala Leu Ala Arg Ser Trp Ser His Pro Gln Phe Glu | |
| 1460 1465 1470 | |
| Lys | |
| Singly underlined: signal peptide sequence; double-underlined: | |
| beginning and end of NPP1; ** = cleavage position at the signal | |
| peptide sequence; bold residues indicate albumin sequence | |
| ENPP5-NPP3-Fc sequence | |
| SEQ. ID NO: 14 | |
| Met Thr Ser Lys Phe Leu Leu Val Ser Phe Ile Leu Ala Ala Leu Ser | |
| 1 5 10 15 | |
| Leu Ser Thr Thr Phe Ser**Lys Gln Gly Ser Cys Arg Lys Lys Cys Phe | |
| 20 25 30 | |
| Asp Ala Ser Phe Arg Gly Leu Glu Asn Cys Arg Cys Asp Val Ala Cys | |
| 35 40 45 | |
| Lys Asp Arg Gly Asp Cys Cys Trp Asp Phe Glu Asp Thr Cys Val Glu | |
| 50 55 60 | |
| Ser Thr Arg Ile Trp Met Cys Asn Lys Phe Arg Cys Gly Glu Arg Leu | |
| 65 70 75 80 | |
| Glu Ala Ser Leu Cys Ser Cys Ser Asp Asp Cys Leu Gln Arg Lys Asp | |
| 85 90 95 | |
| Cys Cys Ala Asp Tyr Lys Ser Val Cys Gln Gly Glu Thr Ser Trp Leu | |
| 100 105 110 | |
| Glu Glu Asn Cys Asp Thr Ala Gln Gln Ser Gln Cys Pro Glu Gly Phe | |
| 115 120 125 | |
| Asp Leu Pro Pro Val Ile Leu Phe Ser Met Asp Gly Phe Arg Ala Glu | |
| 130 135 140 | |
| Tyr Leu Tyr Thr Trp Asp Thr Leu Met Pro Asn Ile Asn Lys Leu Lys | |
| 145 150 155 160 | |
| Thr Cys Gly Ile His Ser Lys Tyr Met Arg Ala Met Tyr Pro Thr Lys | |
| 165 170 175 | |
| Thr Phe Pro Asn His Tyr Thr Ile Val Thr Gly Leu Tyr Pro Glu Ser | |
| 180 185 190 | |
| His Gly Ile Ile Asp Asn Asn Met Tyr Asp Val Asn Leu Asn Lys Asn | |
| 195 200 205 | |
| Phe Ser Leu Ser Ser Lys Glu Gln Asn Asn Pro Ala Trp Trp His Gly | |
| 210 215 220 | |
| Gln Pro Met Trp Leu Thr Ala Met Tyr Gln Gly Leu Lys Ala Ala Thr | |
| 225 230 235 240 | |
| Tyr Phe Trp Pro Gly Ser Glu Val Ala Ile Asn Gly Ser Phe Pro Ser | |
| 245 250 255 | |
| Ile Tyr Met Pro Tyr Asn Gly Ser Val Pro Phe Glu Glu Arg Ile Ser | |
| 260 265 270 | |
| Thr Leu Leu Lys Trp Leu Asp Leu Pro Lys Ala Glu Arg Pro Arg Phe | |
| 275 280 285 | |
| Tyr Thr Met Tyr Phe Glu Glu Pro Asp Ser Ser Gly His Ala Gly Gly | |
| 290 295 300 | |
| Pro Val Ser Ala Arg Val Ile Lys Ala Leu Gln Val Val Asp His Ala | |
| 305 310 315 320 | |
| Phe Gly Met Leu Met Glu Gly Leu Lys Gln Arg Asn Leu His Asn Cys | |
| 325 330 335 | |
| Val Asn Ile Ile Leu Leu Ala Asp His Gly Met Asp Gln Thr Tyr Cys | |
| 340 345 350 | |
| Asn Lys Met Glu Tyr Met Thr Asp Tyr Phe Pro Arg Ile Asn Phe Phe | |
| 355 360 365 | |
| Tyr Met Tyr Glu Gly Pro Ala Pro Arg Ile Arg Ala His Asn Ile Pro | |
| 370 375 380 | |
| His Asp Phe Phe Ser Phe Asn Ser Glu Glu Ile Val Arg Asn Leu Ser | |
| 385 390 395 400 | |
| Cys Arg Lys Pro Asp Gln His Phe Lys Pro Tyr Leu Thr Pro Asp Leu | |
| 405 410 415 | |
| Pro Lys Arg Leu His Tyr Ala Lys Asn Val Arg Ile Asp Lys Val His | |
| 420 425 430 | |
| Leu Phe Val Asp Gln Gln Trp Leu Ala Val Arg Ser Lys Ser Asn Thr | |
| 435 440 445 | |
| Asn Cys Gly Gly Gly Asn His Gly Tyr Asn Asn Glu Phe Arg Ser Met | |
| 450 455 460 | |
| Glu Ala Ile Phe Leu Ala His Gly Pro Ser Phe Lys Glu Lys Thr Glu | |
| 465 470 475 480 | |
| Val Glu Pro Phe Glu Asn Ile Glu Val Tyr Asn Leu Met Cys Asp Leu | |
| 485 490 495 | |
| Leu Arg Ile Gln Pro Ala Pro Asn Asn Gly Thr His Gly Ser Leu Asn | |
| 500 505 510 | |
| His Leu Leu Lys Val Pro Phe Tyr Glu Pro Ser His Ala Glu Glu Val | |
| 515 520 525 | |
| Ser Lys Phe Ser Val Cys Gly Phe Ala Asn Pro Leu Pro Thr Glu Ser | |
| 530 535 540 | |
| Leu Asp Cys Phe Cys Pro His Leu Gln Asn Ser Thr Gln Leu Glu Gln | |
| 545 550 555 560 | |
| Val Asn Gln Met Leu Asn Leu Thr Gln Glu Glu Ile Thr Ala Thr Val | |
| 565 570 575 | |
| Lys Val Asn Leu Pro Phe Gly Arg Pro Arg Val Leu Gln Lys Asn Val | |
| 580 585 590 | |
| Asp His Cys Leu Leu Tyr His Arg Glu Tyr Val Ser Gly Phe Gly Lys | |
| 595 600 605 | |
| Ala Met Arg Met Pro Met Trp Ser Ser Tyr Thr Val Pro Gln Leu Gly | |
| 610 615 620 | |
| Asp Thr Ser Pro Leu Pro Pro Thr Val Pro Asp Cys Leu Arg Ala Asp | |
| 625 630 635 640 | |
| Val Arg Val Pro Pro Ser Glu Ser Gln Lys Cys Ser Phe Tyr Leu Ala | |
| 645 650 655 | |
| Asp Lys Asn Ile Thr His Gly Phe Leu Tyr Pro Pro Ala Ser Asn Arg | |
| 660 665 670 | |
| Thr Ser Asp Ser Gln Tyr Asp Ala Leu Ile Thr Ser Asn Leu Val Pro | |
| 675 680 685 | |
| Met Tyr Glu Glu Phe Arg Lys Met Trp Asp Tyr Phe His Ser Val Leu | |
| 690 695 700 | |
| Leu Ile Lys His Ala Thr Glu Arg Asn Gly Val Asn Val Val Ser Gly | |
| 705 710 715 720 | |
| Pro Ile Phe Asp Tyr Asn Tyr Asp Gly His Phe Asp Ala Pro Asp Glu | |
| 725 730 735 | |
| Ile Thr Lys His Leu Ala Asn Thr Asp Val Pro Ile Pro Thr His Tyr | |
| 740 745 750 | |
| Phe Val Val Leu Thr Ser Cys Lys Asn Lys Ser His Thr Pro Glu Asn | |
| 755 760 765 | |
| Cys Pro Gly Trp Leu Asp Val Leu Pro Phe Ile Ile Pro His Arg Pro | |
| 770 775 780 | |
| Thr Asn Val Glu Ser Cys Pro Glu Gly Lys Pro Glu Ala Leu Trp Val | |
| 785 790 795 800 | |
| Glu Glu Arg Phe Thr Ala His Ile Ala Arg Val Arg Asp Val Glu Leu | |
| 805 810 815 | |
| Leu Thr Gly Leu Asp Phe Tyr Gln Asp Lys Val Gln Pro Val Ser Glu | |
| 820 825 830 | |
| Ile Leu Gln Leu Lys Thr Tyr Leu Pro Thr Phe Glu Thr Thr Ile Asp | |
| 835 840 845 | |
| Lys Thr His Thr Cys Pro Pro Cys Pro Ala Pro Glu Leu Leu Gly Gly | |
| 850 855 860 | |
| Pro Ser Val Phe Leu Phe Pro Pro Lys Pro Lys Asp Thr Leu Met Ile | |
| 865 870 875 880 | |
| Ser Arg Thr Pro Glu Val Thr Cys Val Val Val Asp Val Ser His Glu | |
| 885 890 895 | |
| Asp Pro Glu Val Lys Phe Asn Trp Tyr Val Asp Gly Val Glu Val His | |
| 900 905 910 | |
| Asn Ala Lys Thr Lys Pro Arg Glu Glu Gln Tyr Asn Ser Thr Tyr Arg | |
| 915 920 925 | |
| Val Val Ser Val Leu Thr Val Leu His Gln Asp Trp Leu Asn Gly Lys | |
| 930 935 940 | |
| Glu Tyr Lys Cys Lys Val Ser Asn Lys Ala Leu Pro Ala Pro Ile Glu | |
| 945 950 955 960 | |
| Lys Thr Ile Ser Lys Ala Lys Gly Gln Pro Arg Glu Pro Gln Val Tyr | |
| 965 970 975 | |
| Thr Leu Pro Pro Ser Arg Glu Glu Met Thr Lys Asn Gln Val Ser Leu | |
| 980 985 990 | |
| Thr Cys Leu Val Lys Gly Phe Tyr Pro Ser Asp Ile Ala Val Glu Trp | |
| 995 1000 1005 | |
| Glu Ser Asn Gly Gln Pro Glu Asn Asn Tyr Lys Thr Thr Pro Pro | |
| 1010 1015 1020 | |
| Val Leu Asp Ser Asp Gly Ser Phe Phe Leu Tyr Ser Lys Leu Thr | |
| 1025 1030 1035 | |
| Val Asp Lys Ser Arg Trp Gln Gln Gly Asn Val Phe Ser Cys Ser | |
| 1040 1045 1050 | |
| Val Met His Glu Ala Leu His Asn His Tyr Thr Gln Lys Ser Leu | |
| 1055 1060 1065 | |
| Ser Leu Ser Pro Gly Lys | |
| 1070 | |
| Singly underlined: signal peptide sequence; double-underlined: | |
| beginning and end of NPP33; ** = cleavage position at the signal | |
| peptide sequence; bold residues indicate albumin sequence | |
| ENPP5-NPP3-Albumin sequence | |
| SEQ. ID NO: 15 | |
| Met Thr Ser Lys Phe Leu Leu Val Ser Phe Ile Leu Ala Ala Leu Ser | |
| 1 5 10 15 | |
| Leu Ser Thr Thr Phe Ser**Lys Gln Gly Ser Cys Arg Lys Lys Cys Phe | |
| 20 25 30 | |
| Asp Ala Ser Phe Arg Gly Leu Glu Asn Cys Arg Cys Asp Val Ala Cys | |
| 35 40 45 | |
| Lys Asp Arg Gly Asp Cys Cys Trp Asp Phe Glu Asp Thr Cys Val Glu | |
| 50 55 60 | |
| Ser Thr Arg Ile Trp Met Cys Asn Lys Phe Arg Cys Gly Glu Arg Leu | |
| 65 70 75 80 | |
| Glu Ala Ser Leu Cys Ser Cys Ser Asp Asp Cys Leu Gln Arg Lys Asp | |
| 85 90 95 | |
| Cys Cys Ala Asp Tyr Lys Ser Val Cys Gln Gly Glu Thr Ser Trp Leu | |
| 100 105 110 | |
| Glu Glu Asn Cys Asp Thr Ala Gln Gln Ser Gln Cys Pro Glu Gly Phe | |
| 115 120 125 | |
| Asp Leu Pro Pro Val Ile Leu Phe Ser Met Asp Gly Phe Arg Ala Glu | |
| 130 135 140 | |
| Tyr Leu Tyr Thr Trp Asp Thr Leu Met Pro Asn Ile Asn Lys Leu Lys | |
| 145 150 155 160 | |
| Thr Cys Gly Ile His Ser Lys Tyr Met Arg Ala Met Tyr Pro Thr Lys | |
| 165 170 175 | |
| Thr Phe Pro Asn His Tyr Thr Ile Val Thr Gly Leu Tyr Pro Glu Ser | |
| 180 185 190 | |
| His Gly Ile Ile Asp Asn Asn Met Tyr Asp Val Asn Leu Asn Lys Asn | |
| 195 200 205 | |
| Phe Ser Leu Ser Ser Lys Glu Gln Asn Asn Pro Ala Trp Trp His Gly | |
| 210 215 220 | |
| Gln Pro Met Trp Leu Thr Ala Met Tyr Gln Gly Leu Lys Ala Ala Thr | |
| 225 230 235 240 | |
| Tyr Phe Trp Pro Gly Ser Glu Val Ala Ile Asn Gly Ser Phe Pro Ser | |
| 245 250 255 | |
| Ile Tyr Met Pro Tyr Asn Gly Ser Val Pro Phe Glu Glu Arg Ile Ser | |
| 260 265 270 | |
| Thr Leu Leu Lys Trp Leu Asp Leu Pro Lys Ala Glu Arg Pro Arg Phe | |
| 275 280 285 | |
| Tyr Thr Met Tyr Phe Glu Glu Pro Asp Ser Ser Gly His Ala Gly Gly | |
| 290 295 300 | |
| Pro Val Ser Ala Arg Val Ile Lys Ala Leu Gln Val Val Asp His Ala | |
| 305 310 315 320 | |
| Phe Gly Met Leu Met Glu Gly Leu Lys Gln Arg Asn Leu His Asn Cys | |
| 325 330 335 | |
| Val Asn Ile Ile Leu Leu Ala Asp His Gly Met Asp Gln Thr Tyr Cys | |
| 340 345 350 | |
| Asn Lys Met Glu Tyr Met Thr Asp Tyr Phe Pro Arg Ile Asn Phe Phe | |
| 355 360 365 | |
| Tyr Met Tyr Glu Gly Pro Ala Pro Arg Ile Arg Ala His Asn Ile Pro | |
| 370 375 380 | |
| His Asp Phe Phe Ser Phe Asn Ser Glu Glu Ile Val Arg Asn Leu Ser | |
| 385 390 395 400 | |
| Cys Arg Lys Pro Asp Gln His Phe Lys Pro Tyr Leu Thr Pro Asp Leu | |
| 405 410 415 | |
| Pro Lys Arg Leu His Tyr Ala Lys Asn Val Arg Ile Asp Lys Val His | |
| 420 425 430 | |
| Leu Phe Val Asp Gln Gln Trp Leu Ala Val Arg Ser Lys Ser Asn Thr | |
| 435 440 445 | |
| Asn Cys Gly Gly Gly Asn His Gly Tyr Asn Asn Glu Phe Arg Ser Met | |
| 450 455 460 | |
| Glu Ala Ile Phe Leu Ala His Gly Pro Ser Phe Lys Glu Lys Thr Glu | |
| 465 470 475 480 | |
| Val Glu Pro Phe Glu Asn Ile Glu Val Tyr Asn Leu Met Cys Asp Leu | |
| 485 490 495 | |
| Leu Arg Ile Gln Pro Ala Pro Asn Asn Gly Thr His Gly Ser Leu Asn | |
| 500 505 510 | |
| His Leu Leu Lys Val Pro Phe Tyr Glu Pro Ser His Ala Glu Glu Val | |
| 515 520 525 | |
| Ser Lys Phe Ser Val Cys Gly Phe Ala Asn Pro Leu Pro Thr Glu Ser | |
| 530 535 540 | |
| Leu Asp Cys Phe Cys Pro His Leu Gln Asn Ser Thr Gln Leu Glu Gln | |
| 545 550 555 560 | |
| Val Asn Gln Met Leu Asn Leu Thr Gln Glu Glu Ile Thr Ala Thr Val | |
| 565 570 575 | |
| Lys Val Asn Leu Pro Phe Gly Arg Pro Arg Val Leu Gln Lys Asn Val | |
| 580 585 590 | |
| Asp His Cys Leu Leu Tyr His Arg Glu Tyr Val Ser Gly Phe Gly Lys | |
| 595 600 605 | |
| Ala Met Arg Met Pro Met Trp Ser Ser Tyr Thr Val Pro Gln Leu Gly | |
| 610 615 620 | |
| Asp Thr Ser Pro Leu Pro Pro Thr Val Pro Asp Cys Leu Arg Ala Asp | |
| 625 630 635 640 | |
| Val Arg Val Pro Pro Ser Glu Ser Gln Lys Cys Ser Phe Tyr Leu Ala | |
| 645 650 655 | |
| Asp Lys Asn Ile Thr His Gly Phe Leu Tyr Pro Pro Ala Ser Asn Arg | |
| 660 665 670 | |
| Thr Ser Asp Ser Gln Tyr Asp Ala Leu Ile Thr Ser Asn Leu Val Pro | |
| 675 680 685 | |
| Met Tyr Glu Glu Phe Arg Lys Met Trp Asp Tyr Phe His Ser Val Leu | |
| 690 695 700 | |
| Leu Ile Lys His Ala Thr Glu Arg Asn Gly Val Asn Val Val Ser Gly | |
| 705 710 715 720 | |
| Pro Ile Phe Asp Tyr Asn Tyr Asp Gly His Phe Asp Ala Pro Asp Glu | |
| 725 730 735 | |
| Ile Thr Lys His Leu Ala Asn Thr Asp Val Pro Ile Pro Thr His Tyr | |
| 740 745 750 | |
| Phe Val Val Leu Thr Ser Cys Lys Asn Lys Ser His Thr Pro Glu Asn | |
| 755 760 765 | |
| Cys Pro Gly Trp Leu Asp Val Leu Pro Phe Ile Ile Pro His Arg Pro | |
| 770 775 780 | |
| Thr Asn Val Glu Ser Cys Pro Glu Gly Lys Pro Glu Ala Leu Trp Val | |
| 785 790 795 800 | |
| Glu Glu Arg Phe Thr Ala His Ile Ala Arg Val Arg Asp Val Glu Leu | |
| 805 810 815 | |
| Leu Thr Gly Leu Asp Phe Tyr Gln Asp Lys Val Gln Pro Val Ser Glu | |
| 820 825 830 | |
| Ile Leu Gln Leu Lys Thr Tyr Leu Pro Thr Phe Glu Thr Thr Ile Gly | |
| 835 840 845 | |
| Gly Gly Ser Gly Gly Gly Gly Ser Gly Gly Gly Gly Ser Met Lys Trp | |
| 850 855 860 | |
| Val Thr Phe Leu Leu Leu Leu Phe Val Ser Gly Ser Ala Phe Ser Arg | |
| 865 870 875 880 | |
| Gly Val Phe Arg Arg Glu Ala His Lys Ser Glu Ile Ala His Arg Tyr | |
| 885 890 895 | |
| Asn Asp Leu Gly Glu Gln His Phe Lys Gly Leu Val Leu Ile Ala Phe | |
| 900 905 910 | |
| Ser Gln Tyr Leu Gln Lys Cys Ser Tyr Asp Glu His Ala Lys Leu Val | |
| 915 920 925 | |
| Gln Glu Val Thr Asp Phe Ala Lys Thr Cys Val Ala Asp Glu Ser Ala | |
| 930 935 940 | |
| Ala Asn Cys Asp Lys Ser Leu His Thr Leu Phe Gly Asp Lys Leu Cys | |
| 945 950 955 960 | |
| Ala Ile Pro Asn Leu Arg Glu Asn Tyr Gly Glu Leu Ala Asp Cys Cys | |
| 965 970 975 | |
| Thr Lys Gln Glu Pro Glu Arg Asn Glu Cys Phe Leu Gln His Lys Asp | |
| 980 985 990 | |
| Asp Asn Pro Ser Leu Pro Pro Phe Glu Arg Pro Glu Ala Glu Ala Met | |
| 995 1000 1005 | |
| Cys Thr Ser Phe Lys Glu Asn Pro Thr Thr Phe Met Gly His Tyr | |
| 1010 1015 1020 | |
| Leu His Glu Val Ala Arg Arg His Pro Tyr Phe Tyr Ala Pro Glu | |
| 1025 1030 1035 | |
| Leu Leu Tyr Tyr Ala Glu Gln Tyr Asn Glu Ile Leu Thr Gln Cys | |
| 1040 1045 1050 | |
| Cys Ala Glu Ala Asp Lys Glu Ser Cys Leu Thr Pro Lys Leu Asp | |
| 1055 1060 1065 | |
| Gly Val Lys Glu Lys Ala Leu Val Ser Ser Val Arg Gln Arg Met | |
| 1070 1075 1080 | |
| Lys Cys Ser Ser Met Gln Lys Phe Gly Glu Arg Ala Phe Lys Ala | |
| 1085 1090 1095 | |
| Trp Ala Val Ala Arg Leu Ser Gln Thr Phe Pro Asn Ala Asp Phe | |
| 1100 1105 1110 | |
| Ala Glu Ile Thr Lys Leu Ala Thr Asp Leu Thr Lys Val Asn Lys | |
| 1115 1120 1125 | |
| Glu Cys Cys His Gly Asp Leu Leu Glu Cys Ala Asp Asp Arg Ala | |
| 1130 1135 1140 | |
| Glu Leu Ala Lys Tyr Met Cys Glu Asn Gln Ala Thr Ile Ser Ser | |
| 1145 1150 1155 | |
| Lys Leu Gln Thr Cys Cys Asp Lys Pro Leu Leu Lys Lys Ala His | |
| 1160 1165 1170 | |
| Cys Leu Ser Glu Val Glu His Asp Thr Met Pro Ala Asp Leu Pro | |
| 1175 1180 1185 | |
| Ala Ile Ala Ala Asp Phe Val Glu Asp Gln Glu Val Cys Lys Asn | |
| 1190 1195 1200 | |
| Tyr Ala Glu Ala Lys Asp Val Phe Leu Gly Thr Phe Leu Tyr Glu | |
| 1205 1210 1215 | |
| Tyr Ser Arg Arg His Pro Asp Tyr Ser Val Ser Leu Leu Leu Arg | |
| 1220 1225 1230 | |
| Leu Ala Lys Lys Tyr Glu Ala Thr Leu Glu Lys Cys Cys Ala Glu | |
| 1235 1240 1245 | |
| Ala Asn Pro Pro Ala Cys Tyr Gly Thr Val Leu Ala Glu Phe Gln | |
| 1250 1255 1260 | |
| Pro Leu Val Glu Glu Pro Lys Asn Leu Val Lys Thr Asn Cys Asp | |
| 1265 1270 1275 | |
| Leu Tyr Glu Lys Leu Gly Glu Tyr Gly Phe Gln Asn Ala Ile Leu | |
| 1280 1285 1290 | |
| Val Arg Tyr Thr Gln Lys Ala Pro Gln Val Ser Thr Pro Thr Leu | |
| 1295 1300 1305 | |
| Val Glu Ala Ala Arg Asn Leu Gly Arg Val Gly Thr Lys Cys Cys | |
| 1310 1315 1320 | |
| Thr Leu Pro Glu Asp Gln Arg Leu Pro Cys Val Glu Asp Tyr Leu | |
| 1325 1330 1335 | |
| Ser Ala Ile Leu Asn Arg Val Cys Leu Leu His Glu Lys Thr Pro | |
| 1340 1345 1350 | |
| Val Ser Glu His Val Thr Lys Cys Cys Ser Gly Ser Leu Val Glu | |
| 1355 1360 1365 | |
| Arg Arg Pro Cys Phe Ser Ala Leu Thr Val Asp Glu Thr Tyr Val | |
| 1370 1375 1380 | |
| Pro Lys Glu Phe Lys Ala Glu Thr Phe Thr Phe His Ser Asp Ile | |
| 1385 1390 1395 | |
| Cys Thr Leu Pro Glu Lys Glu Lys Gln Ile Lys Lys Gln Thr Ala | |
| 1400 1405 1410 | |
| Leu Ala Glu Leu Val Lys His Lys Pro Lys Ala Thr Ala Glu Gln | |
| 1415 1420 1425 | |
| Leu Lys Thr Val Met Asp Asp Phe Ala Gln Phe Leu Asp Thr Cys | |
| 1430 1435 1440 | |
| Cys Lys Ala Ala Asp Lys Asp Thr Cys Phe Ser Thr Glu Gly Pro | |
| 1445 1450 1455 | |
| Asn Leu Val Thr Arg Cys Lys Asp Ala Leu Ala | |
| 1460 1465 | |
| Singly underlined: signal peptide sequence; double-underlined: | |
| beginning and end of NPP3; ** = cleavage position at the signal | |
| peptide sequence; bold residues indicate albumin sequence | |
| ENPP5 Protein Export Signal Sequence | |
| SEQ. ID NO: 16 | |
| Met Thr Ser Lys Phe Leu Leu Val Ser Phe Ile Leu Ala Ala Leu Ser | |
| 1 5 10 15 | |
| Leu Ser Thr Thr Phe Ser Xaa | |
| 20 | |
| ENPP5-1-Fc | |
| SEQ. ID NO: 17 | |
| Met Thr Ser Lys Phe Leu Leu Val Ser Phe Ile Leu Ala Ala Leu Ser | |
| 1 5 10 15 | |
| Leu Ser Thr Thr Phe Ser**Gly Leu Lys Pro Ser Cys Ala Lys Glu Val | |
| 20 25 30 | |
| Lys Ser Cys Lys Gly Arg Cys Phe Glu Arg Thr Phe Gly Asn Cys Arg | |
| 35 40 45 | |
| Cys Asp Ala Ala Cys Val Glu Leu Gly Asn Cys Cys Leu Asp Tyr Gln | |
| 50 55 60 | |
| Glu Thr Cys Ile Glu Pro Glu His Ile Trp Thr Cys Asn Lys Phe Arg | |
| 65 70 75 80 | |
| Cys Gly Glu Lys Arg Leu Thr Arg Ser Leu Cys Ala Cys Ser Asp Asp | |
| 85 90 95 | |
| Cys Lys Asp Lys Gly Asp Cys Cys Ile Asn Tyr Ser Ser Val Cys Gln | |
| 100 105 110 | |
| Gly Glu Lys Ser Trp Val Glu Glu Pro Cys Glu Ser Ile Asn Glu Pro | |
| 115 120 125 | |
| Gln Cys Pro Ala Gly Phe Glu Thr Pro Pro Thr Leu Leu Phe Ser Leu | |
| 130 135 140 | |
| Asp Gly Phe Arg Ala Glu Tyr Leu His Thr Trp Gly Gly Leu Leu Pro | |
| 145 150 155 160 | |
| Val Ile Ser Lys Leu Lys Lys Cys Gly Thr Tyr Thr Lys Asn Met Arg | |
| 165 170 175 | |
| Pro Val Tyr Pro Thr Lys Thr Phe Pro Asn His Tyr Ser Ile Val Thr | |
| 180 185 190 | |
| Gly Leu Tyr Pro Glu Ser His Gly Ile Ile Asp Asn Lys Met Tyr Asp | |
| 195 200 205 | |
| Pro Lys Met Asn Ala Ser Phe Ser Leu Lys Ser Lys Glu Lys Phe Asn | |
| 210 215 220 | |
| Pro Glu Trp Tyr Lys Gly Glu Pro Ile Trp Val Thr Ala Lys Tyr Gln | |
| 225 230 235 240 | |
| Gly Leu Lys Ser Gly Thr Phe Phe Trp Pro Gly Ser Asp Val Glu Ile | |
| 245 250 255 | |
| Asn Gly Ile Phe Pro Asp Ile Tyr Lys Met Tyr Asn Gly Ser Val Pro | |
| 260 265 270 | |
| Phe Glu Glu Arg Ile Leu Ala Val Leu Gln Trp Leu Gln Leu Pro Lys | |
| 275 280 285 | |
| Asp Glu Arg Pro His Phe Tyr Thr Leu Tyr Leu Glu Glu Pro Asp Ser | |
| 290 295 300 | |
| Ser Gly His Ser Tyr Gly Pro Val Ser Ser Glu Val Ile Lys Ala Leu | |
| 305 310 315 320 | |
| Gln Arg Val Asp Gly Met Val Gly Met Leu Met Asp Gly Leu Lys Glu | |
| 325 330 335 | |
| Leu Asn Leu His Arg Cys Leu Asn Leu Ile Leu Ile Ser Asp His Gly | |
| 340 345 350 | |
| Met Glu Gln Gly Ser Cys Lys Lys Tyr Ile Tyr Leu Asn Lys Tyr Leu | |
| 355 360 365 | |
| Gly Asp Val Lys Asn Ile Lys Val Ile Tyr Gly Pro Ala Ala Arg Leu | |
| 370 375 380 | |
| Arg Pro Ser Asp Val Pro Asp Lys Tyr Tyr Ser Phe Asn Tyr Glu Gly | |
| 385 390 395 400 | |
| Ile Ala Arg Asn Leu Ser Cys Arg Glu Pro Asn Gln His Phe Lys Pro | |
| 405 410 415 | |
| Tyr Leu Lys His Phe Leu Pro Lys Arg Leu His Phe Ala Lys Ser Asp | |
| 420 425 430 | |
| Arg Ile Glu Pro Leu Thr Phe Tyr Leu Asp Pro Gln Trp Gln Leu Ala | |
| 435 440 445 | |
| Leu Asn Pro Ser Glu Arg Lys Tyr Cys Gly Ser Gly Phe His Gly Ser | |
| 450 455 460 | |
| Asp Asn Val Phe Ser Asn Met Gln Ala Leu Phe Val Gly Tyr Gly Pro | |
| 465 470 475 480 | |
| Gly Phe Lys His Gly Ile Glu Ala Asp Thr Phe Glu Asn Ile Glu Val | |
| 485 490 495 | |
| Tyr Asn Leu Met Cys Asp Leu Leu Asn Leu Thr Pro Ala Pro Asn Asn | |
| 500 505 510 | |
| Gly Thr His Gly Ser Leu Asn His Leu Leu Lys Asn Pro Val Tyr Thr | |
| 515 520 525 | |
| Pro Lys His Pro Lys Glu Val His Pro Leu Val Gln Cys Pro Phe Thr | |
| 530 535 540 | |
| Arg Asn Pro Arg Asp Asn Leu Gly Cys Ser Cys Asn Pro Ser Ile Leu | |
| 545 550 555 560 | |
| Pro Ile Glu Asp Phe Gln Thr Gln Phe Asn Leu Thr Val Ala Glu Glu | |
| 565 570 575 | |
| Lys Ile Ile Lys His Glu Thr Leu Pro Tyr Gly Arg Pro Arg Val Leu | |
| 580 585 590 | |
| Gln Lys Glu Asn Thr Ile Cys Leu Leu Ser Gln His Gln Phe Met Ser | |
| 595 600 605 | |
| Gly Tyr Ser Gln Asp Ile Leu Met Pro Leu Trp Thr Ser Tyr Thr Val | |
| 610 615 620 | |
| Asp Arg Asn Asp Ser Phe Ser Thr Glu Asp Phe Ser Asn Cys Leu Tyr | |
| 625 630 635 640 | |
| Gln Asp Phe Arg Ile Pro Leu Ser Pro Val His Lys Cys Ser Phe Tyr | |
| 645 650 655 | |
| Lys Asn Asn Thr Lys Val Ser Tyr Gly Phe Leu Ser Pro Pro Gln Leu | |
| 660 665 670 | |
| Asn Lys Asn Ser Ser Gly Ile Tyr Ser Glu Ala Leu Leu Thr Thr Asn | |
| 675 680 685 | |
| Ile Val Pro Met Tyr Gln Ser Phe Gln Val Ile Trp Arg Tyr Phe His | |
| 690 695 700 | |
| Asp Thr Leu Leu Arg Lys Tyr Ala Glu Glu Arg Asn Gly Val Asn Val | |
| 705 710 715 720 | |
| Val Ser Gly Pro Val Phe Asp Phe Asp Tyr Asp Gly Arg Cys Asp Ser | |
| 725 730 735 | |
| Leu Glu Asn Leu Arg Gln Lys Arg Arg Val Ile Arg Asn Gln Glu Ile | |
| 740 745 750 | |
| Leu Ile Pro Thr His Phe Phe Ile Val Leu Thr Ser Cys Lys Asp Thr | |
| 755 760 765 | |
| Ser Gln Thr Pro Leu His Cys Glu Asn Leu Asp Thr Leu Ala Phe Ile | |
| 770 775 780 | |
| Leu Pro His Arg Thr Asp Asn Ser Glu Ser Cys Val His Gly Lys His | |
| 785 790 795 800 | |
| Asp Ser Ser Trp Val Glu Glu Leu Leu Met Leu His Arg Ala Arg Ile | |
| 805 810 815 | |
| Thr Asp Val Glu His Ile Thr Gly Leu Ser Phe Tyr Gln Gln Arg Lys | |
| 820 825 830 | |
| Glu Pro Val Ser Asp Ile Leu Lys Leu Lys Thr His Leu Pro Thr Phe | |
| 835 840 845 | |
| Ser Gln Glu Asp Asp Lys Thr His Thr Cys Pro Pro Cys Pro Ala Pro | |
| 850 855 860 | |
| Glu Leu Leu Gly Gly Pro Ser Val Phe Leu Phe Pro Pro Lys Pro Lys | |
| 865 870 875 880 | |
| Asp Thr Leu Met Ile Ser Arg Thr Pro Glu Val Thr Cys Val Val Val | |
| 885 890 895 | |
| Asp Val Ser His Glu Asp Pro Glu Val Lys Phe Asn Trp Tyr Val Asp | |
| 900 905 910 | |
| Gly Val Glu Val His Asn Ala Lys Thr Lys Pro Arg Glu Glu Gln Tyr | |
| 915 920 925 | |
| Asn Ser Thr Tyr Arg Val Val Ser Val Leu Thr Val Leu His Gln Asp | |
| 930 935 940 | |
| Trp Leu Asn Gly Lys Glu Tyr Lys Cys Lys Val Ser Asn Lys Ala Leu | |
| 945 950 955 960 | |
| Pro Ala Pro Ile Glu Lys Thr Ile Ser Lys Ala Lys Gly Gln Pro Arg | |
| 965 970 975 | |
| Glu Pro Gln Val Tyr Thr Leu Pro Pro Ser Arg Glu Glu Met Thr Lys | |
| 980 985 990 | |
| Asn Gln Val Ser Leu Thr Cys Leu Val Lys Gly Phe Tyr Pro Ser Asp | |
| 995 1000 1005 | |
| Ile Ala Val Glu Trp Glu Ser Asn Gly Gln Pro Glu Asn Asn Tyr | |
| 1010 1015 1020 | |
| Lys Thr Thr Pro Pro Val Leu Asp Ser Asp Gly Ser Phe Phe Leu | |
| 1025 1030 1035 | |
| Tyr Ser Lys Leu Thr Val Asp Lys Ser Arg Trp Gln Gln Gly Asn | |
| 1040 1045 1050 | |
| Val Phe Ser Cys Ser Val Met His Glu Ala Leu His Asn His Tyr | |
| 1055 1060 1065 | |
| Thr Gln Lys Ser Leu Ser Leu Ser Pro Gly Lys | |
| 1070 1075 | |
| Singly underlined: signal peptide sequence; double-underlined: | |
| beginning and end of NPP3; ** = cleavage position at the signal | |
| peptide sequence; bold residues indicate Fc sequence | |
| ENPP7-1-Fc Amino Acid Sequence | |
| SEQ. ID NO: 18 | |
| Met Arg Gly Pro Ala Val Leu Leu Thr Val Ala Leu Ala Thr Leu Leu | |
| 1 5 10 15 | |
| Ala Pro Gly Ala Gly Ala**Gly Leu Lys Pro Ser Cys Ala Lys Glu Val | |
| 20 25 30 | |
| Lys Ser Cys Lys Gly Arg Cys Phe Glu Arg Thr Phe Gly Asn Cys Arg | |
| 35 40 45 | |
| Cys Asp Ala Ala Cys Val Glu Leu Gly Asn Cys Cys Leu Asp Tyr Gln | |
| 50 55 60 | |
| Glu Thr Cys Ile Glu Pro Glu His Ile Trp Thr Cys Asn Lys Phe Arg | |
| 65 70 75 80 | |
| Cys Gly Glu Lys Arg Leu Thr Arg Ser Leu Cys Ala Cys Ser Asp Asp | |
| 85 90 95 | |
| Cys Lys Asp Lys Gly Asp Cys Cys Ile Asn Tyr Ser Ser Val Cys Gln | |
| 100 105 110 | |
| Gly Glu Lys Ser Trp Val Glu Glu Pro Cys Glu Ser Ile Asn Glu Pro | |
| 115 120 125 | |
| Gln Cys Pro Ala Gly Phe Glu Thr Pro Pro Thr Leu Leu Phe Ser Leu | |
| 130 135 140 | |
| Asp Gly Phe Arg Ala Glu Tyr Leu His Thr Trp Gly Gly Leu Leu Pro | |
| 145 150 155 160 | |
| Val Ile Ser Lys Leu Lys Lys Cys Gly Thr Tyr Thr Lys Asn Met Arg | |
| 165 170 175 | |
| Pro Val Tyr Pro Thr Lys Thr Phe Pro Asn His Tyr Ser Ile Val Thr | |
| 180 185 190 | |
| Gly Leu Tyr Pro Glu Ser His Gly Ile Ile Asp Asn Lys Met Tyr Asp | |
| 195 200 205 | |
| Pro Lys Met Asn Ala Ser Phe Ser Leu Lys Ser Lys Glu Lys Phe Asn | |
| 210 215 220 | |
| Pro Glu Trp Tyr Lys Gly Glu Pro Ile Trp Val Thr Ala Lys Tyr Gln | |
| 225 230 235 240 | |
| Gly Leu Lys Ser Gly Thr Phe Phe Trp Pro Gly Ser Asp Val Glu Ile | |
| 245 250 255 | |
| Asn Gly Ile Phe Pro Asp Ile Tyr Lys Met Tyr Asn Gly Ser Val Pro | |
| 260 265 270 | |
| Phe Glu Glu Arg Ile Leu Ala Val Leu Gln Trp Leu Gln Leu Pro Lys | |
| 275 280 285 | |
| Asp Glu Arg Pro His Phe Tyr Thr Leu Tyr Leu Glu Glu Pro Asp Ser | |
| 290 295 300 | |
| Ser Gly His Ser Tyr Gly Pro Val Ser Ser Glu Val Ile Lys Ala Leu | |
| 305 310 315 320 | |
| Gln Arg Val Asp Gly Met Val Gly Met Leu Met Asp Gly Leu Lys Glu | |
| 325 330 335 | |
| Leu Asn Leu His Arg Cys Leu Asn Leu Ile Leu Ile Ser Asp His Gly | |
| 340 345 350 | |
| Met Glu Gln Gly Ser Cys Lys Lys Tyr Ile Tyr Leu Asn Lys Tyr Leu | |
| 355 360 365 | |
| Gly Asp Val Lys Asn Ile Lys Val Ile Tyr Gly Pro Ala Ala Arg Leu | |
| 370 375 380 | |
| Arg Pro Ser Asp Val Pro Asp Lys Tyr Tyr Ser Phe Asn Tyr Glu Gly | |
| 385 390 395 400 | |
| Ile Ala Arg Asn Leu Ser Cys Arg Glu Pro Asn Gln His Phe Lys Pro | |
| 405 410 415 | |
| Tyr Leu Lys His Phe Leu Pro Lys Arg Leu His Phe Ala Lys Ser Asp | |
| 420 425 430 | |
| Arg Ile Glu Pro Leu Thr Phe Tyr Leu Asp Pro Gln Trp Gln Leu Ala | |
| 435 440 445 | |
| Leu Asn Pro Ser Glu Arg Lys Tyr Cys Gly Ser Gly Phe His Gly Ser | |
| 450 455 460 | |
| Asp Asn Val Phe Ser Asn Met Gln Ala Leu Phe Val Gly Tyr Gly Pro | |
| 465 470 475 480 | |
| Gly Phe Lys His Gly Ile Glu Ala Asp Thr Phe Glu Asn Ile Glu Val | |
| 485 490 495 | |
| Tyr Asn Leu Met Cys Asp Leu Leu Asn Leu Thr Pro Ala Pro Asn Asn | |
| 500 505 510 | |
| Gly Thr His Gly Ser Leu Asn His Leu Leu Lys Asn Pro Val Tyr Thr | |
| 515 520 525 | |
| Pro Lys His Pro Lys Glu Val His Pro Leu Val Gln Cys Pro Phe Thr | |
| 530 535 540 | |
| Arg Asn Pro Arg Asp Asn Leu Gly Cys Ser Cys Asn Pro Ser Ile Leu | |
| 545 550 555 560 | |
| Pro Ile Glu Asp Phe Gln Thr Gln Phe Asn Leu Thr Val Ala Glu Glu | |
| 565 570 575 | |
| Lys Ile Ile Lys His Glu Thr Leu Pro Tyr Gly Arg Pro Arg Val Leu | |
| 580 585 590 | |
| Gln Lys Glu Asn Thr Ile Cys Leu Leu Ser Gln His Gln Phe Met Ser | |
| 595 600 605 | |
| Gly Tyr Ser Gln Asp Ile Leu Met Pro Leu Trp Thr Ser Tyr Thr Val | |
| 610 615 620 | |
| Asp Arg Asn Asp Ser Phe Ser Thr Glu Asp Phe Ser Asn Cys Leu Tyr | |
| 625 630 635 640 | |
| Gln Asp Phe Arg Ile Pro Leu Ser Pro Val His Lys Cys Ser Phe Tyr | |
| 645 650 655 | |
| Lys Asn Asn Thr Lys Val Ser Tyr Gly Phe Leu Ser Pro Pro Gln Leu | |
| 660 665 670 | |
| Asn Lys Asn Ser Ser Gly Ile Tyr Ser Glu Ala Leu Leu Thr Thr Asn | |
| 675 680 685 | |
| Ile Val Pro Met Tyr Gln Ser Phe Gln Val Ile Trp Arg Tyr Phe His | |
| 690 695 700 | |
| Asp Thr Leu Leu Arg Lys Tyr Ala Glu Glu Arg Asn Gly Val Asn Val | |
| 705 710 715 720 | |
| Val Ser Gly Pro Val Phe Asp Phe Asp Tyr Asp Gly Arg Cys Asp Ser | |
| 725 730 735 | |
| Leu Glu Asn Leu Arg Gln Lys Arg Arg Val Ile Arg Asn Gln Glu Ile | |
| 740 745 750 | |
| Leu Ile Pro Thr His Phe Phe Ile Val Leu Thr Ser Cys Lys Asp Thr | |
| 755 760 765 | |
| Ser Gln Thr Pro Leu His Cys Glu Asn Leu Asp Thr Leu Ala Phe Ile | |
| 770 775 780 | |
| Leu Pro His Arg Thr Asp Asn Ser Glu Ser Cys Val His Gly Lys His | |
| 785 790 795 800 | |
| Asp Ser Ser Trp Val Glu Glu Leu Leu Met Leu His Arg Ala Arg Ile | |
| 805 810 815 | |
| Thr Asp Val Glu His Ile Thr Gly Leu Ser Phe Tyr Gln Gln Arg Lys | |
| 820 825 830 | |
| Glu Pro Val Ser Asp Ile Leu Lys Leu Lys Thr His Leu Pro Thr Phe | |
| 835 840 845 | |
| Ser Gln Glu Asp Leu Ile Asn Asp Lys Thr His Thr Cys Pro Pro Cys | |
| 850 855 860 | |
| Pro Ala Pro Glu Leu Leu Gly Gly Pro Ser Val Phe Leu Phe Pro Pro | |
| 865 870 875 880 | |
| Lys Pro Lys Asp Thr Leu Met Ile Ser Arg Thr Pro Glu Val Thr Cys | |
| 885 890 895 | |
| Val Val Val Asp Val Ser His Glu Asp Pro Glu Val Lys Phe Asn Trp | |
| 900 905 910 | |
| Tyr Val Asp Gly Val Glu Val His Asn Ala Lys Thr Lys Pro Arg Glu | |
| 915 920 925 | |
| Glu Gln Tyr Asn Ser Thr Tyr Arg Val Val Ser Val Leu Thr Val Leu | |
| 930 935 940 | |
| His Gln Asp Trp Leu Asn Gly Lys Glu Tyr Lys Cys Lys Val Ser Asn | |
| 945 950 955 960 | |
| Lys Ala Leu Pro Ala Pro Ile Glu Lys Thr Ile Ser Lys Ala Lys Gly | |
| 965 970 975 | |
| Gln Pro Arg Glu Pro Gln Val Tyr Thr Leu Pro Pro Ser Arg Glu Glu | |
| 980 985 990 | |
| Met Thr Lys Asn Gln Val Ser Leu Thr Cys Leu Val Lys Gly Phe Tyr | |
| 995 1000 1005 | |
| Pro Ser Asp Ile Ala Val Glu Trp Glu Ser Asn Gly Gln Pro Glu | |
| 1010 1015 1020 | |
| Asn Asn Tyr Lys Thr Thr Pro Pro Val Leu Asp Ser Asp Gly Ser | |
| 1025 1030 1035 | |
| Phe Phe Leu Tyr Ser Lys Leu Thr Val Asp Lys Ser Arg Trp Gln | |
| 1040 1045 1050 | |
| Gln Gly Asn Val Phe Ser Cys Ser Val Met His Glu Ala Leu His | |
| 1055 1060 1065 | |
| Asn His Tyr Thr Gln Lys Ser Leu Ser Leu Ser Pro Gly Lys | |
| 1070 1075 1080 | |
| Singly underlined: signal peptide sequence; double-underlined: | |
| beginning and end of NPP1; ** = cleavage position at the signal | |
| peptide sequence; bold residues indicate Fc sequence | |
| ENPP71 (lacking NPP1 N-Terminus GLK) Amino Acid Sequence: | |
| SEQ. ID NO: 19 | |
| Met Arg Gly Pro Ala Val Leu Leu Thr Val Ala Leu Ala Thr Leu Leu | |
| 1 5 10 15 | |
| Ala Pro Gly Ala Gly Ala**Pro Ser Cys Ala Lys Glu Val Lys Ser Cys | |
| 20 25 30 | |
| Lys Gly Arg Cys Phe Glu Arg Thr Phe Gly Asn Cys Arg Cys Asp Ala | |
| 35 40 45 | |
| Ala Cys Val Glu Leu Gly Asn Cys Cys Leu Asp Tyr Gln Glu Thr Cys | |
| 50 55 60 | |
| Ile Glu Pro Glu His Ile Trp Thr Cys Asn Lys Phe Arg Cys Gly Glu | |
| 65 70 75 80 | |
| Lys Arg Leu Thr Arg Ser Leu Cys Ala Cys Ser Asp Asp Cys Lys Asp | |
| 85 90 95 | |
| Lys Gly Asp Cys Cys Ile Asn Tyr Ser Ser Val Cys Gln Gly Glu Lys | |
| 100 105 110 | |
| Ser Trp Val Glu Glu Pro Cys Glu Ser Ile Asn Glu Pro Gln Cys Pro | |
| 115 120 125 | |
| Ala Gly Phe Glu Thr Pro Pro Thr Leu Leu Phe Ser Leu Asp Gly Phe | |
| 130 135 140 | |
| Arg Ala Glu Tyr Leu His Thr Trp Gly Gly Leu Leu Pro Val Ile Ser | |
| 145 150 155 160 | |
| Lys Leu Lys Lys Cys Gly Thr Tyr Thr Lys Asn Met Arg Pro Val Tyr | |
| 165 170 175 | |
| Pro Thr Lys Thr Phe Pro Asn His Tyr Ser Ile Val Thr Gly Leu Tyr | |
| 180 185 190 | |
| Pro Glu Ser His Gly Ile Ile Asp Asn Lys Met Tyr Asp Pro Lys Met | |
| 195 200 205 | |
| Asn Ala Ser Phe Ser Leu Lys Ser Lys Glu Lys Phe Asn Pro Glu Trp | |
| 210 215 220 | |
| Tyr Lys Gly Glu Pro Ile Trp Val Thr Ala Lys Tyr Gln Gly Leu Lys | |
| 225 230 235 240 | |
| Ser Gly Thr Phe Phe Trp Pro Gly Ser Asp Val Glu Ile Asn Gly Ile | |
| 245 250 255 | |
| Phe Pro Asp Ile Tyr Lys Met Tyr Asn Gly Ser Val Pro Phe Glu Glu | |
| 260 265 270 | |
| Arg Ile Leu Ala Val Leu Gln Trp Leu Gln Leu Pro Lys Asp Glu Arg | |
| 275 280 285 | |
| Pro His Phe Tyr Thr Leu Tyr Leu Glu Glu Pro Asp Ser Ser Gly His | |
| 290 295 300 | |
| Ser Tyr Gly Pro Val Ser Ser Glu Val Ile Lys Ala Leu Gln Arg Val | |
| 305 310 315 320 | |
| Asp Gly Met Val Gly Met Leu Met Asp Gly Leu Lys Glu Leu Asn Leu | |
| 325 330 335 | |
| His Arg Cys Leu Asn Leu Ile Leu Ile Ser Asp His Gly Met Glu Gln | |
| 340 345 350 | |
| Gly Ser Cys Lys Lys Tyr Ile Tyr Leu Asn Lys Tyr Leu Gly Asp Val | |
| 355 360 365 | |
| Lys Asn Ile Lys Val Ile Tyr Gly Pro Ala Ala Arg Leu Arg Pro Ser | |
| 370 375 380 | |
| Asp Val Pro Asp Lys Tyr Tyr Ser Phe Asn Tyr Glu Gly Ile Ala Arg | |
| 385 390 395 400 | |
| Asn Leu Ser Cys Arg Glu Pro Asn Gln His Phe Lys Pro Tyr Leu Lys | |
| 405 410 415 | |
| His Phe Leu Pro Lys Arg Leu His Phe Ala Lys Ser Asp Arg Ile Glu | |
| 420 425 430 | |
| Pro Leu Thr Phe Tyr Leu Asp Pro Gln Trp Gln Leu Ala Leu Asn Pro | |
| 435 440 445 | |
| Ser Glu Arg Lys Tyr Cys Gly Ser Gly Phe His Gly Ser Asp Asn Val | |
| 450 455 460 | |
| Phe Ser Asn Met Gln Ala Leu Phe Val Gly Tyr Gly Pro Gly Phe Lys | |
| 465 470 475 480 | |
| His Gly Ile Glu Ala Asp Thr Phe Glu Asn Ile Glu Val Tyr Asn Leu | |
| 485 490 495 | |
| Met Cys Asp Leu Leu Asn Leu Thr Pro Ala Pro Asn Asn Gly Thr His | |
| 500 505 510 | |
| Gly Ser Leu Asn His Leu Leu Lys Asn Pro Val Tyr Thr Pro Lys His | |
| 515 520 525 | |
| Pro Lys Glu Val His Pro Leu Val Gln Cys Pro Phe Thr Arg Asn Pro | |
| 530 535 540 | |
| Arg Asp Asn Leu Gly Cys Ser Cys Asn Pro Ser Ile Leu Pro Ile Glu | |
| 545 550 555 560 | |
| Asp Phe Gln Thr Gln Phe Asn Leu Thr Val Ala Glu Glu Lys Ile Ile | |
| 565 570 575 | |
| Lys His Glu Thr Leu Pro Tyr Gly Arg Pro Arg Val Leu Gln Lys Glu | |
| 580 585 590 | |
| Asn Thr Ile Cys Leu Leu Ser Gln His Gln Phe Met Ser Gly Tyr Ser | |
| 595 600 605 | |
| Gln Asp Ile Leu Met Pro Leu Trp Thr Ser Tyr Thr Val Asp Arg Asn | |
| 610 615 620 | |
| Asp Ser Phe Ser Thr Glu Asp Phe Ser Asn Cys Leu Tyr Gln Asp Phe | |
| 625 630 635 640 | |
| Arg Ile Pro Leu Ser Pro Val His Lys Cys Ser Phe Tyr Lys Asn Asn | |
| 645 650 655 | |
| Thr Lys Val Ser Tyr Gly Phe Leu Ser Pro Pro Gln Leu Asn Lys Asn | |
| 660 665 670 | |
| Ser Ser Gly Ile Tyr Ser Glu Ala Leu Leu Thr Thr Asn Ile Val Pro | |
| 675 680 685 | |
| Met Tyr Gln Ser Phe Gln Val Ile Trp Arg Tyr Phe His Asp Thr Leu | |
| 690 695 700 | |
| Leu Arg Lys Tyr Ala Glu Glu Arg Asn Gly Val Asn Val Val Ser Gly | |
| 705 710 715 720 | |
| Pro Val Phe Asp Phe Asp Tyr Asp Gly Arg Cys Asp Ser Leu Glu Asn | |
| 725 730 735 | |
| Leu Arg Gln Lys Arg Arg Val Ile Arg Asn Gln Glu Ile Leu Ile Pro | |
| 740 745 750 | |
| Thr His Phe Phe Ile Val Leu Thr Ser Cys Lys Asp Thr Ser Gln Thr | |
| 755 760 765 | |
| Pro Leu His Cys Glu Asn Leu Asp Thr Leu Ala Phe Ile Leu Pro His | |
| 770 775 780 | |
| Arg Thr Asp Asn Ser Glu Ser Cys Val His Gly Lys His Asp Ser Ser | |
| 785 790 795 800 | |
| Trp Val Glu Glu Leu Leu Met Leu His Arg Ala Arg Ile Thr Asp Val | |
| 805 810 815 | |
| Glu His Ile Thr Gly Leu Ser Phe Tyr Gln Gln Arg Lys Glu Pro Val | |
| 820 825 830 | |
| Ser Asp Ile Leu Lys Leu Lys Thr His Leu Pro Thr Phe Ser Gln Glu | |
| 835 840 845 | |
| Asp | |
| Singly underlined: signal peptide sequence; double-underlined: | |
| beginning and end of NPP3; ** = cleavage position at the signal | |
| peptide sequence | |
| ENPP71 (lacking NPP1 N-Terminus GLK) - Fc Amino Acid Sequence: | |
| SEQ. ID NO: 20 | |
| Met Arg Gly Pro Ala Val Leu Leu Thr Val Ala Leu Ala Thr Leu Leu | |
| 1 5 10 15 | |
| Ala Pro Gly Ala Gly Ala**Pro Ser Cys Ala Lys Glu Val Lys Ser Cys | |
| 20 25 30 | |
| Lys Gly Arg Cys Phe Glu Arg Thr Phe Gly Asn Cys Arg Cys Asp Ala | |
| 35 40 45 | |
| Ala Cys Val Glu Leu Gly Asn Cys Cys Leu Asp Tyr Gln Glu Thr Cys | |
| 50 55 60 | |
| Ile Glu Pro Glu His Ile Trp Thr Cys Asn Lys Phe Arg Cys Gly Glu | |
| 65 70 75 80 | |
| Lys Arg Leu Thr Arg Ser Leu Cys Ala Cys Ser Asp Asp Cys Lys Asp | |
| 85 90 95 | |
| Lys Gly Asp Cys Cys Ile Asn Tyr Ser Ser Val Cys Gln Gly Glu Lys | |
| 100 105 110 | |
| Ser Trp Val Glu Glu Pro Cys Glu Ser Ile Asn Glu Pro Gln Cys Pro | |
| 115 120 125 | |
| Ala Gly Phe Glu Thr Pro Pro Thr Leu Leu Phe Ser Leu Asp Gly Phe | |
| 130 135 140 | |
| Arg Ala Glu Tyr Leu His Thr Trp Gly Gly Leu Leu Pro Val Ile Ser | |
| 145 150 155 160 | |
| Lys Leu Lys Lys Cys Gly Thr Tyr Thr Lys Asn Met Arg Pro Val Tyr | |
| 165 170 175 | |
| Pro Thr Lys Thr Phe Pro Asn His Tyr Ser Ile Val Thr Gly Leu Tyr | |
| 180 185 190 | |
| Pro Glu Ser His Gly Ile Ile Asp Asn Lys Met Tyr Asp Pro Lys Met | |
| 195 200 205 | |
| Asn Ala Ser Phe Ser Leu Lys Ser Lys Glu Lys Phe Asn Pro Glu Trp | |
| 210 215 220 | |
| Tyr Lys Gly Glu Pro Ile Trp Val Thr Ala Lys Tyr Gln Gly Leu Lys | |
| 225 230 235 240 | |
| Ser Gly Thr Phe Phe Trp Pro Gly Ser Asp Val Glu Ile Asn Gly Ile | |
| 245 250 255 | |
| Phe Pro Asp Ile Tyr Lys Met Tyr Asn Gly Ser Val Pro Phe Glu Glu | |
| 260 265 270 | |
| Arg Ile Leu Ala Val Leu Gln Trp Leu Gln Leu Pro Lys Asp Glu Arg | |
| 275 280 285 | |
| Pro His Phe Tyr Thr Leu Tyr Leu Glu Glu Pro Asp Ser Ser Gly His | |
| 290 295 300 | |
| Ser Tyr Gly Pro Val Ser Ser Glu Val Ile Lys Ala Leu Gln Arg Val | |
| 305 310 315 320 | |
| Asp Gly Met Val Gly Met Leu Met Asp Gly Leu Lys Glu Leu Asn Leu | |
| 325 330 335 | |
| His Arg Cys Leu Asn Leu Ile Leu Ile Ser Asp His Gly Met Glu Gln | |
| 340 345 350 | |
| Gly Ser Cys Lys Lys Tyr Ile Tyr Leu Asn Lys Tyr Leu Gly Asp Val | |
| 355 360 365 | |
| Lys Asn Ile Lys Val Ile Tyr Gly Pro Ala Ala Arg Leu Arg Pro Ser | |
| 370 375 380 | |
| Asp Val Pro Asp Lys Tyr Tyr Ser Phe Asn Tyr Glu Gly Ile Ala Arg | |
| 385 390 395 400 | |
| Asn Leu Ser Cys Arg Glu Pro Asn Gln His Phe Lys Pro Tyr Leu Lys | |
| 405 410 415 | |
| His Phe Leu Pro Lys Arg Leu His Phe Ala Lys Ser Asp Arg Ile Glu | |
| 420 425 430 | |
| Pro Leu Thr Phe Tyr Leu Asp Pro Gln Trp Gln Leu Ala Leu Asn Pro | |
| 435 440 445 | |
| Ser Glu Arg Lys Tyr Cys Gly Ser Gly Phe His Gly Ser Asp Asn Val | |
| 450 455 460 | |
| Phe Ser Asn Met Gln Ala Leu Phe Val Gly Tyr Gly Pro Gly Phe Lys | |
| 465 470 475 480 | |
| His Gly Ile Glu Ala Asp Thr Phe Glu Asn Ile Glu Val Tyr Asn Leu | |
| 485 490 495 | |
| Met Cys Asp Leu Leu Asn Leu Thr Pro Ala Pro Asn Asn Gly Thr His | |
| 500 505 510 | |
| Gly Ser Leu Asn His Leu Leu Lys Asn Pro Val Tyr Thr Pro Lys His | |
| 515 520 525 | |
| Pro Lys Glu Val His Pro Leu Val Gln Cys Pro Phe Thr Arg Asn Pro | |
| 530 535 540 | |
| Arg Asp Asn Leu Gly Cys Ser Cys Asn Pro Ser Ile Leu Pro Ile Glu | |
| 545 550 555 560 | |
| Asp Phe Gln Thr Gln Phe Asn Leu Thr Val Ala Glu Glu Lys Ile Ile | |
| 565 570 575 | |
| Lys His Glu Thr Leu Pro Tyr Gly Arg Pro Arg Val Leu Gln Lys Glu | |
| 580 585 590 | |
| Asn Thr Ile Cys Leu Leu Ser Gln His Gln Phe Met Ser Gly Tyr Ser | |
| 595 600 605 | |
| Gln Asp Ile Leu Met Pro Leu Trp Thr Ser Tyr Thr Val Asp Arg Asn | |
| 610 615 620 | |
| Asp Ser Phe Ser Thr Glu Asp Phe Ser Asn Cys Leu Tyr Gln Asp Phe | |
| 625 630 635 640 | |
| Arg Ile Pro Leu Ser Pro Val His Lys Cys Ser Phe Tyr Lys Asn Asn | |
| 645 650 655 | |
| Thr Lys Val Ser Tyr Gly Phe Leu Ser Pro Pro Gln Leu Asn Lys Asn | |
| 660 665 670 | |
| Ser Ser Gly Ile Tyr Ser Glu Ala Leu Leu Thr Thr Asn Ile Val Pro | |
| 675 680 685 | |
| Met Tyr Gln Ser Phe Gln Val Ile Trp Arg Tyr Phe His Asp Thr Leu | |
| 690 695 700 | |
| Leu Arg Lys Tyr Ala Glu Glu Arg Asn Gly Val Asn Val Val Ser Gly | |
| 705 710 715 720 | |
| Pro Val Phe Asp Phe Asp Tyr Asp Gly Arg Cys Asp Ser Leu Glu Asn | |
| 725 730 735 | |
| Leu Arg Gln Lys Arg Arg Val Ile Arg Asn Gln Glu Ile Leu Ile Pro | |
| 740 745 750 | |
| Thr His Phe Phe Ile Val Leu Thr Ser Cys Lys Asp Thr Ser Gln Thr | |
| 755 760 765 | |
| Pro Leu His Cys Glu Asn Leu Asp Thr Leu Ala Phe Ile Leu Pro His | |
| 770 775 780 | |
| Arg Thr Asp Asn Ser Glu Ser Cys Val His Gly Lys His Asp Ser Ser | |
| 785 790 795 800 | |
| Trp Val Glu Glu Leu Leu Met Leu His Arg Ala Arg Ile Thr Asp Val | |
| 805 810 815 | |
| Glu His Ile Thr Gly Leu Ser Phe Tyr Gln Gln Arg Lys Glu Pro Val | |
| 820 825 830 | |
| Ser Asp Ile Leu Lys Leu Lys Thr His Leu Pro Thr Phe Ser Gln Glu | |
| 835 840 845 | |
| Asp Leu Ile Asn Asp Lys Thr His Thr Cys Pro Pro Cys Pro Ala Pro | |
| 850 855 860 | |
| Glu Leu Leu Gly Gly Pro Ser Val Phe Leu Phe Pro Pro Lys Pro Lys | |
| 865 870 875 880 | |
| Asp Thr Leu Met Ile Ser Arg Thr Pro Glu Val Thr Cys Val Val Val | |
| 885 890 895 | |
| Asp Val Ser His Glu Asp Pro Glu Val Lys Phe Asn Trp Tyr Val Asp | |
| 900 905 910 | |
| Gly Val Glu Val His Asn Ala Lys Thr Lys Pro Arg Glu Glu Gln Tyr | |
| 915 920 925 | |
| Asn Ser Thr Tyr Arg Val Val Ser Val Leu Thr Val Leu His Gln Asp | |
| 930 935 940 | |
| Trp Leu Asn Gly Lys Glu Tyr Lys Cys Lys Val Ser Asn Lys Ala Leu | |
| 945 950 955 960 | |
| Pro Ala Pro Ile Glu Lys Thr Ile Ser Lys Ala Lys Gly Gln Pro Arg | |
| 965 970 975 | |
| Glu Pro Gln Val Tyr Thr Leu Pro Pro Ser Arg Glu Glu Met Thr Lys | |
| 980 985 990 | |
| Asn Gln Val Ser Leu Thr Cys Leu Val Lys Gly Phe Tyr Pro Ser Asp | |
| 995 1000 1005 | |
| Ile Ala Val Glu Trp Glu Ser Asn Gly Gln Pro Glu Asn Asn Tyr | |
| 1010 1015 1020 | |
| Lys Thr Thr Pro Pro Val Leu Asp Ser Asp Gly Ser Phe Phe Leu | |
| 1025 1030 1035 | |
| Tyr Ser Lys Leu Thr Val Asp Lys Ser Arg Trp Gln Gln Gly Asn | |
| 1040 1045 1050 | |
| Val Phe Ser Cys Ser Val Met His Glu Ala Leu His Asn His Tyr | |
| 1055 1060 1065 | |
| Thr Gln Lys Ser Leu Ser Leu Ser Pro Gly Lys | |
| 1070 1075 | |
| Singly underlined: signal peptide sequence; double-underlined: | |
| beginning and end of NPP1; ** = cleavage position at the signal | |
| peptide sequence; bold residues indicate Fc sequence | |
| ENPP7-1 (lacking NPP1 N-Terminus GLK) - ALB Amino Acid Sequence | |
| SEQ. ID NO: 21 | |
| Met Arg Gly Pro Ala Val Leu Leu Thr Val Ala Leu Ala Thr Leu Leu | |
| 1 5 10 15 | |
| Ala Pro Gly Ala Gly Ala**Pro Ser Cys Ala Lys Glu Val Lys Ser Cys | |
| 20 25 30 | |
| Lys Gly Arg Cys Phe Glu Arg Thr Phe Gly Asn Cys Arg Cys Asp Ala | |
| 35 40 45 | |
| Ala Cys Val Glu Leu Gly Asn Cys Cys Leu Asp Tyr Gln Glu Thr Cys | |
| 50 55 60 | |
| Ile Glu Pro Glu His Ile Trp Thr Cys Asn Lys Phe Arg Cys Gly Glu | |
| 65 70 75 80 | |
| Lys Arg Leu Thr Arg Ser Leu Cys Ala Cys Ser Asp Asp Cys Lys Asp | |
| 85 90 95 | |
| Lys Gly Asp Cys Cys Ile Asn Tyr Ser Ser Val Cys Gln Gly Glu Lys | |
| 100 105 110 | |
| Ser Trp Val Glu Glu Pro Cys Glu Ser Ile Asn Glu Pro Gln Cys Pro | |
| 115 120 125 | |
| Ala Gly Phe Glu Thr Pro Pro Thr Leu Leu Phe Ser Leu Asp Gly Phe | |
| 130 135 140 | |
| Arg Ala Glu Tyr Leu His Thr Trp Gly Gly Leu Leu Pro Val Ile Ser | |
| 145 150 155 160 | |
| Lys Leu Lys Lys Cys Gly Thr Tyr Thr Lys Asn Met Arg Pro Val Tyr | |
| 165 170 175 | |
| Pro Thr Lys Thr Phe Pro Asn His Tyr Ser Ile Val Thr Gly Leu Tyr | |
| 180 185 190 | |
| Pro Glu Ser His Gly Ile Ile Asp Asn Lys Met Tyr Asp Pro Lys Met | |
| 195 200 205 | |
| Asn Ala Ser Phe Ser Leu Lys Ser Lys Glu Lys Phe Asn Pro Glu Trp | |
| 210 215 220 | |
| Tyr Lys Gly Glu Pro Ile Trp Val Thr Ala Lys Tyr Gln Gly Leu Lys | |
| 225 230 235 240 | |
| Ser Gly Thr Phe Phe Trp Pro Gly Ser Asp Val Glu Ile Asn Gly Ile | |
| 245 250 255 | |
| Phe Pro Asp Ile Tyr Lys Met Tyr Asn Gly Ser Val Pro Phe Glu Glu | |
| 260 265 270 | |
| Arg Ile Leu Ala Val Leu Gln Trp Leu Gln Leu Pro Lys Asp Glu Arg | |
| 275 280 285 | |
| Pro His Phe Tyr Thr Leu Tyr Leu Glu Glu Pro Asp Ser Ser Gly His | |
| 290 295 300 | |
| Ser Tyr Gly Pro Val Ser Ser Glu Val Ile Lys Ala Leu Gln Arg Val | |
| 305 310 315 320 | |
| Asp Gly Met Val Gly Met Leu Met Asp Gly Leu Lys Glu Leu Asn Leu | |
| 325 330 335 | |
| His Arg Cys Leu Asn Leu Ile Leu Ile Ser Asp His Gly Met Glu Gln | |
| 340 345 350 | |
| Gly Ser Cys Lys Lys Tyr Ile Tyr Leu Asn Lys Tyr Leu Gly Asp Val | |
| 355 360 365 | |
| Lys Asn Ile Lys Val Ile Tyr Gly Pro Ala Ala Arg Leu Arg Pro Ser | |
| 370 375 380 | |
| Asp Val Pro Asp Lys Tyr Tyr Ser Phe Asn Tyr Glu Gly Ile Ala Arg | |
| 385 390 395 400 | |
| Asn Leu Ser Cys Arg Glu Pro Asn Gln His Phe Lys Pro Tyr Leu Lys | |
| 405 410 415 | |
| His Phe Leu Pro Lys Arg Leu His Phe Ala Lys Ser Asp Arg Ile Glu | |
| 420 425 430 | |
| Pro Leu Thr Phe Tyr Leu Asp Pro Gln Trp Gln Leu Ala Leu Asn Pro | |
| 435 440 445 | |
| Ser Glu Arg Lys Tyr Cys Gly Ser Gly Phe His Gly Ser Asp Asn Val | |
| 450 455 460 | |
| Phe Ser Asn Met Gln Ala Leu Phe Val Gly Tyr Gly Pro Gly Phe Lys | |
| 465 470 475 480 | |
| His Gly Ile Glu Ala Asp Thr Phe Glu Asn Ile Glu Val Tyr Asn Leu | |
| 485 490 495 | |
| Met Cys Asp Leu Leu Asn Leu Thr Pro Ala Pro Asn Asn Gly Thr His | |
| 500 505 510 | |
| Gly Ser Leu Asn His Leu Leu Lys Asn Pro Val Tyr Thr Pro Lys His | |
| 515 520 525 | |
| Pro Lys Glu Val His Pro Leu Val Gln Cys Pro Phe Thr Arg Asn Pro | |
| 530 535 540 | |
| Arg Asp Asn Leu Gly Cys Ser Cys Asn Pro Ser Ile Leu Pro Ile Glu | |
| 545 550 555 560 | |
| Asp Phe Gln Thr Gln Phe Asn Leu Thr Val Ala Glu Glu Lys Ile Ile | |
| 565 570 575 | |
| Lys His Glu Thr Leu Pro Tyr Gly Arg Pro Arg Val Leu Gln Lys Glu | |
| 580 585 590 | |
| Asn Thr Ile Cys Leu Leu Ser Gln His Gln Phe Met Ser Gly Tyr Ser | |
| 595 600 605 | |
| Gln Asp Ile Leu Met Pro Leu Trp Thr Ser Tyr Thr Val Asp Arg Asn | |
| 610 615 620 | |
| Asp Ser Phe Ser Thr Glu Asp Phe Ser Asn Cys Leu Tyr Gln Asp Phe | |
| 625 630 635 640 | |
| Arg Ile Pro Leu Ser Pro Val His Lys Cys Ser Phe Tyr Lys Asn Asn | |
| 645 650 655 | |
| Thr Lys Val Ser Tyr Gly Phe Leu Ser Pro Pro Gln Leu Asn Lys Asn | |
| 660 665 670 | |
| Ser Ser Gly Ile Tyr Ser Glu Ala Leu Leu Thr Thr Asn Ile Val Pro | |
| 675 680 685 | |
| Met Tyr Gln Ser Phe Gln Val Ile Trp Arg Tyr Phe His Asp Thr Leu | |
| 690 695 700 | |
| Leu Arg Lys Tyr Ala Glu Glu Arg Asn Gly Val Asn Val Val Ser Gly | |
| 705 710 715 720 | |
| Pro Val Phe Asp Phe Asp Tyr Asp Gly Arg Cys Asp Ser Leu Glu Asn | |
| 725 730 735 | |
| Leu Arg Gln Lys Arg Arg Val Ile Arg Asn Gln Glu Ile Leu Ile Pro | |
| 740 745 750 | |
| Thr His Phe Phe Ile Val Leu Thr Ser Cys Lys Asp Thr Ser Gln Thr | |
| 755 760 765 | |
| Pro Leu His Cys Glu Asn Leu Asp Thr Leu Ala Phe Ile Leu Pro His | |
| 770 775 780 | |
| Arg Thr Asp Asn Ser Glu Ser Cys Val His Gly Lys His Asp Ser Ser | |
| 785 790 795 800 | |
| Trp Val Glu Glu Leu Leu Met Leu His Arg Ala Arg Ile Thr Asp Val | |
| 805 810 815 | |
| Glu His Ile Thr Gly Leu Ser Phe Tyr Gln Gln Arg Lys Glu Pro Val | |
| 820 825 830 | |
| Ser Asp Ile Leu Lys Leu Lys Thr His Leu Pro Thr Phe Ser Gln Glu | |
| 835 840 845 | |
| Asp Arg Ser Gly Ser Gly Gly Ser Met Lys Trp Val Thr Phe Leu Leu | |
| 850 855 860 | |
| Leu Leu Phe Val Ser Gly Ser Ala Phe Ser Arg Gly Val Phe Arg Arg | |
| 865 870 875 880 | |
| Glu Ala His Lys Ser Glu Ile Ala His Arg Tyr Asn Asp Leu Gly Glu | |
| 885 890 895 | |
| Gln His Phe Lys Gly Leu Val Leu Ile Ala Phe Ser Gln Tyr Leu Gln | |
| 900 905 910 | |
| Lys Cys Ser Tyr Asp Glu His Ala Lys Leu Val Gln Glu Val Thr Asp | |
| 915 920 925 | |
| Phe Ala Lys Thr Cys Val Ala Asp Glu Ser Ala Ala Asn Cys Asp Lys | |
| 930 935 940 | |
| Ser Leu His Thr Leu Phe Gly Asp Lys Leu Cys Ala Ile Pro Asn Leu | |
| 945 950 955 960 | |
| Arg Glu Asn Tyr Gly Glu Leu Ala Asp Cys Cys Thr Lys Gln Glu Pro | |
| 965 970 975 | |
| Glu Arg Asn Glu Cys Phe Leu Gln His Lys Asp Asp Asn Pro Ser Leu | |
| 980 985 990 | |
| Pro Pro Phe Glu Arg Pro Glu Ala Glu Ala Met Cys Thr Ser Phe Lys | |
| 995 1000 1005 | |
| Glu Asn Pro Thr Thr Phe Met Gly His Tyr Leu His Glu Val Ala | |
| 1010 1015 1020 | |
| Arg Arg His Pro Tyr Phe Tyr Ala Pro Glu Leu Leu Tyr Tyr Ala | |
| 1025 1030 1035 | |
| Glu Gln Tyr Asn Glu Ile Leu Thr Gln Cys Cys Ala Glu Ala Asp | |
| 1040 1045 1050 | |
| Lys Glu Ser Cys Leu Thr Pro Lys Leu Asp Gly Val Lys Glu Lys | |
| 1055 1060 1065 | |
| Ala Leu Val Ser Ser Val Arg Gln Arg Met Lys Cys Ser Ser Met | |
| 1070 1075 1080 | |
| Gln Lys Phe Gly Glu Arg Ala Phe Lys Ala Trp Ala Val Ala Arg | |
| 1085 1090 1095 | |
| Leu Ser Gln Thr Phe Pro Asn Ala Asp Phe Ala Glu Ile Thr Lys | |
| 1100 1105 1110 | |
| Leu Ala Thr Asp Leu Thr Lys Val Asn Lys Glu Cys Cys His Gly | |
| 1115 1120 1125 | |
| Asp Leu Leu Glu Cys Ala Asp Asp Arg Ala Glu Leu Ala Lys Tyr | |
| 1130 1135 1140 | |
| Met Cys Glu Asn Gln Ala Thr Ile Ser Ser Lys Leu Gln Thr Cys | |
| 1145 1150 1155 | |
| Cys Asp Lys Pro Leu Leu Lys Lys Ala His Cys Leu Ser Glu Val | |
| 1160 1165 1170 | |
| Glu His Asp Thr Met Pro Ala Asp Leu Pro Ala Ile Ala Ala Asp | |
| 1175 1180 1185 | |
| Phe Val Glu Asp Gln Glu Val Cys Lys Asn Tyr Ala Glu Ala Lys | |
| 1190 1195 1200 | |
| Asp Val Phe Leu Gly Thr Phe Leu Tyr Glu Tyr Ser Arg Arg His | |
| 1205 1210 1215 | |
| Pro Asp Tyr Ser Val Ser Leu Leu Leu Arg Leu Ala Lys Lys Tyr | |
| 1220 1225 1230 | |
| Glu Ala Thr Leu Glu Lys Cys Cys Ala Glu Ala Asn Pro Pro Ala | |
| 1235 1240 1245 | |
| Cys Tyr Gly Thr Val Leu Ala Glu Phe Gln Pro Leu Val Glu Glu | |
| 1250 1255 1260 | |
| Pro Lys Asn Leu Val Lys Thr Asn Cys Asp Leu Tyr Glu Lys Leu | |
| 1265 1270 1275 | |
| Gly Glu Tyr Gly Phe Gln Asn Ala Ile Leu Val Arg Tyr Thr Gln | |
| 1280 1285 1290 | |
| Lys Ala Pro Gln Val Ser Thr Pro Thr Leu Val Glu Ala Ala Arg | |
| 1295 1300 1305 | |
| Asn Leu Gly Arg Val Gly Thr Lys Cys Cys Thr Leu Pro Glu Asp | |
| 1310 1315 1320 | |
| Gln Arg Leu Pro Cys Val Glu Asp Tyr Leu Ser Ala Ile Leu Asn | |
| 1325 1330 1335 | |
| Arg Val Cys Leu Leu His Glu Lys Thr Pro Val Ser Glu His Val | |
| 1340 1345 1350 | |
| Thr Lys Cys Cys Ser Gly Ser Leu Val Glu Arg Arg Pro Cys Phe | |
| 1355 1360 1365 | |
| Ser Ala Leu Thr Val Asp Glu Thr Tyr Val Pro Lys Glu Phe Lys | |
| 1370 1375 1380 | |
| Ala Glu Thr Phe Thr Phe His Ser Asp Ile Cys Thr Leu Pro Glu | |
| 1385 1390 1395 | |
| Lys Glu Lys Gln Ile Lys Lys Gln Thr Ala Leu Ala Glu Leu Val | |
| 1400 1405 1410 | |
| Lys His Lys Pro Lys Ala Thr Ala Glu Gln Leu Lys Thr Val Met | |
| 1415 1420 1425 | |
| Asp Asp Phe Ala Gln Phe Leu Asp Thr Cys Cys Lys Ala Ala Asp | |
| 1430 1435 1440 | |
| Lys Asp Thr Cys Phe Ser Thr Glu Gly Pro Asn Leu Val Thr Arg | |
| 1445 1450 1455 | |
| Cys Lys Asp Ala Leu Ala Arg Ser Trp Ser His Pro Gln Phe Glu | |
| 1460 1465 1470 | |
| Lys | |
| Singly underlined: signal peptide sequence; double-underlined: | |
| beginning and end of NPP1; ** = cleavage position at the signal | |
| peptide sequence; bold residues indicate albumin sequence | |
| ENPP7-NPP3-Fc sequence: | |
| SEQ. ID NO: 22 | |
| Met Arg Gly Pro Ala Val Leu Leu Thr Val Ala Leu Ala Thr Leu Leu | |
| 1 5 10 15 | |
| Ala Pro Gly Ala**Lys Gln Gly Ser Cys Arg Lys Lys Cys Phe Asp Ala | |
| 20 25 30 | |
| Ser Phe Arg Gly Leu Glu Asn Cys Arg Cys Asp Val Ala Cys Lys Asp | |
| 35 40 45 | |
| Arg Gly Asp Cys Cys Trp Asp Phe Glu Asp Thr Cys Val Glu Ser Thr | |
| 50 55 60 | |
| Arg Ile Trp Met Cys Asn Lys Phe Arg Cys Gly Glu Arg Leu Glu Ala | |
| 65 70 75 80 | |
| Ser Leu Cys Ser Cys Ser Asp Asp Cys Leu Gln Arg Lys Asp Cys Cys | |
| 85 90 95 | |
| Ala Asp Tyr Lys Ser Val Cys Gln Gly Glu Thr Ser Trp Leu Glu Glu | |
| 100 105 110 | |
| Asn Cys Asp Thr Ala Gln Gln Ser Gln Cys Pro Glu Gly Phe Asp Leu | |
| 115 120 125 | |
| Pro Pro Val Ile Leu Phe Ser Met Asp Gly Phe Arg Ala Glu Tyr Leu | |
| 130 135 140 | |
| Tyr Thr Trp Asp Thr Leu Met Pro Asn Ile Asn Lys Leu Lys Thr Cys | |
| 145 150 155 160 | |
| Gly Ile His Ser Lys Tyr Met Arg Ala Met Tyr Pro Thr Lys Thr Phe | |
| 165 170 175 | |
| Pro Asn His Tyr Thr Ile Val Thr Gly Leu Tyr Pro Glu Ser His Gly | |
| 180 185 190 | |
| Ile Ile Asp Asn Asn Met Tyr Asp Val Asn Leu Asn Lys Asn Phe Ser | |
| 195 200 205 | |
| Leu Ser Ser Lys Glu Gln Asn Asn Pro Ala Trp Trp His Gly Gln Pro | |
| 210 215 220 | |
| Met Trp Leu Thr Ala Met Tyr Gln Gly Leu Lys Ala Ala Thr Tyr Phe | |
| 225 230 235 240 | |
| Trp Pro Gly Ser Glu Val Ala Ile Asn Gly Ser Phe Pro Ser Ile Tyr | |
| 245 250 255 | |
| Met Pro Tyr Asn Gly Ser Val Pro Phe Glu Glu Arg Ile Ser Thr Leu | |
| 260 265 270 | |
| Leu Lys Trp Leu Asp Leu Pro Lys Ala Glu Arg Pro Arg Phe Tyr Thr | |
| 275 280 285 | |
| Met Tyr Phe Glu Glu Pro Asp Ser Ser Gly His Ala Gly Gly Pro Val | |
| 290 295 300 | |
| Ser Ala Arg Val Ile Lys Ala Leu Gln Val Val Asp His Ala Phe Gly | |
| 305 310 315 320 | |
| Met Leu Met Glu Gly Leu Lys Gln Arg Asn Leu His Asn Cys Val Asn | |
| 325 330 335 | |
| Ile Ile Leu Leu Ala Asp His Gly Met Asp Gln Thr Tyr Cys Asn Lys | |
| 340 345 350 | |
| Met Glu Tyr Met Thr Asp Tyr Phe Pro Arg Ile Asn Phe Phe Tyr Met | |
| 355 360 365 | |
| Tyr Glu Gly Pro Ala Pro Arg Ile Arg Ala His Asn Ile Pro His Asp | |
| 370 375 380 | |
| Phe Phe Ser Phe Asn Ser Glu Glu Ile Val Arg Asn Leu Ser Cys Arg | |
| 385 390 395 400 | |
| Lys Pro Asp Gln His Phe Lys Pro Tyr Leu Thr Pro Asp Leu Pro Lys | |
| 405 410 415 | |
| Arg Leu His Tyr Ala Lys Asn Val Arg Ile Asp Lys Val His Leu Phe | |
| 420 425 430 | |
| Val Asp Gln Gln Trp Leu Ala Val Arg Ser Lys Ser Asn Thr Asn Cys | |
| 435 440 445 | |
| Gly Gly Gly Asn His Gly Tyr Asn Asn Glu Phe Arg Ser Met Glu Ala | |
| 450 455 460 | |
| Ile Phe Leu Ala His Gly Pro Ser Phe Lys Glu Lys Thr Glu Val Glu | |
| 465 470 475 480 | |
| Pro Phe Glu Asn Ile Glu Val Tyr Asn Leu Met Cys Asp Leu Leu Arg | |
| 485 490 495 | |
| Ile Gln Pro Ala Pro Asn Asn Gly Thr His Gly Ser Leu Asn His Leu | |
| 500 505 510 | |
| Leu Lys Val Pro Phe Tyr Glu Pro Ser His Ala Glu Glu Val Ser Lys | |
| 515 520 525 | |
| Phe Ser Val Cys Gly Phe Ala Asn Pro Leu Pro Thr Glu Ser Leu Asp | |
| 530 535 540 | |
| Cys Phe Cys Pro His Leu Gln Asn Ser Thr Gln Leu Glu Gln Val Asn | |
| 545 550 555 560 | |
| Gln Met Leu Asn Leu Thr Gln Glu Glu Ile Thr Ala Thr Val Lys Val | |
| 565 570 575 | |
| Asn Leu Pro Phe Gly Arg Pro Arg Val Leu Gln Lys Asn Val Asp His | |
| 580 585 590 | |
| Cys Leu Leu Tyr His Arg Glu Tyr Val Ser Gly Phe Gly Lys Ala Met | |
| 595 600 605 | |
| Arg Met Pro Met Trp Ser Ser Tyr Thr Val Pro Gln Leu Gly Asp Thr | |
| 610 615 620 | |
| Ser Pro Leu Pro Pro Thr Val Pro Asp Cys Leu Arg Ala Asp Val Arg | |
| 625 630 635 640 | |
| Val Pro Pro Ser Glu Ser Gln Lys Cys Ser Phe Tyr Leu Ala Asp Lys | |
| 645 650 655 | |
| Asn Ile Thr His Gly Phe Leu Tyr Pro Pro Ala Ser Asn Arg Thr Ser | |
| 660 665 670 | |
| Asp Ser Gln Tyr Asp Ala Leu Ile Thr Ser Asn Leu Val Pro Met Tyr | |
| 675 680 685 | |
| Glu Glu Phe Arg Lys Met Trp Asp Tyr Phe His Ser Val Leu Leu Ile | |
| 690 695 700 | |
| Lys His Ala Thr Glu Arg Asn Gly Val Asn Val Val Ser Gly Pro Ile | |
| 705 710 715 720 | |
| Phe Asp Tyr Asn Tyr Asp Gly His Phe Asp Ala Pro Asp Glu Ile Thr | |
| 725 730 735 | |
| Lys His Leu Ala Asn Thr Asp Val Pro Ile Pro Thr His Tyr Phe Val | |
| 740 745 750 | |
| Val Leu Thr Ser Cys Lys Asn Lys Ser His Thr Pro Glu Asn Cys Pro | |
| 755 760 765 | |
| Gly Trp Leu Asp Val Leu Pro Phe Ile Ile Pro His Arg Pro Thr Asn | |
| 770 775 780 | |
| Val Glu Ser Cys Pro Glu Gly Lys Pro Glu Ala Leu Trp Val Glu Glu | |
| 785 790 795 800 | |
| Arg Phe Thr Ala His Ile Ala Arg Val Arg Asp Val Glu Leu Leu Thr | |
| 805 810 815 | |
| Gly Leu Asp Phe Tyr Gln Asp Lys Val Gln Pro Val Ser Glu Ile Leu | |
| 820 825 830 | |
| Gln Leu Lys Thr Tyr Leu Pro Thr Phe Glu Thr Thr Ile Asp Lys Thr | |
| 835 840 845 | |
| His Thr Cys Pro Pro Cys Pro Ala Pro Glu Leu Leu Gly Gly Pro Ser | |
| 850 855 860 | |
| Val Phe Leu Phe Pro Pro Lys Pro Lys Asp Thr Leu Met Ile Ser Arg | |
| 865 870 875 880 | |
| Thr Pro Glu Val Thr Cys Val Val Val Asp Val Ser His Glu Asp Pro | |
| 885 890 895 | |
| Glu Val Lys Phe Asn Trp Tyr Val Asp Gly Val Glu Val His Asn Ala | |
| 900 905 910 | |
| Lys Thr Lys Pro Arg Glu Glu Gln Tyr Asn Ser Thr Tyr Arg Val Val | |
| 915 920 925 | |
| Ser Val Leu Thr Val Leu His Gln Asp Trp Leu Asn Gly Lys Glu Tyr | |
| 930 935 940 | |
| Lys Cys Lys Val Ser Asn Lys Ala Leu Pro Ala Pro Ile Glu Lys Thr | |
| 945 950 955 960 | |
| Ile Ser Lys Ala Lys Gly Gln Pro Arg Glu Pro Gln Val Tyr Thr Leu | |
| 965 970 975 | |
| Pro Pro Ser Arg Glu Glu Met Thr Lys Asn Gln Val Ser Leu Thr Cys | |
| 980 985 990 | |
| Leu Val Lys Gly Phe Tyr Pro Ser Asp Ile Ala Val Glu Trp Glu Ser | |
| 995 1000 1005 | |
| Asn Gly Gln Pro Glu Asn Asn Tyr Lys Thr Thr Pro Pro Val Leu | |
| 1010 1015 1020 | |
| Asp Ser Asp Gly Ser Phe Phe Leu Tyr Ser Lys Leu Thr Val Asp | |
| 1025 1030 1035 | |
| Lys Ser Arg Trp Gln Gln Gly Asn Val Phe Ser Cys Ser Val Met | |
| 1040 1045 1050 | |
| His Glu Ala Leu His Asn His Tyr Thr Gln Lys Ser Leu Ser Leu | |
| 1055 1060 1065 | |
| Ser Pro Gly Lys | |
| 1070 | |
| Singly underlined: signal peptide sequence; double-underlined: | |
| beginning and end of NPP3; ** = cleavage position at the signal | |
| peptide sequence; bold residues indicate Fc sequence | |
| ENPP7-1-Albumin | |
| SEQ. ID NO: 23 | |
| Met Arg Gly Pro Ala Val Leu Leu Thr Val Ala Leu Ala Thr Leu Leu | |
| 1 5 10 15 | |
| Ala Pro Gly Ala Gly Leu Lys**Pro Ser Cys Ala Lys Glu Val Lys Ser | |
| 20 25 30 | |
| Cys Lys Gly Arg Cys Phe Glu Arg Thr Phe Gly Asn Cys Arg Cys Asp | |
| 35 40 45 | |
| Ala Ala Cys Val Glu Leu Gly Asn Cys Cys Leu Asp Tyr Gln Glu Thr | |
| 50 55 60 | |
| Cys Ile Glu Pro Glu His Ile Trp Thr Cys Asn Lys Phe Arg Cys Gly | |
| 65 70 75 80 | |
| Glu Lys Arg Leu Thr Arg Ser Leu Cys Ala Cys Ser Asp Asp Cys Lys | |
| 85 90 95 | |
| Asp Lys Gly Asp Cys Cys Ile Asn Tyr Ser Ser Val Cys Gln Gly Glu | |
| 100 105 110 | |
| Lys Ser Trp Val Glu Glu Pro Cys Glu Ser Ile Asn Glu Pro Gln Cys | |
| 115 120 125 | |
| Pro Ala Gly Phe Glu Thr Pro Pro Thr Leu Leu Phe Ser Leu Asp Gly | |
| 130 135 140 | |
| Phe Arg Ala Glu Tyr Leu His Thr Trp Gly Gly Leu Leu Pro Val Ile | |
| 145 150 155 160 | |
| Ser Lys Leu Lys Lys Cys Gly Thr Tyr Thr Lys Asn Met Arg Pro Val | |
| 165 170 175 | |
| Tyr Pro Thr Lys Thr Phe Pro Asn His Tyr Ser Ile Val Thr Gly Leu | |
| 180 185 190 | |
| Tyr Pro Glu Ser His Gly Ile Ile Asp Asn Lys Met Tyr Asp Pro Lys | |
| 195 200 205 | |
| Met Asn Ala Ser Phe Ser Leu Lys Ser Lys Glu Lys Phe Asn Pro Glu | |
| 210 215 220 | |
| Trp Tyr Lys Gly Glu Pro Ile Trp Val Thr Ala Lys Tyr Gln Gly Leu | |
| 225 230 235 240 | |
| Lys Ser Gly Thr Phe Phe Trp Pro Gly Ser Asp Val Glu Ile Asn Gly | |
| 245 250 255 | |
| Ile Phe Pro Asp Ile Tyr Lys Met Tyr Asn Gly Ser Val Pro Phe Glu | |
| 260 265 270 | |
| Glu Arg Ile Leu Ala Val Leu Gln Trp Leu Gln Leu Pro Lys Asp Glu | |
| 275 280 285 | |
| Arg Pro His Phe Tyr Thr Leu Tyr Leu Glu Glu Pro Asp Ser Ser Gly | |
| 290 295 300 | |
| His Ser Tyr Gly Pro Val Ser Ser Glu Val Ile Lys Ala Leu Gln Arg | |
| 305 310 315 320 | |
| Val Asp Gly Met Val Gly Met Leu Met Asp Gly Leu Lys Glu Leu Asn | |
| 325 330 335 | |
| Leu His Arg Cys Leu Asn Leu Ile Leu Ile Ser Asp His Gly Met Glu | |
| 340 345 350 | |
| Gln Gly Ser Cys Lys Lys Tyr Ile Tyr Leu Asn Lys Tyr Leu Gly Asp | |
| 355 360 365 | |
| Val Lys Asn Ile Lys Val Ile Tyr Gly Pro Ala Ala Arg Leu Arg Pro | |
| 370 375 380 | |
| Ser Asp Val Pro Asp Lys Tyr Tyr Ser Phe Asn Tyr Glu Gly Ile Ala | |
| 385 390 395 400 | |
| Arg Asn Leu Ser Cys Arg Glu Pro Asn Gln His Phe Lys Pro Tyr Leu | |
| 405 410 415 | |
| Lys His Phe Leu Pro Lys Arg Leu His Phe Ala Lys Ser Asp Arg Ile | |
| 420 425 430 | |
| Glu Pro Leu Thr Phe Tyr Leu Asp Pro Gln Trp Gln Leu Ala Leu Asn | |
| 435 440 445 | |
| Pro Ser Glu Arg Lys Tyr Cys Gly Ser Gly Phe His Gly Ser Asp Asn | |
| 450 455 460 | |
| Val Phe Ser Asn Met Gln Ala Leu Phe Val Gly Tyr Gly Pro Gly Phe | |
| 465 470 475 480 | |
| Lys His Gly Ile Glu Ala Asp Thr Phe Glu Asn Ile Glu Val Tyr Asn | |
| 485 490 495 | |
| Leu Met Cys Asp Leu Leu Asn Leu Thr Pro Ala Pro Asn Asn Gly Thr | |
| 500 505 510 | |
| His Gly Ser Leu Asn His Leu Leu Lys Asn Pro Val Tyr Thr Pro Lys | |
| 515 520 525 | |
| His Pro Lys Glu Val His Pro Leu Val Gln Cys Pro Phe Thr Arg Asn | |
| 530 535 540 | |
| Pro Arg Asp Asn Leu Gly Cys Ser Cys Asn Pro Ser Ile Leu Pro Ile | |
| 545 550 555 560 | |
| Glu Asp Phe Gln Thr Gln Phe Asn Leu Thr Val Ala Glu Glu Lys Ile | |
| 565 570 575 | |
| Ile Lys His Glu Thr Leu Pro Tyr Gly Arg Pro Arg Val Leu Gln Lys | |
| 580 585 590 | |
| Glu Asn Thr Ile Cys Leu Leu Ser Gln His Gln Phe Met Ser Gly Tyr | |
| 595 600 605 | |
| Ser Gln Asp Ile Leu Met Pro Leu Trp Thr Ser Tyr Thr Val Asp Arg | |
| 610 615 620 | |
| Asn Asp Ser Phe Ser Thr Glu Asp Phe Ser Asn Cys Leu Tyr Gln Asp | |
| 625 630 635 640 | |
| Phe Arg Ile Pro Leu Ser Pro Val His Lys Cys Ser Phe Tyr Lys Asn | |
| 645 650 655 | |
| Asn Thr Lys Val Ser Tyr Gly Phe Leu Ser Pro Pro Gln Leu Asn Lys | |
| 660 665 670 | |
| Asn Ser Ser Gly Ile Tyr Ser Glu Ala Leu Leu Thr Thr Asn Ile Val | |
| 675 680 685 | |
| Pro Met Tyr Gln Ser Phe Gln Val Ile Trp Arg Tyr Phe His Asp Thr | |
| 690 695 700 | |
| Leu Leu Arg Lys Tyr Ala Glu Glu Arg Asn Gly Val Asn Val Val Ser | |
| 705 710 715 720 | |
| Gly Pro Val Phe Asp Phe Asp Tyr Asp Gly Arg Cys Asp Ser Leu Glu | |
| 725 730 735 | |
| Asn Leu Arg Gln Lys Arg Arg Val Ile Arg Asn Gln Glu Ile Leu Ile | |
| 740 745 750 | |
| Pro Thr His Phe Phe Ile Val Leu Thr Ser Cys Lys Asp Thr Ser Gln | |
| 755 760 765 | |
| Thr Pro Leu His Cys Glu Asn Leu Asp Thr Leu Ala Phe Ile Leu Pro | |
| 770 775 780 | |
| His Arg Thr Asp Asn Ser Glu Ser Cys Val His Gly Lys His Asp Ser | |
| 785 790 795 800 | |
| Ser Trp Val Glu Glu Leu Leu Met Leu His Arg Ala Arg Ile Thr Asp | |
| 805 810 815 | |
| Val Glu His Ile Thr Gly Leu Ser Phe Tyr Gln Gln Arg Lys Glu Pro | |
| 820 825 830 | |
| Val Ser Asp Ile Leu Lys Leu Lys Thr His Leu Pro Thr Phe Ser Gln | |
| 835 840 845 | |
| Glu Asp Gly Gly Ser Gly Gly Ser Met Lys Trp Val Thr Phe Leu Leu | |
| 850 855 860 | |
| Leu Leu Phe Val Ser Gly Ser Ala Phe Ser Arg Gly Val Phe Arg Arg | |
| 865 870 875 880 | |
| Glu Ala His Lys Ser Glu Ile Ala His Arg Tyr Asn Asp Leu Gly Glu | |
| 885 890 895 | |
| Gln His Phe Lys Gly Leu Val Leu Ile Ala Phe Ser Gln Tyr Leu Gln | |
| 900 905 910 | |
| Lys Cys Ser Tyr Asp Glu His Ala Lys Leu Val Gln Glu Val Thr Asp | |
| 915 920 925 | |
| Phe Ala Lys Thr Cys Val Ala Asp Glu Ser Ala Ala Asn Cys Asp Lys | |
| 930 935 940 | |
| Ser Leu His Thr Leu Phe Gly Asp Lys Leu Cys Ala Ile Pro Asn Leu | |
| 945 950 955 960 | |
| Arg Glu Asn Tyr Gly Glu Leu Ala Asp Cys Cys Thr Lys Gln Glu Pro | |
| 965 970 975 | |
| Glu Arg Asn Glu Cys Phe Leu Gln His Lys Asp Asp Asn Pro Ser Leu | |
| 980 985 990 | |
| Pro Pro Phe Glu Arg Pro Glu Ala Glu Ala Met Cys Thr Ser Phe Lys | |
| 995 1000 1005 | |
| Glu Asn Pro Thr Thr Phe Met Gly His Tyr Leu His Glu Val Ala | |
| 1010 1015 1020 | |
| Arg Arg His Pro Tyr Phe Tyr Ala Pro Glu Leu Leu Tyr Tyr Ala | |
| 1025 1030 1035 | |
| Glu Gln Tyr Asn Glu Ile Leu Thr Gln Cys Cys Ala Glu Ala Asp | |
| 1040 1045 1050 | |
| Lys Glu Ser Cys Leu Thr Pro Lys Leu Asp Gly Val Lys Glu Lys | |
| 1055 1060 1065 | |
| Ala Leu Val Ser Ser Val Arg Gln Arg Met Lys Cys Ser Ser Met | |
| 1070 1075 1080 | |
| Gln Lys Phe Gly Glu Arg Ala Phe Lys Ala Trp Ala Val Ala Arg | |
| 1085 1090 1095 | |
| Leu Ser Gln Thr Phe Pro Asn Ala Asp Phe Ala Glu Ile Thr Lys | |
| 1100 1105 1110 | |
| Leu Ala Thr Asp Leu Thr Lys Val Asn Lys Glu Cys Cys His Gly | |
| 1115 1120 1125 | |
| Asp Leu Leu Glu Cys Ala Asp Asp Arg Ala Glu Leu Ala Lys Tyr | |
| 1130 1135 1140 | |
| Met Cys Glu Asn Gln Ala Thr Ile Ser Ser Lys Leu Gln Thr Cys | |
| 1145 1150 1155 | |
| Cys Asp Lys Pro Leu Leu Lys Lys Ala His Cys Leu Ser Glu Val | |
| 1160 1165 1170 | |
| Glu His Asp Thr Met Pro Ala Asp Leu Pro Ala Ile Ala Ala Asp | |
| 1175 1180 1185 | |
| Phe Val Glu Asp Gln Glu Val Cys Lys Asn Tyr Ala Glu Ala Lys | |
| 1190 1195 1200 | |
| Asp Val Phe Leu Gly Thr Phe Leu Tyr Glu Tyr Ser Arg Arg His | |
| 1205 1210 1215 | |
| Pro Asp Tyr Ser Val Ser Leu Leu Leu Arg Leu Ala Lys Lys Tyr | |
| 1220 1225 1230 | |
| Glu Ala Thr Leu Glu Lys Cys Cys Ala Glu Ala Asn Pro Pro Ala | |
| 1235 1240 1245 | |
| Cys Tyr Gly Thr Val Leu Ala Glu Phe Gln Pro Leu Val Glu Glu | |
| 1250 1255 1260 | |
| Pro Lys Asn Leu Val Lys Thr Asn Cys Asp Leu Tyr Glu Lys Leu | |
| 1265 1270 1275 | |
| Gly Glu Tyr Gly Phe Gln Asn Ala Ile Leu Val Arg Tyr Thr Gln | |
| 1280 1285 1290 | |
| Lys Ala Pro Gln Val Ser Thr Pro Thr Leu Val Glu Ala Ala Arg | |
| 1295 1300 1305 | |
| Asn Leu Gly Arg Val Gly Thr Lys Cys Cys Thr Leu Pro Glu Asp | |
| 1310 1315 1320 | |
| Gln Arg Leu Pro Cys Val Glu Asp Tyr Leu Ser Ala Ile Leu Asn | |
| 1325 1330 1335 | |
| Arg Val Cys Leu Leu His Glu Lys Thr Pro Val Ser Glu His Val | |
| 1340 1345 1350 | |
| Thr Lys Cys Cys Ser Gly Ser Leu Val Glu Arg Arg Pro Cys Phe | |
| 1355 1360 1365 | |
| Ser Ala Leu Thr Val Asp Glu Thr Tyr Val Pro Lys Glu Phe Lys | |
| 1370 1375 1380 | |
| Ala Glu Thr Phe Thr Phe His Ser Asp Ile Cys Thr Leu | |
| 1385 1390 1395 | |
| Singly underlined: signal peptide sequence; double-underlined: | |
| beginning and end of NPP3; ** = cleavage position at the signal | |
| peptide sequence; bold residues indicate Fc sequence | |
| ENPP7-NPP3-Albumin | |
| SEQ. ID NO: 24 | |
| Met Arg Gly Pro Ala Val Leu Leu Thr Val Ala Leu Ala Thr Leu Leu | |
| 1 5 10 15 | |
| Ala Pro Gly Ala**Lys Gln Gly Ser Cys Arg Lys Lys Cys Phe Asp Ala | |
| 20 25 30 | |
| Ser Phe Arg Gly Leu Glu Asn Cys Arg Cys Asp Val Ala Cys Lys Asp | |
| 35 40 45 | |
| Arg Gly Asp Cys Cys Trp Asp Phe Glu Asp Thr Cys Val Glu Ser Thr | |
| 50 55 60 | |
| Arg Ile Trp Met Cys Asn Lys Phe Arg Cys Gly Glu Arg Leu Glu Ala | |
| 65 70 75 80 | |
| Ser Leu Cys Ser Cys Ser Asp Asp Cys Leu Gln Arg Lys Asp Cys Cys | |
| 85 90 95 | |
| Ala Asp Tyr Lys Ser Val Cys Gln Gly Glu Thr Ser Trp Leu Glu Glu | |
| 100 105 110 | |
| Asn Cys Asp Thr Ala Gln Gln Ser Gln Cys Pro Glu Gly Phe Asp Leu | |
| 115 120 125 | |
| Pro Pro Val Ile Leu Phe Ser Met Asp Gly Phe Arg Ala Glu Tyr Leu | |
| 130 135 140 | |
| Tyr Thr Trp Asp Thr Leu Met Pro Asn Ile Asn Lys Leu Lys Thr Cys | |
| 145 150 155 160 | |
| Gly Ile His Ser Lys Tyr Met Arg Ala Met Tyr Pro Thr Lys Thr Phe | |
| 165 170 175 | |
| Pro Asn His Tyr Thr Ile Val Thr Gly Leu Tyr Pro Glu Ser His Gly | |
| 180 185 190 | |
| Ile Ile Asp Asn Asn Met Tyr Asp Val Asn Leu Asn Lys Asn Phe Ser | |
| 195 200 205 | |
| Leu Ser Ser Lys Glu Gln Asn Asn Pro Ala Trp Trp His Gly Gln Pro | |
| 210 215 220 | |
| Met Trp Leu Thr Ala Met Tyr Gln Gly Leu Lys Ala Ala Thr Tyr Phe | |
| 225 230 235 240 | |
| Trp Pro Gly Ser Glu Val Ala Ile Asn Gly Ser Phe Pro Ser Ile Tyr | |
| 245 250 255 | |
| Met Pro Tyr Asn Gly Ser Val Pro Phe Glu Glu Arg Ile Ser Thr Leu | |
| 260 265 270 | |
| Leu Lys Trp Leu Asp Leu Pro Lys Ala Glu Arg Pro Arg Phe Tyr Thr | |
| 275 280 285 | |
| Met Tyr Phe Glu Glu Pro Asp Ser Ser Gly His Ala Gly Gly Pro Val | |
| 290 295 300 | |
| Ser Ala Arg Val Ile Lys Ala Leu Gln Val Val Asp His Ala Phe Gly | |
| 305 310 315 320 | |
| Met Leu Met Glu Gly Leu Lys Gln Arg Asn Leu His Asn Cys Val Asn | |
| 325 330 335 | |
| Ile Ile Leu Leu Ala Asp His Gly Met Asp Gln Thr Tyr Cys Asn Lys | |
| 340 345 350 | |
| Met Glu Tyr Met Thr Asp Tyr Phe Pro Arg Ile Asn Phe Phe Tyr Met | |
| 355 360 365 | |
| Tyr Glu Gly Pro Ala Pro Arg Ile Arg Ala His Asn Ile Pro His Asp | |
| 370 375 380 | |
| Phe Phe Ser Phe Asn Ser Glu Glu Ile Val Arg Asn Leu Ser Cys Arg | |
| 385 390 395 400 | |
| Lys Pro Asp Gln His Phe Lys Pro Tyr Leu Thr Pro Asp Leu Pro Lys | |
| 405 410 415 | |
| Arg Leu His Tyr Ala Lys Asn Val Arg Ile Asp Lys Val His Leu Phe | |
| 420 425 430 | |
| Val Asp Gln Gln Trp Leu Ala Val Arg Ser Lys Ser Asn Thr Asn Cys | |
| 435 440 445 | |
| Gly Gly Gly Asn His Gly Tyr Asn Asn Glu Phe Arg Ser Met Glu Ala | |
| 450 455 460 | |
| Ile Phe Leu Ala His Gly Pro Ser Phe Lys Glu Lys Thr Glu Val Glu | |
| 465 470 475 480 | |
| Pro Phe Glu Asn Ile Glu Val Tyr Asn Leu Met Cys Asp Leu Leu Arg | |
| 485 490 495 | |
| Ile Gln Pro Ala Pro Asn Asn Gly Thr His Gly Ser Leu Asn His Leu | |
| 500 505 510 | |
| Leu Lys Val Pro Phe Tyr Glu Pro Ser His Ala Glu Glu Val Ser Lys | |
| 515 520 525 | |
| Phe Ser Val Cys Gly Phe Ala Asn Pro Leu Pro Thr Glu Ser Leu Asp | |
| 530 535 540 | |
| Cys Phe Cys Pro His Leu Gln Asn Ser Thr Gln Leu Glu Gln Val Asn | |
| 545 550 555 560 | |
| Gln Met Leu Asn Leu Thr Gln Glu Glu Ile Thr Ala Thr Val Lys Val | |
| 565 570 575 | |
| Asn Leu Pro Phe Gly Arg Pro Arg Val Leu Gln Lys Asn Val Asp His | |
| 580 585 590 | |
| Cys Leu Leu Tyr His Arg Glu Tyr Val Ser Gly Phe Gly Lys Ala Met | |
| 595 600 605 | |
| Arg Met Pro Met Trp Ser Ser Tyr Thr Val Pro Gln Leu Gly Asp Thr | |
| 610 615 620 | |
| Ser Pro Leu Pro Pro Thr Val Pro Asp Cys Leu Arg Ala Asp Val Arg | |
| 625 630 635 640 | |
| Val Pro Pro Ser Glu Ser Gln Lys Cys Ser Phe Tyr Leu Ala Asp Lys | |
| 645 650 655 | |
| Asn Ile Thr His Gly Phe Leu Tyr Pro Pro Ala Ser Asn Arg Thr Ser | |
| 660 665 670 | |
| Asp Ser Gln Tyr Asp Ala Leu Ile Thr Ser Asn Leu Val Pro Met Tyr | |
| 675 680 685 | |
| Glu Glu Phe Arg Lys Met Trp Asp Tyr Phe His Ser Val Leu Leu Ile | |
| 690 695 700 | |
| Lys His Ala Thr Glu Arg Asn Gly Val Asn Val Val Ser Gly Pro Ile | |
| 705 710 715 720 | |
| Phe Asp Tyr Asn Tyr Asp Gly His Phe Asp Ala Pro Asp Glu Ile Thr | |
| 725 730 735 | |
| Lys His Leu Ala Asn Thr Asp Val Pro Ile Pro Thr His Tyr Phe Val | |
| 740 745 750 | |
| Val Leu Thr Ser Cys Lys Asn Lys Ser His Thr Pro Glu Asn Cys Pro | |
| 755 760 765 | |
| Gly Trp Leu Asp Val Leu Pro Phe Ile Ile Pro His Arg Pro Thr Asn | |
| 770 775 780 | |
| Val Glu Ser Cys Pro Glu Gly Lys Pro Glu Ala Leu Trp Val Glu Glu | |
| 785 790 795 800 | |
| Arg Phe Thr Ala His Ile Ala Arg Val Arg Asp Val Glu Leu Leu Thr | |
| 805 810 815 | |
| Gly Leu Asp Phe Tyr Gln Asp Lys Val Gln Pro Val Ser Glu Ile Leu | |
| 820 825 830 | |
| Gln Leu Lys Thr Tyr Leu Pro Thr Phe Glu Thr Thr Ile Gly Gly Gly | |
| 835 840 845 | |
| Ser Gly Gly Gly Gly Ser Gly Gly Gly Gly Ser Met Lys Trp Val Thr | |
| 850 855 860 | |
| Phe Leu Leu Leu Leu Phe Val Ser Gly Ser Ala Phe Ser Arg Gly Val | |
| 865 870 875 880 | |
| Phe Arg Arg Glu Ala His Lys Ser Glu Ile Ala His Arg Tyr Asn Asp | |
| 885 890 895 | |
| Leu Gly Glu Gln His Phe Lys Gly Leu Val Leu Ile Ala Phe Ser Gln | |
| 900 905 910 | |
| Tyr Leu Gln Lys Cys Ser Tyr Asp Glu His Ala Lys Leu Val Gln Glu | |
| 915 920 925 | |
| Val Thr Asp Phe Ala Lys Thr Cys Val Ala Asp Glu Ser Ala Ala Asn | |
| 930 935 940 | |
| Cys Asp Lys Ser Leu His Thr Leu Phe Gly Asp Lys Leu Cys Ala Ile | |
| 945 950 955 960 | |
| Pro Asn Leu Arg Glu Asn Tyr Gly Glu Leu Ala Asp Cys Cys Thr Lys | |
| 965 970 975 | |
| Gln Glu Pro Glu Arg Asn Glu Cys Phe Leu Gln His Lys Asp Asp Asn | |
| 980 985 990 | |
| Pro Ser Leu Pro Pro Phe Glu Arg Pro Glu Ala Glu Ala Met Cys Thr | |
| 995 1000 1005 | |
| Ser Phe Lys Glu Asn Pro Thr Thr Phe Met Gly His Tyr Leu His | |
| 1010 1015 1020 | |
| Glu Val Ala Arg Arg His Pro Tyr Phe Tyr Ala Pro Glu Leu Leu | |
| 1025 1030 1035 | |
| Tyr Tyr Ala Glu Gln Tyr Asn Glu Ile Leu Thr Gln Cys Cys Ala | |
| 1040 1045 1050 | |
| Glu Ala Asp Lys Glu Ser Cys Leu Thr Pro Lys Leu Asp Gly Val | |
| 1055 1060 1065 | |
| Lys Glu Lys Ala Leu Val Ser Ser Val Arg Gln Arg Met Lys Cys | |
| 1070 1075 1080 | |
| Ser Ser Met Gln Lys Phe Gly Glu Arg Ala Phe Lys Ala Trp Ala | |
| 1085 1090 1095 | |
| Val Ala Arg Leu Ser Gln Thr Phe Pro Asn Ala Asp Phe Ala Glu | |
| 1100 1105 1110 | |
| Ile Thr Lys Leu Ala Thr Asp Leu Thr Lys Val Asn Lys Glu Cys | |
| 1115 1120 1125 | |
| Cys His Gly Asp Leu Leu Glu Cys Ala Asp Asp Arg Ala Glu Leu | |
| 1130 1135 1140 | |
| Ala Lys Tyr Met Cys Glu Asn Gln Ala Thr Ile Ser Ser Lys Leu | |
| 1145 1150 1155 | |
| Gln Thr Cys Cys Asp Lys Pro Leu Leu Lys Lys Ala His Cys Leu | |
| 1160 1165 1170 | |
| Ser Glu Val Glu His Asp Thr Met Pro Ala Asp Leu Pro Ala Ile | |
| 1175 1180 1185 | |
| Ala Ala Asp Phe Val Glu Asp Gln Glu Val Cys Lys Asn Tyr Ala | |
| 1190 1195 1200 | |
| Glu Ala Lys Asp Val Phe Leu Gly Thr Phe Leu Tyr Glu Tyr Ser | |
| 1205 1210 1215 | |
| Arg Arg His Pro Asp Tyr Ser Val Ser Leu Leu Leu Arg Leu Ala | |
| 1220 1225 1230 | |
| Lys Lys Tyr Glu Ala Thr Leu Glu Lys Cys Cys Ala Glu Ala Asn | |
| 1235 1240 1245 | |
| Pro Pro Ala Cys Tyr Gly Thr Val Leu Ala Glu Phe Gln Pro Leu | |
| 1250 1255 1260 | |
| Val Glu Glu Pro Lys Asn Leu Val Lys Thr Asn Cys Asp Leu Tyr | |
| 1265 1270 1275 | |
| Glu Lys Leu Gly Glu Tyr Gly Phe Gln Asn Ala Ile Leu Val Arg | |
| 1280 1285 1290 | |
| Tyr Thr Gln Lys Ala Pro Gln Val Ser Thr Pro Thr Leu Val Glu | |
| 1295 1300 1305 | |
| Ala Ala Arg Asn Leu Gly Arg Val Gly Thr Lys Cys Cys Thr Leu | |
| 1310 1315 1320 | |
| Pro Glu Asp Gln Arg Leu Pro Cys Val Glu Asp Tyr Leu Ser Ala | |
| 1325 1330 1335 | |
| Ile Leu Asn Arg Val Cys Leu Leu His Glu Lys Thr Pro Val Ser | |
| 1340 1345 1350 | |
| Glu His Val Thr Lys Cys Cys Ser Gly Ser Leu Val Glu Arg Arg | |
| 1355 1360 1365 | |
| Pro Cys Phe Ser Ala Leu Thr Val Asp Glu Thr Tyr Val Pro Lys | |
| 1370 1375 1380 | |
| Glu Phe Lys Ala Glu Thr Phe Thr Phe His Ser Asp Ile Cys Thr | |
| 1385 1390 1395 | |
| Leu Pro Glu Lys Glu Lys Gln Ile Lys Lys Gln Thr Ala Leu Ala | |
| 1400 1405 1410 | |
| Glu Leu Val Lys His Lys Pro Lys Ala Thr Ala Glu Gln Leu Lys | |
| 1415 1420 1425 | |
| Thr Val Met Asp Asp Phe Ala Gln Phe Leu Asp Thr Cys Cys Lys | |
| 1430 1435 1440 | |
| Ala Ala Asp Lys Asp Thr Cys Phe Ser Thr Glu Gly Pro Asn Leu | |
| 1445 1450 1455 | |
| Val Thr Arg Cys Lys Asp Ala Leu Ala | |
| 1460 1465 | |
| Singly underlined: signal peptide sequence; double-underlined: | |
| beginning and end of NPP3; ** = cleavage position at the signal | |
| peptide sequence; bold residues indicate albumin sequence | |
| ENPP7-ENPP3-Albumin | |
| SEQ. ID NO: 25 | |
| Met Arg Gly Pro Ala Val Leu Leu Thr Val Ala Leu Ala Thr Leu Leu | |
| 1 5 10 15 | |
| Ala Pro Gly Ala**Lys Gln Gly Ser Cys Arg Lys Lys Cys Phe Asp Ala | |
| 20 25 30 | |
| Ser Phe Arg Gly Leu Glu Asn Cys Arg Cys Asp Val Ala Cys Lys Asp | |
| 35 40 45 | |
| Arg Gly Asp Cys Cys Trp Asp Phe Glu Asp Thr Cys Val Glu Ser Thr | |
| 50 55 60 | |
| Arg Ile Trp Met Cys Asn Lys Phe Arg Cys Gly Glu Arg Leu Glu Ala | |
| 65 70 75 80 | |
| Ser Leu Cys Ser Cys Ser Asp Asp Cys Leu Gln Arg Lys Asp Cys Cys | |
| 85 90 95 | |
| Ala Asp Tyr Lys Ser Val Cys Gln Gly Glu Thr Ser Trp Leu Glu Glu | |
| 100 105 110 | |
| Asn Cys Asp Thr Ala Gln Gln Ser Gln Cys Pro Glu Gly Phe Asp Leu | |
| 115 120 125 | |
| Pro Pro Val Ile Leu Phe Ser Met Asp Gly Phe Arg Ala Glu Tyr Leu | |
| 130 135 140 | |
| Tyr Thr Trp Asp Thr Leu Met Pro Asn Ile Asn Lys Leu Lys Thr Cys | |
| 145 150 155 160 | |
| Gly Ile His Ser Lys Tyr Met Arg Ala Met Tyr Pro Thr Lys Thr Phe | |
| 165 170 175 | |
| Pro Asn His Tyr Thr Ile Val Thr Gly Leu Tyr Pro Glu Ser His Gly | |
| 180 185 190 | |
| Ile Ile Asp Asn Asn Met Tyr Asp Val Asn Leu Asn Lys Asn Phe Ser | |
| 195 200 205 | |
| Leu Ser Ser Lys Glu Gln Asn Asn Pro Ala Trp Trp His Gly Gln Pro | |
| 210 215 220 | |
| Met Trp Leu Thr Ala Met Tyr Gln Gly Leu Lys Ala Ala Thr Tyr Phe | |
| 225 230 235 240 | |
| Trp Pro Gly Ser Glu Val Ala Ile Asn Gly Ser Phe Pro Ser Ile Tyr | |
| 245 250 255 | |
| Met Pro Tyr Asn Gly Ser Val Pro Phe Glu Glu Arg Ile Ser Thr Leu | |
| 260 265 270 | |
| Leu Lys Trp Leu Asp Leu Pro Lys Ala Glu Arg Pro Arg Phe Tyr Thr | |
| 275 280 285 | |
| Met Tyr Phe Glu Glu Pro Asp Ser Ser Gly His Ala Gly Gly Pro Val | |
| 290 295 300 | |
| Ser Ala Arg Val Ile Lys Ala Leu Gln Val Val Asp His Ala Phe Gly | |
| 305 310 315 320 | |
| Met Leu Met Glu Gly Leu Lys Gln Arg Asn Leu His Asn Cys Val Asn | |
| 325 330 335 | |
| Ile Ile Leu Leu Ala Asp His Gly Met Asp Gln Thr Tyr Cys Asn Lys | |
| 340 345 350 | |
| Met Glu Tyr Met Thr Asp Tyr Phe Pro Arg Ile Asn Phe Phe Tyr Met | |
| 355 360 365 | |
| Tyr Glu Gly Pro Ala Pro Arg Ile Arg Ala His Asn Ile Pro His Asp | |
| 370 375 380 | |
| Phe Phe Ser Phe Asn Ser Glu Glu Ile Val Arg Asn Leu Ser Cys Arg | |
| 385 390 395 400 | |
| Lys Pro Asp Gln His Phe Lys Pro Tyr Leu Thr Pro Asp Leu Pro Lys | |
| 405 410 415 | |
| Arg Leu His Tyr Ala Lys Asn Val Arg Ile Asp Lys Val His Leu Phe | |
| 420 425 430 | |
| Val Asp Gln Gln Trp Leu Ala Val Arg Ser Lys Ser Asn Thr Asn Cys | |
| 435 440 445 | |
| Gly Gly Gly Asn His Gly Tyr Asn Asn Glu Phe Arg Ser Met Glu Ala | |
| 450 455 460 | |
| Ile Phe Leu Ala His Gly Pro Ser Phe Lys Glu Lys Thr Glu Val Glu | |
| 465 470 475 480 | |
| Pro Phe Glu Asn Ile Glu Val Tyr Asn Leu Met Cys Asp Leu Leu Arg | |
| 485 490 495 | |
| Ile Gln Pro Ala Pro Asn Asn Gly Thr His Gly Ser Leu Asn His Leu | |
| 500 505 510 | |
| Leu Lys Val Pro Phe Tyr Glu Pro Ser His Ala Glu Glu Val Ser Lys | |
| 515 520 525 | |
| Phe Ser Val Cys Gly Phe Ala Asn Pro Leu Pro Thr Glu Ser Leu Asp | |
| 530 535 540 | |
| Cys Phe Cys Pro His Leu Gln Asn Ser Thr Gln Leu Glu Gln Val Asn | |
| 545 550 555 560 | |
| Gln Met Leu Asn Leu Thr Gln Glu Glu Ile Thr Ala Thr Val Lys Val | |
| 565 570 575 | |
| Asn Leu Pro Phe Gly Arg Pro Arg Val Leu Gln Lys Asn Val Asp His | |
| 580 585 590 | |
| Cys Leu Leu Tyr His Arg Glu Tyr Val Ser Gly Phe Gly Lys Ala Met | |
| 595 600 605 | |
| Arg Met Pro Met Trp Ser Ser Tyr Thr Val Pro Gln Leu Gly Asp Thr | |
| 610 615 620 | |
| Ser Pro Leu Pro Pro Thr Val Pro Asp Cys Leu Arg Ala Asp Val Arg | |
| 625 630 635 640 | |
| Val Pro Pro Ser Glu Ser Gln Lys Cys Ser Phe Tyr Leu Ala Asp Lys | |
| 645 650 655 | |
| Asn Ile Thr His Gly Phe Leu Tyr Pro Pro Ala Ser Asn Arg Thr Ser | |
| 660 665 670 | |
| Asp Ser Gln Tyr Asp Ala Leu Ile Thr Ser Asn Leu Val Pro Met Tyr | |
| 675 680 685 | |
| Glu Glu Phe Arg Lys Met Trp Asp Tyr Phe His Ser Val Leu Leu Ile | |
| 690 695 700 | |
| Lys His Ala Thr Glu Arg Asn Gly Val Asn Val Val Ser Gly Pro Ile | |
| 705 710 715 720 | |
| Phe Asp Tyr Asn Tyr Asp Gly His Phe Asp Ala Pro Asp Glu Ile Thr | |
| 725 730 735 | |
| Lys His Leu Ala Asn Thr Asp Val Pro Ile Pro Thr His Tyr Phe Val | |
| 740 745 750 | |
| Val Leu Thr Ser Cys Lys Asn Lys Ser His Thr Pro Glu Asn Cys Pro | |
| 755 760 765 | |
| Gly Trp Leu Asp Val Leu Pro Phe Ile Ile Pro His Arg Pro Thr Asn | |
| 770 775 780 | |
| Val Glu Ser Cys Pro Glu Gly Lys Pro Glu Ala Leu Trp Val Glu Glu | |
| 785 790 795 800 | |
| Arg Phe Thr Ala His Ile Ala Arg Val Arg Asp Val Glu Leu Leu Thr | |
| 805 810 815 | |
| Gly Leu Asp Phe Tyr Gln Asp Lys Val Gln Pro Val Ser Glu Ile Leu | |
| 820 825 830 | |
| Gln Leu Lys Thr Tyr Leu Pro Thr Phe Glu Thr Thr Ile Asp Lys Thr | |
| 835 840 845 | |
| His Thr Cys Pro Pro Cys Pro Ala Pro Glu Leu Leu Gly Gly Pro Ser | |
| 850 855 860 | |
| Val Phe Leu Phe Pro Pro Lys Pro Lys Asp Thr Leu Met Ile Ser Arg | |
| 865 870 875 880 | |
| Thr Pro Glu Val Thr Gly Gly Gly Ser Gly Gly Gly Gly Ser Gly Gly | |
| 885 890 895 | |
| Gly Gly Ser Met Lys Trp Val Thr Phe Leu Leu Leu Leu Phe Val Ser | |
| 900 905 910 | |
| Gly Ser Ala Phe Ser Arg Gly Val Phe Arg Arg Glu Ala His Lys Ser | |
| 915 920 925 | |
| Glu Ile Ala His Arg Tyr Asn Asp Leu Gly Glu Gln His Phe Lys Gly | |
| 930 935 940 | |
| Leu Val Leu Ile Ala Phe Ser Gln Tyr Leu Gln Lys Cys Ser Tyr Asp | |
| 945 950 955 960 | |
| Glu His Ala Lys Leu Val Gln Glu Val Thr Asp Phe Ala Lys Thr Cys | |
| 965 970 975 | |
| Val Ala Asp Glu Ser Ala Ala Asn Cys Asp Lys Ser Leu His Thr Leu | |
| 980 985 990 | |
| Phe Gly Asp Lys Leu Cys Ala Ile Pro Asn Leu Arg Glu Asn Tyr Gly | |
| 995 1000 1005 | |
| Glu Leu Ala Asp Cys Cys Thr Lys Gln Glu Pro Glu Arg Asn Glu | |
| 1010 1015 1020 | |
| Cys Phe Leu Gln His Lys Asp Asp Asn Pro Ser Leu Pro Pro Phe | |
| 1025 1030 1035 | |
| Glu Arg Pro Glu Ala Glu Ala Met Cys Thr Ser Phe Lys Glu Asn | |
| 1040 1045 1050 | |
| Pro Thr Thr Phe Met Gly His Tyr Leu His Glu Val Ala Arg Arg | |
| 1055 1060 1065 | |
| His Pro Tyr Phe Tyr Ala Pro Glu Leu Leu Tyr Tyr Ala Glu Gln | |
| 1070 1075 1080 | |
| Tyr Asn Glu Ile Leu Thr Gln Cys Cys Ala Glu Ala Asp Lys Glu | |
| 1085 1090 1095 | |
| Ser Cys Leu Thr Pro Lys Leu Asp Gly Val Lys Glu Lys Ala Leu | |
| 1100 1105 1110 | |
| Val Ser Ser Val Arg Gln Arg Met Lys Cys Ser Ser Met Gln Lys | |
| 1115 1120 1125 | |
| Phe Gly Glu Arg Ala Phe Lys Ala Trp Ala Val Ala Arg Leu Ser | |
| 1130 1135 1140 | |
| Gln Thr Phe Pro Asn Ala Asp Phe Ala Glu Ile Thr Lys Leu Ala | |
| 1145 1150 1155 | |
| Thr Asp Leu Thr Lys Val Asn Lys Glu Cys Cys His Gly Asp Leu | |
| 1160 1165 1170 | |
| Leu Glu Cys Ala Asp Asp Arg Ala Glu Leu Ala Lys Tyr Met Cys | |
| 1175 1180 1185 | |
| Glu Asn Gln Ala Thr Ile Ser Ser Lys Leu Gln Thr Cys Cys Asp | |
| 1190 1195 1200 | |
| Lys Pro Leu Leu Lys Lys Ala His Cys Leu Ser Glu Val Glu His | |
| 1205 1210 1215 | |
| Asp Thr Met Pro Ala Asp Leu Pro Ala Ile Ala Ala Asp Phe Val | |
| 1220 1225 1230 | |
| Glu Asp Gln Glu Val Cys Lys Asn Tyr Ala Glu Ala Lys Asp Val | |
| 1235 1240 1245 | |
| Phe Leu Gly Thr Phe Leu Tyr Glu Tyr Ser Arg Arg His Pro Asp | |
| 1250 1255 1260 | |
| Tyr Ser Val Ser Leu Leu Leu Arg Leu Ala Lys Lys Tyr Glu Ala | |
| 1265 1270 1275 | |
| Thr Leu Glu Lys Cys Cys Ala Glu Ala Asn Pro Pro Ala Cys Tyr | |
| 1280 1285 1290 | |
| Gly Thr Val Leu Ala Glu Phe Gln Pro Leu Val Glu Glu Pro Lys | |
| 1295 1300 1305 | |
| Asn Leu Val Lys Thr Asn Cys Asp Leu Tyr Glu Lys Leu Gly Glu | |
| 1310 1315 1320 | |
| Tyr Gly Phe Gln Asn Ala Ile Leu Val Arg Tyr Thr Gln Lys Ala | |
| 1325 1330 1335 | |
| Pro Gln Val Ser Thr Pro Thr Leu Val Glu Ala Ala Arg Asn Leu | |
| 1340 1345 1350 | |
| Gly Arg Val Gly Thr Lys Cys Cys Thr Leu Pro Glu Asp Gln Arg | |
| 1355 1360 1365 | |
| Leu Pro Cys Val Glu Asp Tyr Leu Ser Ala Ile Leu Asn Arg Val | |
| 1370 1375 1380 | |
| Cys Leu Leu His Glu Lys Thr Pro Val Ser Glu His Val Thr Lys | |
| 1385 1390 1395 | |
| Cys Cys Ser Gly Ser Leu Val Glu Arg Arg Pro Cys Phe Ser Ala | |
| 1400 1405 1410 | |
| Leu Thr Val Asp Glu Thr Tyr Val Pro Lys Glu Phe Lys Ala Glu | |
| 1415 1420 1425 | |
| Thr Phe Thr Phe His Ser Asp Ile Cys Thr Leu Pro Glu Lys Glu | |
| 1430 1435 1440 | |
| Lys Gln Ile Lys Lys Gln Thr Ala Leu Ala Glu Leu Val Lys His | |
| 1445 1450 1455 | |
| Lys Pro Lys Ala Thr Ala Glu Gln Leu Lys Thr Val Met Asp Asp | |
| 1460 1465 1470 | |
| Phe Ala Gln Phe Leu Asp Thr Cys Cys Lys Ala Ala Asp Lys Asp | |
| 1475 1480 1485 | |
| Thr Cys Phe Ser Thr Glu Gly Pro Asn Leu Val Thr Arg Cys Lys | |
| 1490 1495 1500 | |
| Asp Ala Leu Ala | |
| 1505 | |
| Singly underlined: signal peptide sequence; double-underlined: | |
| beginning and end of NPP3; ** = cleavage position at the signal | |
| peptide sequence; bold residues indicate albumin sequence | |
| ENPP71-GLK Amino Acid Sequence | |
| SEQ. ID NO: 26 | |
| Met Arg Gly Pro Ala Val Leu Leu Thr Val Ala Leu Ala Thr Leu Leu | |
| 1 5 10 15 | |
| Ala Pro Gly Ala Gly Ala**Gly Leu Lys Pro Ser Cys Ala Lys Glu Val | |
| 20 25 30 | |
| Lys Ser Cys Lys Gly Arg Cys Phe Glu Arg Thr Phe Gly Asn Cys Arg | |
| 35 40 45 | |
| Cys Asp Ala Ala Cys Val Glu Leu Gly Asn Cys Cys Leu Asp Tyr Gln | |
| 50 55 60 | |
| Glu Thr Cys Ile Glu Pro Glu His Ile Trp Thr Cys Asn Lys Phe Arg | |
| 65 70 75 80 | |
| Cys Gly Glu Lys Arg Leu Thr Arg Ser Leu Cys Ala Cys Ser Asp Asp | |
| 85 90 95 | |
| Cys Lys Asp Lys Gly Asp Cys Cys Ile Asn Tyr Ser Ser Val Cys Gln | |
| 100 105 110 | |
| Gly Glu Lys Ser Trp Val Glu Glu Pro Cys Glu Ser Ile Asn Glu Pro | |
| 115 120 125 | |
| Gln Cys Pro Ala Gly Phe Glu Thr Pro Pro Thr Leu Leu Phe Ser Leu | |
| 130 135 140 | |
| Asp Gly Phe Arg Ala Glu Tyr Leu His Thr Trp Gly Gly Leu Leu Pro | |
| 145 150 155 160 | |
| Val Ile Ser Lys Leu Lys Lys Cys Gly Thr Tyr Thr Lys Asn Met Arg | |
| 165 170 175 | |
| Pro Val Tyr Pro Thr Lys Thr Phe Pro Asn His Tyr Ser Ile Val Thr | |
| 180 185 190 | |
| Gly Leu Tyr Pro Glu Ser His Gly Ile Ile Asp Asn Lys Met Tyr Asp | |
| 195 200 205 | |
| Pro Lys Met Asn Ala Ser Phe Ser Leu Lys Ser Lys Glu Lys Phe Asn | |
| 210 215 220 | |
| Pro Glu Trp Tyr Lys Gly Glu Pro Ile Trp Val Thr Ala Lys Tyr Gln | |
| 225 230 235 240 | |
| Gly Leu Lys Ser Gly Thr Phe Phe Trp Pro Gly Ser Asp Val Glu Ile | |
| 245 250 255 | |
| Asn Gly Ile Phe Pro Asp Ile Tyr Lys Met Tyr Asn Gly Ser Val Pro | |
| 260 265 270 | |
| Phe Glu Glu Arg Ile Leu Ala Val Leu Gln Trp Leu Gln Leu Pro Lys | |
| 275 280 285 | |
| Asp Glu Arg Pro His Phe Tyr Thr Leu Tyr Leu Glu Glu Pro Asp Ser | |
| 290 295 300 | |
| Ser Gly His Ser Tyr Gly Pro Val Ser Ser Glu Val Ile Lys Ala Leu | |
| 305 310 315 320 | |
| Gln Arg Val Asp Gly Met Val Gly Met Leu Met Asp Gly Leu Lys Glu | |
| 325 330 335 | |
| Leu Asn Leu His Arg Cys Leu Asn Leu Ile Leu Ile Ser Asp His Gly | |
| 340 345 350 | |
| Met Glu Gln Gly Ser Cys Lys Lys Tyr Ile Tyr Leu Asn Lys Tyr Leu | |
| 355 360 365 | |
| Gly Asp Val Lys Asn Ile Lys Val Ile Tyr Gly Pro Ala Ala Arg Leu | |
| 370 375 380 | |
| Arg Pro Ser Asp Val Pro Asp Lys Tyr Tyr Ser Phe Asn Tyr Glu Gly | |
| 385 390 395 400 | |
| Ile Ala Arg Asn Leu Ser Cys Arg Glu Pro Asn Gln His Phe Lys Pro | |
| 405 410 415 | |
| Tyr Leu Lys His Phe Leu Pro Lys Arg Leu His Phe Ala Lys Ser Asp | |
| 420 425 430 | |
| Arg Ile Glu Pro Leu Thr Phe Tyr Leu Asp Pro Gln Trp Gln Leu Ala | |
| 435 440 445 | |
| Leu Asn Pro Ser Glu Arg Lys Tyr Cys Gly Ser Gly Phe His Gly Ser | |
| 450 455 460 | |
| Asp Asn Val Phe Ser Asn Met Gln Ala Leu Phe Val Gly Tyr Gly Pro | |
| 465 470 475 480 | |
| Gly Phe Lys His Gly Ile Glu Ala Asp Thr Phe Glu Asn Ile Glu Val | |
| 485 490 495 | |
| Tyr Asn Leu Met Cys Asp Leu Leu Asn Leu Thr Pro Ala Pro Asn Asn | |
| 500 505 510 | |
| Gly Thr His Gly Ser Leu Asn His Leu Leu Lys Asn Pro Val Tyr Thr | |
| 515 520 525 | |
| Pro Lys His Pro Lys Glu Val His Pro Leu Val Gln Cys Pro Phe Thr | |
| 530 535 540 | |
| Arg Asn Pro Arg Asp Asn Leu Gly Cys Ser Cys Asn Pro Ser Ile Leu | |
| 545 550 555 560 | |
| Pro Ile Glu Asp Phe Gln Thr Gln Phe Asn Leu Thr Val Ala Glu Glu | |
| 565 570 575 | |
| Lys Ile Ile Lys His Glu Thr Leu Pro Tyr Gly Arg Pro Arg Val Leu | |
| 580 585 590 | |
| Gln Lys Glu Asn Thr Ile Cys Leu Leu Ser Gln His Gln Phe Met Ser | |
| 595 600 605 | |
| Gly Tyr Ser Gln Asp Ile Leu Met Pro Leu Trp Thr Ser Tyr Thr Val | |
| 610 615 620 | |
| Asp Arg Asn Asp Ser Phe Ser Thr Glu Asp Phe Ser Asn Cys Leu Tyr | |
| 625 630 635 640 | |
| Gln Asp Phe Arg Ile Pro Leu Ser Pro Val His Lys Cys Ser Phe Tyr | |
| 645 650 655 | |
| Lys Asn Asn Thr Lys Val Ser Tyr Gly Phe Leu Ser Pro Pro Gln Leu | |
| 660 665 670 | |
| Asn Lys Asn Ser Ser Gly Ile Tyr Ser Glu Ala Leu Leu Thr Thr Asn | |
| 675 680 685 | |
| Ile Val Pro Met Tyr Gln Ser Phe Gln Val Ile Trp Arg Tyr Phe His | |
| 690 695 700 | |
| Asp Thr Leu Leu Arg Lys Tyr Ala Glu Glu Arg Asn Gly Val Asn Val | |
| 705 710 715 720 | |
| Val Ser Gly Pro Val Phe Asp Phe Asp Tyr Asp Gly Arg Cys Asp Ser | |
| 725 730 735 | |
| Leu Glu Asn Leu Arg Gln Lys Arg Arg Val Ile Arg Asn Gln Glu Ile | |
| 740 745 750 | |
| Leu Ile Pro Thr His Phe Phe Ile Val Leu Thr Ser Cys Lys Asp Thr | |
| 755 760 765 | |
| Ser Gln Thr Pro Leu His Cys Glu Asn Leu Asp Thr Leu Ala Phe Ile | |
| 770 775 780 | |
| Leu Pro His Arg Thr Asp Asn Ser Glu Ser Cys Val His Gly Lys His | |
| 785 790 795 800 | |
| Asp Ser Ser Trp Val Glu Glu Leu Leu Met Leu His Arg Ala Arg Ile | |
| 805 810 815 | |
| Thr Asp Val Glu His Ile Thr Gly Leu Ser Phe Tyr Gln Gln Arg Lys | |
| 820 825 830 | |
| Glu Pro Val Ser Asp Ile Leu Lys Leu Lys Thr His Leu Pro Thr Phe | |
| 835 840 845 | |
| Ser Gln Glu Asp | |
| 850 | |
| Singly underlined: signal peptide sequence; double-underlined: | |
| beginning and end of NPP1; ** = cleavage position at the signal | |
| peptide sequence | |
| ENPP121 Amino Acid Sequence | |
| SEQ. ID NO: 27 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly**Phe Thr Ala Gly | |
| 85 90 95 | |
| Leu Lys Pro Ser Cys Ala Lys Glu Val Lys Ser Cys Lys Gly Arg Cys | |
| 100 105 110 | |
| Phe Glu Arg Thr Phe Gly Asn Cys Arg Cys Asp Ala Ala Cys Val Glu | |
| 115 120 125 | |
| Leu Gly Asn Cys Cys Leu Asp Tyr Gln Glu Thr Cys Ile Glu Pro Glu | |
| 130 135 140 | |
| His Ile Trp Thr Cys Asn Lys Phe Arg Cys Gly Glu Lys Arg Leu Thr | |
| 145 150 155 160 | |
| Arg Ser Leu Cys Ala Cys Ser Asp Asp Cys Lys Asp Lys Gly Asp Cys | |
| 165 170 175 | |
| Cys Ile Asn Tyr Ser Ser Val Cys Gln Gly Glu Lys Ser Trp Val Glu | |
| 180 185 190 | |
| Glu Pro Cys Glu Ser Ile Asn Glu Pro Gln Cys Pro Ala Gly Phe Glu | |
| 195 200 205 | |
| Thr Pro Pro Thr Leu Leu Phe Ser Leu Asp Gly Phe Arg Ala Glu Tyr | |
| 210 215 220 | |
| Leu His Thr Trp Gly Gly Leu Leu Pro Val Ile Ser Lys Leu Lys Lys | |
| 225 230 235 240 | |
| Cys Gly Thr Tyr Thr Lys Asn Met Arg Pro Val Tyr Pro Thr Lys Thr | |
| 245 250 255 | |
| Phe Pro Asn His Tyr Ser Ile Val Thr Gly Leu Tyr Pro Glu Ser His | |
| 260 265 270 | |
| Gly Ile Ile Asp Asn Lys Met Tyr Asp Pro Lys Met Asn Ala Ser Phe | |
| 275 280 285 | |
| Ser Leu Lys Ser Lys Glu Lys Phe Asn Pro Glu Trp Tyr Lys Gly Glu | |
| 290 295 300 | |
| Pro Ile Trp Val Thr Ala Lys Tyr Gln Gly Leu Lys Ser Gly Thr Phe | |
| 305 310 315 320 | |
| Phe Trp Pro Gly Ser Asp Val Glu Ile Asn Gly Ile Phe Pro Asp Ile | |
| 325 330 335 | |
| Tyr Lys Met Tyr Asn Gly Ser Val Pro Phe Glu Glu Arg Ile Leu Ala | |
| 340 345 350 | |
| Val Leu Gln Trp Leu Gln Leu Pro Lys Asp Glu Arg Pro His Phe Tyr | |
| 355 360 365 | |
| Thr Leu Tyr Leu Glu Glu Pro Asp Ser Ser Gly His Ser Tyr Gly Pro | |
| 370 375 380 | |
| Val Ser Ser Glu Val Ile Lys Ala Leu Gln Arg Val Asp Gly Met Val | |
| 385 390 395 400 | |
| Gly Met Leu Met Asp Gly Leu Lys Glu Leu Asn Leu His Arg Cys Leu | |
| 405 410 415 | |
| Asn Leu Ile Leu Ile Ser Asp His Gly Met Glu Gln Gly Ser Cys Lys | |
| 420 425 430 | |
| Lys Tyr Ile Tyr Leu Asn Lys Tyr Leu Gly Asp Val Lys Asn Ile Lys | |
| 435 440 445 | |
| Val Ile Tyr Gly Pro Ala Ala Arg Leu Arg Pro Ser Asp Val Pro Asp | |
| 450 455 460 | |
| Lys Tyr Tyr Ser Phe Asn Tyr Glu Gly Ile Ala Arg Asn Leu Ser Cys | |
| 465 470 475 480 | |
| Arg Glu Pro Asn Gln His Phe Lys Pro Tyr Leu Lys His Phe Leu Pro | |
| 485 490 495 | |
| Lys Arg Leu His Phe Ala Lys Ser Asp Arg Ile Glu Pro Leu Thr Phe | |
| 500 505 510 | |
| Tyr Leu Asp Pro Gln Trp Gln Leu Ala Leu Asn Pro Ser Glu Arg Lys | |
| 515 520 525 | |
| Tyr Cys Gly Ser Gly Phe His Gly Ser Asp Asn Val Phe Ser Asn Met | |
| 530 535 540 | |
| Gln Ala Leu Phe Val Gly Tyr Gly Pro Gly Phe Lys His Gly Ile Glu | |
| 545 550 555 560 | |
| Ala Asp Thr Phe Glu Asn Ile Glu Val Tyr Asn Leu Met Cys Asp Leu | |
| 565 570 575 | |
| Leu Asn Leu Thr Pro Ala Pro Asn Asn Gly Thr His Gly Ser Leu Asn | |
| 580 585 590 | |
| His Leu Leu Lys Asn Pro Val Tyr Thr Pro Lys His Pro Lys Glu Val | |
| 595 600 605 | |
| His Pro Leu Val Gln Cys Pro Phe Thr Arg Asn Pro Arg Asp Asn Leu | |
| 610 615 620 | |
| Gly Cys Ser Cys Asn Pro Ser Ile Leu Pro Ile Glu Asp Phe Gln Thr | |
| 625 630 635 640 | |
| Gln Phe Asn Leu Thr Val Ala Glu Glu Lys Ile Ile Lys His Glu Thr | |
| 645 650 655 | |
| Leu Pro Tyr Gly Arg Pro Arg Val Leu Gln Lys Glu Asn Thr Ile Cys | |
| 660 665 670 | |
| Leu Leu Ser Gln His Gln Phe Met Ser Gly Tyr Ser Gln Asp Ile Leu | |
| 675 680 685 | |
| Met Pro Leu Trp Thr Ser Tyr Thr Val Asp Arg Asn Asp Ser Phe Ser | |
| 690 695 700 | |
| Thr Glu Asp Phe Ser Asn Cys Leu Tyr Gln Asp Phe Arg Ile Pro Leu | |
| 705 710 715 720 | |
| Ser Pro Val His Lys Cys Ser Phe Tyr Lys Asn Asn Thr Lys Val Ser | |
| 725 730 735 | |
| Tyr Gly Phe Leu Ser Pro Pro Gln Leu Asn Lys Asn Ser Ser Gly Ile | |
| 740 745 750 | |
| Tyr Ser Glu Ala Leu Leu Thr Thr Asn Ile Val Pro Met Tyr Gln Ser | |
| 755 760 765 | |
| Phe Gln Val Ile Trp Arg Tyr Phe His Asp Thr Leu Leu Arg Lys Tyr | |
| 770 775 780 | |
| Ala Glu Glu Arg Asn Gly Val Asn Val Val Ser Gly Pro Val Phe Asp | |
| 785 790 795 800 | |
| Phe Asp Tyr Asp Gly Arg Cys Asp Ser Leu Glu Asn Leu Arg Gln Lys | |
| 805 810 815 | |
| Arg Arg Val Ile Arg Asn Gln Glu Ile Leu Ile Pro Thr His Phe Phe | |
| 820 825 830 | |
| Ile Val Leu Thr Ser Cys Lys Asp Thr Ser Gln Thr Pro Leu His Cys | |
| 835 840 845 | |
| Glu Asn Leu Asp Thr Leu Ala Phe Ile Leu Pro His Arg Thr Asp Asn | |
| 850 855 860 | |
| Ser Glu Ser Cys Val His Gly Lys His Asp Ser Ser Trp Val Glu Glu | |
| 865 870 875 880 | |
| Leu Leu Met Leu His Arg Ala Arg Ile Thr Asp Val Glu His Ile Thr | |
| 885 890 895 | |
| Gly Leu Ser Phe Tyr Gln Gln Arg Lys Glu Pro Val Ser Asp Ile Leu | |
| 900 905 910 | |
| Lys Leu Lys Thr His Leu Pro Thr Phe Ser Gln Glu Asp | |
| 915 920 925 | |
| Singly underlined: signal peptide sequence; double-underlined: | |
| beginning and end of NPP1; ** = cleavage position at the signal | |
| peptide sequence | |
| ENPP121-Fc Amino Acid Sequence | |
| SEQ. ID. NO: 28 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly**Phe Thr Ala Gly | |
| 85 90 95 | |
| Leu Lys Pro Ser Cys Ala Lys Glu Val Lys Ser Cys Lys Gly Arg Cys | |
| 100 105 110 | |
| Phe Glu Arg Thr Phe Gly Asn Cys Arg Cys Asp Ala Ala Cys Val Glu | |
| 115 120 125 | |
| Leu Gly Asn Cys Cys Leu Asp Tyr Gln Glu Thr Cys Ile Glu Pro Glu | |
| 130 135 140 | |
| His Ile Trp Thr Cys Asn Lys Phe Arg Cys Gly Glu Lys Arg Leu Thr | |
| 145 150 155 160 | |
| Arg Ser Leu Cys Ala Cys Ser Asp Asp Cys Lys Asp Lys Gly Asp Cys | |
| 165 170 175 | |
| Cys Ile Asn Tyr Ser Ser Val Cys Gln Gly Glu Lys Ser Trp Val Glu | |
| 180 185 190 | |
| Glu Pro Cys Glu Ser Ile Asn Glu Pro Gln Cys Pro Ala Gly Phe Glu | |
| 195 200 205 | |
| Thr Pro Pro Thr Leu Leu Phe Ser Leu Asp Gly Phe Arg Ala Glu Tyr | |
| 210 215 220 | |
| Leu His Thr Trp Gly Gly Leu Leu Pro Val Ile Ser Lys Leu Lys Lys | |
| 225 230 235 240 | |
| Cys Gly Thr Tyr Thr Lys Asn Met Arg Pro Val Tyr Pro Thr Lys Thr | |
| 245 250 255 | |
| Phe Pro Asn His Tyr Ser Ile Val Thr Gly Leu Tyr Pro Glu Ser His | |
| 260 265 270 | |
| Gly Ile Ile Asp Asn Lys Met Tyr Asp Pro Lys Met Asn Ala Ser Phe | |
| 275 280 285 | |
| Ser Leu Lys Ser Lys Glu Lys Phe Asn Pro Glu Trp Tyr Lys Gly Glu | |
| 290 295 300 | |
| Pro Ile Trp Val Thr Ala Lys Tyr Gln Gly Leu Lys Ser Gly Thr Phe | |
| 305 310 315 320 | |
| Phe Trp Pro Gly Ser Asp Val Glu Ile Asn Gly Ile Phe Pro Asp Ile | |
| 325 330 335 | |
| Tyr Lys Met Tyr Asn Gly Ser Val Pro Phe Glu Glu Arg Ile Leu Ala | |
| 340 345 350 | |
| Val Leu Gln Trp Leu Gln Leu Pro Lys Asp Glu Arg Pro His Phe Tyr | |
| 355 360 365 | |
| Thr Leu Tyr Leu Glu Glu Pro Asp Ser Ser Gly His Ser Tyr Gly Pro | |
| 370 375 380 | |
| Val Ser Ser Glu Val Ile Lys Ala Leu Gln Arg Val Asp Gly Met Val | |
| 385 390 395 400 | |
| Gly Met Leu Met Asp Gly Leu Lys Glu Leu Asn Leu His Arg Cys Leu | |
| 405 410 415 | |
| Asn Leu Ile Leu Ile Ser Asp His Gly Met Glu Gln Gly Ser Cys Lys | |
| 420 425 430 | |
| Lys Tyr Ile Tyr Leu Asn Lys Tyr Leu Gly Asp Val Lys Asn Ile Lys | |
| 435 440 445 | |
| Val Ile Tyr Gly Pro Ala Ala Arg Leu Arg Pro Ser Asp Val Pro Asp | |
| 450 455 460 | |
| Lys Tyr Tyr Ser Phe Asn Tyr Glu Gly Ile Ala Arg Asn Leu Ser Cys | |
| 465 470 475 480 | |
| Arg Glu Pro Asn Gln His Phe Lys Pro Tyr Leu Lys His Phe Leu Pro | |
| 485 490 495 | |
| Lys Arg Leu His Phe Ala Lys Ser Asp Arg Ile Glu Pro Leu Thr Phe | |
| 500 505 510 | |
| Tyr Leu Asp Pro Gln Trp Gln Leu Ala Leu Asn Pro Ser Glu Arg Lys | |
| 515 520 525 | |
| Tyr Cys Gly Ser Gly Phe His Gly Ser Asp Asn Val Phe Ser Asn Met | |
| 530 535 540 | |
| Gln Ala Leu Phe Val Gly Tyr Gly Pro Gly Phe Lys His Gly Ile Glu | |
| 545 550 555 560 | |
| Ala Asp Thr Phe Glu Asn Ile Glu Val Tyr Asn Leu Met Cys Asp Leu | |
| 565 570 575 | |
| Leu Asn Leu Thr Pro Ala Pro Asn Asn Gly Thr His Gly Ser Leu Asn | |
| 580 585 590 | |
| His Leu Leu Lys Asn Pro Val Tyr Thr Pro Lys His Pro Lys Glu Val | |
| 595 600 605 | |
| His Pro Leu Val Gln Cys Pro Phe Thr Arg Asn Pro Arg Asp Asn Leu | |
| 610 615 620 | |
| Gly Cys Ser Cys Asn Pro Ser Ile Leu Pro Ile Glu Asp Phe Gln Thr | |
| 625 630 635 640 | |
| Gln Phe Asn Leu Thr Val Ala Glu Glu Lys Ile Ile Lys His Glu Thr | |
| 645 650 655 | |
| Leu Pro Tyr Gly Arg Pro Arg Val Leu Gln Lys Glu Asn Thr Ile Cys | |
| 660 665 670 | |
| Leu Leu Ser Gln His Gln Phe Met Ser Gly Tyr Ser Gln Asp Ile Leu | |
| 675 680 685 | |
| Met Pro Leu Trp Thr Ser Tyr Thr Val Asp Arg Asn Asp Ser Phe Ser | |
| 690 695 700 | |
| Thr Glu Asp Phe Ser Asn Cys Leu Tyr Gln Asp Phe Arg Ile Pro Leu | |
| 705 710 715 720 | |
| Ser Pro Val His Lys Cys Ser Phe Tyr Lys Asn Asn Thr Lys Val Ser | |
| 725 730 735 | |
| Tyr Gly Phe Leu Ser Pro Pro Gln Leu Asn Lys Asn Ser Ser Gly Ile | |
| 740 745 750 | |
| Tyr Ser Glu Ala Leu Leu Thr Thr Asn Ile Val Pro Met Tyr Gln Ser | |
| 755 760 765 | |
| Phe Gln Val Ile Trp Arg Tyr Phe His Asp Thr Leu Leu Arg Lys Tyr | |
| 770 775 780 | |
| Ala Glu Glu Arg Asn Gly Val Asn Val Val Ser Gly Pro Val Phe Asp | |
| 785 790 795 800 | |
| Phe Asp Tyr Asp Gly Arg Cys Asp Ser Leu Glu Asn Leu Arg Gln Lys | |
| 805 810 815 | |
| Arg Arg Val Ile Arg Asn Gln Glu Ile Leu Ile Pro Thr His Phe Phe | |
| 820 825 830 | |
| Ile Val Leu Thr Ser Cys Lys Asp Thr Ser Gln Thr Pro Leu His Cys | |
| 835 840 845 | |
| Glu Asn Leu Asp Thr Leu Ala Phe Ile Leu Pro His Arg Thr Asp Asn | |
| 850 855 860 | |
| Ser Glu Ser Cys Val His Gly Lys His Asp Ser Ser Trp Val Glu Glu | |
| 865 870 875 880 | |
| Leu Leu Met Leu His Arg Ala Arg Ile Thr Asp Val Glu His Ile Thr | |
| 885 890 895 | |
| Gly Leu Ser Phe Tyr Gln Gln Arg Lys Glu Pro Val Ser Asp Ile Leu | |
| 900 905 910 | |
| Lys Leu Lys Thr His Leu Pro Thr Phe Ser Gln Glu Asp Leu Ile Asn | |
| 915 920 925 | |
| Asp Lys Thr His Thr Cys Pro Pro Cys Pro Ala Pro Glu Leu Leu Gly | |
| 930 935 940 | |
| Gly Pro Ser Val Phe Leu Phe Pro Pro Lys Pro Lys Asp Thr Leu Met | |
| 945 950 955 960 | |
| Ile Ser Arg Thr Pro Glu Val Thr Cys Val Val Val Asp Val Ser His | |
| 965 970 975 | |
| Glu Asp Pro Glu Val Lys Phe Asn Trp Tyr Val Asp Gly Val Glu Val | |
| 980 985 990 | |
| His Asn Ala Lys Thr Lys Pro Arg Glu Glu Gln Tyr Asn Ser Thr Tyr | |
| 995 1000 1005 | |
| Arg Val Val Ser Val Leu Thr Val Leu His Gln Asp Trp Leu Asn | |
| 1010 1015 1020 | |
| Gly Lys Glu Tyr Lys Cys Lys Val Ser Asn Lys Ala Leu Pro Ala | |
| 1025 1030 1035 | |
| Pro Ile Glu Lys Thr Ile Ser Lys Ala Lys Gly Gln Pro Arg Glu | |
| 1040 1045 1050 | |
| Pro Gln Val Tyr Thr Leu Pro Pro Ser Arg Glu Glu Met Thr Lys | |
| 1055 1060 1065 | |
| Asn Gln Val Ser Leu Thr Cys Leu Val Lys Gly Phe Tyr Pro Ser | |
| 1070 1075 1080 | |
| Asp Ile Ala Val Glu Trp Glu Ser Asn Gly Gln Pro Glu Asn Asn | |
| 1085 1090 1095 | |
| Tyr Lys Thr Thr Pro Pro Val Leu Asp Ser Asp Gly Ser Phe Phe | |
| 1100 1105 1110 | |
| Leu Tyr Ser Lys Leu Thr Val Asp Lys Ser Arg Trp Gln Gln Gly | |
| 1115 1120 1125 | |
| Asn Val Phe Ser Cys Ser Val Met His Glu Ala Leu His Asn His | |
| 1130 1135 1140 | |
| Tyr Thr Gln Lys Ser Leu Ser Leu Ser Pro Gly Lys | |
| 1145 1150 1155 | |
| Singly underlined: signal peptide sequence; double-underlined: | |
| beginning and end of NPP1; ** = cleavage position at the signal | |
| peptide sequence; bold residues indicate Fc sequence | |
| ENPP121-ALB Amino Acid Sequence: | |
| SEQ. ID NO: 29 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly**Phe Thr Ala Gly | |
| 85 90 95 | |
| Leu Lys Pro Ser Cys Ala Lys Glu Val Lys Ser Cys Lys Gly Arg Cys | |
| 100 105 110 | |
| Phe Glu Arg Thr Phe Gly Asn Cys Arg Cys Asp Ala Ala Cys Val Glu | |
| 115 120 125 | |
| Leu Gly Asn Cys Cys Leu Asp Tyr Gln Glu Thr Cys Ile Glu Pro Glu | |
| 130 135 140 | |
| His Ile Trp Thr Cys Asn Lys Phe Arg Cys Gly Glu Lys Arg Leu Thr | |
| 145 150 155 160 | |
| Arg Ser Leu Cys Ala Cys Ser Asp Asp Cys Lys Asp Lys Gly Asp Cys | |
| 165 170 175 | |
| Cys Ile Asn Tyr Ser Ser Val Cys Gln Gly Glu Lys Ser Trp Val Glu | |
| 180 185 190 | |
| Glu Pro Cys Glu Ser Ile Asn Glu Pro Gln Cys Pro Ala Gly Phe Glu | |
| 195 200 205 | |
| Thr Pro Pro Thr Leu Leu Phe Ser Leu Asp Gly Phe Arg Ala Glu Tyr | |
| 210 215 220 | |
| Leu His Thr Trp Gly Gly Leu Leu Pro Val Ile Ser Lys Leu Lys Lys | |
| 225 230 235 240 | |
| Cys Gly Thr Tyr Thr Lys Asn Met Arg Pro Val Tyr Pro Thr Lys Thr | |
| 245 250 255 | |
| Phe Pro Asn His Tyr Ser Ile Val Thr Gly Leu Tyr Pro Glu Ser His | |
| 260 265 270 | |
| Gly Ile Ile Asp Asn Lys Met Tyr Asp Pro Lys Met Asn Ala Ser Phe | |
| 275 280 285 | |
| Ser Leu Lys Ser Lys Glu Lys Phe Asn Pro Glu Trp Tyr Lys Gly Glu | |
| 290 295 300 | |
| Pro Ile Trp Val Thr Ala Lys Tyr Gln Gly Leu Lys Ser Gly Thr Phe | |
| 305 310 315 320 | |
| Phe Trp Pro Gly Ser Asp Val Glu Ile Asn Gly Ile Phe Pro Asp Ile | |
| 325 330 335 | |
| Tyr Lys Met Tyr Asn Gly Ser Val Pro Phe Glu Glu Arg Ile Leu Ala | |
| 340 345 350 | |
| Val Leu Gln Trp Leu Gln Leu Pro Lys Asp Glu Arg Pro His Phe Tyr | |
| 355 360 365 | |
| Thr Leu Tyr Leu Glu Glu Pro Asp Ser Ser Gly His Ser Tyr Gly Pro | |
| 370 375 380 | |
| Val Ser Ser Glu Val Ile Lys Ala Leu Gln Arg Val Asp Gly Met Val | |
| 385 390 395 400 | |
| Gly Met Leu Met Asp Gly Leu Lys Glu Leu Asn Leu His Arg Cys Leu | |
| 405 410 415 | |
| Asn Leu Ile Leu Ile Ser Asp His Gly Met Glu Gln Gly Ser Cys Lys | |
| 420 425 430 | |
| Lys Tyr Ile Tyr Leu Asn Lys Tyr Leu Gly Asp Val Lys Asn Ile Lys | |
| 435 440 445 | |
| Val Ile Tyr Gly Pro Ala Ala Arg Leu Arg Pro Ser Asp Val Pro Asp | |
| 450 455 460 | |
| Lys Tyr Tyr Ser Phe Asn Tyr Glu Gly Ile Ala Arg Asn Leu Ser Cys | |
| 465 470 475 480 | |
| Arg Glu Pro Asn Gln His Phe Lys Pro Tyr Leu Lys His Phe Leu Pro | |
| 485 490 495 | |
| Lys Arg Leu His Phe Ala Lys Ser Asp Arg Ile Glu Pro Leu Thr Phe | |
| 500 505 510 | |
| Tyr Leu Asp Pro Gln Trp Gln Leu Ala Leu Asn Pro Ser Glu Arg Lys | |
| 515 520 525 | |
| Tyr Cys Gly Ser Gly Phe His Gly Ser Asp Asn Val Phe Ser Asn Met | |
| 530 535 540 | |
| Gln Ala Leu Phe Val Gly Tyr Gly Pro Gly Phe Lys His Gly Ile Glu | |
| 545 550 555 560 | |
| Ala Asp Thr Phe Glu Asn Ile Glu Val Tyr Asn Leu Met Cys Asp Leu | |
| 565 570 575 | |
| Leu Asn Leu Thr Pro Ala Pro Asn Asn Gly Thr His Gly Ser Leu Asn | |
| 580 585 590 | |
| His Leu Leu Lys Asn Pro Val Tyr Thr Pro Lys His Pro Lys Glu Val | |
| 595 600 605 | |
| His Pro Leu Val Gln Cys Pro Phe Thr Arg Asn Pro Arg Asp Asn Leu | |
| 610 615 620 | |
| Gly Cys Ser Cys Asn Pro Ser Ile Leu Pro Ile Glu Asp Phe Gln Thr | |
| 625 630 635 640 | |
| Gln Phe Asn Leu Thr Val Ala Glu Glu Lys Ile Ile Lys His Glu Thr | |
| 645 650 655 | |
| Leu Pro Tyr Gly Arg Pro Arg Val Leu Gln Lys Glu Asn Thr Ile Cys | |
| 660 665 670 | |
| Leu Leu Ser Gln His Gln Phe Met Ser Gly Tyr Ser Gln Asp Ile Leu | |
| 675 680 685 | |
| Met Pro Leu Trp Thr Ser Tyr Thr Val Asp Arg Asn Asp Ser Phe Ser | |
| 690 695 700 | |
| Thr Glu Asp Phe Ser Asn Cys Leu Tyr Gln Asp Phe Arg Ile Pro Leu | |
| 705 710 715 720 | |
| Ser Pro Val His Lys Cys Ser Phe Tyr Lys Asn Asn Thr Lys Val Ser | |
| 725 730 735 | |
| Tyr Gly Phe Leu Ser Pro Pro Gln Leu Asn Lys Asn Ser Ser Gly Ile | |
| 740 745 750 | |
| Tyr Ser Glu Ala Leu Leu Thr Thr Asn Ile Val Pro Met Tyr Gln Ser | |
| 755 760 765 | |
| Phe Gln Val Ile Trp Arg Tyr Phe His Asp Thr Leu Leu Arg Lys Tyr | |
| 770 775 780 | |
| Ala Glu Glu Arg Asn Gly Val Asn Val Val Ser Gly Pro Val Phe Asp | |
| 785 790 795 800 | |
| Phe Asp Tyr Asp Gly Arg Cys Asp Ser Leu Glu Asn Leu Arg Gln Lys | |
| 805 810 815 | |
| Arg Arg Val Ile Arg Asn Gln Glu Ile Leu Ile Pro Thr His Phe Phe | |
| 820 825 830 | |
| Ile Val Leu Thr Ser Cys Lys Asp Thr Ser Gln Thr Pro Leu His Cys | |
| 835 840 845 | |
| Glu Asn Leu Asp Thr Leu Ala Phe Ile Leu Pro His Arg Thr Asp Asn | |
| 850 855 860 | |
| Ser Glu Ser Cys Val His Gly Lys His Asp Ser Ser Trp Val Glu Glu | |
| 865 870 875 880 | |
| Leu Leu Met Leu His Arg Ala Arg Ile Thr Asp Val Glu His Ile Thr | |
| 885 890 895 | |
| Gly Leu Ser Phe Tyr Gln Gln Arg Lys Glu Pro Val Ser Asp Ile Leu | |
| 900 905 910 | |
| Lys Leu Lys Thr His Leu Pro Thr Phe Ser Gln Glu Asp Arg Ser Gly | |
| 915 920 925 | |
| Ser Gly Gly Ser Met Lys Trp Val Thr Phe Leu Leu Leu Leu Phe Val | |
| 930 935 940 | |
| Ser Gly Ser Ala Phe Ser Arg Gly Val Phe Arg Arg Glu Ala His Lys | |
| 945 950 955 960 | |
| Ser Glu Ile Ala His Arg Tyr Asn Asp Leu Gly Glu Gln His Phe Lys | |
| 965 970 975 | |
| Gly Leu Val Leu Ile Ala Phe Ser Gln Tyr Leu Gln Lys Cys Ser Tyr | |
| 980 985 990 | |
| Asp Glu His Ala Lys Leu Val Gln Glu Val Thr Asp Phe Ala Lys Thr | |
| 995 1000 1005 | |
| Cys Val Ala Asp Glu Ser Ala Ala Asn Cys Asp Lys Ser Leu His | |
| 1010 1015 1020 | |
| Thr Leu Phe Gly Asp Lys Leu Cys Ala Ile Pro Asn Leu Arg Glu | |
| 1025 1030 1035 | |
| Asn Tyr Gly Glu Leu Ala Asp Cys Cys Thr Lys Gln Glu Pro Glu | |
| 1040 1045 1050 | |
| Arg Asn Glu Cys Phe Leu Gln His Lys Asp Asp Asn Pro Ser Leu | |
| 1055 1060 1065 | |
| Pro Pro Phe Glu Arg Pro Glu Ala Glu Ala Met Cys Thr Ser Phe | |
| 1070 1075 1080 | |
| Lys Glu Asn Pro Thr Thr Phe Met Gly His Tyr Leu His Glu Val | |
| 1085 1090 1095 | |
| Ala Arg Arg His Pro Tyr Phe Tyr Ala Pro Glu Leu Leu Tyr Tyr | |
| 1100 1105 1110 | |
| Ala Glu Gln Tyr Asn Glu Ile Leu Thr Gln Cys Cys Ala Glu Ala | |
| 1115 1120 1125 | |
| Asp Lys Glu Ser Cys Leu Thr Pro Lys Leu Asp Gly Val Lys Glu | |
| 1130 1135 1140 | |
| Lys Ala Leu Val Ser Ser Val Arg Gln Arg Met Lys Cys Ser Ser | |
| 1145 1150 1155 | |
| Met Gln Lys Phe Gly Glu Arg Ala Phe Lys Ala Trp Ala Val Ala | |
| 1160 1165 1170 | |
| Arg Leu Ser Gln Thr Phe Pro Asn Ala Asp Phe Ala Glu Ile Thr | |
| 1175 1180 1185 | |
| Lys Leu Ala Thr Asp Leu Thr Lys Val Asn Lys Glu Cys Cys His | |
| 1190 1195 1200 | |
| Gly Asp Leu Leu Glu Cys Ala Asp Asp Arg Ala Glu Leu Ala Lys | |
| 1205 1210 1215 | |
| Tyr Met Cys Glu Asn Gln Ala Thr Ile Ser Ser Lys Leu Gln Thr | |
| 1220 1225 1230 | |
| Cys Cys Asp Lys Pro Leu Leu Lys Lys Ala His Cys Leu Ser Glu | |
| 1235 1240 1245 | |
| Val Glu His Asp Thr Met Pro Ala Asp Leu Pro Ala Ile Ala Ala | |
| 1250 1255 1260 | |
| Asp Phe Val Glu Asp Gln Glu Val Cys Lys Asn Tyr Ala Glu Ala | |
| 1265 1270 1275 | |
| Lys Asp Val Phe Leu Gly Thr Phe Leu Tyr Glu Tyr Ser Arg Arg | |
| 1280 1285 1290 | |
| His Pro Asp Tyr Ser Val Ser Leu Leu Leu Arg Leu Ala Lys Lys | |
| 1295 1300 1305 | |
| Tyr Glu Ala Thr Leu Glu Lys Cys Cys Ala Glu Ala Asn Pro Pro | |
| 1310 1315 1320 | |
| Ala Cys Tyr Gly Thr Val Leu Ala Glu Phe Gln Pro Leu Val Glu | |
| 1325 1330 1335 | |
| Glu Pro Lys Asn Leu Val Lys Thr Asn Cys Asp Leu Tyr Glu Lys | |
| 1340 1345 1350 | |
| Leu Gly Glu Tyr Gly Phe Gln Asn Ala Ile Leu Val Arg Tyr Thr | |
| 1355 1360 1365 | |
| Gln Lys Ala Pro Gln Val Ser Thr Pro Thr Leu Val Glu Ala Ala | |
| 1370 1375 1380 | |
| Arg Asn Leu Gly Arg Val Gly Thr Lys Cys Cys Thr Leu Pro Glu | |
| 1385 1390 1395 | |
| Asp Gln Arg Leu Pro Cys Val Glu Asp Tyr Leu Ser Ala Ile Leu | |
| 1400 1405 1410 | |
| Asn Arg Val Cys Leu Leu His Glu Lys Thr Pro Val Ser Glu His | |
| 1415 1420 1425 | |
| Val Thr Lys Cys Cys Ser Gly Ser Leu Val Glu Arg Arg Pro Cys | |
| 1430 1435 1440 | |
| Phe Ser Ala Leu Thr Val Asp Glu Thr Tyr Val Pro Lys Glu Phe | |
| 1445 1450 1455 | |
| Lys Ala Glu Thr Phe Thr Phe His Ser Asp Ile Cys Thr Leu Pro | |
| 1460 1465 1470 | |
| Glu Lys Glu Lys Gln Ile Lys Lys Gln Thr Ala Leu Ala Glu Leu | |
| 1475 1480 1485 | |
| Val Lys His Lys Pro Lys Ala Thr Ala Glu Gln Leu Lys Thr Val | |
| 1490 1495 1500 | |
| Met Asp Asp Phe Ala Gln Phe Leu Asp Thr Cys Cys Lys Ala Ala | |
| 1505 1510 1515 | |
| Asp Lys Asp Thr Cys Phe Ser Thr Glu Gly Pro Asn Leu Val Thr | |
| 1520 1525 1530 | |
| Arg Cys Lys Asp Ala Leu Ala Arg Ser Trp Ser His Pro Gln Phe | |
| 1535 1540 1545 | |
| Glu Lys | |
| 1550 | |
| Singly underlined: signal peptide sequence; double-underlined: | |
| beginning and end of NPP1; ** = cleavage position at the signal | |
| peptide sequence; bold residues indicate albumin sequence | |
| ENPP121-NPP3-Fc sequence | |
| SEQ. ID NO: 30 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly Phe Thr Ala**Lys | |
| 85 90 95 | |
| Gln Gly Ser Cys Arg Lys Lys Cys Phe Asp Ala Ser Phe Arg Gly Leu | |
| 100 105 110 | |
| Glu Asn Cys Arg Cys Asp Val Ala Cys Lys Asp Arg Gly Asp Cys Cys | |
| 115 120 125 | |
| Trp Asp Phe Glu Asp Thr Cys Val Glu Ser Thr Arg Ile Trp Met Cys | |
| 130 135 140 | |
| Asn Lys Phe Arg Cys Gly Glu Arg Leu Glu Ala Ser Leu Cys Ser Cys | |
| 145 150 155 160 | |
| Ser Asp Asp Cys Leu Gln Arg Lys Asp Cys Cys Ala Asp Tyr Lys Ser | |
| 165 170 175 | |
| Val Cys Gln Gly Glu Thr Ser Trp Leu Glu Glu Asn Cys Asp Thr Ala | |
| 180 185 190 | |
| Gln Gln Ser Gln Cys Pro Glu Gly Phe Asp Leu Pro Pro Val Ile Leu | |
| 195 200 205 | |
| Phe Ser Met Asp Gly Phe Arg Ala Glu Tyr Leu Tyr Thr Trp Asp Thr | |
| 210 215 220 | |
| Leu Met Pro Asn Ile Asn Lys Leu Lys Thr Cys Gly Ile His Ser Lys | |
| 225 230 235 240 | |
| Tyr Met Arg Ala Met Tyr Pro Thr Lys Thr Phe Pro Asn His Tyr Thr | |
| 245 250 255 | |
| Ile Val Thr Gly Leu Tyr Pro Glu Ser His Gly Ile Ile Asp Asn Asn | |
| 260 265 270 | |
| Met Tyr Asp Val Asn Leu Asn Lys Asn Phe Ser Leu Ser Ser Lys Glu | |
| 275 280 285 | |
| Gln Asn Asn Pro Ala Trp Trp His Gly Gln Pro Met Trp Leu Thr Ala | |
| 290 295 300 | |
| Met Tyr Gln Gly Leu Lys Ala Ala Thr Tyr Phe Trp Pro Gly Ser Glu | |
| 305 310 315 320 | |
| Val Ala Ile Asn Gly Ser Phe Pro Ser Ile Tyr Met Pro Tyr Asn Gly | |
| 325 330 335 | |
| Ser Val Pro Phe Glu Glu Arg Ile Ser Thr Leu Leu Lys Trp Leu Asp | |
| 340 345 350 | |
| Leu Pro Lys Ala Glu Arg Pro Arg Phe Tyr Thr Met Tyr Phe Glu Glu | |
| 355 360 365 | |
| Pro Asp Ser Ser Gly His Ala Gly Gly Pro Val Ser Ala Arg Val Ile | |
| 370 375 380 | |
| Lys Ala Leu Gln Val Val Asp His Ala Phe Gly Met Leu Met Glu Gly | |
| 385 390 395 400 | |
| Leu Lys Gln Arg Asn Leu His Asn Cys Val Asn Ile Ile Leu Leu Ala | |
| 405 410 415 | |
| Asp His Gly Met Asp Gln Thr Tyr Cys Asn Lys Met Glu Tyr Met Thr | |
| 420 425 430 | |
| Asp Tyr Phe Pro Arg Ile Asn Phe Phe Tyr Met Tyr Glu Gly Pro Ala | |
| 435 440 445 | |
| Pro Arg Ile Arg Ala His Asn Ile Pro His Asp Phe Phe Ser Phe Asn | |
| 450 455 460 | |
| Ser Glu Glu Ile Val Arg Asn Leu Ser Cys Arg Lys Pro Asp Gln His | |
| 465 470 475 480 | |
| Phe Lys Pro Tyr Leu Thr Pro Asp Leu Pro Lys Arg Leu His Tyr Ala | |
| 485 490 495 | |
| Lys Asn Val Arg Ile Asp Lys Val His Leu Phe Val Asp Gln Gln Trp | |
| 500 505 510 | |
| Leu Ala Val Arg Ser Lys Ser Asn Thr Asn Cys Gly Gly Gly Asn His | |
| 515 520 525 | |
| Gly Tyr Asn Asn Glu Phe Arg Ser Met Glu Ala Ile Phe Leu Ala His | |
| 530 535 540 | |
| Gly Pro Ser Phe Lys Glu Lys Thr Glu Val Glu Pro Phe Glu Asn Ile | |
| 545 550 555 560 | |
| Glu Val Tyr Asn Leu Met Cys Asp Leu Leu Arg Ile Gln Pro Ala Pro | |
| 565 570 575 | |
| Asn Asn Gly Thr His Gly Ser Leu Asn His Leu Leu Lys Val Pro Phe | |
| 580 585 590 | |
| Tyr Glu Pro Ser His Ala Glu Glu Val Ser Lys Phe Ser Val Cys Gly | |
| 595 600 605 | |
| Phe Ala Asn Pro Leu Pro Thr Glu Ser Leu Asp Cys Phe Cys Pro His | |
| 610 615 620 | |
| Leu Gln Asn Ser Thr Gln Leu Glu Gln Val Asn Gln Met Leu Asn Leu | |
| 625 630 635 640 | |
| Thr Gln Glu Glu Ile Thr Ala Thr Val Lys Val Asn Leu Pro Phe Gly | |
| 645 650 655 | |
| Arg Pro Arg Val Leu Gln Lys Asn Val Asp His Cys Leu Leu Tyr His | |
| 660 665 670 | |
| Arg Glu Tyr Val Ser Gly Phe Gly Lys Ala Met Arg Met Pro Met Trp | |
| 675 680 685 | |
| Ser Ser Tyr Thr Val Pro Gln Leu Gly Asp Thr Ser Pro Leu Pro Pro | |
| 690 695 700 | |
| Thr Val Pro Asp Cys Leu Arg Ala Asp Val Arg Val Pro Pro Ser Glu | |
| 705 710 715 720 | |
| Ser Gln Lys Cys Ser Phe Tyr Leu Ala Asp Lys Asn Ile Thr His Gly | |
| 725 730 735 | |
| Phe Leu Tyr Pro Pro Ala Ser Asn Arg Thr Ser Asp Ser Gln Tyr Asp | |
| 740 745 750 | |
| Ala Leu Ile Thr Ser Asn Leu Val Pro Met Tyr Glu Glu Phe Arg Lys | |
| 755 760 765 | |
| Met Trp Asp Tyr Phe His Ser Val Leu Leu Ile Lys His Ala Thr Glu | |
| 770 775 780 | |
| Arg Asn Gly Val Asn Val Val Ser Gly Pro Ile Phe Asp Tyr Asn Tyr | |
| 785 790 795 800 | |
| Asp Gly His Phe Asp Ala Pro Asp Glu Ile Thr Lys His Leu Ala Asn | |
| 805 810 815 | |
| Thr Asp Val Pro Ile Pro Thr His Tyr Phe Val Val Leu Thr Ser Cys | |
| 820 825 830 | |
| Lys Asn Lys Ser His Thr Pro Glu Asn Cys Pro Gly Trp Leu Asp Val | |
| 835 840 845 | |
| Leu Pro Phe Ile Ile Pro His Arg Pro Thr Asn Val Glu Ser Cys Pro | |
| 850 855 860 | |
| Glu Gly Lys Pro Glu Ala Leu Trp Val Glu Glu Arg Phe Thr Ala His | |
| 865 870 875 880 | |
| Ile Ala Arg Val Arg Asp Val Glu Leu Leu Thr Gly Leu Asp Phe Tyr | |
| 885 890 895 | |
| Gln Asp Lys Val Gln Pro Val Ser Glu Ile Leu Gln Leu Lys Thr Tyr | |
| 900 905 910 | |
| Leu Pro Thr Phe Glu Thr Thr Ile Asp Lys Thr His Thr Cys Pro Pro | |
| 915 920 925 | |
| Cys Pro Ala Pro Glu Leu Leu Gly Gly Pro Ser Val Phe Leu Phe Pro | |
| 930 935 940 | |
| Pro Lys Pro Lys Asp Thr Leu Met Ile Ser Arg Thr Pro Glu Val Thr | |
| 945 950 955 960 | |
| Cys Val Val Val Asp Val Ser His Glu Asp Pro Glu Val Lys Phe Asn | |
| 965 970 975 | |
| Trp Tyr Val Asp Gly Val Glu Val His Asn Ala Lys Thr Lys Pro Arg | |
| 980 985 990 | |
| Glu Glu Gln Tyr Asn Ser Thr Tyr Arg Val Val Ser Val Leu Thr Val | |
| 995 1000 1005 | |
| Leu His Gln Asp Trp Leu Asn Gly Lys Glu Tyr Lys Cys Lys Val | |
| 1010 1015 1020 | |
| Ser Asn Lys Ala Leu Pro Ala Pro Ile Glu Lys Thr Ile Ser Lys | |
| 1025 1030 1035 | |
| Ala Lys Gly Gln Pro Arg Glu Pro Gln Val Tyr Thr Leu Pro Pro | |
| 1040 1045 1050 | |
| Ser Arg Glu Glu Met Thr Lys Asn Gln Val Ser Leu Thr Cys Leu | |
| 1055 1060 1065 | |
| Val Lys Gly Phe Tyr Pro Ser Asp Ile Ala Val Glu Trp Glu Ser | |
| 1070 1075 1080 | |
| Asn Gly Gln Pro Glu Asn Asn Tyr Lys Thr Thr Pro Pro Val Leu | |
| 1085 1090 1095 | |
| Asp Ser Asp Gly Ser Phe Phe Leu Tyr Ser Lys Leu Thr Val Asp | |
| 1100 1105 1110 | |
| Lys Ser Arg Trp Gln Gln Gly Asn Val Phe Ser Cys Ser Val Met | |
| 1115 1120 1125 | |
| His Glu Ala Leu His Asn His Tyr Thr Gln Lys Ser Leu Ser Leu | |
| 1130 1135 1140 | |
| Ser Pro Gly Lys | |
| 1145 | |
| Singly underlined: signal peptide sequence; double-underlined: | |
| beginning and end of NPP1; ** = cleavage position at the signal | |
| peptide sequence; bold residues indicate Fc sequence | |
| ENPP121-NPP3-Albumin sequence | |
| SEQ. ID NO: 31 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly Phe Thr Ala**Lys | |
| 85 90 95 | |
| Gln Gly Ser Cys Arg Lys Lys Cys Phe Asp Ala Ser Phe Arg Gly Leu | |
| 100 105 110 | |
| Glu Asn Cys Arg Cys Asp Val Ala Cys Lys Asp Arg Gly Asp Cys Cys | |
| 115 120 125 | |
| Trp Asp Phe Glu Asp Thr Cys Val Glu Ser Thr Arg Ile Trp Met Cys | |
| 130 135 140 | |
| Asn Lys Phe Arg Cys Gly Glu Arg Leu Glu Ala Ser Leu Cys Ser Cys | |
| 145 150 155 160 | |
| Ser Asp Asp Cys Leu Gln Arg Lys Asp Cys Cys Ala Asp Tyr Lys Ser | |
| 165 170 175 | |
| Val Cys Gln Gly Glu Thr Ser Trp Leu Glu Glu Asn Cys Asp Thr Ala | |
| 180 185 190 | |
| Gln Gln Ser Gln Cys Pro Glu Gly Phe Asp Leu Pro Pro Val Ile Leu | |
| 195 200 205 | |
| Phe Ser Met Asp Gly Phe Arg Ala Glu Tyr Leu Tyr Thr Trp Asp Thr | |
| 210 215 220 | |
| Leu Met Pro Asn Ile Asn Lys Leu Lys Thr Cys Gly Ile His Ser Lys | |
| 225 230 235 240 | |
| Tyr Met Arg Ala Met Tyr Pro Thr Lys Thr Phe Pro Asn His Tyr Thr | |
| 245 250 255 | |
| Ile Val Thr Gly Leu Tyr Pro Glu Ser His Gly Ile Ile Asp Asn Asn | |
| 260 265 270 | |
| Met Tyr Asp Val Asn Leu Asn Lys Asn Phe Ser Leu Ser Ser Lys Glu | |
| 275 280 285 | |
| Gln Asn Asn Pro Ala Trp Trp His Gly Gln Pro Met Trp Leu Thr Ala | |
| 290 295 300 | |
| Met Tyr Gln Gly Leu Lys Ala Ala Thr Tyr Phe Trp Pro Gly Ser Glu | |
| 305 310 315 320 | |
| Val Ala Ile Asn Gly Ser Phe Pro Ser Ile Tyr Met Pro Tyr Asn Gly | |
| 325 330 335 | |
| Ser Val Pro Phe Glu Glu Arg Ile Ser Thr Leu Leu Lys Trp Leu Asp | |
| 340 345 350 | |
| Leu Pro Lys Ala Glu Arg Pro Arg Phe Tyr Thr Met Tyr Phe Glu Glu | |
| 355 360 365 | |
| Pro Asp Ser Ser Gly His Ala Gly Gly Pro Val Ser Ala Arg Val Ile | |
| 370 375 380 | |
| Lys Ala Leu Gln Val Val Asp His Ala Phe Gly Met Leu Met Glu Gly | |
| 385 390 395 400 | |
| Leu Lys Gln Arg Asn Leu His Asn Cys Val Asn Ile Ile Leu Leu Ala | |
| 405 410 415 | |
| Asp His Gly Met Asp Gln Thr Tyr Cys Asn Lys Met Glu Tyr Met Thr | |
| 420 425 430 | |
| Asp Tyr Phe Pro Arg Ile Asn Phe Phe Tyr Met Tyr Glu Gly Pro Ala | |
| 435 440 445 | |
| Pro Arg Ile Arg Ala His Asn Ile Pro His Asp Phe Phe Ser Phe Asn | |
| 450 455 460 | |
| Ser Glu Glu Ile Val Arg Asn Leu Ser Cys Arg Lys Pro Asp Gln His | |
| 465 470 475 480 | |
| Phe Lys Pro Tyr Leu Thr Pro Asp Leu Pro Lys Arg Leu His Tyr Ala | |
| 485 490 495 | |
| Lys Asn Val Arg Ile Asp Lys Val His Leu Phe Val Asp Gln Gln Trp | |
| 500 505 510 | |
| Leu Ala Val Arg Ser Lys Ser Asn Thr Asn Cys Gly Gly Gly Asn His | |
| 515 520 525 | |
| Gly Tyr Asn Asn Glu Phe Arg Ser Met Glu Ala Ile Phe Leu Ala His | |
| 530 535 540 | |
| Gly Pro Ser Phe Lys Glu Lys Thr Glu Val Glu Pro Phe Glu Asn Ile | |
| 545 550 555 560 | |
| Glu Val Tyr Asn Leu Met Cys Asp Leu Leu Arg Ile Gln Pro Ala Pro | |
| 565 570 575 | |
| Asn Asn Gly Thr His Gly Ser Leu Asn His Leu Leu Lys Val Pro Phe | |
| 580 585 590 | |
| Tyr Glu Pro Ser His Ala Glu Glu Val Ser Lys Phe Ser Val Cys Gly | |
| 595 600 605 | |
| Phe Ala Asn Pro Leu Pro Thr Glu Ser Leu Asp Cys Phe Cys Pro His | |
| 610 615 620 | |
| Leu Gln Asn Ser Thr Gln Leu Glu Gln Val Asn Gln Met Leu Asn Leu | |
| 625 630 635 640 | |
| Thr Gln Glu Glu Ile Thr Ala Thr Val Lys Val Asn Leu Pro Phe Gly | |
| 645 650 655 | |
| Arg Pro Arg Val Leu Gln Lys Asn Val Asp His Cys Leu Leu Tyr His | |
| 660 665 670 | |
| Arg Glu Tyr Val Ser Gly Phe Gly Lys Ala Met Arg Met Pro Met Trp | |
| 675 680 685 | |
| Ser Ser Tyr Thr Val Pro Gln Leu Gly Asp Thr Ser Pro Leu Pro Pro | |
| 690 695 700 | |
| Thr Val Pro Asp Cys Leu Arg Ala Asp Val Arg Val Pro Pro Ser Glu | |
| 705 710 715 720 | |
| Ser Gln Lys Cys Ser Phe Tyr Leu Ala Asp Lys Asn Ile Thr His Gly | |
| 725 730 735 | |
| Phe Leu Tyr Pro Pro Ala Ser Asn Arg Thr Ser Asp Ser Gln Tyr Asp | |
| 740 745 750 | |
| Ala Leu Ile Thr Ser Asn Leu Val Pro Met Tyr Glu Glu Phe Arg Lys | |
| 755 760 765 | |
| Met Trp Asp Tyr Phe His Ser Val Leu Leu Ile Lys His Ala Thr Glu | |
| 770 775 780 | |
| Arg Asn Gly Val Asn Val Val Ser Gly Pro Ile Phe Asp Tyr Asn Tyr | |
| 785 790 795 800 | |
| Asp Gly His Phe Asp Ala Pro Asp Glu Ile Thr Lys His Leu Ala Asn | |
| 805 810 815 | |
| Thr Asp Val Pro Ile Pro Thr His Tyr Phe Val Val Leu Thr Ser Cys | |
| 820 825 830 | |
| Lys Asn Lys Ser His Thr Pro Glu Asn Cys Pro Gly Trp Leu Asp Val | |
| 835 840 845 | |
| Leu Pro Phe Ile Ile Pro His Arg Pro Thr Asn Val Glu Ser Cys Pro | |
| 850 855 860 | |
| Glu Gly Lys Pro Glu Ala Leu Trp Val Glu Glu Arg Phe Thr Ala His | |
| 865 870 875 880 | |
| Ile Ala Arg Val Arg Asp Val Glu Leu Leu Thr Gly Leu Asp Phe Tyr | |
| 885 890 895 | |
| Gln Asp Lys Val Gln Pro Val Ser Glu Ile Leu Gln Leu Lys Thr Tyr | |
| 900 905 910 | |
| Leu Pro Thr Phe Glu Thr Thr Ile Gly Gly Gly Ser Gly Gly Gly Gly | |
| 915 920 925 | |
| Ser Gly Gly Gly Gly Ser Met Lys Trp Val Thr Phe Leu Leu Leu Leu | |
| 930 935 940 | |
| Phe Val Ser Gly Ser Ala Phe Ser Arg Gly Val Phe Arg Arg Glu Ala | |
| 945 950 955 960 | |
| His Lys Ser Glu Ile Ala His Arg Tyr Asn Asp Leu Gly Glu Gln His | |
| 965 970 975 | |
| Phe Lys Gly Leu Val Leu Ile Ala Phe Ser Gln Tyr Leu Gln Lys Cys | |
| 980 985 990 | |
| Ser Tyr Asp Glu His Ala Lys Leu Val Gln Glu Val Thr Asp Phe Ala | |
| 995 1000 1005 | |
| Lys Thr Cys Val Ala Asp Glu Ser Ala Ala Asn Cys Asp Lys Ser | |
| 1010 1015 1020 | |
| Leu His Thr Leu Phe Gly Asp Lys Leu Cys Ala Ile Pro Asn Leu | |
| 1025 1030 1035 | |
| Arg Glu Asn Tyr Gly Glu Leu Ala Asp Cys Cys Thr Lys Gln Glu | |
| 1040 1045 1050 | |
| Pro Glu Arg Asn Glu Cys Phe Leu Gln His Lys Asp Asp Asn Pro | |
| 1055 1060 1065 | |
| Ser Leu Pro Pro Phe Glu Arg Pro Glu Ala Glu Ala Met Cys Thr | |
| 1070 1075 1080 | |
| Ser Phe Lys Glu Asn Pro Thr Thr Phe Met Gly His Tyr Leu His | |
| 1085 1090 1095 | |
| Glu Val Ala Arg Arg His Pro Tyr Phe Tyr Ala Pro Glu Leu Leu | |
| 1100 1105 1110 | |
| Tyr Tyr Ala Glu Gln Tyr Asn Glu Ile Leu Thr Gln Cys Cys Ala | |
| 1115 1120 1125 | |
| Glu Ala Asp Lys Glu Ser Cys Leu Thr Pro Lys Leu Asp Gly Val | |
| 1130 1135 1140 | |
| Lys Glu Lys Ala Leu Val Ser Ser Val Arg Gln Arg Met Lys Cys | |
| 1145 1150 1155 | |
| Ser Ser Met Gln Lys Phe Gly Glu Arg Ala Phe Lys Ala Trp Ala | |
| 1160 1165 1170 | |
| Val Ala Arg Leu Ser Gln Thr Phe Pro Asn Ala Asp Phe Ala Glu | |
| 1175 1180 1185 | |
| Ile Thr Lys Leu Ala Thr Asp Leu Thr Lys Val Asn Lys Glu Cys | |
| 1190 1195 1200 | |
| Cys His Gly Asp Leu Leu Glu Cys Ala Asp Asp Arg Ala Glu Leu | |
| 1205 1210 1215 | |
| Ala Lys Tyr Met Cys Glu Asn Gln Ala Thr Ile Ser Ser Lys Leu | |
| 1220 1225 1230 | |
| Gln Thr Cys Cys Asp Lys Pro Leu Leu Lys Lys Ala His Cys Leu | |
| 1235 1240 1245 | |
| Ser Glu Val Glu His Asp Thr Met Pro Ala Asp Leu Pro Ala Ile | |
| 1250 1255 1260 | |
| Ala Ala Asp Phe Val Glu Asp Gln Glu Val Cys Lys Asn Tyr Ala | |
| 1265 1270 1275 | |
| Glu Ala Lys Asp Val Phe Leu Gly Thr Phe Leu Tyr Glu Tyr Ser | |
| 1280 1285 1290 | |
| Arg Arg His Pro Asp Tyr Ser Val Ser Leu Leu Leu Arg Leu Ala | |
| 1295 1300 1305 | |
| Lys Lys Tyr Glu Ala Thr Leu Glu Lys Cys Cys Ala Glu Ala Asn | |
| 1310 1315 1320 | |
| Pro Pro Ala Cys Tyr Gly Thr Val Leu Ala Glu Phe Gln Pro Leu | |
| 1325 1330 1335 | |
| Val Glu Glu Pro Lys Asn Leu Val Lys Thr Asn Cys Asp Leu Tyr | |
| 1340 1345 1350 | |
| Glu Lys Leu Gly Glu Tyr Gly Phe Gln Asn Ala Ile Leu Val Arg | |
| 1355 1360 1365 | |
| Tyr Thr Gln Lys Ala Pro Gln Val Ser Thr Pro Thr Leu Val Glu | |
| 1370 1375 1380 | |
| Ala Ala Arg Asn Leu Gly Arg Val Gly Thr Lys Cys Cys Thr Leu | |
| 1385 1390 1395 | |
| Pro Glu Asp Gln Arg Leu Pro Cys Val Glu Asp Tyr Leu Ser Ala | |
| 1400 1405 1410 | |
| Ile Leu Asn Arg Val Cys Leu Leu His Glu Lys Thr Pro Val Ser | |
| 1415 1420 1425 | |
| Glu His Val Thr Lys Cys Cys Ser Gly Ser Leu Val Glu Arg Arg | |
| 1430 1435 1440 | |
| Pro Cys Phe Ser Ala Leu Thr Val Asp Glu Thr Tyr Val Pro Lys | |
| 1445 1450 1455 | |
| Glu Phe Lys Ala Glu Thr Phe Thr Phe His Ser Asp Ile Cys Thr | |
| 1460 1465 1470 | |
| Leu Pro Glu Lys Glu Lys Gln Ile Lys Lys Gln Thr Ala Leu Ala | |
| 1475 1480 1485 | |
| Glu Leu Val Lys His Lys Pro Lys Ala Thr Ala Glu Gln Leu Lys | |
| 1490 1495 1500 | |
| Thr Val Met Asp Asp Phe Ala Gln Phe Leu Asp Thr Cys Cys Lys | |
| 1505 1510 1515 | |
| Ala Ala Asp Lys Asp Thr Cys Phe Ser Thr Glu Gly Pro Asn Leu | |
| 1520 1525 1530 | |
| Val Thr Arg Cys Lys Asp Ala Leu Ala | |
| 1535 1540 | |
| Singly underlined: signal peptide sequence; double-underlined: | |
| beginning and end of NPP3; ** = cleavage position at the signal | |
| peptide sequence; bold residues indicate albumin sequence | |
| ENPP121GLK Protein Export Signal Sequence | |
| SEQ. ID NO: 32 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly Phe Thr Ala Gly | |
| 85 90 95 | |
| Leu Lys | |
| Albumin Sequence | |
| SEQ. ID NO: 33 | |
| Gly Gly Gly Gly Ser Gly Gly Gly Gly Ser Gly Gly Gly Gly Ser Met | |
| 1 5 10 15 | |
| Lys Trp Val Thr Phe Leu Leu Leu Leu Phe Val Ser Gly Ser Ala Phe | |
| 20 25 30 | |
| Ser Arg Gly Val Phe Arg Arg Glu Ala His Lys Ser Glu Ile Ala His | |
| 35 40 45 | |
| Arg Tyr Asn Asp Leu Gly Glu Gln His Phe Lys Gly Leu Val Leu Ile | |
| 50 55 60 | |
| Ala Phe Ser Gln Tyr Leu Gln Lys Cys Ser Tyr Asp Glu His Ala Lys | |
| 65 70 75 80 | |
| Leu Val Gln Glu Val Thr Asp Phe Ala Lys Thr Cys Val Ala Asp Glu | |
| 85 90 95 | |
| Ser Ala Ala Asn Cys Asp Lys Ser Leu His Thr Leu Phe Gly Asp Lys | |
| 100 105 110 | |
| Leu Cys Ala Ile Pro Asn Leu Arg Glu Asn Tyr Gly Glu Leu Ala Asp | |
| 115 120 125 | |
| Cys Cys Thr Lys Gln Glu Pro Glu Arg Asn Glu Cys Phe Leu Gln His | |
| 130 135 140 | |
| Lys Asp Asp Asn Pro Ser Leu Pro Pro Phe Glu Arg Pro Glu Ala Glu | |
| 145 150 155 160 | |
| Ala Met Cys Thr Ser Phe Lys Glu Asn Pro Thr Thr Phe Met Gly His | |
| 165 170 175 | |
| Tyr Leu His Glu Val Ala Arg Arg His Pro Tyr Phe Tyr Ala Pro Glu | |
| 180 185 190 | |
| Leu Leu Tyr Tyr Ala Glu Gln Tyr Asn Glu Ile Leu Thr Gln Cys Cys | |
| 195 200 205 | |
| Ala Glu Ala Asp Lys Glu Ser Cys Leu Thr Pro Lys Leu Asp Gly Val | |
| 210 215 220 | |
| Lys Glu Lys Ala Leu Val Ser Ser Val Arg Gln Arg Met Lys Cys Ser | |
| 225 230 235 240 | |
| Ser Met Gln Lys Phe Gly Glu Arg Ala Phe Lys Ala Trp Ala Val Ala | |
| 245 250 255 | |
| Arg Leu Ser Gln Thr Phe Pro Asn Ala Asp Phe Ala Glu Ile Thr Lys | |
| 260 265 270 | |
| Leu Ala Thr Asp Leu Thr Lys Val Asn Lys Glu Cys Cys His Gly Asp | |
| 275 280 285 | |
| Leu Leu Glu Cys Ala Asp Asp Arg Ala Glu Leu Ala Lys Tyr Met Cys | |
| 290 295 300 | |
| Glu Asn Gln Ala Thr Ile Ser Ser Lys Leu Gln Thr Cys Cys Asp Lys | |
| 305 310 315 320 | |
| Pro Leu Leu Lys Lys Ala His Cys Leu Ser Glu Val Glu His Asp Thr | |
| 325 330 335 | |
| Met Pro Ala Asp Leu Pro Ala Ile Ala Ala Asp Phe Val Glu Asp Gln | |
| 340 345 350 | |
| Glu Val Cys Lys Asn Tyr Ala Glu Ala Lys Asp Val Phe Leu Gly Thr | |
| 355 360 365 | |
| Phe Leu Tyr Glu Tyr Ser Arg Arg His Pro Asp Tyr Ser Val Ser Leu | |
| 370 375 380 | |
| Leu Leu Arg Leu Ala Lys Lys Tyr Glu Ala Thr Leu Glu Lys Cys Cys | |
| 385 390 395 400 | |
| Ala Glu Ala Asn Pro Pro Ala Cys Tyr Gly Thr Val Leu Ala Glu Phe | |
| 405 410 415 | |
| Gln Pro Leu Val Glu Glu Pro Lys Asn Leu Val Lys Thr Asn Cys Asp | |
| 420 425 430 | |
| Leu Tyr Glu Lys Leu Gly Glu Tyr Gly Phe Gln Asn Ala Ile Leu Val | |
| 435 440 445 | |
| Arg Tyr Thr Gln Lys Ala Pro Gln Val Ser Thr Pro Thr Leu Val Glu | |
| 450 455 460 | |
| Ala Ala Arg Asn Leu Gly Arg Val Gly Thr Lys Cys Cys Thr Leu Pro | |
| 465 470 475 480 | |
| Glu Asp Gln Arg Leu Pro Cys Val Glu Asp Tyr Leu Ser Ala Ile Leu | |
| 485 490 495 | |
| Asn Arg Val Cys Leu Leu His Glu Lys Thr Pro Val Ser Glu His Val | |
| 500 505 510 | |
| Thr Lys Cys Cys Ser Gly Ser Leu Val Glu Arg Arg Pro Cys Phe Ser | |
| 515 520 525 | |
| Ala Leu Thr Val Asp Glu Thr Tyr Val Pro Lys Glu Phe Lys Ala Glu | |
| 530 535 540 | |
| Thr Phe Thr Phe His Ser Asp Ile Cys Thr Leu Pro Glu Lys Glu Lys | |
| 545 550 555 560 | |
| Gln Ile Lys Lys Gln Thr Ala Leu Ala Glu Leu Val Lys His Lys Pro | |
| 565 570 575 | |
| Lys Ala Thr Ala Glu Gln Leu Lys Thr Val Met Asp Asp Phe Ala Gln | |
| 580 585 590 | |
| Phe Leu Asp Thr Cys Cys Lys Ala Ala Asp Lys Asp Thr Cys Phe Ser | |
| 595 600 605 | |
| Thr Glu Gly Pro Asn Leu Val Thr Arg Cys Lys Asp Ala Leu Ala | |
| 610 615 620 | |
| Human IgG Fc domain, Fc | |
| SEQ. ID NO: 34 | |
| Asp Lys Thr His Thr Cys Pro Pro Cys Pro Ala Pro Glu Leu Leu Gly | |
| 1 5 10 15 | |
| Gly Pro Ser Val Phe Leu Phe Pro Pro Lys Pro Lys Asp Thr Leu Met | |
| 20 25 30 | |
| Ile Ser Arg Thr Pro Glu Val Thr Cys Val Val Val Asp Val Ser His | |
| 35 40 45 | |
| Glu Asp Pro Glu Val Lys Phe Asn Trp Tyr Val Asp Gly Val Glu Val | |
| 50 55 60 | |
| His Asn Ala Lys Thr Lys Pro Arg Glu Glu Gln Tyr Asn Ser Thr Tyr | |
| 65 70 75 80 | |
| Arg Val Val Ser Val Leu Thr Val Leu His Gln Asp Trp Leu Asn Gly | |
| 85 90 95 | |
| Lys Glu Tyr Lys Cys Lys Val Ser Asn Lys Ala Leu Pro Ala Pro Ile | |
| 100 105 110 | |
| Glu Lys Thr Ile Ser Lys Ala Lys Gly Gln Pro Arg Glu Pro Gln Val | |
| 115 120 125 | |
| Tyr Thr Leu Pro Pro Ser Arg Glu Glu Met Thr Lys Asn Gln Val Ser | |
| 130 135 140 | |
| Leu Thr Cys Leu Val Lys Gly Phe Tyr Pro Ser Asp Ile Ala Val Glu | |
| 145 150 155 160 | |
| Trp Glu Ser Asn Gly Gln Pro Glu Asn Asn Tyr Lys Thr Thr Pro Pro | |
| 165 170 175 | |
| Val Leu Asp Ser Asp Gly Ser Phe Phe Leu Tyr Ser Lys Leu Thr Val | |
| 180 185 190 | |
| Asp Lys Ser Arg Trp Gln Gln Gly Asn Val Phe Ser Cys Ser Val Met | |
| 195 200 205 | |
| His Glu Ala Leu His Asn His Tyr Thr Gln Lys Ser Leu Ser Leu Ser | |
| 210 215 220 | |
| Pro Gly Lys | |
| 225 | |
| Albumin Sequence | |
| SEQ. ID NO: 35 | |
| Met Lys Trp Val Thr Phe Leu Leu Leu Leu Phe Val Ser Gly Ser Ala | |
| 1 5 10 15 | |
| Phe Ser Arg Gly Val Phe Arg Arg Glu Ala His Lys Ser Glu Ile Ala | |
| 20 25 30 | |
| His Arg Tyr Asn Asp Leu Gly Glu Gln His Phe Lys Gly Leu Val Leu | |
| 35 40 45 | |
| Ile Ala Phe Ser Gln Tyr Leu Gln Lys Cys Ser Tyr Asp Glu His Ala | |
| 50 55 60 | |
| Lys Leu Val Gln Glu Val Thr Asp Phe Ala Lys Thr Cys Val Ala Asp | |
| 65 70 75 80 | |
| Glu Ser Ala Ala Asn Cys Asp Lys Ser Leu His Thr Leu Phe Gly Asp | |
| 85 90 95 | |
| Lys Leu Cys Ala Ile Pro Asn Leu Arg Glu Asn Tyr Gly Glu Leu Ala | |
| 100 105 110 | |
| Asp Cys Cys Thr Lys Gln Glu Pro Glu Arg Asn Glu Cys Phe Leu Gln | |
| 115 120 125 | |
| His Lys Asp Asp Asn Pro Ser Leu Pro Pro Phe Glu Arg Pro Glu Ala | |
| 130 135 140 | |
| Glu Ala Met Cys Thr Ser Phe Lys Glu Asn Pro Thr Thr Phe Met Gly | |
| 145 150 155 160 | |
| His Tyr Leu His Glu Val Ala Arg Arg His Pro Tyr Phe Tyr Ala Pro | |
| 165 170 175 | |
| Glu Leu Leu Tyr Tyr Ala Glu Gln Tyr Asn Glu Ile Leu Thr Gln Cys | |
| 180 185 190 | |
| Cys Ala Glu Ala Asp Lys Glu Ser Cys Leu Thr Pro Lys Leu Asp Gly | |
| 195 200 205 | |
| Val Lys Glu Lys Ala Leu Val Ser Ser Val Arg Gln Arg Met Lys Cys | |
| 210 215 220 | |
| Ser Ser Met Gln Lys Phe Gly Glu Arg Ala Phe Lys Ala Trp Ala Val | |
| 225 230 235 240 | |
| Ala Arg Leu Ser Gln Thr Phe Pro Asn Ala Asp Phe Ala Glu Ile Thr | |
| 245 250 255 | |
| Lys Leu Ala Thr Asp Leu Thr Lys Val Asn Lys Glu Cys Cys His Gly | |
| 260 265 270 | |
| Asp Leu Leu Glu Cys Ala Asp Asp Arg Ala Glu Leu Ala Lys Tyr Met | |
| 275 280 285 | |
| Cys Glu Asn Gln Ala Thr Ile Ser Ser Lys Leu Gln Thr Cys Cys Asp | |
| 290 295 300 | |
| Lys Pro Leu Leu Lys Lys Ala His Cys Leu Ser Glu Val Glu His Asp | |
| 305 310 315 320 | |
| Thr Met Pro Ala Asp Leu Pro Ala Ile Ala Ala Asp Phe Val Glu Asp | |
| 325 330 335 | |
| Gln Glu Val Cys Lys Asn Tyr Ala Glu Ala Lys Asp Val Phe Leu Gly | |
| 340 345 350 | |
| Thr Phe Leu Tyr Glu Tyr Ser Arg Arg His Pro Asp Tyr Ser Val Ser | |
| 355 360 365 | |
| Leu Leu Leu Arg Leu Ala Lys Lys Tyr Glu Ala Thr Leu Glu Lys Cys | |
| 370 375 380 | |
| Cys Ala Glu Ala Asn Pro Pro Ala Cys Tyr Gly Thr Val Leu Ala Glu | |
| 385 390 395 400 | |
| Phe Gln Pro Leu Val Glu Glu Pro Lys Asn Leu Val Lys Thr Asn Cys | |
| 405 410 415 | |
| Asp Leu Tyr Glu Lys Leu Gly Glu Tyr Gly Phe Gln Asn Ala Ile Leu | |
| 420 425 430 | |
| Val Arg Tyr Thr Gln Lys Ala Pro Gln Val Ser Thr Pro Thr Leu Val | |
| 435 440 445 | |
| Glu Ala Ala Arg Asn Leu Gly Arg Val Gly Thr Lys Cys Cys Thr Leu | |
| 450 455 460 | |
| Pro Glu Asp Gln Arg Leu Pro Cys Val Glu Asp Tyr Leu Ser Ala Ile | |
| 465 470 475 480 | |
| Leu Asn Arg Val Cys Leu Leu His Glu Lys Thr Pro Val Ser Glu His | |
| 485 490 495 | |
| Val Thr Lys Cys Cys Ser Gly Ser Leu Val Glu Arg Arg Pro Cys Phe | |
| 500 505 510 | |
| Ser Ala Leu Thr Val Asp Glu Thr Tyr Val Pro Lys Glu Phe Lys Ala | |
| 515 520 525 | |
| Glu Thr Phe Thr Phe His Ser Asp Ile Cys Thr Leu Pro Glu Lys Glu | |
| 530 535 540 | |
| Lys Gln Ile Lys Lys Gln Thr Ala Leu Ala Glu Leu Val Lys His Lys | |
| 545 550 555 560 | |
| Pro Lys Ala Thr Ala Glu Gln Leu Lys Thr Val Met Asp Asp Phe Ala | |
| 565 570 575 | |
| Gln Phe Leu Asp Thr Cys Cys Lys Ala Ala Asp Lys Asp Thr Cys Phe | |
| 580 585 590 | |
| Ser Thr Glu Gly Pro Asn Leu Val Thr Arg Cys Lys Asp Ala Leu Ala | |
| 595 600 605 | |
| Arg Ser Trp Ser His Pro Gln Phe Glu Lys | |
| 610 615 | |
| ENPP2 Signal Peptide | |
| SEQ. ID NO: 36 | |
| Leu Phe Thr Phe Ala Val Gly Val Asn Ile Cys Leu Gly | |
| 1 5 10 15 | |
| Phe Thr Ala | |
| Signal Sequence ENPP7 | |
| SEQ. ID NO: 37 | |
| Met Arg Gly Pro Ala Val Leu Leu Thr Val Ala Leu Ala Thr Leu Leu | |
| 1 5 10 15 | |
| Ala Pro Gly Ala | |
| 20 | |
| Signal sequence ENPP7 | |
| SEQ. ID NO: 38 | |
| Met Arg Gly Pro Ala Val Leu Leu Thr Val Ala Leu Ala Thr Leu Leu | |
| 1 5 10 15 | |
| Ala Pro Gly Ala Gly Ala | |
| 20 | |
| Signal Sequence ENPP1-2-1 | |
| SEQ. ID NO: 39 | |
| Met Glu Arg Asp Gly Cys Ala Gly Gly Gly Ser Arg Gly Gly Glu Gly | |
| 1 5 10 15 | |
| Gly Arg Ala Pro Arg Glu Gly Pro Ala Gly Asn Gly Arg Asp Arg Gly | |
| 20 25 30 | |
| Arg Ser His Ala Ala Glu Ala Pro Gly Asp Pro Gln Ala Ala Ala Ser | |
| 35 40 45 | |
| Leu Leu Ala Pro Met Asp Val Gly Glu Glu Pro Leu Glu Lys Ala Ala | |
| 50 55 60 | |
| Arg Ala Arg Thr Ala Lys Asp Pro Asn Thr Tyr Lys Ile Ile Ser Leu | |
| 65 70 75 80 | |
| Phe Thr Phe Ala Val Gly Val Asn Ile Cys Leu Gly Phe Thr Ala | |
| 85 90 95 | |
| exENPP3 | |
| SEQ. ID NO: 40 | |
| Leu Leu Val Ile Met Ser Leu Gly Leu Gly Leu Gly Leu Gly Leu Arg | |
| 1 5 10 15 | |
| Lys | |
| Signal Sequence ENPP5: | |
| SEQ. ID NO: 41 | |
| Met Thr Ser Lys Phe Leu Leu Val Ser Phe Ile Leu Ala Ala Leu Ser | |
| 1 5 10 15 | |
| Leu Ser Thr Thr Phe Ser | |
| 20 | |
| Azurocidin-ENPP1-FC Nucleotide sequence | |
| SEQ ID NO: 42 | |
| ggtaccgccaccatgacaagactgacagtgctggctctgctggccggactgttggcctcttctagagctg | |
| ctccttcctgcgccaaagaagtgaagtcctgcaagggcagatgcttcgagcggaccttcggcaactgtag | |
| atgtgacgccgcttgcgtggaactgggcaactgctgcctggactaccaagagacatgcatcgagcccgag | |
| cacatctggacctgcaacaagttcagatgcggcgagaagcggctgaccagatctctgtgcgcctgctctg | |
| acgactgcaaggacaagggcgactgctgcatcaactactcctctgtgtgccagggcgagaagtcctgggt | |
| tgaagaaccctgcgagtccatcaacgagcctcagtgtcctgccggcttcgagacacctcctactctgctg | |
| ttctccctggatggcttcagagccgagtacctgcatacttggggaggcctgctgccagtgatctccaagc | |
| tgaagaagtgcggcacctacaccaagaacatgaggcctgtgtaccctaccaagacattccccaaccacta | |
| ctccatcgtgaccggcctgtatcctgagagccacggcatcatcgacaacaagatgtacgaccccaagatg | |
| aacgcctccttcagcctgaagtccaaagagaagttcaaccccgagtggtataagggcgagcctatctggg | |
| tcaccgctaagtaccagggactgaagtctggcaccttcttttggcctggctccgacgtggaaatcaacgg | |
| catcttccccgacatctataagatgtacaacggctccgtgcctttcgaggaacgcattctggctgttctg | |
| cagtggctgcagctgcctaaggatgagaggcctcacttctacaccctgtacctggaagaacctgactcct | |
| ccggccactcttatggccctgtgtcctctgaagtgatcaaggccctgcagcgagtggacggaatggtcgg | |
| aatgctgatggacggcctgaaagagctgaacctgcacagatgcctgaacctgatcctgatctccgaccac | |
| ggcatggaacaggggagctgcaagaagtacatctacctgaacaagtacctgggcgacgtgaagaacatca | |
| aagtgatctacggcccagccgccagactgaggccttctgatgtgcctgacaagtactactccttcaacta | |
| cgagggaatcgcccggaacctgtcctgcagagagcctaaccagcacttcaagccctacctgaagcacttt | |
| ctgcctaagcggctgcacttcgccaagtctgacagaatcgagcccctgaccttctatctggaccctcagt | |
| ggcagctggccctgaatcctagcgagagaaagtactgtggctccggcttccacggctccgacaacgtgtt | |
| ctctaatatgcaggccctgttcgtcggctacggccctggctttaaacacggcatcgaggccgacaccttc | |
| gagaacatcgaggtgtacaatctgatgtgtgacctgctgaatctgacccctgctcctaacaacggcaccc | |
| acggatctctgaaccatctgctgaagaatcccgtgtacacccctaagcaccccaaagaggttcaccctct | |
| ggtccagtgtcctttcaccagaaatcctcgggacaacctgggctgctcttgcaacccttctatcctgcct | |
| atcgaggactttcagacccagttcaacctgaccgtggccgaggaaaagatcatcaagcacgagacactgc | |
| cctacggcagacctagagtgctgcagaaagagaacaccatctgcctgctgtcccagcaccagttcatgtc | |
| cggctactcccaggacatcctgatgcctctgtggacctcctacaccgtggaccggaacgatagcttctcc | |
| accgaggacttcagcaactgcctgtaccaggatttcagaatccctctgagccccgtgcacaagtgcagct | |
| tctacaagaacaacaccaaggtgtcctacggcttcctgtctcctccacagctgaacaagaactccagcgg | |
| catctactctgaggccctgctgaccaccaacatcgtgcccatgtaccagtccttccaagtgatctggcgg | |
| tacttccacgacaccctgctgaggaagtacgccgaagaaagaaacggcgtgaacgtggtgtctggccccg | |
| tgttcgacttcgactacgacggcagatgcgactctctggaaaacctgcggcagaaaagacgagtgatccg | |
| gaatcaagagatcctgattcctacacacttctttatcgtgctgaccagctgcaaggatacctctcagacc | |
| cctctgcactgcgagaatctggacaccctggccttcattctgcctcacagaaccgacaactccgagtcct | |
| gtgtgcacggcaagcacgactcctcttgggtcgaagaactgctgatgctgcaccgggccagaatcaccga | |
| tgtggaacacatcaccggcctgagcttctaccagcagcggaaagaacctgtgtccgatatcctgaagctg | |
| aaaacccatctgccaaccttcagccaagaggacctgatcaacgacaagacccacacctgtcctccatgtc | |
| ctgctccagaactgctcggaggcccctctgtgttcctgtttccacctaagccaaaggacacactgatgat | |
| ctctcggacccctgaagtgacctgcgtggtggtggatgtgtctcacgaagatcccgaagtcaagttcaat | |
| tggtacgtggacggcgtggaagtgcacaacgccaagaccaagcctagagaggaacagtacaactccacct | |
| acagagtggtgtccgtgctgactgtgctgcaccaggattggctgaacggcaaagagtacaagtgcaaagt | |
| gtccaacaaggctctgcccgctcctatcgaaaagaccatctccaaggctaagggccagcctcgggaacct | |
| caggtttacaccctgcctccatctcgggaagagatgaccaagaaccaggtgtccctgacctgcctggtca | |
| agggcttctacccttccgatatcgccgtggaatgggagtccaatggccagcctgagaacaactacaagac | |
| aacccctcctgtgctggacagcgacggctcattcttcctgtactctaagctgacagtggacaagtcccgg | |
| tggcagcaaggcaatgtgttttcctgctctgtgatgcacgaggccctccacaatcactacacccagaagt | |
| ccctgtctctgtcccctggcaaatgatagctcgag | |
| Legend: blue = restriction site; bold = start/stop codon; green = | |
| Kozak sequence; underlined =nucleotide sequence of signal peptide. | |
| Azurocidin-ENPP1-Albumin Nucleotide sequence | |
| SEQ ID NO: 43 | |
| atgacaagactgacagtgctggctctgctggccggactgttggcctcttctagagctgctccttcctgcg | |
| ccaaagaagtgaagtcctgcaagggcagatgcttcgagcggaccttcggcaactgtagatgtgacgccgc | |
| ttgcgtggaactgggcaactgctgcctggactaccaagagacatgcatcgagcccgagcacatctggacc | |
| tgcaacaagttcagatgcggcgagaagcggctgaccagatctctgtgcgcctgctctgacgactgcaagg | |
| acaagggcgactgctgcatcaactactcctctgtgtgccagggcgagaagtcctgggttgaagaaccctg | |
| cgagtccatcaacgagcctcagtgtcctgccggcttcgagacacctcctactctgctgttctccctggat | |
| ggcttcagagccgagtacctgcatacttggggaggcctgctgccagtgatctccaagctgaagaagtgcg | |
| gcacctacaccaagaacatgaggcctgtgtaccctaccaagacattccccaaccactactccatcgtgac | |
| cggcctgtatcctgagagccacggcatcatcgacaacaagatgtacgaccccaagatgaacgcctccttc | |
| agcctgaagtccaaagagaagttcaaccccgagtggtataagggcgagcctatctgggtcaccgctaagt | |
| accagggactgaagtctggcaccttcttttggcctggctccgacgtggaaatcaacggcatcttccccga | |
| catctataagatgtacaacggctccgtgcctttcgaggaacgcattctggctgttctgcagtggctgcag | |
| ctgcctaaggatgagaggcctcacttctacaccctgtacctggaagaacctgactcctccggccactctt | |
| atggccctgtgtcctctgaagtgatcaaggccctgcagcgagtggacggaatggtcggaatgctgatgga | |
| cggcctgaaagagctgaacctgcacagatgcctgaacctgatcctgatctccgaccacggcatggaacag | |
| gggagctgcaagaagtacatctacctgaacaagtacctgggcgacgtgaagaacatcaaagtgatctacg | |
| gcccagccgccagactgaggccttctgatgtgcctgacaagtactactccttcaactacgagggaatcgc | |
| ccggaacctgtcctgcagagagcctaaccagcacttcaagccctacctgaagcactttctgcctaagcgg | |
| ctgcacttcgccaagtctgacagaatcgagcccctgaccttctatctggaccctcagtggcagctggccc | |
| tgaatcctagcgagagaaagtactgtggctccggcttccacggctccgacaacgtgttctctaatatgca | |
| ggccctgttcgtcggctacggccctggctttaaacacggcatcgaggccgacaccttcgagaacatcgag | |
| gtgtacaatctgatgtgtgacctgctgaatctgacccctgctcctaacaacggcacccacggatctctga | |
| accatctgctgaagaatcccgtgtacacccctaagcaccccaaagaggttcaccctctggtccagtgtcc | |
| tttcaccagaaatcctcgggacaacctgggctgctcttgcaacccttctatcctgcctatcgaggacttt | |
| cagacccagttcaacctgaccgtggccgaggaaaagatcatcaagcacgagacactgccctacggcagac | |
| ctagagtgctgcagaaagagaacaccatctgcctgctgtcccagcaccagttcatgtccggctactccca | |
| ggacatcctgatgcctctgtggacctcctacaccgtggaccggaacgatagcttctccaccgaggacttc | |
| agcaactgcctgtaccaggatttcagaatccctctgagccccgtgcacaagtgcagcttctacaagaaca | |
| acaccaaggtgtcctacggcttcctgtctcctccacagctgaacaagaactccagcggcatctactctga | |
| ggccctgctgaccaccaacatcgtgcccatgtaccagtccttccaagtgatctggcggtacttccacgac | |
| accctgctgaggaagtacgccgaagaaagaaacggcgtgaacgtggtgtctggccccgtgttcgacttcg | |
| actacgacggcagatgcgactctctggaaaacctgcggcagaaaagacgagtgatccggaatcaagagat | |
| cctgattcctacacacttctttatcgtgctgaccagctgcaaggatacctctcagacccctctgcactgc | |
| gagaatctggacaccctggccttcattctgcctcacagaaccgacaactccgagtcctgtgtgcacggca | |
| agcacgactcctcttgggtcgaagaactgctgatgctgcaccgggccagaatcaccgatgtggaacacat | |
| caccggcctgagcttctaccagcagcggaaagaacctgtgtccgatatcctgaagctgaaaacccatctg | |
| ccaaccttcagccaagaggacctgatcaacatgaagtgggtgaccttcctgctgctgctgttcgtgagcg | |
| gcagcgccttcagcagaggcgtgttcagaagagaggcccacaagagcgagatcgcccacagatacaacga | |
| cctgggcgagcagcacttcaagggcctggtgctgatcgccttcagccagtacctgcagaagtgcagctac | |
| gacgagcacgccaagctggtgcaggaggtgaccgacttcgccaagacctgcgtggccgacgagagcgccg | |
| ccaactgcgacaagagcctgcacaccctgttcggcgacaagctgtgcgccatccccaacctgagagagaa | |
| ctacggcgagctggccgactgctgcaccaagcaggagcccgagagaaacgagtgcttcctgcagcacaag | |
| gacgacaaccccagcctgccccccttcgagagacccgaggccgaggccatgtgcaccagcttcaaggaga | |
| accccaccaccttcatgggccactacctgcacgaggtggccagaagacacccctacttctacgcccccga | |
| gctgctgtactacgccgagcagtacaacgagatcctgacccagtgctgcgccgaggccgacaaggagagc | |
| tgcctgacccccaagctggacggcgtgaaggagaaggccctggtgagcagcgtgagacagagaatgaagt | |
| gcagcagcatgcagaagttcggcgagagagccttcaaggcctgggccgtggccagactgagccagacctt | |
| ccccaacgccgacttcgccgagatcaccaagctggccaccgacctgaccaaggtgaacaaggagtgctgc | |
| cacggcgacctgctggagtgcgccgacgacagagccgagctggccaagtacatgtgcgagaaccaggcca | |
| ccatcagcagcaagctgcagacctgctgcgacaagcccctgctgaagaaggcccactgcctgagcgaggt | |
| ggagcacgacaccatgcccgccgacctgcccgccatcgccgccgacttcgtggaggaccaggaggtgtgc | |
| aagaactacgccgaggccaaggacgtgttcctgggcaccttcctgtacgagtacagcagaagacaccccg | |
| actacagcgtgagcctgctgctgagactggccaagaagtacgaggccaccctggagaagtgctgcgccga | |
| ggccaacccccccgcctgctacggcaccgtgctggccgagttccagcccctggtggaggagcccaagaac | |
| ctggtgaagaccaactgcgacctgtacgagaagctgggcgagtacggcttccagaacgccatcctggtga | |
| gatacacccagaaggccccccaggtgagcacccccaccctggtggaggccgccagaaacctgggcagagt | |
| gggcaccaagtgctgcaccctgcccgaggaccagagactgccctgcgtggaggactacctgagcgccatc | |
| ctgaacagagtgtgcctgctgcacgagaagacccccgtgagcgagcacgtgaccaagtgctgcagcggca | |
| gcctggtggagagaagaccctgcttcagcgccctgaccgtggacgagacctacgtgcccaaggagttcaa | |
| ggccgagaccttcaccttccacagcgacatctgcaccctgcccgagaaggagaagcagatcaagaagcag | |
| accgccctggccgagctggtgaagcacaagcccaaggccaccgccgagcagctgaagaccgtgatggacg | |
| acttcgcccagttcctggacacctgctgcaaggccgccgacaaggacacctgcttcagcaccgagggccc | |
| caacctggtgaccagatgcaaggacgccctggccagaagctggagccacccccagttcgagaag | |
| Azurocidin-ENPP1 Nucleotide sequence | |
| SEQ ID NO: 44 | |
| atgacaagactgacagtgctggctctgctggccggactgttggcctcttctagagctgctccttcctgcg | |
| ccaaagaagtgaagtcctgcaagggcagatgcttcgagcggaccttcggcaactgtagatgtgacgccgc | |
| ttgcgtggaactgggcaactgctgcctggactaccaagagacatgcatcgagcccgagcacatctggacc | |
| tgcaacaagttcagatgcggcgagaagcggctgaccagatctctgtgcgcctgctctgacgactgcaagg | |
| acaagggcgactgctgcatcaactactcctctgtgtgccagggcgagaagtcctgggttgaagaaccctg | |
| cgagtccatcaacgagcctcagtgtcctgccggcttcgagacacctcctactctgctgttctccctggat | |
| ggcttcagagccgagtacctgcatacttggggaggcctgctgccagtgatctccaagctgaagaagtgcg | |
| gcacctacaccaagaacatgaggcctgtgtaccctaccaagacattccccaaccactactccatcgtgac | |
| cggcctgtatcctgagagccacggcatcatcgacaacaagatgtacgaccccaagatgaacgcctccttc | |
| agcctgaagtccaaagagaagttcaaccccgagtggtataagggcgagcctatctgggtcaccgctaagt | |
| accagggactgaagtctggcaccttcttttggcctggctccgacgtggaaatcaacggcatcttccccga | |
| catctataagatgtacaacggctccgtgcctttcgaggaacgcattctggctgttctgcagtggctgcag | |
| ctgcctaaggatgagaggcctcacttctacaccctgtacctggaagaacctgactcctccggccactctt | |
| atggccctgtgtcctctgaagtgatcaaggccctgcagcgagtggacggaatggtcggaatgctgatgga | |
| cggcctgaaagagctgaacctgcacagatgcctgaacctgatcctgatctccgaccacggcatggaacag | |
| gggagctgcaagaagtacatctacctgaacaagtacctgggcgacgtgaagaacatcaaagtgatctacg | |
| gcccagccgccagactgaggccttctgatgtgcctgacaagtactactccttcaactacgagggaatcgc | |
| ccggaacctgtcctgcagagagcctaaccagcacttcaagccctacctgaagcactttctgcctaagcgg | |
| ctgcacttcgccaagtctgacagaatcgagcccctgaccttctatctggaccctcagtggcagctggccc | |
| tgaatcctagcgagagaaagtactgtggctccggcttccacggctccgacaacgtgttctctaatatgca | |
| ggccctgttcgtcggctacggccctggctttaaacacggcatcgaggccgacaccttcgagaacatcgag | |
| gtgtacaatctgatgtgtgacctgctgaatctgacccctgctcctaacaacggcacccacggatctctga | |
| accatctgctgaagaatcccgtgtacacccctaagcaccccaaagaggttcaccctctggtccagtgtcc | |
| tttcaccagaaatcctcgggacaacctgggctgctcttgcaacccttctatcctgcctatcgaggacttt | |
| cagacccagttcaacctgaccgtggccgaggaaaagatcatcaagcacgagacactgccctacggcagac | |
| ctagagtgctgcagaaagagaacaccatctgcctgctgtcccagcaccagttcatgtccggctactccca | |
| ggacatcctgatgcctctgtggacctcctacaccgtggaccggaacgatagcttctccaccgaggacttc | |
| agcaactgcctgtaccaggatttcagaatccctctgagccccgtgcacaagtgcagcttctacaagaaca | |
| acaccaaggtgtcctacggcttcctgtctcctccacagctgaacaagaactccagcggcatctactctga | |
| ggccctgctgaccaccaacatcgtgcccatgtaccagtccttccaagtgatctggcggtacttccacgac | |
| accctgctgaggaagtacgccgaagaaagaaacggcgtgaacgtggtgtctggccccgtgttcgacttcg | |
| actacgacggcagatgcgactctctggaaaacctgcggcagaaaagacgagtgatccggaatcaagagat | |
| cctgattcctacacacttctttatcgtgctgaccagctgcaaggatacctctcagacccctctgcactgc | |
| gagaatctggacaccctggccttcattctgcctcacagaaccgacaactccgagtcctgtgtgcacggca | |
| agcacgactcctcttgggtcgaagaactgctgatgctgcaccgggccagaatcaccgatgtggaacacat | |
| caccggcctgagcttctaccagcagcggaaagaacctgtgtccgatatcctgaagctgaaaacccatctg | |
| ccaaccttcagccaagaggac | |
| Azurocidin-ENPP3-FC Nucleotide sequence | |
| SEQ ID NO: 45 | |
| atgaccagactgaccgtgctggccctgctggccggcctgctggccagcagcagagccgccaagcagggca | |
| gctgcagaaagaagtgcttcgacgccagcttcagaggcctggagaactgcagatgcgacgtggcctgcaa | |
| ggacagaggcgactgctgctgggacttcgaggacacctgcgtggagagcaccagaatctggatgtgcaac | |
| aagttcagatgcggcgagaccagactggaggccagcctgtgcagctgcagcgacgactgcctgcagagaa | |
| aggactgctgcgccgactacaagagcgtgtgccagggcgagaccagctggctggaggagaactgcgacac | |
| cgcccagcagagccagtgccccgagggcttcgacctgccccccgtgatcctgttcagcatggacggcttc | |
| agagccgagtacctgtacacctgggacaccctgatgcccaacatcaacaagctgaagacctgcggcatcc | |
| acagcaagtacatgagagccatgtaccccaccaagaccttccccaaccactacaccatcgtgaccggcct | |
| gtaccccgagagccacggcatcatcgacaacaacatgtacgacgtgaacctgaacaagaacttcagcctg | |
| agcagcaaggagcagaacaaccccgcctggtggcacggccagcccatgaacctgaccgccatgtaccagg | |
| gcctgaaggccgccacctacttctggcccggcagcgaggtggccatcaacggcagcttccccagcatcta | |
| catgccctacaacggcagcgtgcccttcgaggagagaatcagcaccctgctgaagtggctggacctgccc | |
| aaggccgagagacccagattctacaccatgtacttcgaggagcccgacagcagcggccacgccggcggcc | |
| ccgtgagcgccagagtgatcaaggccctgcaggtggtggaccacgccttcggcatgctgatggagggcct | |
| gaagcagagaaacctgcacaactgcgtgaacatcatcctgctggccgaccacggcatggaccagacctac | |
| tgcaacaagatggagtacatgaccgactacttccccagaatcaacttcttctacatgtacgagggccccg | |
| cccccagaatcagagcccacaacatcccccacgacttcttcagcttcaacagcgaggagatcgtgagaaa | |
| cctgagctgcagaaagcccgaccagcacttcaagccctacctgacccccgacctgcccaagagactgcac | |
| tacgccaagaacgtgagaatcgacaaggtgcacctgttcgtggaccagcagtggctggccgtgagaagca | |
| agagcaacaccaactgcggcggcggcaaccacggctacaacaacgagttcagaagcatggaggccatctt | |
| cctggcccacggccccagcttcaaggagaagaccgaggtggagcccttcgagaacatcgaggtgtacaac | |
| ctgatgtgcgacctgctgagaatccagcccgcccccaacaacggcacccacggcagcctgaaccacctgc | |
| tgaaggtgcccttctacgagcccagccacgccgaggaggtgagcaagttcagcgtgtgcggcttcgccaa | |
| ccccctgcccaccgagagcctggactgcttctgcccccacctgcagaacagcacccagctggagcaggtg | |
| aaccagatgctgaacctgacccaggaggagatcaccgccaccgtgaaggtgaacctgcccttcggcagac | |
| ccagagtgctgcagaagaacgtggaccactgcctgctgtaccacagagagtacgtgagcggcttcggcaa | |
| ggccatgagaatgcccatgtggagcagctacaccgtgccccagctgggcgacaccagccccctgcccccc | |
| accgtgcccgactgcctgagagccgacgtgagagtgccccccagcgagagccagaagtgcagcttctacc | |
| tggccgacaagaacatcacccacggcttcctgtacccccccgccagcaacagaaccagcgacagccagta | |
| cgacgccctgatcaccagcaacctggtgcccatgtacgaggagttcagaaagatgtgggactacttccac | |
| agcgtgctgctgatcaagcacgccaccgagagaaacggcgtgaacgtggtgagcggccccatcttcgact | |
| acaactacgacggccacttcgacgcccccgacgagatcaccaagcacctggccaacaccgacgtgcccat | |
| ccccacccactacttcgtggtgctgaccagctgcaagaacaagagccacacccccgagaactgccccggc | |
| tggctggacgtgctgcccttcatcatcccccacagacccaccaacgtggagagctgccccgagggcaagc | |
| ccgaggccctgtgggtggaggagagattcaccgcccacatcgccagagtgagagacgtggagctgctgac | |
| cggcctggacttctaccaggacaaggtgcagcccgtgagcgagatcctgcagctgaagacctacctgccc | |
| accttcgagaccaccatcgacaagacccacacctgccccccctgccccgcccccgagctgctgggcggcc | |
| ccagcgtgttcctgttcccccccaagcccaaggacaccctgatgatcagcagaacccccgaggtgacctg | |
| cgtggtggtggacgtgagccacgaggaccccgaggtgaagttcaactggtacgtggacggcgtggaggtg | |
| cacaacgccaagaccaagcccagagaggagcagtacaacagcacctacagagtggtgagcgtgctgaccg | |
| tgctgcaccaggactggctgaacggcaaggagtacaagtgcaaggtgagcaacaaggccctgcccgcccc | |
| catcgagaagaccatcagcaaggccaagggccagcccagagagccccaggtgtacaccctgccccccagc | |
| agagaggagatgaccaagaaccaggtgagcctgacctgcctggtgaagggcttctaccccagcgacatcg | |
| ccgtggagtgggagagcaacggccagcccgagaacaactacaagaccaccccccccgtgctggacagcga | |
| cggcagcttcttcctgtacagcaagctgaccgtggacaagagcagatggcagcagggcaacgtgttcagc | |
| tgcagcgtgatgcacgaggccctgcacaaccactacacccagaagagcctgagcctgagccccggcaag | |
| Azurocidin-ENPP3-Albumin Nucleotide sequence | |
| SEQ ID NO: 46 | |
| atgaccagactgaccgtgctggccctgctggccggcctgctggccagcagcagagccgccaagcagggca | |
| gctgcagaaagaagtgcttcgacgccagcttcagaggcctggagaactgcagatgcgacgtggcctgcaa | |
| ggacagaggcgactgctgctgggacttcgaggacacctgcgtggagagcaccagaatctggatgtgcaac | |
| aagttcagatgcggcgagaccagactggaggccagcctgtgcagctgcagcgacgactgcctgcagagaa | |
| aggactgctgcgccgactacaagagcgtgtgccagggcgagaccagctggctggaggagaactgcgacac | |
| cgcccagcagagccagtgccccgagggcttcgacctgccccccgtgatcctgttcagcatggacggcttc | |
| agagccgagtacctgtacacctgggacaccctgatgcccaacatcaacaagctgaagacctgcggcatcc | |
| acagcaagtacatgagagccatgtaccccaccaagaccttccccaaccactacaccatcgtgaccggcct | |
| gtaccccgagagccacggcatcatcgacaacaacatgtacgacgtgaacctgaacaagaacttcagcctg | |
| agcagcaaggagcagaacaaccccgcctggtggcacggccagcccatgaacctgaccgccatgtaccagg | |
| gcctgaaggccgccacctacttctggcccggcagcgaggtggccatcaacggcagcttccccagcatcta | |
| catgccctacaacggcagcgtgcccttcgaggagagaatcagcaccctgctgaagtggctggacctgccc | |
| aaggccgagagacccagattctacaccatgtacttcgaggagcccgacagcagcggccacgccggcggcc | |
| ccgtgagcgccagagtgatcaaggccctgcaggtggtggaccacgccttcggcatgctgatggagggcct | |
| gaagcagagaaacctgcacaactgcgtgaacatcatcctgctggccgaccacggcatggaccagacctac | |
| tgcaacaagatggagtacatgaccgactacttccccagaatcaacttcttctacatgtacgagggccccg | |
| cccccagaatcagagcccacaacatcccccacgacttcttcagcttcaacagcgaggagatcgtgagaaa | |
| cctgagctgcagaaagcccgaccagcacttcaagccctacctgacccccgacctgcccaagagactgcac | |
| tacgccaagaacgtgagaatcgacaaggtgcacctgttcgtggaccagcagtggctggccgtgagaagca | |
| agagcaacaccaactgcggcggcggcaaccacggctacaacaacgagttcagaagcatggaggccatctt | |
| cctggcccacggccccagcttcaaggagaagaccgaggtggagcccttcgagaacatcgaggtgtacaac | |
| ctgatgtgcgacctgctgagaatccagcccgcccccaacaacggcacccacggcagcctgaaccacctgc | |
| tgaaggtgcccttctacgagcccagccacgccgaggaggtgagcaagttcagcgtgtgcggcttcgccaa | |
| ccccctgcccaccgagagcctggactgcttctgcccccacctgcagaacagcacccagctggagcaggtg | |
| aaccagatgctgaacctgacccaggaggagatcaccgccaccgtgaaggtgaacctgcccttcggcagac | |
| ccagagtgctgcagaagaacgtggaccactgcctgctgtaccacagagagtacgtgagcggcttcggcaa | |
| ggccatgagaatgcccatgtggagcagctacaccgtgccccagctgggcgacaccagccccctgcccccc | |
| accgtgcccgactgcctgagagccgacgtgagagtgccccccagcgagagccagaagtgcagcttctacc | |
| tggccgacaagaacatcacccacggcttcctgtacccccccgccagcaacagaaccagcgacagccagta | |
| cgacgccctgatcaccagcaacctggtgcccatgtacgaggagttcagaaagatgtgggactacttccac | |
| agcgtgctgctgatcaagcacgccaccgagagaaacggcgtgaacgtggtgagcggccccatcttcgact | |
| acaactacgacggccacttcgacgcccccgacgagatcaccaagcacctggccaacaccgacgtgcccat | |
| ccccacccactacttcgtggtgctgaccagctgcaagaacaagagccacacccccgagaactgccccggc | |
| tggctggacgtgctgcccttcatcatcccccacagacccaccaacgtggagagctgccccgagggcaagc | |
| ccgaggccctgtgggtggaggagagattcaccgcccacatcgccagagtgagagacgtggagctgctgac | |
| cggcctggacttctaccaggacaaggtgcagcccgtgagcgagatcctgcagctgaagacctacctgccc | |
| accttcgagaccaccatcatgaagtgggtgaccttcctgctgctgctgttcgtgagcggcagcgccttca | |
| gcagaggcgtgttcagaagagaggcccacaagagcgagatcgcccacagatacaacgacctgggcgagca | |
| gcacttcaagggcctggtgctgatcgccttcagccagtacctgcagaagtgcagctacgacgagcacgcc | |
| aagctggtgcaggaggtgaccgacttcgccaagacctgcgtggccgacgagagcgccgccaactgcgaca | |
| agagcctgcacaccctgttcggcgacaagctgtgcgccatccccaacctgagagagaactacggcgagct | |
| ggccgactgctgcaccaagcaggagcccgagagaaacgagtgcttcctgcagcacaaggacgacaacccc | |
| agcctgccccccttcgagagacccgaggccgaggccatgtgcaccagcttcaaggagaaccccaccacct | |
| tcatgggccactacctgcacgaggtggccagaagacacccctacttctacgcccccgagctgctgtacta | |
| cgccgagcagtacaacgagatcctgacccagtgctgcgccgaggccgacaaggagagctgcctgaccccc | |
| aagctggacggcgtgaaggagaaggccctggtgagcagcgtgagacagagaatgaagtgcagcagcatgc | |
| agaagttcggcgagagagccttcaaggcctgggccgtggccagactgagccagaccttccccaacgccga | |
| cttcgccgagatcaccaagctggccaccgacctgaccaaggtgaacaaggagtgctgccacggcgacctg | |
| ctggagtgcgccgacgacagagccgagctggccaagtacatgtgcgagaaccaggccaccatcagcagca | |
| agctgcagacctgctgcgacaagcccctgctgaagaaggcccactgcctgagcgaggtggagcacgacac | |
| catgcccgccgacctgcccgccatcgccgccgacttcgtggaggaccaggaggtgtgcaagaactacgcc | |
| gaggccaaggacgtgttcctgggcaccttcctgtacgagtacagcagaagacaccccgactacagcgtga | |
| gcctgctgctgagactggccaagaagtacgaggccaccctggagaagtgctgcgccgaggccaacccccc | |
| cgcctgctacggcaccgtgctggccgagttccagcccctggtggaggagcccaagaacctggtgaagacc | |
| aactgcgacctgtacgagaagctgggcgagtacggcttccagaacgccatcctggtgagatacacccaga | |
| aggccccccaggtgagcacccccaccctggtggaggccgccagaaacctgggcagagtgggcaccaagtg | |
| ctgcaccctgcccgaggaccagagactgccctgcgtggaggactacctgagcgccatcctgaacagagtg | |
| tgcctgctgcacgagaagacccccgtgagcgagcacgtgaccaagtgctgcagcggcagcctggtggaga | |
| gaagaccctgcttcagcgccctgaccgtggacgagacctacgtgcccaaggagttcaaggccgagacctt | |
| caccttccacagcgacatctgcaccctgcccgagaaggagaagcagatcaagaagcagaccgccctggcc | |
| gagctggtgaagcacaagcccaaggccaccgccgagcagctgaagaccgtgatggacgacttcgcccagt | |
| tcctggacacctgctgcaaggccgccgacaaggacacctgcttcagcaccgagggccccaacctggtgac | |
| cagatgcaaggacgccctggccagaagctggagccacccccagttcgagaag | |
| Azurocidin-ENPP3-Nucleotide sequence | |
| SEQ ID NO: 47 | |
| atgaccagactgaccgtgctggccctgctggccggcctgctggccagcagcagagccgccaagcagggca | |
| gctgcagaaagaagtgcttcgacgccagcttcagaggcctggagaactgcagatgcgacgtggcctgcaa | |
| ggacagaggcgactgctgctgggacttcgaggacacctgcgtggagagcaccagaatctggatgtgcaac | |
| aagttcagatgcggcgagaccagactggaggccagcctgtgcagctgcagcgacgactgcctgcagagaa | |
| aggactgctgcgccgactacaagagcgtgtgccagggcgagaccagctggctggaggagaactgcgacac | |
| cgcccagcagagccagtgccccgagggcttcgacctgccccccgtgatcctgttcagcatggacggcttc | |
| agagccgagtacctgtacacctgggacaccctgatgcccaacatcaacaagctgaagacctgcggcatcc | |
| acagcaagtacatgagagccatgtaccccaccaagaccttccccaaccactacaccatcgtgaccggcct | |
| gtaccccgagagccacggcatcatcgacaacaacatgtacgacgtgaacctgaacaagaacttcagcctg | |
| agcagcaaggagcagaacaaccccgcctggtggcacggccagcccatgaacctgaccgccatgtaccagg | |
| gcctgaaggccgccacctacttctggcccggcagcgaggtggccatcaacggcagcttccccagcatcta | |
| catgccctacaacggcagcgtgcccttcgaggagagaatcagcaccctgctgaagtggctggacctgccc | |
| aaggccgagagacccagattctacaccatgtacttcgaggagcccgacagcagcggccacgccggcggcc | |
| ccgtgagcgccagagtgatcaaggccctgcaggtggtggaccacgccttcggcatgctgatggagggcct | |
| gaagcagagaaacctgcacaactgcgtgaacatcatcctgctggccgaccacggcatggaccagacctac | |
| tgcaacaagatggagtacatgaccgactacttccccagaatcaacttcttctacatgtacgagggccccg | |
| cccccagaatcagagcccacaacatcccccacgacttcttcagcttcaacagcgaggagatcgtgagaaa | |
| cctgagctgcagaaagcccgaccagcacttcaagccctacctgacccccgacctgcccaagagactgcac | |
| tacgccaagaacgtgagaatcgacaaggtgcacctgttcgtggaccagcagtggctggccgtgagaagca | |
| agagcaacaccaactgcggcggcggcaaccacggctacaacaacgagttcagaagcatggaggccatctt | |
| cctggcccacggccccagcttcaaggagaagaccgaggtggagcccttcgagaacatcgaggtgtacaac | |
| ctgatgtgcgacctgctgagaatccagcccgcccccaacaacggcacccacggcagcctgaaccacctgc | |
| tgaaggtgcccttctacgagcccagccacgccgaggaggtgagcaagttcagcgtgtgcggcttcgccaa | |
| ccccctgcccaccgagagcctggactgcttctgcccccacctgcagaacagcacccagctggagcaggtg | |
| aaccagatgctgaacctgacccaggaggagatcaccgccaccgtgaaggtgaacctgcccttcggcagac | |
| ccagagtgctgcagaagaacgtggaccactgcctgctgtaccacagagagtacgtgagcggcttcggcaa | |
| ggccatgagaatgcccatgtggagcagctacaccgtgccccagctgggcgacaccagccccctgcccccc | |
| accgtgcccgactgcctgagagccgacgtgagagtgccccccagcgagagccagaagtgcagcttctacc | |
| tggccgacaagaacatcacccacggcttcctgtacccccccgccagcaacagaaccagcgacagccagta | |
| cgacgccctgatcaccagcaacctggtgcccatgtacgaggagttcagaaagatgtgggactacttccac | |
| agcgtgctgctgatcaagcacgccaccgagagaaacggcgtgaacgtggtgagcggccccatcttcgact | |
| acaactacgacggccacttcgacgcccccgacgagatcaccaagcacctggccaacaccgacgtgcccat | |
| ccccacccactacttcgtggtgctgaccagctgcaagaacaagagccacacccccgagaactgccccggc | |
| tggctggacgtgctgcccttcatcatcccccacagacccaccaacgtggagagctgccccgagggcaagc | |
| ccgaggccctgtgggtggaggagagattcaccgcccacatcgccagagtgagagacgtggagctgctgac | |
| cggcctggacttctaccaggacaaggtgcagcccgtgagcgagatcctgcagctgaagacctacctgccc | |
| accttcgagaccaccatc | |
| ENPP7-1-Fc Nucleotide sequence | |
| SEQ. ID NO: 48 | |
| atgagaggac ctgccgtcct gctgaccgtc gccctggcta ccttgctggc ccctggtgct | 60 |
| ggtgcaccca gctgcgccaa agaagtgaag tcctgcaagg gccggtgctt cgagcggacc | 120 |
| ttcggcaact gcagatgcga cgccgcctgt gtggaactgg gcaactgctg cctggactac | 180 |
| caggaaacct gcatcgagcc cgagcacatc tggacctgca acaagttcag atgcggcgag | 240 |
| aagcggctga ccagatccct gtgtgcctgc agcgacgact gcaaggacaa gggcgactgc | 300 |
| tgcatcaact acagcagcgt gtgccagggc gagaagtcct gggtggaaga accctgcgag | 360 |
| agcatcaacg agccccagtg ccctgccggc ttcgagacac ctcctaccct gctgttcagc | 420 |
| ctggacggct ttcgggccga gtacctgcac acatggggag gcctgctgcc cgtgatcagc | 480 |
| aagctgaaga agtgcggcac ctacaccaag aacatgcggc ccgtgtaccc caccaagacc | 540 |
| ttccccaacc actactccat cgtgaccggc ctgtaccccg agagccacgg catcatcgac | 600 |
| aacaagatgt acgaccccaa gatgaacgcc agcttcagcc tgaagtccaa agagaagttc | 660 |
| aaccccgagt ggtataaggg cgagcccatc tgggtcaccg ccaagtacca gggcctgaaa | 720 |
| agcggcacat tcttttggcc cggcagcgac gtggaaatca acggcatctt ccccgacatc | 780 |
| tataagatgt acaacggcag cgtgcccttc gaggaacgga tcctggctgt gctgcagtgg | 840 |
| ctgcagctgc ccaaggatga gcggccccac ttctacaccc tgtacctgga agaacctgac | 900 |
| agcagcggcc acagctacgg ccctgtgtcc agcgaagtga tcaaggccct gcagcgggtg | 960 |
| gacggcatgg tgggaatgct gatggacggc ctgaaagagc tgaacctgca cagatgcctg | 1020 |
| aacctgatcc tgatcagcga ccacggcatg gaacagggat cctgcaagaa gtacatctac | 1080 |
| ctgaacaagt acctgggcga cgtgaagaac atcaaagtga tctacggccc agccgccaga | 1140 |
| ctgaggccta gcgacgtgcc cgacaagtac tacagcttca actacgaggg aatcgcccgg | 1200 |
| aacctgagct gcagagagcc caaccagcac ttcaagccct acctgaagca cttcctgccc | 1260 |
| aagcggctgc acttcgccaa gagcgacaga atcgagcccc tgaccttcta cctggacccc | 1320 |
| cagtggcagc tggccctgaa tcccagcgag agaaagtact gcggcagcgg cttccacggc | 1380 |
| tccgacaacg tgttcagcaa catgcaggcc ctgttcgtgg gctacggacc cggctttaag | 1440 |
| cacggcatcg aggccgacac cttcgagaac atcgaggtgt acaatctgat gtgcgacctg | 1500 |
| ctgaatctga cccctgcccc caacaatggc acccacggca gcctgaacca tctgctgaag | 1560 |
| aaccccgtgt acacccctaa gcaccccaaa gaggtgcacc ccctggtgca gtgccccttc | 1620 |
| accagaaacc ccagagacaa cctgggctgt agctgcaacc ccagcatcct gcccatcgag | 1680 |
| gacttccaga cccagttcaa cctgaccgtg gccgaggaaa agatcatcaa gcacgagaca | 1740 |
| ctgccctacg gcagaccccg ggtgctgcag aaagagaaca ccatctgcct gctgagccag | 1800 |
| caccagttca tgagcggcta ctcccaggac atcctgatgc ccctgtggac cagctacacc | 1860 |
| gtggaccgga acgacagctt ctccaccgag gatttcagca actgcctgta ccaggatttc | 1920 |
| cggatccccc tgagccccgt gcacaagtgc agcttctaca agaacaacac caaggtgtcc | 1980 |
| tacggcttcc tgagccctcc ccagctgaac aagaacagct ccggcatcta cagcgaggcc | 2040 |
| ctgctgacta ccaacatcgt gcccatgtac cagagcttcc aagtgatctg gcggtacttc | 2100 |
| cacgacaccc tgctgcggaa gtacgccgaa gaacggaacg gcgtgaacgt ggtgtccggc | 2160 |
| ccagtgttcg acttcgacta cgacggcaga tgtgacagcc tggaaaatct gcggcagaaa | 2220 |
| agaagagtga tccggaacca ggaaattctg atccctaccc acttctttat cgtgctgaca | 2280 |
| agctgcaagg ataccagcca gacccccctg cactgcgaga acctggatac cctggccttc | 2340 |
| atcctgcctc accggaccga caacagcgag agctgtgtgc acggcaagca cgacagctct | 2400 |
| tgggtggaag aactgctgat gctgcaccgg gccagaatca ccgatgtgga acacatcacc | 2460 |
| ggcctgagct tttaccagca gcggaaagaa cccgtgtccg atatcctgaa gctgaaaacc | 2520 |
| catctgccca ccttcagcca ggaagatgac aagacccaca cttgcccccc ctgcccagct | 2580 |
| cctgaactgc tgggaggacc ctctgtgttc ctgttccccc caaagcccaa ggacaccctg | 2640 |
| atgatctcta ggacccccga agtcacttgc gtcgtcgtcg acgtgtccca cgaggaccct | 2700 |
| gaagtcaagt tcaactggta cgtcgacggt gtcgaagtcc acaacgccaa gaccaagccc | 2760 |
| agggaagaac agtacaactc tacctaccgc gtcgtcagcg tcctgaccgt cctgcaccag | 2820 |
| gactggctga acggaaagga atacaagtgc aaggtgtcca acaaggccct gcctgccccc | 2880 |
| atcgaaaaga ccatctctaa ggccaaggga cagccccgcg aaccccaggt ctacaccctg | 2940 |
| ccaccctcta gggaagaaat gaccaagaac caggtgtccc tgacctgcct ggtcaaggga | 3000 |
| ttctacccct ctgacatcgc cgtcgaatgg gaatctaacg gacagcccga aaacaactac | 3060 |
| aagaccaccc cccctgtcct ggactctgac ggatcattct tcctgtactc taagctgact | 3120 |
| gtcgacaagt ctaggtggca gcagggaaac gtgttctctt gctctgtcat gcacgaagcc | 3180 |
| ctgcacaacc actacaccca gaagtctctg tctctgtccc ccggaaag | 3228 |
| ENPP7-NPP1 Albumin Nucleotide sequence: | |
| SEQ. ID NO: 49 | |
| atgagaggac ctgccgtcct gctgaccgtc gccctggcta ccttgctggc ccctggtgct | 60 |
| ggtgcaccca gctgcgccaa agaagtgaag tcctgcaagg gccggtgctt cgagcggacc | 120 |
| ttcggcaact gcagatgcga cgccgcctgt gtggaactgg gcaactgctg cctggactac | 180 |
| caggaaacct gcatcgagcc cgagcacatc tggacctgca acaagttcag atgcggcgag | 240 |
| aagcggctga ccagatccct gtgtgcctgc agcgacgact gcaaggacaa gggcgactgc | 300 |
| tgcatcaact acagcagcgt gtgccagggc gagaagtcct gggtggaaga accctgcgag | 360 |
| agcatcaacg agccccagtg ccctgccggc ttcgagacac ctcctaccct gctgttcagc | 420 |
| ctggacggct ttcgggccga gtacctgcac acatggggag gcctgctgcc cgtgatcagc | 480 |
| aagctgaaga agtgcggcac ctacaccaag aacatgcggc ccgtgtaccc caccaagacc | 540 |
| ttccccaacc actactccat cgtgaccggc ctgtaccccg agagccacgg catcatcgac | 600 |
| aacaagatgt acgaccccaa gatgaacgcc agcttcagcc tgaagtccaa agagaagttc | 660 |
| aaccccgagt ggtataaggg cgagcccatc tgggtcaccg ccaagtacca gggcctgaaa | 720 |
| agcggcacat tcttttggcc cggcagcgac gtggaaatca acggcatctt ccccgacatc | 780 |
| tataagatgt acaacggcag cgtgcccttc gaggaacgga tcctggctgt gctgcagtgg | 840 |
| ctgcagctgc ccaaggatga gcggccccac ttctacaccc tgtacctgga agaacctgac | 900 |
| agcagcggcc acagctacgg ccctgtgtcc agcgaagtga tcaaggccct gcagcgggtg | 960 |
| gacggcatgg tgggaatgct gatggacggc ctgaaagagc tgaacctgca cagatgcctg | 1020 |
| aacctgatcc tgatcagcga ccacggcatg gaacagggat cctgcaagaa gtacatctac | 1080 |
| ctgaacaagt acctgggcga cgtgaagaac atcaaagtga tctacggccc agccgccaga | 1140 |
| ctgaggccta gcgacgtgcc cgacaagtac tacagcttca actacgaggg aatcgcccgg | 1200 |
| aacctgagct gcagagagcc caaccagcac ttcaagccct acctgaagca cttcctgccc | 1260 |
| aagcggctgc acttcgccaa gagcgacaga atcgagcccc tgaccttcta cctggacccc | 1320 |
| cagtggcagc tggccctgaa tcccagcgag agaaagtact gcggcagcgg cttccacggc | 1380 |
| tccgacaacg tgttcagcaa catgcaggcc ctgttcgtgg gctacggacc cggctttaag | 1440 |
| cacggcatcg aggccgacac cttcgagaac atcgaggtgt acaatctgat gtgcgacctg | 1500 |
| ctgaatctga cccctgcccc caacaatggc acccacggca gcctgaacca tctgctgaag | 1560 |
| aaccccgtgt acacccctaa gcaccccaaa gaggtgcacc ccctggtgca gtgccccttc | 1620 |
| accagaaacc ccagagacaa cctgggctgt agctgcaacc ccagcatcct gcccatcgag | 1680 |
| gacttccaga cccagttcaa cctgaccgtg gccgaggaaa agatcatcaa gcacgagaca | 1740 |
| ctgccctacg gcagaccccg ggtgctgcag aaagagaaca ccatctgcct gctgagccag | 1800 |
| caccagttca tgagcggcta ctcccaggac atcctgatgc ccctgtggac cagctacacc | 1860 |
| gtggaccgga acgacagctt ctccaccgag gatttcagca actgcctgta ccaggatttc | 1920 |
| cggatccccc tgagccccgt gcacaagtgc agcttctaca agaacaacac caaggtgtcc | 1980 |
| tacggcttcc tgagccctcc ccagctgaac aagaacagct ccggcatcta cagcgaggcc | 2040 |
| ctgctgacta ccaacatcgt gcccatgtac cagagcttcc aagtgatctg gcggtacttc | 2100 |
| cacgacaccc tgctgcggaa gtacgccgaa gaacggaacg gcgtgaacgt ggtgtccggc | 2160 |
| ccagtgttcg acttcgacta cgacggcaga tgtgacagcc tggaaaatct gcggcagaaa | 2220 |
| agaagagtga tccggaacca ggaaattctg atccctaccc acttctttat cgtgctgaca | 2280 |
| agctgcaagg ataccagcca gacccccctg cactgcgaga acctggatac cctggccttc | 2340 |
| atcctgcctc accggaccga caacagcgag agctgtgtgc acggcaagca cgacagctct | 2400 |
| tgggtggaag aactgctgat gctgcaccgg gccagaatca ccgatgtgga acacatcacc | 2460 |
| ggcctgagct tttaccagca gcggaaagaa cccgtgtccg atatcctgaa gctgaaaacc | 2520 |
| catctgccca ccttcagcca ggaagatggt ggaggaggct ctggtggagg cggtagcgga | 2580 |
| ggcggagggt cgggaggttc tggatcaatg aagtgggtaa cctttatttc ccttcttttt | 2640 |
| ctctttagct cggcttattc caggggtgtg tttcgtcgag atgcacacaa gagtgaggtt | 2700 |
| gctcatcggt ttaaagattt gggagaagaa aatttcaaag ccttggtgtt gattgccttt | 2760 |
| gctcagtatc ttcagcagtg tccatttgaa gatcatgtaa aattagtgaa tgaagtaact | 2820 |
| gaatttgcaa aaacatgtgt tgctgatgag tcagctgaaa attgtgacaa atcacttcat | 2880 |
| accctttttg gagacaaatt atgcacagtt gcaactcttc gtgaaaccta tggtgaaatg | 2940 |
| gctgactgct gtgcaaaaca agaacctgag agaaatgaat gcttcttgca acacaaagat | 3000 |
| gacaacccaa acctcccccg attggtgaga ccagaggttg atgtgatgtg cactgctttt | 3060 |
| catgacaatg aagagacatt tttgaaaaaa tacttatatg aaattgccag aagacatcct | 3120 |
| tacttttatg ccccggaact ccttttcttt gctaaaaggt ataaagctgc ttttacagaa | 3180 |
| tgttgccaag ctgctgataa agctgcctgc ctgttgccaa agctcgatga acttcgggat | 3240 |
| gaagggaagg cttcgtctgc caaacagaga ctcaagtgtg ccagtctcca aaaatttgga | 3300 |
| gaaagagctt tcaaagcatg ggcagtagct cgcctgagcc agagatttcc caaagctgag | 3360 |
| tttgcagaag tttccaagtt agtgacagat cttaccaaag tccacacgga atgctgccat | 3420 |
| ggagatctgc ttgaatgtgc tgatgacagg gcggaccttg ccaagtatat ctgtgaaaat | 3480 |
| caagattcga tctccagtaa actgaaggaa tgctgtgaaa aacctctgtt ggaaaaatcc | 3540 |
| cactgcattg ccgaagtgga aaatgatgag atgcctgctg acttgccttc attagctgct | 3600 |
| gattttgttg aaagtaagga tgtttgcaaa aactatgctg aggcaaagga tgtcttcctg | 3660 |
| ggcatgtttt tgtatgaata tgcaagaagg catcctgatt actctgtcgt gctgctgctg | 3720 |
| agacttgcca agacatatga aaccactcta gagaagtgct gtgccgctgc agatcctcat | 3780 |
| gaatgctatg ccaaagtgtt cgatgaattt aaacctcttg tggaagagcc tcagaattta | 3840 |
| atcaaacaaa attgtgagct ttttgagcag cttggagagt acaaattcca gaatgcgcta | 3900 |
| ttagttcgtt acaccaagaa agtaccccaa gtgtcaactc caactcttgt agaggtctca | 3960 |
| agaaacctag gaaaagtggg cagcaaatgt tgtaaacatc ctgaagcaaa aagaatgccc | 4020 |
| tgtgcagaag actatctatc cgtggtcctg aaccagttat gtgtgttgca tgagaaaacg | 4080 |
| ccagtaagtg acagagtcac caaatgctgc acagaatcct tggtgaacag gcgaccatgc | 4140 |
| ttttcagctc tggaagtcga tgaaacatac gttcccaaag agtttaatgc tgaaacattc | 4200 |
| accttccatg cagatatatg cacactttct gagaaggaga gacaaatcaa gaaacaaact | 4260 |
| gcacttgttg agctcgtgaa acacaagccc aaggcaacaa aagagcaact gaaagctgtt | 4320 |
| atggatgatt tcgcagcttt tgtagagaag tgctgcaagg ctgacgataa ggagacctgc | 4380 |
| tttgccgagg agggtaaaaa acttgttgct gcaagtcaag ctgccttagg ctta | 4434 |
| Nucleotide sequence of NPP121-NPP3-Fc | |
| SEQ. ID NO: 50 | |
| atggaaaggg acggatgcgc cggtggtgga tctcgcggag gcgaaggtgg aagggcccct | 60 |
| agggaaggac ctgccggaaa cggaagggac aggggacgct ctcacgccgc tgaagctcca | 120 |
| ggcgaccctc aggccgctgc ctctctgctg gctcctatgg acgtcggaga agaacccctg | 180 |
| gaaaaggccg ccagggccag gactgccaag gaccccaaca cctacaagat catctccctc | 240 |
| ttcactttcg ccgtcggagt caacatctgc ctgggattca ccgccgaaaa gcaaggcagc | 300 |
| tgcaggaaga agtgctttga tgcatcattt agaggactgg agaactgccg gtgtgatgtg | 360 |
| gcatgtaaag accgaggtga ttgctgctgg gattttgaag acacctgtgt ggaatcaact | 420 |
| cgaatatgga tgtgcaataa atttcgttgt ggagagacca gattagaggc cagcctttgc | 480 |
| tcttgttcag atgactgttt gcagaggaaa gattgctgtg ctgactataa gagtgtttgc | 540 |
| caaggagaaa cctcatggct ggaagaaaac tgtgacacag cccagcagtc tcagtgccca | 600 |
| gaagggtttg acctgccacc agttatcttg ttttctatgg atggatttag agctgaatat | 660 |
| ttatacacat gggatacttt aatgccaaat atcaataaac tgaaaacatg tggaattcat | 720 |
| tcaaaataca tgagagctat gtatcctacc aaaaccttcc caaatcatta caccattgtc | 780 |
| acgggcttgt atccagagtc acatggcatc attgacaata atatgtatga tgtaaatctc | 840 |
| aacaagaatt tttcactttc ttcaaaggaa caaaataatc cagcctggtg gcatgggcaa | 900 |
| ccaatgtggc tgacagcaat gtatcaaggt ttaaaagccg ctacctactt ttggcccgga | 960 |
| tcagaagtgg ctataaatgg ctcctttcct tccatataca tgccttacaa cggaagtgtc | 1020 |
| ccatttgaag agaggatttc tacactgtta aaatggctgg acctgcccaa agctgaaaga | 1080 |
| cccaggtttt ataccatgta ttttgaagaa cctgattcct ctggacatgc aggtggacca | 1140 |
| gtcagtgcca gagtaattaa agccttacag gtagtagatc atgcttttgg gatgttgatg | 1200 |
| gaaggcctga agcagcggaa tttgcacaac tgtgtcaata tcatccttct ggctgaccat | 1260 |
| ggaatggacc agacttattg taacaagatg gaatacatga ctgattattt tcccagaata | 1320 |
| aacttcttct acatgtacga agggcctgcc ccccgcatcc gagctcataa tatacctcat | 1380 |
| gactttttta gttttaattc tgaggaaatt gttagaaacc tcagttgccg aaaacctgat | 1440 |
| cagcatttca agccctattt gactcctgat ttgccaaagc gactgcacta tgccaagaac | 1500 |
| gtcagaatcg acaaagttca tctctttgtg gatcaacagt ggctggctgt taggagtaaa | 1560 |
| tcaaatacaa attgtggagg aggcaaccat ggttataaca atgagtttag gagcatggag | 1620 |
| gctatctttc tggcacatgg acccagtttt aaagagaaga ctgaagttga accatttgaa | 1680 |
| aatattgaag tctataacct aatgtgtgat cttctacgca ttcaaccagc accaaacaat | 1740 |
| ggaacccatg gtagtttaaa ccatcttctg aaggtgcctt tttatgagcc atcccatgca | 1800 |
| gaggaggtgt caaagttttc tgtttgtggc tttgctaatc cattgcccac agagtctctt | 1860 |
| gactgtttct gccctcacct acaaaatagt actcagctgg aacaagtgaa tcagatgcta | 1920 |
| aatctcaccc aagaagaaat aacagcaaca gtgaaagtaa atttgccatt tgggaggcct | 1980 |
| agggtactgc agaagaacgt ggaccactgt ctcctttacc acagggaata tgtcagtgga | 2040 |
| tttggaaaag ctatgaggat gcccatgtgg agttcataca cagtccccca gttgggagac | 2100 |
| acatcgcctc tgcctcccac tgtcccagac tgtctgcggg ctgatgtcag ggttcctcct | 2160 |
| tctgagagcc aaaaatgttc cttctattta gcagacaaga atatcaccca cggcttcctc | 2220 |
| tatcctcctg ccagcaatag aacatcagat agccaatatg atgctttaat tactagcaat | 2280 |
| ttggtaccta tgtatgaaga attcagaaaa atgtgggact acttccacag tgttcttctt | 2340 |
| ataaaacatg ccacagaaag aaatggagta aatgtggtta gtggaccaat atttgattat | 2400 |
| aattatgatg gccattttga tgctccagat gaaattacca aacatttagc caacactgat | 2460 |
| gttcccatcc caacacacta ctttgtggtg ctgaccagtt gtaaaaacaa gagccacaca | 2520 |
| ccggaaaact gccctgggtg gctggatgtc ctacccttta tcatccctca ccgacctacc | 2580 |
| aacgtggaga gctgtcctga aggtaaacca gaagctcttt gggttgaaga aagatttaca | 2640 |
| gctcacattg cccgggtccg tgatgtagaa cttctcactg ggcttgactt ctatcaggat | 2700 |
| aaagtgcagc ctgtctctga aattttgcaa ctaaagacat atttaccaac atttgaaacc | 2760 |
| actattgaca aaactcacac atgcccaccg tgcccagcac ctgaactcct ggggggaccg | 2820 |
| tcagtcttcc tcttcccccc aaaacccaag gacaccctca tgatctcccg gacccctgag | 2880 |
| gtcacatgcg tggtggtgga cgtgagccac gaagaccctg aggtcaagtt caactggtac | 2940 |
| gtggacggcg tggaggtgca taatgccaag acaaagccgc gggaggagca gtacaacagc | 3000 |
| acgtaccgtg tggtcagcgt cctcaccgtc ctgcaccagg actggctgaa tggcaaggag | 3060 |
| tacaagtgca aggtctccaa caaagccctc ccagccccca tcgagaaaac catctccaaa | 3120 |
| gccaaagggc agccccgaga accacaggtg tacaccctgc ccccatcccg ggaggagatg | 3180 |
| accaagaacc aggtcagcct gacctgcctg gtcaaaggct tctatcccag cgacatcgcc | 3240 |
| gtggagtggg agagcaatgg gcagccggag aacaactaca agaccacgcc tcccgtgctg | 3300 |
| gactccgacg gctccttctt cctctatagc aagctcaccg tggacaagag caggtggcag | 3360 |
| caggggaacg tcttctcatg ctccgtgatg catgaggctc tgcacaacca ctacacgcag | 3420 |
| aagagcctct ccctgtcccc gggtaaa | 3447 |
| Nucleotide sequence of NPP121-NPP3-Fc | |
| SEQ. ID NO: 51 | |
| atggaaaggg acggatgcgc cggtggtgga tctcgcggag gcgaaggtgg aagggcccct | 60 |
| agggaaggac ctgccggaaa cggaagggac aggggacgct ctcacgccgc tgaagctcca | 120 |
| ggcgaccctc aggccgctgc ctctctgctg gctcctatgg acgtcggaga agaacccctg | 180 |
| gaaaaggccg ccagggccag gactgccaag gaccccaaca cctacaagat catctccctc | 240 |
| ttcactttcg ccgtcggagt caacatctgc ctgggattca ccgccgaaaa gcaaggcagc | 300 |
| tgcaggaaga agtgctttga tgcatcattt agaggactgg agaactgccg gtgtgatgtg | 360 |
| gcatgtaaag accgaggtga ttgctgctgg gattttgaag acacctgtgt ggaatcaact | 420 |
| cgaatatgga tgtgcaataa atttcgttgt ggagagacca gattagaggc cagcctttgc | 480 |
| tcttgttcag atgactgttt gcagaggaaa gattgctgtg ctgactataa gagtgtttgc | 540 |
| caaggagaaa cctcatggct ggaagaaaac tgtgacacag cccagcagtc tcagtgccca | 600 |
| gaagggtttg acctgccacc agttatcttg ttttctatgg atggatttag agctgaatat | 660 |
| ttatacacat gggatacttt aatgccaaat atcaataaac tgaaaacatg tggaattcat | 720 |
| tcaaaataca tgagagctat gtatcctacc aaaaccttcc caaatcatta caccattgtc | 780 |
| acgggcttgt atccagagtc acatggcatc attgacaata atatgtatga tgtaaatctc | 840 |
| aacaagaatt tttcactttc ttcaaaggaa caaaataatc cagcctggtg gcatgggcaa | 900 |
| ccaatgtggc tgacagcaat gtatcaaggt ttaaaagccg ctacctactt ttggcccgga | 960 |
| tcagaagtgg ctataaatgg ctcctttcct tccatataca tgccttacaa cggaagtgtc | 1020 |
| ccatttgaag agaggatttc tacactgtta aaatggctgg acctgcccaa agctgaaaga | 1080 |
| cccaggtttt ataccatgta ttttgaagaa cctgattcct ctggacatgc aggtggacca | 1140 |
| gtcagtgcca gagtaattaa agccttacag gtagtagatc atgcttttgg gatgttgatg | 1200 |
| gaaggcctga agcagcggaa tttgcacaac tgtgtcaata tcatccttct ggctgaccat | 1260 |
| ggaatggacc agacttattg taacaagatg gaatacatga ctgattattt tcccagaata | 1320 |
| aacttcttct acatgtacga agggcctgcc ccccgcatcc gagctcataa tatacctcat | 1380 |
| gactttttta gttttaattc tgaggaaatt gttagaaacc tcagttgccg aaaacctgat | 1440 |
| cagcatttca agccctattt gactcctgat ttgccaaagc gactgcacta tgccaagaac | 1500 |
| gtcagaatcg acaaagttca tctctttgtg gatcaacagt ggctggctgt taggagtaaa | 1560 |
| tcaaatacaa attgtggagg aggcaaccat ggttataaca atgagtttag gagcatggag | 1620 |
| gctatctttc tggcacatgg acccagtttt aaagagaaga ctgaagttga accatttgaa | 1680 |
| aatattgaag tctataacct aatgtgtgat cttctacgca ttcaaccagc accaaacaat | 1740 |
| ggaacccatg gtagtttaaa ccatcttctg aaggtgcctt tttatgagcc atcccatgca | 1800 |
| gaggaggtgt caaagttttc tgtttgtggc tttgctaatc cattgcccac agagtctctt | 1860 |
| gactgtttct gccctcacct acaaaatagt actcagctgg aacaagtgaa tcagatgcta | 1920 |
| aatctcaccc aagaagaaat aacagcaaca gtgaaagtaa atttgccatt tgggaggcct | 1980 |
| agggtactgc agaagaacgt ggaccactgt ctcctttacc acagggaata tgtcagtgga | 2040 |
| tttggaaaag ctatgaggat gcccatgtgg agttcataca cagtccccca gttgggagac | 2100 |
| acatcgcctc tgcctcccac tgtcccagac tgtctgcggg ctgatgtcag ggttcctcct | 2160 |
| tctgagagcc aaaaatgttc cttctattta gcagacaaga atatcaccca cggcttcctc | 2220 |
| tatcctcctg ccagcaatag aacatcagat agccaatatg atgctttaat tactagcaat | 2280 |
| ttggtaccta tgtatgaaga attcagaaaa atgtgggact acttccacag tgttcttctt | 2340 |
| ataaaacatg ccacagaaag aaatggagta aatgtggtta gtggaccaat atttgattat | 2400 |
| aattatgatg gccattttga tgctccagat gaaattacca aacatttagc caacactgat | 2460 |
| gttcccatcc caacacacta ctttgtggtg ctgaccagtt gtaaaaacaa gagccacaca | 2520 |
| ccggaaaact gccctgggtg gctggatgtc ctacccttta tcatccctca ccgacctacc | 2580 |
| aacgtggaga gctgtcctga aggtaaacca gaagctcttt gggttgaaga aagatttaca | 2640 |
| gctcacattg cccgggtccg tgatgtagaa cttctcactg ggcttgactt ctatcaggat | 2700 |
| aaagtgcagc ctgtctctga aattttgcaa ctaaagacat atttaccaac atttgaaacc | 2760 |
| actattggtg gaggaggctc tggtggaggc ggtagcggag gcggagggtc gatgaagtgg | 2820 |
| gtaaccttta tttcccttct ttttctcttt agctcggctt attccagggg tgtgtttcgt | 2880 |
| cgagatgcac acaagagtga ggttgctcat cggtttaaag atttgggaga agaaaatttc | 2940 |
| aaagccttgg tgttgattgc ctttgctcag tatcttcagc agtgtccatt tgaagatcat | 3000 |
| gtaaaattag tgaatgaagt aactgaattt gcaaaaacat gtgttgctga tgagtcagct | 3060 |
| gaaaattgtg acaaatcact tcataccctt tttggagaca aattatgcac agttgcaact | 3120 |
| cttcgtgaaa cctatggtga aatggctgac tgctgtgcaa aacaagaacc tgagagaaat | 3180 |
| gaatgcttct tgcaacacaa agatgacaac ccaaacctcc cccgattggt gagaccagag | 3240 |
| gttgatgtga tgtgcactgc ttttcatgac aatgaagaga catttttgaa aaaatactta | 3300 |
| tatgaaattg ccagaagaca tccttacttt tatgccccgg aactcctttt ctttgctaaa | 3360 |
| aggtataaag ctgcttttac agaatgttgc caagctgctg ataaagctgc ctgcctgttg | 3420 |
| ccaaagctcg atgaacttcg ggatgaaggg aaggcttcgt ctgccaaaca gagactcaag | 3480 |
| tgtgccagtc tccaaaaatt tggagaaaga gctttcaaag catgggcagt agctcgcctg | 3540 |
| agccagagat ttcccaaagc tgagtttgca gaagtttcca agttagtgac agatcttacc | 3600 |
| aaagtccaca cggaatgctg ccatggagat ctgcttgaat gtgctgatga cagggcggac | 3660 |
| cttgccaagt atatctgtga aaatcaagat tcgatctcca gtaaactgaa ggaatgctgt | 3720 |
| gaaaaacctc tgttggaaaa atcccactgc attgccgaag tggaaaatga tgagatgcct | 3780 |
| gctgacttgc cttcattagc tgctgatttt gttgaaagta aggatgtttg caaaaactat | 3840 |
| gctgaggcaa aggatgtctt cctgggcatg tttttgtatg aatatgcaag aaggcatcct | 3900 |
| gattactctg tcgtgctgct gctgagactt gccaagacat atgaaaccac tctagagaag | 3960 |
| tgctgtgccg ctgcagatcc tcatgaatgc tatgccaaag tgttcgatga atttaaacct | 4020 |
| cttgtggaag agcctcagaa tttaatcaaa caaaattgtg agctttttga gcagcttgga | 4080 |
| gagtacaaat tccagaatgc gctattagtt cgttacacca agaaagtacc ccaagtgtca | 4140 |
| actccaactc ttgtagaggt ctcaagaaac ctaggaaaag tgggcagcaa atgttgtaaa | 4200 |
| catcctgaag caaaaagaat gccctgtgca gaagactatc tatccgtggt cctgaaccag | 4260 |
| ttatgtgtgt tgcatgagaa aacgccagta agtgacagag tcaccaaatg ctgcacagaa | 4320 |
| tccttggtga acaggcgacc atgcttttca gctctggaag tcgatgaaac atacgttccc | 4380 |
| aaagagttta atgctgaaac attcaccttc catgcagata tatgcacact ttctgagaag | 4440 |
| gagagacaaa tcaagaaaca aactgcactt gttgagctcg tgaaacacaa gcccaaggca | 4500 |
| acaaaagagc aactgaaagc tgttatggat gatttcgcag cttttgtaga gaagtgctgc | 4560 |
| aaggctgacg ataaggagac ctgctttgcc gaggagggta aaaaacttgt tgctgcaagt | 4620 |
| caagctgcct taggctta | 4638 |
| Nucleotide sequence of hNPP3-hFc-pcDNA3 | |
| SEQ. ID NO: 52 | |
| gacggatcgg gagatctccc gatcccctat ggtcgactct cagtacaatc tgctctgatg | 60 |
| ccgcatagtt aagccagtat ctgctccctg cttgtgtgtt ggaggtcgct gagtagtgcg | 120 |
| cgagcaaaat ttaagctaca acaaggcaag gcttgaccga caattgcatg aagaatctgc | 180 |
| ttagggttag gcgttttgcg ctgcttcgcg atgtacgggc cagatatacg cgttgacatt | 240 |
| gattattgac tagttattaa tagtaatcaa ttacggggtc attagttcat agcccatata | 300 |
| tggagttccg cgttacataa cttacggtaa atggcccgcc tggctgaccg cccaacgacc | 360 |
| cccgcccatt gacgtcaata atgacgtatg ttcccatagt aacgccaata gggactttcc | 420 |
| attgacgtca atgggtggac tatttacggt aaactgccca cttggcagta catcaagtgt | 480 |
| atcatatgcc aagtacgccc cctattgacg tcaatgacgg taaatggccc gcctggcatt | 540 |
| atgcccagta catgacctta tgggactttc ctacttggca gtacatctac gtattagtca | 600 |
| tcgctattac catggtgatg cggttttggc agtacatcaa tgggcgtgga tagcggtttg | 660 |
| actcacgggg atttccaagt ctccacccca ttgacgtcaa tgggagtttg ttttggcacc | 720 |
| aaaatcaacg ggactttcca aaatgtcgta acaactccgc cccattgacg caaatgggcg | 780 |
| gtaggcgtgt acggtgggag gtctatataa gcagagctct ctggctaact agagaaccca | 840 |
| ctgcttactg gcttatcgaa attaatacga ctcactatag ggagacccaa gcttatggaa | 900 |
| agggacggat gcgccggtgg tggatctcgc ggaggcgaag gtggaagggc ccctagggaa | 960 |
| ggacctgccg gaaacggaag ggacagggga cgctctcacg ccgctgaagc tccaggcgac | 1020 |
| cctcaggccg ctgcctctct gctggctcct atggacgtcg gagaagaacc cctggaaaag | 1080 |
| gccgccaggg ccaggactgc caaggacccc aacacctaca agatcatctc cctcttcact | 1140 |
| ttcgccgtcg gagtcaacat ctgcctggga ttcaccgccg aaaagcaagg cagctgcagg | 1200 |
| aagaagtgct ttgatgcatc atttagagga ctggagaact gccggtgtga tgtggcatgt | 1260 |
| aaagaccgag gtgattgctg ctgggatttt gaagacacct gtgtggaatc aactcgaata | 1320 |
| tggatgtgca ataaatttcg ttgtggagag accagattag aggccagcct ttgctcttgt | 1380 |
| tcagatgact gtttgcagag gaaagattgc tgtgctgact ataagagtgt ttgccaagga | 1440 |
| gaaacctcat ggctggaaga aaactgtgac acagcccagc agtctcagtg cccagaaggg | 1500 |
| tttgacctgc caccagttat cttgttttct atggatggat ttagagctga atatttatac | 1560 |
| acatgggata ctttaatgcc aaatatcaat aaactgaaaa catgtggaat tcattcaaaa | 1620 |
| tacatgagag ctatgtatcc taccaaaacc ttcccaaatc attacaccat tgtcacgggc | 1680 |
| ttgtatccag agtcacatgg catcattgac aataatatgt atgatgtaaa tctcaacaag | 1740 |
| aatttttcac tttcttcaaa ggaacaaaat aatccagcct ggtggcatgg gcaaccaatg | 1800 |
| tggctgacag caatgtatca aggtttaaaa gccgctacct acttttggcc cggatcagaa | 1860 |
| gtggctataa atggctcctt tccttccata tacatgcctt acaacggaag tgtcccattt | 1920 |
| gaagagagga tttctacact gttaaaatgg ctggacctgc ccaaagctga aagacccagg | 1980 |
| ttttatacca tgtattttga agaacctgat tcctctggac atgcaggtgg accagtcagt | 2040 |
| gccagagtaa ttaaagcctt acaggtagta gatcatgctt ttgggatgtt gatggaaggc | 2100 |
| ctgaagcagc ggaatttgca caactgtgtc aatatcatcc ttctggctga ccatggaatg | 2160 |
| gaccagactt attgtaacaa gatggaatac atgactgatt attttcccag aataaacttc | 2220 |
| ttctacatgt acgaagggcc tgccccccgc atccgagctc ataatatacc tcatgacttt | 2280 |
| tttagtttta attctgagga aattgttaga aacctcagtt gccgaaaacc tgatcagcat | 2340 |
| ttcaagccct atttgactcc tgatttgcca aagcgactgc actatgccaa gaacgtcaga | 2400 |
| atcgacaaag ttcatctctt tgtggatcaa cagtggctgg ctgttaggag taaatcaaat | 2460 |
| acaaattgtg gaggaggcaa ccatggttat aacaatgagt ttaggagcat ggaggctatc | 2520 |
| tttctggcac atggacccag ttttaaagag aagactgaag ttgaaccatt tgaaaatatt | 2580 |
| gaagtctata acctaatgtg tgatcttcta cgcattcaac cagcaccaaa caatggaacc | 2640 |
| catggtagtt taaaccatct tctgaaggtg cctttttatg agccatccca tgcagaggag | 2700 |
| gtgtcaaagt tttctgtttg tggctttgct aatccattgc ccacagagtc tcttgactgt | 2760 |
| ttctgccctc acctacaaaa tagtactcag ctggaacaag tgaatcagat gctaaatctc | 2820 |
| acccaagaag aaataacagc aacagtgaaa gtaaatttgc catttgggag gcctagggta | 2880 |
| ctgcagaaga acgtggacca ctgtctcctt taccacaggg aatatgtcag tggatttgga | 2940 |
| aaagctatga ggatgcccat gtggagttca tacacagtcc cccagttggg agacacatcg | 3000 |
| cctctgcctc ccactgtccc agactgtctg cgggctgatg tcagggttcc tccttctgag | 3060 |
| agccaaaaat gttccttcta tttagcagac aagaatatca cccacggctt cctctatcct | 3120 |
| cctgccagca atagaacatc agatagccaa tatgatgctt taattactag caatttggta | 3180 |
| cctatgtatg aagaattcag aaaaatgtgg gactacttcc acagtgttct tcttataaaa | 3240 |
| catgccacag aaagaaatgg agtaaatgtg gttagtggac caatatttga ttataattat | 3300 |
| gatggccatt ttgatgctcc agatgaaatt accaaacatt tagccaacac tgatgttccc | 3360 |
| atcccaacac actactttgt ggtgctgacc agttgtaaaa acaagagcca cacaccggaa | 3420 |
| aactgccctg ggtggctgga tgtcctaccc tttatcatcc ctcaccgacc taccaacgtg | 3480 |
| gagagctgtc ctgaaggtaa accagaagct ctttgggttg aagaaagatt tacagctcac | 3540 |
| attgcccggg tccgtgatgt agaacttctc actgggcttg acttctatca ggataaagtg | 3600 |
| cagcctgtct ctgaaatttt gcaactaaag acatatttac caacatttga aaccactatt | 3660 |
| gacaaaactc acacatgccc accgtgccca gcacctgaac tcctgggggg accgtcagtc | 3720 |
| ttcctcttcc ccccaaaacc caaggacacc ctcatgatct cccggacccc tgaggtcaca | 3780 |
| tgcgtggtgg tggacgtgag ccacgaagac cctgaggtca agttcaactg gtacgtggac | 3840 |
| ggcgtggagg tgcataatgc caagacaaag ccgcgggagg agcagtacaa cagcacgtac | 3900 |
| cgtgtggtca gcgtcctcac cgtcctgcac caggactggc tgaatggcaa ggagtacaag | 3960 |
| tgcaaggtct ccaacaaagc cctcccagcc cccatcgaga aaaccatctc caaagccaaa | 4020 |
| gggcagcccc gagaaccaca ggtgtacacc ctgcccccat cccgggagga gatgaccaag | 4080 |
| aaccaggtca gcctgacctg cctggtcaaa ggcttctatc ccagcgacat cgccgtggag | 4140 |
| tgggagagca atgggcagcc ggagaacaac tacaagacca cgcctcccgt gctggactcc | 4200 |
| gacggctcct tcttcctcta tagcaagctc accgtggaca agagcaggtg gcagcagggg | 4260 |
| aacgtcttct catgctccgt gatgcatgag gctctgcaca accactacac gcagaagagc | 4320 |
| ctctccctgt ccccgggtaa atgaaattct gcagatatcc atcacactgg cggccgctcg | 4380 |
| agcatgcatc tagagggccc tattctatag tgtcacctaa atgctagagc tcgctgatca | 4440 |
| gcctcgactg tgccttctag ttgccagcca tctgttgttt gcccctcccc cgtgccttcc | 4500 |
| ttgaccctgg aaggtgccac tcccactgtc ctttcctaat aaaatgagga aattgcatcg | 4560 |
| cattgtctga gtaggtgtca ttctattctg gggggtgggg tggggcagga cagcaagggg | 4620 |
| gaggattggg aagacaatag caggcatgct ggggatgcgg tgggctctat ggcttctgag | 4680 |
| gcggaaagaa ccagctgggg ctctaggggg tatccccacg cgccctgtag cggcgcatta | 4740 |
| agcgcggcgg gtgtggtggt tacgcgcagc gtgaccgcta cacttgccag cgccctagcg | 4800 |
| cccgctcctt tcgctttctt cccttccttt ctcgccacgt tcgccggctt tccccgtcaa | 4860 |
| gctctaaatc ggggcatccc tttagggttc cgatttagtg ctttacggca cctcgacccc | 4920 |
| aaaaaacttg attagggtga tggttcacgt agtgggccat cgccctgata gacggttttt | 4980 |
| cgccctttga cgttggagtc cacgttcttt aatagtggac tcttgttcca aactggaaca | 5040 |
| acactcaacc ctatctcggt ctattctttt gatttataag ggattttggg gatttcggcc | 5100 |
| tattggttaa aaaatgagct gatttaacaa aaatttaacg cgaattaatt ctgtggaatg | 5160 |
| tgtgtcagtt agggtgtgga aagtccccag gctccccagg caggcagaag tatgcaaagc | 5220 |
| atgcatctca attagtcagc aaccaggtgt ggaaagtccc caggctcccc agcaggcaga | 5280 |
| agtatgcaaa gcatgcatct caattagtca gcaaccatag tcccgcccct aactccgccc | 5340 |
| atcccgcccc taactccgcc cagttccgcc cattctccgc cccatggctg actaattttt | 5400 |
| tttatttatg cagaggccga ggccgcctct gcctctgagc tattccagaa gtagtgagga | 5460 |
| ggcttttttg gaggcctagg cttttgcaaa aagctcccgg gagcttgtat atccattttc | 5520 |
| ggatctgatc aagagacagg atgaggatcg tttcgcatga ttgaacaaga tggattgcac | 5580 |
| gcaggttctc cggccgcttg ggtggagagg ctattcggct atgactgggc acaacagaca | 5640 |
| atcggctgct ctgatgccgc cgtgttccgg ctgtcagcgc aggggcgccc ggttcttttt | 5700 |
| gtcaagaccg acctgtccgg tgccctgaat gaactgcagg acgaggcagc gcggctatcg | 5760 |
| tggctggcca cgacgggcgt tccttgcgca gctgtgctcg acgttgtcac tgaagcggga | 5820 |
| agggactggc tgctattggg cgaagtgccg gggcaggatc tcctgtcatc tcaccttgct | 5880 |
| cctgccgaga aagtatccat catggctgat gcaatgcggc ggctgcatac gcttgatccg | 5940 |
| gctacctgcc cattcgacca ccaagcgaaa catcgcatcg agcgagcacg tactcggatg | 6000 |
| gaagccggtc ttgtcgatca ggatgatctg gacgaagagc atcaggggct cgcgccagcc | 6060 |
| gaactgttcg ccaggctcaa ggcgcgcatg cccgacggcg aggatctcgt cgtgacccat | 6120 |
| ggcgatgcct gcttgccgaa tatcatggtg gaaaatggcc gcttttctgg attcatcgac | 6180 |
| tgtggccggc tgggtgtggc ggaccgctat caggacatag cgttggctac ccgtgatatt | 6240 |
| gctgaagagc ttggcggcga atgggctgac cgcttcctcg tgctttacgg tatcgccgct | 6300 |
| cccgattcgc agcgcatcgc cttctatcgc cttcttgacg agttcttctg agcgggactc | 6360 |
| tggggttcga aatgaccgac caagcgacgc ccaacctgcc atcacgagat ttcgattcca | 6420 |
| ccgccgcctt ctatgaaagg ttgggcttcg gaatcgtttt ccgggacgcc ggctggatga | 6480 |
| tcctccagcg cggggatctc atgctggagt tcttcgccca ccccaacttg tttattgcag | 6540 |
| cttataatgg ttacaaataa agcaatagca tcacaaattt cacaaataaa gcattttttt | 6600 |
| cactgcattc tagttgtggt ttgtccaaac tcatcaatgt atcttatcat gtctgtatac | 6660 |
| cgtcgacctc tagctagagc ttggcgtaat catggtcata gctgtttcct gtgtgaaatt | 6720 |
| gttatccgct cacaattcca cacaacatac gagccggaag cataaagtgt aaagcctggg | 6780 |
| gtgcctaatg agtgagctaa ctcacattaa ttgcgttgcg ctcactgccc gctttccagt | 6840 |
| cgggaaacct gtcgtgccag ctgcattaat gaatcggcca acgcgcgggg agaggcggtt | 6900 |
| tgcgtattgg gcgctcttcc gcttcctcgc tcactgactc gctgcgctcg gtcgttcggc | 6960 |
| tgcggcgagc ggtatcagct cactcaaagg cggtaatacg gttatccaca gaatcagggg | 7020 |
| ataacgcagg aaagaacatg tgagcaaaag gccagcaaaa ggccaggaac cgtaaaaagg | 7080 |
| ccgcgttgct ggcgtttttc cataggctcc gcccccctga cgagcatcac aaaaatcgac | 7140 |
| gctcaagtca gaggtggcga aacccgacag gactataaag ataccaggcg tttccccctg | 7200 |
| gaagctccct cgtgcgctct cctgttccga ccctgccgct taccggatac ctgtccgcct | 7260 |
| ttctcccttc gggaagcgtg gcgctttctc aatgctcacg ctgtaggtat ctcagttcgg | 7320 |
| tgtaggtcgt tcgctccaag ctgggctgtg tgcacgaacc ccccgttcag cccgaccgct | 7380 |
| gcgccttatc cggtaactat cgtcttgagt ccaacccggt aagacacgac ttatcgccac | 7440 |
| tggcagcagc cactggtaac aggattagca gagcgaggta tgtaggcggt gctacagagt | 7500 |
| tcttgaagtg gtggcctaac tacggctaca ctagaaggac agtatttggt atctgcgctc | 7560 |
| tgctgaagcc agttaccttc ggaaaaagag ttggtagctc ttgatccggc aaacaaacca | 7620 |
| ccgctggtag cggtggtttt tttgtttgca agcagcagat tacgcgcaga aaaaaaggat | 7680 |
| ctcaagaaga tcctttgatc ttttctacgg ggtctgacgc tcagtggaac gaaaactcac | 7740 |
| gttaagggat tttggtcatg agattatcaa aaaggatctt cacctagatc cttttaaatt | 7800 |
| aaaaatgaag ttttaaatca atctaaagta tatatgagta aacttggtct gacagttacc | 7860 |
| aatgcttaat cagtgaggca cctatctcag cgatctgtct atttcgttca tccatagttg | 7920 |
| cctgactccc cgtcgtgtag ataactacga tacgggaggg cttaccatct ggccccagtg | 7980 |
| ctgcaatgat accgcgagac ccacgctcac cggctccaga tttatcagca ataaaccagc | 8040 |
| cagccggaag ggccgagcgc agaagtggtc ctgcaacttt atccgcctcc atccagtcta | 8100 |
| ttaattgttg ccgggaagct agagtaagta gttcgccagt taatagtttg cgcaacgttg | 8160 |
| ttgccattgc tacaggcatc gtggtgtcac gctcgtcgtt tggtatggct tcattcagct | 8220 |
| ccggttccca acgatcaagg cgagttacat gatcccccat gttgtgcaaa aaagcggtta | 8280 |
| gctccttcgg tcctccgatc gttgtcagaa gtaagttggc cgcagtgtta tcactcatgg | 8340 |
| ttatggcagc actgcataat tctcttactg tcatgccatc cgtaagatgc ttttctgtga | 8400 |
| ctggtgagta ctcaaccaag tcattctgag aatagtgtat gcggcgaccg agttgctctt | 8460 |
| gcccggcgtc aatacgggat aataccgcgc cacatagcag aactttaaaa gtgctcatca | 8520 |
| ttggaaaacg ttcttcgggg cgaaaactct caaggatctt accgctgttg agatccagtt | 8580 |
| cgatgtaacc cactcgtgca cccaactgat cttcagcatc ttttactttc accagcgttt | 8640 |
| ctgggtgagc aaaaacagga aggcaaaatg ccgcaaaaaa gggaataagg gcgacacgga | 8700 |
| aatgttgaat actcatactc ttcctttttc aatattattg aagcatttat cagggttatt | 8760 |
| gtctcatgag cggatacata tttgaatgta tttagaaaaa taaacaaata ggggttccgc | 8820 |
| gcacatttcc ccgaaaagtg ccacctgacg tc | 8852 |
| ENPP121-Fc-Nucleotide sequence | |
| SEQ. ID NO: 53 | |
| atggaaaggg acggatgcgc cggtggtgga tctcgcggag gcgaaggtgg aagggcccct | 60 |
| agggaaggac ctgccggaaa cggaagggac aggggacgct ctcacgccgc tgaagctcca | 120 |
| ggcgaccctc aggccgctgc ctctctgctg gctcctatgg acgtcggaga agaacccctg | 180 |
| gaaaaggccg ccagggccag gactgccaag gaccccaaca cctacaagat catctccctc | 240 |
| ttcactttcg ccgtcggagt caacatctgc ctgggattca ccgccggact gaagcccagc | 300 |
| tgcgccaaag aagtgaagtc ctgcaagggc cggtgcttcg agcggacctt cggcaactgc | 360 |
| agatgcgacg ccgcctgtgt ggaactgggc aactgctgcc tggactacca ggaaacctgc | 420 |
| atcgagcccg agcacatctg gacctgcaac aagttcagat gcggcgagaa gcggctgacc | 480 |
| agatccctgt gtgcctgcag cgacgactgc aaggacaagg gcgactgctg catcaactac | 540 |
| agcagcgtgt gccagggcga gaagtcctgg gtggaagaac cctgcgagag catcaacgag | 600 |
| ccccagtgcc ctgccggctt cgagacacct cctaccctgc tgttcagcct ggacggcttt | 660 |
| cgggccgagt acctgcacac atggggaggc ctgctgcccg tgatcagcaa gctgaagaag | 720 |
| tgcggcacct acaccaagaa catgcggccc gtgtacccca ccaagacctt ccccaaccac | 780 |
| tactccatcg tgaccggcct gtaccccgag agccacggca tcatcgacaa caagatgtac | 840 |
| gaccccaaga tgaacgccag cttcagcctg aagtccaaag agaagttcaa ccccgagtgg | 900 |
| tataagggcg agcccatctg ggtcaccgcc aagtaccagg gcctgaaaag cggcacattc | 960 |
| ttttggcccg gcagcgacgt ggaaatcaac ggcatcttcc ccgacatcta taagatgtac | 1020 |
| aacggcagcg tgcccttcga ggaacggatc ctggctgtgc tgcagtggct gcagctgccc | 1080 |
| aaggatgagc ggccccactt ctacaccctg tacctggaag aacctgacag cagcggccac | 1140 |
| agctacggcc ctgtgtccag cgaagtgatc aaggccctgc agcgggtgga cggcatggtg | 1200 |
| ggaatgctga tggacggcct gaaagagctg aacctgcaca gatgcctgaa cctgatcctg | 1260 |
| atcagcgacc acggcatgga acagggatcc tgcaagaagt acatctacct gaacaagtac | 1320 |
| ctgggcgacg tgaagaacat caaagtgatc tacggcccag ccgccagact gaggcctagc | 1380 |
| gacgtgcccg acaagtacta cagcttcaac tacgagggaa tcgcccggaa cctgagctgc | 1440 |
| agagagccca accagcactt caagccctac ctgaagcact tcctgcccaa gcggctgcac | 1500 |
| ttcgccaaga gcgacagaat cgagcccctg accttctacc tggaccccca gtggcagctg | 1560 |
| gccctgaatc ccagcgagag aaagtactgc ggcagcggct tccacggctc cgacaacgtg | 1620 |
| ttcagcaaca tgcaggccct gttcgtgggc tacggacccg gctttaagca cggcatcgag | 1680 |
| gccgacacct tcgagaacat cgaggtgtac aatctgatgt gcgacctgct gaatctgacc | 1740 |
| cctgccccca acaatggcac ccacggcagc ctgaaccatc tgctgaagaa ccccgtgtac | 1800 |
| acccctaagc accccaaaga ggtgcacccc ctggtgcagt gccccttcac cagaaacccc | 1860 |
| agagacaacc tgggctgtag ctgcaacccc agcatcctgc ccatcgagga cttccagacc | 1920 |
| cagttcaacc tgaccgtggc cgaggaaaag atcatcaagc acgagacact gccctacggc | 1980 |
| agaccccggg tgctgcagaa agagaacacc atctgcctgc tgagccagca ccagttcatg | 2040 |
| agcggctact cccaggacat cctgatgccc ctgtggacca gctacaccgt ggaccggaac | 2100 |
| gacagcttct ccaccgagga tttcagcaac tgcctgtacc aggatttccg gatccccctg | 2160 |
| agccccgtgc acaagtgcag cttctacaag aacaacacca aggtgtccta cggcttcctg | 2220 |
| agccctcccc agctgaacaa gaacagctcc ggcatctaca gcgaggccct gctgactacc | 2280 |
| aacatcgtgc ccatgtacca gagcttccaa gtgatctggc ggtacttcca cgacaccctg | 2340 |
| ctgcggaagt acgccgaaga acggaacggc gtgaacgtgg tgtccggccc agtgttcgac | 2400 |
| ttcgactacg acggcagatg tgacagcctg gaaaatctgc ggcagaaaag aagagtgatc | 2460 |
| cggaaccagg aaattctgat ccctacccac ttctttatcg tgctgacaag ctgcaaggat | 2520 |
| accagccaga cccccctgca ctgcgagaac ctggataccc tggccttcat cctgcctcac | 2580 |
| cggaccgaca acagcgagag ctgtgtgcac ggcaagcacg acagctcttg ggtggaagaa | 2640 |
| ctgctgatgc tgcaccgggc cagaatcacc gatgtggaac acatcaccgg cctgagcttt | 2700 |
| taccagcagc ggaaagaacc cgtgtccgat atcctgaagc tgaaaaccca tctgcccacc | 2760 |
| ttcagccagg aagatgacaa gacccacact tgccccccct gcccagctcc tgaactgctg | 2820 |
| ggaggaccct ctgtgttcct gttcccccca aagcccaagg acaccctgat gatctctagg | 2880 |
| acccccgaag tcacttgcgt cgtcgtcgac gtgtcccacg aggaccctga agtcaagttc | 2940 |
| aactggtacg tcgacggtgt cgaagtccac aacgccaaga ccaagcccag ggaagaacag | 3000 |
| tacaactcta cctaccgcgt cgtcagcgtc ctgaccgtcc tgcaccagga ctggctgaac | 3060 |
| ggaaaggaat acaagtgcaa ggtgtccaac aaggccctgc ctgcccccat cgaaaagacc | 3120 |
| atctctaagg ccaagggaca gccccgcgaa ccccaggtct acaccctgcc accctctagg | 3180 |
| gaagaaatga ccaagaacca ggtgtccctg acctgcctgg tcaagggatt ctacccctct | 3240 |
| gacatcgccg tcgaatggga atctaacgga cagcccgaaa acaactacaa gaccaccccc | 3300 |
| cctgtcctgg actctgacgg atcattcttc ctgtactcta agctgactgt cgacaagtct | 3360 |
| aggtggcagc agggaaacgt gttctcttgc tctgtcatgc acgaagccct gcacaaccac | 3420 |
| tacacccaga agtctctgtc tctgtccccc ggaaag | 3456 |
| ENPP121- Albumin Nucleotide sequence | |
| SEQ. ID NO: 54 | |
| atggaaaggg acggatgcgc cggtggtgga tctcgcggag gcgaaggtgg aagggcccct | 60 |
| agggaaggac ctgccggaaa cggaagggac aggggacgct ctcacgccgc tgaagctcca | 120 |
| ggcgaccctc aggccgctgc ctctctgctg gctcctatgg acgtcggaga agaacccctg | 180 |
| gaaaaggccg ccagggccag gactgccaag gaccccaaca cctacaagat catctccctc | 240 |
| ttcactttcg ccgtcggagt caacatctgc ctgggattca ccgccggact gaagcccagc | 300 |
| tgcgccaaag aagtgaagtc ctgcaagggc cggtgcttcg agcggacctt cggcaactgc | 360 |
| agatgcgacg ccgcctgtgt ggaactgggc aactgctgcc tggactacca ggaaacctgc | 420 |
| atcgagcccg agcacatctg gacctgcaac aagttcagat gcggcgagaa gcggctgacc | 480 |
| agatccctgt gtgcctgcag cgacgactgc aaggacaagg gcgactgctg catcaactac | 540 |
| agcagcgtgt gccagggcga gaagtcctgg gtggaagaac cctgcgagag catcaacgag | 600 |
| ccccagtgcc ctgccggctt cgagacacct cctaccctgc tgttcagcct ggacggcttt | 660 |
| cgggccgagt acctgcacac atggggaggc ctgctgcccg tgatcagcaa gctgaagaag | 720 |
| tgcggcacct acaccaagaa catgcggccc gtgtacccca ccaagacctt ccccaaccac | 780 |
| tactccatcg tgaccggcct gtaccccgag agccacggca tcatcgacaa caagatgtac | 840 |
| gaccccaaga tgaacgccag cttcagcctg aagtccaaag agaagttcaa ccccgagtgg | 900 |
| tataagggcg agcccatctg ggtcaccgcc aagtaccagg gcctgaaaag cggcacattc | 960 |
| ttttggcccg gcagcgacgt ggaaatcaac ggcatcttcc ccgacatcta taagatgtac | 1020 |
| aacggcagcg tgcccttcga ggaacggatc ctggctgtgc tgcagtggct gcagctgccc | 1080 |
| aaggatgagc ggccccactt ctacaccctg tacctggaag aacctgacag cagcggccac | 1140 |
| agctacggcc ctgtgtccag cgaagtgatc aaggccctgc agcgggtgga cggcatggtg | 1200 |
| ggaatgctga tggacggcct gaaagagctg aacctgcaca gatgcctgaa cctgatcctg | 1260 |
| atcagcgacc acggcatgga acagggatcc tgcaagaagt acatctacct gaacaagtac | 1320 |
| ctgggcgacg tgaagaacat caaagtgatc tacggcccag ccgccagact gaggcctagc | 1380 |
| gacgtgcccg acaagtacta cagcttcaac tacgagggaa tcgcccggaa cctgagctgc | 1440 |
| agagagccca accagcactt caagccctac ctgaagcact tcctgcccaa gcggctgcac | 1500 |
| ttcgccaaga gcgacagaat cgagcccctg accttctacc tggaccccca gtggcagctg | 1560 |
| gccctgaatc ccagcgagag aaagtactgc ggcagcggct tccacggctc cgacaacgtg | 1620 |
| ttcagcaaca tgcaggccct gttcgtgggc tacggacccg gctttaagca cggcatcgag | 1680 |
| gccgacacct tcgagaacat cgaggtgtac aatctgatgt gcgacctgct gaatctgacc | 1740 |
| cctgccccca acaatggcac ccacggcagc ctgaaccatc tgctgaagaa ccccgtgtac | 1800 |
| acccctaagc accccaaaga ggtgcacccc ctggtgcagt gccccttcac cagaaacccc | 1860 |
| agagacaacc tgggctgtag ctgcaacccc agcatcctgc ccatcgagga cttccagacc | 1920 |
| cagttcaacc tgaccgtggc cgaggaaaag atcatcaagc acgagacact gccctacggc | 1980 |
| agaccccggg tgctgcagaa agagaacacc atctgcctgc tgagccagca ccagttcatg | 2040 |
| agcggctact cccaggacat cctgatgccc ctgtggacca gctacaccgt ggaccggaac | 2100 |
| gacagcttct ccaccgagga tttcagcaac tgcctgtacc aggatttccg gatccccctg | 2160 |
| agccccgtgc acaagtgcag cttctacaag aacaacacca aggtgtccta cggcttcctg | 2220 |
| agccctcccc agctgaacaa gaacagctcc ggcatctaca gcgaggccct gctgactacc | 2280 |
| aacatcgtgc ccatgtacca gagcttccaa gtgatctggc ggtacttcca cgacaccctg | 2340 |
| ctgcggaagt acgccgaaga acggaacggc gtgaacgtgg tgtccggccc agtgttcgac | 2400 |
| ttcgactacg acggcagatg tgacagcctg gaaaatctgc ggcagaaaag aagagtgatc | 2460 |
| cggaaccagg aaattctgat ccctacccac ttctttatcg tgctgacaag ctgcaaggat | 2520 |
| accagccaga cccccctgca ctgcgagaac ctggataccc tggccttcat cctgcctcac | 2580 |
| cggaccgaca acagcgagag ctgtgtgcac ggcaagcacg acagctcttg ggtggaagaa | 2640 |
| ctgctgatgc tgcaccgggc cagaatcacc gatgtggaac acatcaccgg cctgagcttt | 2700 |
| taccagcagc ggaaagaacc cgtgtccgat atcctgaagc tgaaaaccca tctgcccacc | 2760 |
| ttcagccagg aagatggtgg aggaggctct ggtggaggcg gtagcggagg cggagggtcg | 2820 |
| ggaggttctg gatcaatgaa gtgggtaacc tttatttccc ttctttttct ctttagctcg | 2880 |
| gcttattcca ggggtgtgtt tcgtcgagat gcacacaaga gtgaggttgc tcatcggttt | 2940 |
| aaagatttgg gagaagaaaa tttcaaagcc ttggtgttga ttgcctttgc tcagtatctt | 3000 |
| cagcagtgtc catttgaaga tcatgtaaaa ttagtgaatg aagtaactga atttgcaaaa | 3060 |
| acatgtgttg ctgatgagtc agctgaaaat tgtgacaaat cacttcatac cctttttgga | 3120 |
| gacaaattat gcacagttgc aactcttcgt gaaacctatg gtgaaatggc tgactgctgt | 3180 |
| gcaaaacaag aacctgagag aaatgaatgc ttcttgcaac acaaagatga caacccaaac | 3240 |
| ctcccccgat tggtgagacc agaggttgat gtgatgtgca ctgcttttca tgacaatgaa | 3300 |
| gagacatttt tgaaaaaata cttatatgaa attgccagaa gacatcctta cttttatgcc | 3360 |
| ccggaactcc ttttctttgc taaaaggtat aaagctgctt ttacagaatg ttgccaagct | 3420 |
| gctgataaag ctgcctgcct gttgccaaag ctcgatgaac ttcgggatga agggaaggct | 3480 |
| tcgtctgcca aacagagact caagtgtgcc agtctccaaa aatttggaga aagagctttc | 3540 |
| aaagcatggg cagtagctcg cctgagccag agatttccca aagctgagtt tgcagaagtt | 3600 |
| tccaagttag tgacagatct taccaaagtc cacacggaat gctgccatgg agatctgctt | 3660 |
| gaatgtgctg atgacagggc ggaccttgcc aagtatatct gtgaaaatca agattcgatc | 3720 |
| tccagtaaac tgaaggaatg ctgtgaaaaa cctctgttgg aaaaatccca ctgcattgcc | 3780 |
| gaagtggaaa atgatgagat gcctgctgac ttgccttcat tagctgctga ttttgttgaa | 3840 |
| agtaaggatg tttgcaaaaa ctatgctgag gcaaaggatg tcttcctggg catgtttttg | 3900 |
| tatgaatatg caagaaggca tcctgattac tctgtcgtgc tgctgctgag acttgccaag | 3960 |
| acatatgaaa ccactctaga gaagtgctgt gccgctgcag atcctcatga atgctatgcc | 4020 |
| aaagtgttcg atgaatttaa acctcttgtg gaagagcctc agaatttaat caaacaaaat | 4080 |
| tgtgagcttt ttgagcagct tggagagtac aaattccaga atgcgctatt agttcgttac | 4140 |
| accaagaaag taccccaagt gtcaactcca actcttgtag aggtctcaag aaacctagga | 4200 |
| aaagtgggca gcaaatgttg taaacatcct gaagcaaaaa gaatgccctg tgcagaagac | 4260 |
| tatctatccg tggtcctgaa ccagttatgt gtgttgcatg agaaaacgcc agtaagtgac | 4320 |
| agagtcacca aatgctgcac agaatccttg gtgaacaggc gaccatgctt ttcagctctg | 4380 |
| gaagtcgatg aaacatacgt tcccaaagag tttaatgctg aaacattcac cttccatgca | 4440 |
| gatatatgca cactttctga gaaggagaga caaatcaaga aacaaactgc acttgttgag | 4500 |
| ctcgtgaaac acaagcccaa ggcaacaaaa gagcaactga aagctgttat ggatgatttc | 4560 |
| gcagcttttg tagagaagtg ctgcaaggct gacgataagg agacctgctt tgccgaggag | 4620 |
| ggtaaaaaac ttgttgctgc aagtcaagct gccttaggct ta | 4662 |
| ENPP3 Nucleotide sequence | |
| SEQ. ID NO: 55 | |
| atggaatcta cgttgacttt agcaacggaa caacctgtta agaagaacac tcttaagaaa | 60 |
| tataaaatag cttgcattgt tcttcttgct ttgctggtga tcatgtcact tggattaggc | 120 |
| ctggggcttg gactcaggaa actggaaaag caaggcagct gcaggaagaa gtgctttgat | 180 |
| gcatcattta gaggactgga gaactgccgg tgtgatgtgg catgtaaaga ccgaggtgat | 240 |
| tgctgctggg attttgaaga cacctgtgtg gaatcaactc gaatatggat gtgcaataaa | 300 |
| tttcgttgtg gagagaccag attagaggcc agcctttgct cttgttcaga tgactgtttg | 360 |
| cagaggaaag attgctgtgc tgactataag agtgtttgcc aaggagaaac ctcatggctg | 420 |
| gaagaaaact gtgacacagc ccagcagtct cagtgcccag aagggtttga cctgccacca | 480 |
| gttatcttgt tttctatgga tggatttaga gctgaatatt tatacacatg ggatacttta | 540 |
| atgccaaata tcaataaact gaaaacatgt ggaattcatt caaaatacat gagagctatg | 600 |
| tatcctacca aaaccttccc aaatcattac accattgtca cgggcttgta tccagagtca | 660 |
| catggcatca ttgacaataa tatgtatgat gtaaatctca acaagaattt ttcactttct | 720 |
| tcaaaggaac aaaataatcc agcctggtgg catgggcaac caatgtggct gacagcaatg | 780 |
| tatcaaggtt taaaagccgc tacctacttt tggcccggat cagaagtggc tataaatggc | 840 |
| tcctttcctt ccatatacat gccttacaac ggaagtgtcc catttgaaga gaggatttct | 900 |
| acactgttaa aatggctgga cctgcccaaa gctgaaagac ccaggtttta taccatgtat | 960 |
| tttgaagaac ctgattcctc tggacatgca ggtggaccag tcagtgccag agtaattaaa | 1020 |
| gccttacagg tagtagatca tgcttttggg atgttgatgg aaggcctgaa gcagcggaat | 1080 |
| ttgcacaact gtgtcaatat catccttctg gctgaccatg gaatggacca gacttattgt | 1140 |
| aacaagatgg aatacatgac tgattatttt cccagaataa acttcttcta catgtacgaa | 1200 |
| gggcctgccc cccgcatccg agctcataat atacctcatg acttttttag ttttaattct | 1260 |
| gaggaaattg ttagaaacct cagttgccga aaacctgatc agcatttcaa gccctatttg | 1320 |
| actcctgatt tgccaaagcg actgcactat gccaagaacg tcagaatcga caaagttcat | 1380 |
| ctctttgtgg atcaacagtg gctggctgtt aggagtaaat caaatacaaa ttgtggagga | 1440 |
| ggcaaccatg gttataacaa tgagtttagg agcatggagg ctatctttct ggcacatgga | 1500 |
| cccagtttta aagagaagac tgaagttgaa ccatttgaaa atattgaagt ctataaccta | 1560 |
| atgtgtgatc ttctacgcat tcaaccagca ccaaacaatg gaacccatgg tagtttaaac | 1620 |
| catcttctga aggtgccttt ttatgagcca tcccatgcag aggaggtgtc aaagttttct | 1680 |
| gtttgtggct ttgctaatcc attgcccaca gagtctcttg actgtttctg ccctcaccta | 1740 |
| caaaatagta ctcagctgga acaagtgaat cagatgctaa atctcaccca agaagaaata | 1800 |
| acagcaacag tgaaagtaaa tttgccattt gggaggccta gggtactgca gaagaacgtg | 1860 |
| gaccactgtc tcctttacca cagggaatat gtcagtggat ttggaaaagc tatgaggatg | 1920 |
| cccatgtgga gttcatacac agtcccccag ttgggagaca catcgcctct gcctcccact | 1980 |
| gtcccagact gtctgcgggc tgatgtcagg gttcctcctt ctgagagcca aaaatgttcc | 2040 |
| ttctatttag cagacaagaa tatcacccac ggcttcctct atcctcctgc cagcaataga | 2100 |
| acatcagata gccaatatga tgctttaatt actagcaatt tggtacctat gtatgaagaa | 2160 |
| ttcagaaaaa tgtgggacta cttccacagt gttcttctta taaaacatgc cacagaaaga | 2220 |
| aatggagtaa atgtggttag tggaccaata tttgattata attatgatgg ccattttgat | 2280 |
| gctccagatg aaattaccaa acatttagcc aacactgatg ttcccatccc aacacactac | 2340 |
| tttgtggtgc tgaccagttg taaaaacaag agccacacac cggaaaactg ccctgggtgg | 2400 |
| ctggatgtcc taccctttat catccctcac cgacctacca acgtggagag ctgtcctgaa | 2460 |
| ggtaaaccag aagctctttg ggttgaagaa agatttacag ctcacattgc ccgggtccgt | 2520 |
| gatgtagaac ttctcactgg gcttgacttc tatcaggata aagtgcagcc tgtctctgaa | 2580 |
| attttgcaac taaagacata tttaccaaca tttgaaacca ctatt | 2625 |
| ENPP1 Nucleotide sequence: | |
| SEQ. ID NO: 56 | |
| atggaacggg acggctgtgc cggcggagga tcaagaggcg gagaaggcgg cagagcccct | 60 |
| agagaaggac ctgccggcaa cggcagagac agaggcagat ctcatgccgc cgaagcccct | 120 |
| ggcgatcctc aggctgctgc ttctctgctg gcccccatgg atgtgggcga ggaacctctg | 180 |
| gaaaaggccg ccagagccag aaccgccaag gaccccaaca cctacaaggt gctgagcctg | 240 |
| gtgctgtccg tgtgcgtgct gaccaccatc ctgggctgca tcttcggcct gaagcccagc | 300 |
| tgcgccaaag aagtgaagtc ctgcaagggc cggtgcttcg agcggacctt cggcaactgc | 360 |
| agatgcgacg ccgcctgtgt ggaactgggc aactgctgcc tggactacca ggaaacctgc | 420 |
| atcgagcccg agcacatctg gacctgcaac aagttcagat gcggcgagaa gcggctgacc | 480 |
| agatccctgt gtgcctgcag cgacgactgc aaggacaagg gcgactgctg catcaactac | 540 |
| agcagcgtgt gccagggcga gaagtcctgg gtggaagaac cctgcgagag catcaacgag | 600 |
| ccccagtgcc ctgccggctt cgagacacct cctaccctgc tgttcagcct ggacggcttt | 660 |
| cgggccgagt acctgcacac atggggaggc ctgctgcccg tgatcagcaa gctgaagaag | 720 |
| tgcggcacct acaccaagaa catgcggccc gtgtacccca ccaagacctt ccccaaccac | 780 |
| tactccatcg tgaccggcct gtaccccgag agccacggca tcatcgacaa caagatgtac | 840 |
| gaccccaaga tgaacgccag cttcagcctg aagtccaaag agaagttcaa ccccgagtgg | 900 |
| tataagggcg agcccatctg ggtcaccgcc aagtaccagg gcctgaaaag cggcacattc | 960 |
| ttttggcccg gcagcgacgt ggaaatcaac ggcatcttcc ccgacatcta taagatgtac | 1020 |
| aacggcagcg tgcccttcga ggaacggatc ctggctgtgc tgcagtggct gcagctgccc | 1080 |
| aaggatgagc ggccccactt ctacaccctg tacctggaag aacctgacag cagcggccac | 1140 |
| agctacggcc ctgtgtccag cgaagtgatc aaggccctgc agcgggtgga cggcatggtg | 1200 |
| ggaatgctga tggacggcct gaaagagctg aacctgcaca gatgcctgaa cctgatcctg | 1260 |
| atcagcgacc acggcatgga acagggatcc tgcaagaagt acatctacct gaacaagtac | 1320 |
| ctgggcgacg tgaagaacat caaagtgatc tacggcccag ccgccagact gaggcctagc | 1380 |
| gacgtgcccg acaagtacta cagcttcaac tacgagggaa tcgcccggaa cctgagctgc | 1440 |
| agagagccca accagcactt caagccctac ctgaagcact tcctgcccaa gcggctgcac | 1500 |
| ttcgccaaga gcgacagaat cgagcccctg accttctacc tggaccccca gtggcagctg | 1560 |
| gccctgaatc ccagcgagag aaagtactgc ggcagcggct tccacggctc cgacaacgtg | 1620 |
| ttcagcaaca tgcaggccct gttcgtgggc tacggacccg gctttaagca cggcatcgag | 1680 |
| gccgacacct tcgagaacat cgaggtgtac aatctgatgt gcgacctgct gaatctgacc | 1740 |
| cctgccccca acaatggcac ccacggcagc ctgaaccatc tgctgaagaa ccccgtgtac | 1800 |
| acccctaagc accccaaaga ggtgcacccc ctggtgcagt gccccttcac cagaaacccc | 1860 |
| agagacaacc tgggctgtag ctgcaacccc agcatcctgc ccatcgagga cttccagacc | 1920 |
| cagttcaacc tgaccgtggc cgaggaaaag atcatcaagc acgagacact gccctacggc | 1980 |
| agaccccggg tgctgcagaa agagaacacc atctgcctgc tgagccagca ccagttcatg | 2040 |
| agcggctact cccaggacat cctgatgccc ctgtggacca gctacaccgt ggaccggaac | 2100 |
| gacagcttct ccaccgagga tttcagcaac tgcctgtacc aggatttccg gatccccctg | 2160 |
| agccccgtgc acaagtgcag cttctacaag aacaacacca aggtgtccta cggcttcctg | 2220 |
| agccctcccc agctgaacaa gaacagctcc ggcatctaca gcgaggccct gctgactacc | 2280 |
| aacatcgtgc ccatgtacca gagcttccaa gtgatctggc ggtacttcca cgacaccctg | 2340 |
| ctgcggaagt acgccgaaga acggaacggc gtgaacgtgg tgtccggccc agtgttcgac | 2400 |
| ttcgactacg acggcagatg tgacagcctg gaaaatctgc ggcagaaaag aagagtgatc | 2460 |
| cggaaccagg aaattctgat ccctacccac ttctttatcg tgctgacaag ctgcaaggat | 2520 |
| accagccaga cccccctgca ctgcgagaac ctggataccc tggccttcat cctgcctcac | 2580 |
| cggaccgaca acagcgagag ctgtgtgcac ggcaagcacg acagctcttg ggtggaagaa | 2640 |
| ctgctgatgc tgcaccgggc cagaatcacc gatgtggaac acatcaccgg cctgagcttt | 2700 |
| taccagcagc ggaaagaacc cgtgtccgat atcctgaagc tgaaaaccca tctgcccacc | 2760 |
| ttcagccagg aagat | 2775 |

Linker
SEQ. ID NO: 57
Asp Ser Ser
Linker
SEQ. ID NO: 58
Glu Ser Ser
Linker
SEQ. ID NO: 59
Arg Gln Gln
Linker
SEQ. ID NO: 60
Lys Arg
Linker
SEQ. ID NO: 61
(Arg)ₘ; m = 0-15
Linker
SEQ. ID NO: 62
Asp Ser Ser Ser Glu Glu Lys Phe Leu Arg Arg Ile Gly Arg Phe Gly
Linker
SEQ. ID NO: 63
Glu Glu Glu Glu Glu Glu Glu Pro Arg Gly Asp Thr
1 5 10
Linker
SEQ. ID NO: 64
Ala Pro Trp His Leu Ser Ser Gln Tyr Ser Arg Thr
1 5 10
Linker
SEQ. ID NO: 65
Ser Thr Leu Pro Ile Pro His Glu Phe Ser Arg Glu
1 5 10
Linker
SEQ. ID NO: 66
Val Thr Lys His Leu Asn Gln Ile Ser Gln Ser Tyr
1 5 10
Linker
SEQ. ID NO: 67
(Glu)ₘ; m = 1-15
Linker
SEQ. ID NO: 68
Leu Ile Asn
Linker
SEQ. ID NO: 69
Gly Gly Ser Gly Gly Ser
1 5
Linker
SEQ. ID NO: 70
Arg Ser Gly Ser Gly Gly Ser
1 5
Linker
SEQ. ID NO: 71
(Asp)ₘ; m = 1-15
1
Linker
SEQ. ID NO: 72
Leu Val Ile Met Ser Leu Gly Leu Gly Leu Gly Leu Gly Leu Arg Lys
1 5 10 15
Linker
SEQ. ID NO: 73
Val Ile Met Ser Leu Gly Leu Gly Leu Gly Leu Gly Leu Arg Lys
1 5 10 15
Linker
SEQ. ID NO: 74
Ile Met Ser Leu Gly Leu Gly Leu Gly Leu Gly Leu Arg Lys
1 5 10
Linker
SEQ. ID NO: 75
Met Ser Leu Gly Leu Gly Leu Gly Leu Gly Leu Arg Lys
1 5 10
Linker
SEQ. ID NO: 76
Ser Leu Gly Leu Gly Leu Gly Leu Gly Leu Arg Lys
1 5 10
Linker
SEQ. ID NO: 77
Leu Gly Leu Gly Leu Gly Leu Gly Leu Arg Lys
1 5 10
Linker
SEQ. ID NO: 78
Gly Leu Gly Leu Gly Leu Gly Leu Arg Lys
1 5 10
Linker
SEQ. ID NO: 79
Leu Gly Leu Gly Leu Gly Leu Arg Lys
1 5
Linker
SEQ. ID NO: 80
Gly Leu Gly Leu Gly Leu Arg Lys
1 5
Linker
SEQ. ID NO: 81
Leu Gly Leu Gly Leu Arg Lys
1 5
Linker
SEQ. ID NO: 82
Gly Leu Gly Leu Arg Lys
1 5
Linker
SEQ. ID NO: 83
Leu Gly Leu Arg Lys
1 5
Linker
SEQ. ID NO: 84
Gly Leu Arg Lys
1
Linker
SEQ. ID NO: 85
Leu Arg Lys
1
Linker
SEQ. ID NO: 86
Arg Lys
1
Linker
SEQ. ID NO: 87
(Lys)ₘ; m = 0-15
1
Linker
SEQ. ID NO: 88
Dₘ; m = 1-15
1
Soluble NPP1-Fc fusion protein sequence
SEQ ID NO: 89
Phe Thr Ala Gly Leu Lys Pro Ser Cys Ala Lys Glu Val Lys Ser Cys
Lys Gly Arg Cys Phe Glu Arg Thr Phe Gly Asn Cys Arg Cys Asp Ala
Ala Cys Val Glu Leu Gly Asn Cys Cys Leu Asp Tyr Gln Glu Thr Cys
Ile Glu Pro Glu His Ile Trp Thr Cys Asn Lys Phe Arg Cys Gly Glu
Lys Arg Leu Thr Arg Ser Leu Cys Ala Cys Ser Asp Asp Cys Lys Asp
Lys Gly Asp Cys Cys Ile Asn Tyr Ser Ser Val Cys Gln Gly Glu Lys
Ser Trp Val Glu Glu Pro Cys Glu Ser Ile Asn Glu Pro Gln Cys Pro
Ala Gly Phe Glu Thr Pro Pro Thr Leu Leu Phe Ser Leu Asp Gly Phe
Arg Ala Glu Tyr Leu His Thr Trp Gly Gly Leu Leu Pro Val Ile Ser
Lys Leu Lys Lys Cys Gly Thr Tyr Thr Lys Asn Met Arg Pro Val Tyr
Pro Thr Lys Thr Phe Pro Asn His Tyr Ser Ile Val Thr Gly Leu Tyr
Pro Glu Ser His Gly Ile Ile Asp Asn Lys Met Tyr Asp Pro Lys Met
Asn Ala Ser Phe Ser Leu Lys Ser Lys Glu Lys Phe Asn Pro Glu Trp
Tyr Lys Gly Glu Pro Ile Trp Val Thr Ala Lys Tyr Gln Gly Leu Lys
Ser Gly Thr Phe Phe Trp Pro Gly Ser Asp Val Glu Ile Asn Gly Ile
Phe Pro Asp Ile Tyr Lys Met Tyr Asn Gly Ser Val Pro Phe Glu Glu
Arg Ile Leu Ala Val Leu Gln Trp Leu Gln Leu Pro Lys Asp Glu Arg
Pro His Phe Tyr Thr Leu Tyr Leu Glu Glu Pro Asp Ser Ser Gly His
Ser Tyr Gly Pro Val Ser Ser Glu Val Ile Lys Ala Leu Gln Arg Val
Asp Gly Met Val Gly Met Leu Met Asp Gly Leu Lys Glu Leu Asn Leu
His Arg Cys Leu Asn Leu Ile Leu Ile Ser Asp His Gly Met Glu Gln
Gly Ser Cys Lys Lys Tyr Ile Tyr Leu Asn Lys Tyr Leu Gly Asp Val
Lys Asn Ile Lys Val Ile Tyr Gly Pro Ala Ala Arg Leu Arg Pro Ser
Asp Val Pro Asp Lys Tyr Tyr Ser Phe Asn Tyr Glu Gly Ile Ala Arg
Asn Leu Ser Cys Arg Glu Pro Asn Gln His Phe Lys Pro Tyr Leu Lys
His Phe Leu Pro Lys Arg Leu His Phe Ala Lys Ser Asp Arg Ile Glu
Pro Leu Thr Phe Tyr Leu Asp Pro Gln Trp Gln Leu Ala Leu Asn Pro
Ser Glu Arg Lys Tyr Cys Gly Ser Gly Phe His Gly Ser Asp Asn Val
Phe Ser Asn Met Gln Ala Leu Phe Val Gly Tyr Gly Pro Gly Phe Lys
His Gly Ile Glu Ala Asp Thr Phe Glu Asn Ile Glu Val Tyr Asn Leu
Met Cys Asp Leu Leu Asn Leu Thr Pro Ala Pro Asn Asn Gly Thr His
Gly Ser Leu Asn His Leu Leu Lys Asn Pro Val Tyr Thr Pro Lys His
Pro Leu His Cys Glu Asn Leu Asp Thr Leu Ala Phe Ile Leu Pro His
Arg Thr Asp Asn Ser Glu Ser Cys Val His Gly Lys His Asp Ser Ser
Trp Val Glu Glu Leu Leu Met Leu His Arg Ala Arg Ile Thr Asp Val
Glu His Ile Thr Gly Leu Ser Phe Tyr Gln Gln Arg Lys Glu Pro Val
Ser Asp Ile Leu Lys Leu Lys Thr His Leu Pro Thr Phe Ser Gln Glu
Asp Leu Ile Asn Asp Lys Thr His Thr Cys Pro Pro Cys Pro Ala Pro
Glu Leu Leu Gly Gly Pro Ser Val Phe Leu Phe Pro Pro Lys Pro Lys
Asp Thr Leu Met Ile Ser Arg Thr Pro Glu Val Thr Cys Val Val Val
Asp Val Ser His Glu Asp Pro Glu Val Lys Phe Asn Trp Tyr Val Asp
Gly Val Glu Val His Asn Ala Lys Thr Lys Pro Arg Glu Glu Gln Tyr
Asn Ser Thr Tyr Arg Val Val Ser Val Leu Thr Val Leu His Gln Asp
Trp Leu Asn Gly Lys Glu Tyr Lys Cys Lys Val Ser Asn Lys Ala Leu
Pro Ala Pro Ile Glu Lys Thr Ile Ser Lys Ala Lys Gly Gln Pro Arg
Glu Pro Gln Val Tyr Thr Leu Pro Pro Ser Arg Glu Glu Met Thr Lys
Asn Gln Val Ser Leu Thr Cys Leu Val Lys Gly Phe Tyr Pro Ser Asp
Ile Ala Val Glu Trp Glu Ser Asn Gly Gln Pro Glu Asn Asn Tyr Lys
Thr Thr Pro Pro Val Leu Asp Ser Asp Gly Ser Phe Phe Leu Tyr Ser
Lys Leu Thr Val Asp Lys Ser Arg Trp Gln Gln Gly Asn Val Phe Ser
Cys Ser Val Met His Glu Ala Leu His Asn His Tyr Thr Gln Lys Ser
Leu Ser Leu Ser Pro Gly Lys
double-underlined: beginning and end of NPP1; bold residues
indicate Fc sequence
Nucleotide sequence of solube NPP1-Fc
SEQ ID NO: 90
ttca ccgccggact gaagcccagc
tgcgccaaag aagtgaagtc ctgcaagggc cggtgcttcg agcggacctt cggcaactgc
agatgcgacg ccgcctgtgt ggaactgggc aactgctgcc tggactacca ggaaacctgc
atcgagcccg agcacatctg gacctgcaac aagttcagat gcggcgagaa gcggctgacc
agatccctgt gtgcctgcag cgacgactgc aaggacaagg gcgactgctg catcaactac
agcagcgtgt gccagggcga gaagtcctgg gtggaagaac cctgcgagag catcaacgag
ccccagtgcc ctgccggctt cgagacacct cctaccctgc tgttcagcct ggacggcttt
cgggccgagt acctgcacac atggggaggc ctgctgcccg tgatcagcaa gctgaagaag
tgcggcacct acaccaagaa catgcggccc gtgtacccca ccaagacctt ccccaaccac
tactccatcg tgaccggcct gtaccccgag agccacggca tcatcgacaa caagatgtac
gaccccaaga tgaacgccag cttcagcctg aagtccaaag agaagttcaa ccccgagtgg
tataagggcg agcccatctg ggtcaccgcc aagtaccagg gcctgaaaag cggcacattc
ttttggcccg gcagcgacgt ggaaatcaac ggcatcttcc ccgacatcta taagatgtac
aacggcagcg tgcccttcga ggaacggatc ctggctgtgc tgcagtggct gcagctgccc
aaggatgagc ggccccactt ctacaccctg tacctggaag aacctgacag cagcggccac
agctacggcc ctgtgtccag cgaagtgatc aaggccctgc agcgggtgga cggcatggtg
ggaatgctga tggacggcct gaaagagctg aacctgcaca gatgcctgaa cctgatcctg
atcagcgacc acggcatgga acagggatcc tgcaagaagt acatctacct gaacaagtac
ctgggcgacg tgaagaacat caaagtgatc tacggcccag ccgccagact gaggcctagc
gacgtgcccg acaagtacta cagcttcaac tacgagggaa tcgcccggaa cctgagctgc
agagagccca accagcactt caagccctac ctgaagcact tcctgcccaa gcggctgcac
ttcgccaaga gcgacagaat cgagcccctg accttctacc tggaccccca gtggcagctg
gccctgaatc ccagcgagag aaagtactgc ggcagcggct tccacggctc cgacaacgtg
ttcagcaaca tgcaggccct gttcgtgggc tacggacccg gctttaagca cggcatcgag
gccgacacct tcgagaacat cgaggtgtac aatctgatgt gcgacctgct gaatctgacc
cctgccccca acaatggcac ccacggcagc ctgaaccatc tgctgaagaa ccccgtgtac
acccctaagc accccaaaga ggtgcacccc ctggtgcagt gccccttcac cagaaacccc
agagacaacc tgggctgtag ctgcaacccc agcatcctgc ccatcgagga cttccagacc
cagttcaacc tgaccgtggc cgaggaaaag atcatcaagc acgagacact gccctacggc
agaccccggg tgctgcagaa agagaacacc atctgcctgc tgagccagca ccagttcatg
agcggctact cccaggacat cctgatgccc ctgtggacca gctacaccgt ggaccggaac
gacagcttct ccaccgagga tttcagcaac tgcctgtacc aggatttccg gatccccctg
agccccgtgc acaagtgcag cttctacaag aacaacacca aggtgtccta cggcttcctg
agccctcccc agctgaacaa gaacagctcc ggcatctaca gcgaggccct gctgactacc
aacatcgtgc ccatgtacca gagcttccaa gtgatctggc ggtacttcca cgacaccctg
ctgcggaagt acgccgaaga acggaacggc gtgaacgtgg tgtccggccc agtgttcgac
ttcgactacg acggcagatg tgacagcctg gaaaatctgc ggcagaaaag aagagtgatc
cggaaccagg aaattctgat ccctacccac ttctttatcg tgctgacaag ctgcaaggat
accagccaga cccccctgca ctgcgagaac ctggataccc tggccttcat cctgcctcac
cggaccgaca acagcgagag ctgtgtgcac ggcaagcacg acagctcttg ggtggaagaa
ctgctgatgc tgcaccgggc cagaatcacc gatgtggaac acatcaccgg cctgagcttt
taccagcagc ggaaagaacc cgtgtccgat atcctgaagc tgaaaaccca tctgcccacc
ttcagccagg aagatgacaa gacccacact tgccccccct gcccagctcc tgaactgctg
ggaggaccct ctgtgttcct gttcccccca aagcccaagg acaccctgat gatctctagg
acccccgaag tcacttgcgt cgtcgtcgac gtgtcccacg aggaccctga agtcaagttc
aactggtacg tcgacggtgt cgaagtccac aacgccaaga ccaagcccag ggaagaacag
tacaactcta cctaccgcgt cgtcagcgtc ctgaccgtcc tgcaccagga ctggctgaac
ggaaaggaat acaagtgcaa ggtgtccaac aaggccctgc ctgcccccat cgaaaagacc
atctctaagg ccaagggaca gccccgcgaa ccccaggtct acaccctgcc accctctagg
gaagaaatga ccaagaacca ggtgtccctg acctgcctgg tcaagggatt ctacccctct
gacatcgccg tcgaatggga atctaacgga cagcccgaaa acaactacaa gaccaccccc
cctgtcctgg actctgacgg atcattcttc ctgtactcta agctgactgt cgacaagtct
aggtggcagc agggaaacgt gttctcttgc tctgtcatgc acgaagccct gcacaaccac
tacacccaga agtctctgtc tctgtccccc ggaaag
Soluble NPP1-(GLK)-Fc fusion protein sequence
SEQ ID NO: 91
Gly Leu Lys Pro Ser Cys Ala Lys Glu Val Lys Ser Cys Lys Gly Arg
Cys Phe Glu Arg Thr Phe Gly Asn Cys Arg Cys Asp Ala Ala Cys Val
Glu Leu Gly Asn Cys Cys Leu Asp Tyr Gln Glu Thr Cys Ile Glu Pro
Glu His Ile Trp Thr Cys Asn Lys Phe Arg Cys Gly Glu Lys Arg Leu
Thr Arg Ser Leu Cys Ala Cys Ser Asp Asp Cys Lys Asp Lys Gly Asp
Cys Cys Ile Asn Tyr Ser Ser Val Cys Gln Gly Glu Lys Ser Trp Val
Glu Glu Pro Cys Glu Ser Ile Asn Glu Pro Gln Cys Pro Ala Gly Phe
Glu Thr Pro Pro Thr Leu Leu Phe Ser Leu Asp Gly Phe Arg Ala Glu
Tyr Leu His Thr Trp Gly Gly Leu Leu Pro Val Ile Ser Lys Leu Lys
Lys Cys Gly Thr Tyr Thr Lys Asn Met Arg Pro Val Tyr Pro Thr Lys
Thr Phe Pro Asn His Tyr Ser Ile Val Thr Gly Leu Tyr Pro Glu Ser
His Gly Ile Ile Asp Asn Lys Met Tyr Asp Pro Lys Met Asn Ala Ser
Phe Ser Leu Lys Ser Lys Glu Lys Phe Asn Pro Glu Trp Tyr Lys Gly
Glu Pro Ile Trp Val Thr Ala Lys Tyr Gln Gly Leu Lys Ser Gly Thr
Phe Phe Trp Pro Gly Ser Asp Val Glu Ile Asn Gly Ile Phe Pro Asp
Ile Tyr Lys Met Tyr Asn Gly Ser Val Pro Phe Glu Glu Arg Ile Leu
Ala Val Leu Gln Trp Leu Gln Leu Pro Lys Asp Glu Arg Pro His Phe
Tyr Thr Leu Tyr Leu Glu Glu Pro Asp Ser Ser Gly His Ser Tyr Gly
Pro Val Ser Ser Glu Val Ile Lys Ala Leu Gln Arg Val Asp Gly Met
Val Gly Met Leu Met Asp Gly Leu Lys Glu Leu Asn Leu His Arg Cys
Leu Asn Leu Ile Leu Ile Ser Asp His Gly Met Glu Gln Gly Ser Cys
Lys Lys Tyr Ile Tyr Leu Asn Lys Tyr Leu Gly Asp Val Lys Asn Ile
Lys Val Ile Tyr Gly Pro Ala Ala Arg Leu Arg Pro Ser Asp Val Pro
Asp Lys Tyr Tyr Ser Phe Asn Tyr Glu Gly Ile Ala Arg Asn Leu Ser
Cys Arg Glu Pro Asn Gln His Phe Lys Pro Tyr Leu Lys His Phe Leu
Pro Lys Arg Leu His Phe Ala Lys Ser Asp Arg Ile Glu Pro Leu Thr
Phe Tyr Leu Asp Pro Gln Trp Gln Leu Ala Leu Asn Pro Ser Glu Arg
Lys Tyr Cys Gly Ser Gly Phe His Gly Ser Asp Asn Val Phe Ser Asn
Met Gln Ala Leu Phe Val Gly Tyr Gly Pro Gly Phe Lys His Gly Ile
Glu Ala Asp Thr Phe Glu Asn Ile Glu Val Tyr Asn Leu Met Cys Asp
Leu Leu Asn Leu Thr Pro Ala Pro Asn Asn Gly Thr His Gly Ser Leu
Asn His Leu Leu Lys Asn Pro Val Tyr Thr Pro Lys His Pro Lys Glu
Val His Pro Leu Val Gln Cys Pro Phe Thr Arg Asn Pro Arg Asp Asn
Leu Gly Cys Ser Cys Asn Pro Ser Ile Leu Pro Ile Glu Asp Phe Gln
Thr Gln Phe Asn Leu Thr Val Ala Glu Glu Lys Ile Ile Lys His Glu
Thr Leu Pro Tyr Gly Arg Pro Arg Val Leu Gln Lys Glu Asn Thr Ile
Cys Leu Leu Ser Gln His Gln Phe Met Ser Gly Tyr Ser Gln Asp Ile
Leu Met Pro Leu Trp Thr Ser Tyr Thr Val Asp Arg Asn Asp Ser Phe
Ser Thr Glu Asp Phe Ser Asn Cys Leu Tyr Gln Asp Phe Arg Ile Pro
Leu Ser Pro Val His Lys Cys Ser Phe Tyr Lys Asn Asn Thr Lys Val
Ser Tyr Gly Phe Leu Ser Pro Pro Gln Leu Asn Lys Asn Ser Ser Gly
Ile Tyr Ser Glu Ala Leu Leu Thr Thr Asn Ile Val Pro Met Tyr Gln
Ser Phe Gln Val Ile Trp Arg Tyr Phe His Asp Thr Leu Leu Arg Lys
Tyr Ala Glu Glu Arg Asn Gly Val Asn Val Val Ser Gly Pro Val Phe
Asp Phe Asp Tyr Asp Gly Arg Cys Asp Ser Leu Glu Asn Leu Arg Gln
Lys Arg Arg Val Ile Arg Asn Gln Glu Ile Leu Ile Pro Thr His Phe
Phe Ile Val Leu Thr Ser Cys Lys Asp Thr Ser Gln Thr Pro Leu His
Cys Glu Asn Leu Asp Thr Leu Ala Phe Ile Leu Pro His Arg Thr Asp
Asn Ser Glu Ser Cys Val His Gly Lys His Asp Ser Ser Trp Val Glu
Glu Leu Leu Met Leu His Arg Ala Arg Ile Thr Asp Val Glu His Ile
Thr Gly Leu Ser Phe Tyr Gln Gln Arg Lys Glu Pro Val Ser Asp Ile
Leu Lys Leu Lys Thr His Leu Pro Thr Phe Ser Gln Glu Asp Leu Ile
Asn Asp Lys Thr His Thr Cys Pro Pro Cys Pro Ala Pro Glu Leu Leu
Gly Gly Pro Ser Val Phe Leu Phe Pro Pro Lys Pro Lys Asp Thr Leu
Met Ile Ser Arg Thr Pro Glu Val Thr Cys Val Val Val Asp Val Ser
His Glu Asp Pro Glu Val Lys Phe Asn Trp Tyr Val Asp Gly Val Glu
Val His Asn Ala Lys Thr Lys Pro Arg Glu Glu Gln Tyr Asn Ser Thr
Tyr Arg Val Val Ser Val Leu Thr Val Leu His Gln Asp Trp Leu Asn
Gly Lys Glu Tyr Lys Cys Lys Val Ser Asn Lys Ala Leu Pro Ala Pro
Ile Glu Lys Thr Ile Ser Lys Ala Lys Gly Gln Pro Arg Glu Pro Gln
Val Tyr Thr Leu Pro Pro Ser Arg Glu Glu Met Thr Lys Asn Gln Val
Ser Leu Thr Cys Leu Val Lys Gly Phe Tyr Pro Ser Asp Ile Ala Val
Glu Trp Glu Ser Asn Gly Gln Pro Glu Asn Asn Tyr Lys Thr Thr Pro
Pro Val Leu Asp Ser Asp Gly Ser Phe Phe Leu Tyr Ser Lys Leu Thr
Val Asp Lys Ser Arg Trp Gln Gln Gly Asn Val Phe Ser Cys Ser Val
Met His Glu Ala Leu His Asn His Tyr Thr Gln Lys Ser Leu Ser Leu
Ser Pro Gly Lys
double-underlined: beginning and end of NPP1; bold residues
indicate Fc sequence
Soluble NPP1-Fc fusion protein sequence
SEQ ID NO: 92
Pro Ser Cys Ala Lys Glu Val Lys Ser Cys Lys Gly Arg Cys Phe Glu
Arg Thr Phe Gly Asn Cys Arg Cys Asp Ala Ala Cys Val Glu Leu Gly
Asn Cys Cys Leu Asp Tyr Gln Glu Thr Cys Ile Glu Pro Glu His Ile
Trp Thr Cys Asn Lys Phe Arg Cys Gly Glu Lys Arg Leu Thr Arg Ser
Leu Cys Ala Cys Ser Asp Asp Cys Lys Asp Lys Gly Asp Cys Cys Ile
Asn Tyr Ser Ser Val Cys Gln Gly Glu Lys Ser Trp Val Glu Glu Pro
Cys Glu Ser Ile Asn Glu Pro Gln Cys Pro Ala Gly Phe Glu Thr Pro
Pro Thr Leu Leu Phe Ser Leu Asp Gly Phe Arg Ala Glu Tyr Leu His
Thr Trp Gly Gly Leu Leu Pro Val Ile Ser Lys Leu Lys Lys Cys Gly
Thr Tyr Thr Lys Asn Met Arg Pro Val Tyr Pro Thr Lys Thr Phe Pro
Asn His Tyr Ser Ile Val Thr Gly Leu Tyr Pro Glu Ser His Gly Ile
Ile Asp Asn Lys Met Tyr Asp Pro Lys Met Asn Ala Ser Phe Ser Leu
Lys Ser Lys Glu Lys Phe Asn Pro Glu Trp Tyr Lys Gly Glu Pro Ile
Trp Val Thr Ala Lys Tyr Gln Gly Leu Lys Ser Gly Thr Phe Phe Trp
Pro Gly Ser Asp Val Glu Ile Asn Gly Ile Phe Pro Asp Ile Tyr Lys
Met Tyr Asn Gly Ser Val Pro Phe Glu Glu Arg Ile Leu Ala Val Leu
Gln Trp Leu Gln Leu Pro Lys Asp Glu Arg Pro His Phe Tyr Thr Leu
Tyr Leu Glu Glu Pro Asp Ser Ser Gly His Ser Tyr Gly Pro Val Ser
Ser Glu Val Ile Lys Ala Leu Gln Arg Val Asp Gly Met Val Gly Met
Leu Met Asp Gly Leu Lys Glu Leu Asn Leu His Arg Cys Leu Asn Leu
Ile Leu Ile Ser Asp His Gly Met Glu Gln Gly Ser Cys Lys Lys Tyr
Ile Tyr Leu Asn Lys Tyr Leu Gly Asp Val Lys Asn Ile Lys Val Ile
Tyr Gly Pro Ala Ala Arg Leu Arg Pro Ser Asp Val Pro Asp Lys Tyr
Tyr Ser Phe Asn Tyr Glu Gly Ile Ala Arg Asn Leu Ser Cys Arg Glu
Pro Asn Gln His Phe Lys Pro Tyr Leu Lys His Phe Leu Pro Lys Arg
Leu His Phe Ala Lys Ser Asp Arg Ile Glu Pro Leu Thr Phe Tyr Leu
Asp Pro Gln Trp Gln Leu Ala Leu Asn Pro Ser Glu Arg Lys Tyr Cys
Gly Ser Gly Phe His Gly Ser Asp Asn Val Phe Ser Asn Met Gln Ala
Leu Phe Val Gly Tyr Gly Pro Gly Phe Lys His Gly Ile Glu Ala Asp
Thr Phe Glu Asn Ile Glu Val Tyr Asn Leu Met Cys Asp Leu Leu Asn
Leu Thr Pro Ala Pro Asn Asn Gly Thr His Gly Ser Leu Asn His Leu
Leu Lys Asn Pro Val Tyr Thr Pro Lys His Pro Lys Glu Val His Pro
Leu Val Gln Cys Pro Phe Thr Arg Asn Pro Arg Asp Asn Leu Gly Cys
Ser Cys Asn Pro Ser Ile Leu Pro Ile Glu Asp Phe Gln Thr Gln Phe
Asn Leu Thr Val Ala Glu Glu Lys Ile Ile Lys His Glu Thr Leu Pro
Tyr Gly Arg Pro Arg Val Leu Gln Lys Glu Asn Thr Ile Cys Leu Leu
Ser Gln His Gln Phe Met Ser Gly Tyr Ser Gln Asp Ile Leu Met Pro
Leu Trp Thr Ser Tyr Thr Val Asp Arg Asn Asp Ser Phe Ser Thr Glu
Asp Phe Ser Asn Cys Leu Tyr Gln Asp Phe Arg Ile Pro Leu Ser Pro
Val His Lys Cys Ser Phe Tyr Lys Asn Asn Thr Lys Val Ser Tyr Gly
Phe Leu Ser Pro Pro Gln Leu Asn Lys Asn Ser Ser Gly Ile Tyr Ser
Glu Ala Leu Leu Thr Thr Asn Ile Val Pro Met Tyr Gln Ser Phe Gln
Val Ile Trp Arg Tyr Phe His Asp Thr Leu Leu Arg Lys Tyr Ala Glu
Glu Arg Asn Gly Val Asn Val Val Ser Gly Pro Val Phe Asp Phe Asp
Tyr Asp Gly Arg Cys Asp Ser Leu Glu Asn Leu Arg Gln Lys Arg Arg
Val Ile Arg Asn Gln Glu Ile Leu Ile Pro Thr His Phe Phe Ile Val
Leu Thr Ser Cys Lys Asp Thr Ser Gln Thr Pro Leu His Cys Glu Asn
Leu Asp Thr Leu Ala Phe Ile Leu Pro His Arg Thr Asp Asn Ser Glu
Ser Cys Val His Gly Lys His Asp Ser Ser Trp Val Glu Glu Leu Leu
Met Leu His Arg Ala Arg Ile Thr Asp Val Glu His Ile Thr Gly Leu
Ser Phe Tyr Gln Gln Arg Lys Glu Pro Val Ser Asp Ile Leu Lys Leu
Lys Thr His Leu Pro Thr Phe Ser Gln Glu Asp Leu Ile Asn Asp Lys
Thr His Thr Cys Pro Pro Cys Pro Ala Pro Glu Leu Leu Gly Gly Pro
Ser Val Phe Leu Phe Pro Pro Lys Pro Lys Asp Thr Leu Met Ile Ser
Arg Thr Pro Glu Val Thr Cys Val Val Val Asp Val Ser His Glu Asp
Pro Glu Val Lys Phe Asn Trp Tyr Val Asp Gly Val Glu Val His Asn
Ala Lys Thr Lys Pro Arg Glu Glu Gln Tyr Asn Ser Thr Tyr Arg Val
Val Ser Val Leu Thr Val Leu His Gln Asp Trp Leu Asn Gly Lys Glu
Tyr Lys Cys Lys Val Ser Asn Lys Ala Leu Pro Ala Pro Ile Glu Lys
Thr Ile Ser Lys Ala Lys Gly Gln Pro Arg Glu Pro Gln Val Tyr Thr
Leu Pro Pro Ser Arg Glu Glu Met Thr Lys Asn Gln Val Ser Leu Thr
Cys Leu Val Lys Gly Phe Tyr Pro Ser Asp Ile Ala Val Glu Trp Glu
Ser Asn Gly Gln Pro Glu Asn Asn Tyr Lys Thr Thr Pro Pro Val Leu
Asp Ser Asp Gly Ser Phe Phe Leu Tyr Ser Lys Leu Thr Val Asp Lys
Ser Arg Trp Gin Gin Gly Asn Val Phe Ser Cys Ser Val Met His Glu
Ala Leu His Asn His Tyr Thr Gin Lys Ser Leu Ser Leu Ser Pro Gly
Lys
double-underlined: beginning and end of NPP1; bold residues
indicate Fc sequence
Soluble NPP1-Fc fusion protein sequence
SEQ ID NO: 93
Ala Pro Ser Cys Ala Lys Glu Val Lys Ser Cys Lys Gly Arg Cys Phe
Glu Arg Thr Phe Gly Asn Cys Arg Cys Asp Ala Ala Cys Val Glu Leu
Gly Asn Cys Cys Leu Asp Tyr Gln Glu Thr Cys Ile Glu Pro Glu His
Ile Trp Thr Cys Asn Lys Phe Arg Cys Gly Glu Lys Arg Leu Thr Arg
Ser Leu Cys Ala Cys Ser Asp Asp Cys Lys Asp Lys Gly Asp Cys Cys
Ile Asn Tyr Ser Ser Val Cys Gln Gly Glu Lys Ser Trp Val Glu Glu
Pro Cys Glu Ser Ile Asn Glu Pro Gln Cys Pro Ala Gly Phe Glu Thr
Pro Pro Thr Leu Leu Phe Ser Leu Asp Gly Phe Arg Ala Glu Tyr Leu
His Thr Trp Gly Gly Leu Leu Pro Val Ile Ser Lys Leu Lys Lys Cys
Gly Thr Tyr Thr Lys Asn Met Arg Pro Val Tyr Pro Thr Lys Thr Phe
Pro Asn His Tyr Ser Ile Val Thr Gly Leu Tyr Pro Glu Ser His Gly
Ile Ile Asp Asn Lys Met Tyr Asp Pro Lys Met Asn Ala Ser Phe Ser
Leu Lys Ser Lys Glu Lys Phe Asn Pro Glu Trp Tyr Lys Gly Glu Pro
Ile Trp Val Thr Ala Lys Tyr Gln Gly Leu Lys Ser Gly Thr Phe Phe
Trp Pro Gly Ser Asp Val Glu Ile Asn Gly Ile Phe Pro Asp Ile Tyr
Lys Met Tyr Asn Gly Ser Val Pro Phe Glu Glu Arg Ile Leu Ala Val
Leu Gln Trp Leu Gln Leu Pro Lys Asp Glu Arg Pro His Phe Tyr Thr
Leu Tyr Leu Glu Glu Pro Asp Ser Ser Gly His Ser Tyr Gly Pro Val
Ser Ser Glu Val Ile Lys Ala Leu Gln Arg Val Asp Gly Met Val Gly
Met Leu Met Asp Gly Leu Lys Glu Leu Asn Leu His Arg Cys Leu Asn
Leu Ile Leu Ile Ser Asp His Gly Met Glu Gln Gly Ser Cys Lys Lys
Tyr Ile Tyr Leu Asn Lys Tyr Leu Gly Asp Val Lys Asn Ile Lys Val
Ile Tyr Gly Pro Ala Ala Arg Leu Arg Pro Ser Asp Val Pro Asp Lys
Tyr Tyr Ser Phe Asn Tyr Glu Gly Ile Ala Arg Asn Leu Ser Cys Arg
Glu Pro Asn Gln His Phe Lys Pro Tyr Leu Lys His Phe Leu Pro Lys
Arg Leu His Phe Ala Lys Ser Asp Arg Ile Glu Pro Leu Thr Phe Tyr
Leu Asp Pro Gln Trp Gln Leu Ala Leu Asn Pro Ser Glu Arg Lys Tyr
Cys Gly Ser Gly Phe His Gly Ser Asp Asn Val Phe Ser Asn Met Gln
Ala Leu Phe Val Gly Tyr Gly Pro Gly Phe Lys His Gly Ile Glu Ala
Asp Thr Phe Glu Asn Ile Glu Val Tyr Asn Leu Met Cys Asp Leu Leu
Asn Leu Thr Pro Ala Pro Asn Asn Gly Thr His Gly Ser Leu Asn His
Leu Leu Lys Asn Pro Val Tyr Thr Pro Lys His Pro Lys Glu Val His
Pro Leu Val Gln Cys Pro Phe Thr Arg Asn Pro Arg Asp Asn Leu Gly
Cys Ser Cys Asn Pro Ser Ile Leu Pro Ile Glu Asp Phe Gln Thr Gln
Phe Asn Leu Thr Val Ala Glu Glu Lys Ile Ile Lys His Glu Thr Leu
Pro Tyr Gly Arg Pro Arg Val Leu Gln Lys Glu Asn Thr Ile Cys Leu
Leu Ser Gln His Gln Phe Met Ser Gly Tyr Ser Gln Asp Ile Leu Met
Pro Leu Trp Thr Ser Tyr Thr Val Asp Arg Asn Asp Ser Phe Ser Thr
Glu Asp Phe Ser Asn Cys Leu Tyr Gln Asp Phe Arg Ile Pro Leu Ser
Pro Val His Lys Cys Ser Phe Tyr Lys Asn Asn Thr Lys Val Ser Tyr
Gly Phe Leu Ser Pro Pro Gln Leu Asn Lys Asn Ser Ser Gly Ile Tyr
Ser Glu Ala Leu Leu Thr Thr Asn Ile Val Pro Met Tyr Gln Ser Phe
Gln Val Ile Trp Arg Tyr Phe His Asp Thr Leu Leu Arg Lys Tyr Ala
Glu Glu Arg Asn Gly Val Asn Val Val Ser Gly Pro Val Phe Asp Phe
Asp Tyr Asp Gly Arg Cys Asp Ser Leu Glu Asn Leu Arg Gln Lys Arg
Arg Val Ile Arg Asn Gln Glu Ile Leu Ile Pro Thr His Phe Phe Ile
Val Leu Thr Ser Cys Lys Asp Thr Ser Gln Thr Pro Leu His Cys Glu
Asn Leu Asp Thr Leu Ala Phe Ile Leu Pro His Arg Thr Asp Asn Ser
Glu Ser Cys Val His Gly Lys His Asp Ser Ser Trp Val Glu Glu Leu
Leu Met Leu His Arg Ala Arg Ile Thr Asp Val Glu His Ile Thr Gly
Leu Ser Phe Tyr Gln Gln Arg Lys Glu Pro Val Ser Asp Ile Leu Lys
Leu Lys Thr His Leu Pro Thr Phe Ser Gln Glu Asp Leu Ile Asn Asp
Lys Thr His Thr Cys Pro Pro Cys Pro Ala Pro Glu Leu Leu Gly Gly
Pro Ser Val Phe Leu Phe Pro Pro Lys Pro Lys Asp Thr Leu Met Ile
Ser Arg Thr Pro Glu Val Thr Cys Val Val Val Asp Val Ser His Glu
Asp Pro Glu Val Lys Phe Asn Trp Tyr Val Asp Gly Val Glu Val His
Asn Ala Lys Thr Lys Pro Arg Glu Glu Gln Tyr Asn Ser Thr Tyr Arg
Val Val Ser Val Leu Thr Val Leu His Gln Asp Trp Leu Asn Gly Lys
Glu Tyr Lys Cys Lys Val Ser Asn Lys Ala Leu Pro Ala Pro Ile Glu
Lys Thr Ile Ser Lys Ala Lys Gly Gln Pro Arg Glu Pro Gln Val Tyr
Thr Leu Pro Pro Ser Arg Glu Glu Met Thr Lys Asn Gln Val Ser Leu
Thr Cys Leu Val Lys Gly Phe Tyr Pro Ser Asp Ile Ala Val Glu Trp
Glu Ser Asn Gly Gln Pro Glu Asn Asn Tyr Lys Thr Thr Pro Pro Val
Leu Asp Ser Asp Gly Ser Phe Phe Leu Tyr Ser Lys Leu Thr Val Asp
Lys Ser Arg Trp Gln Gln Gly Asn Val Phe Ser Cys Ser Val Met His
Glu Ala Leu His Asn His Tyr Thr Gln Lys Ser Leu Ser Leu Ser Pro
Gly Lys
double-underlined: beginning and end of NPP1; bold residues
indicate Fc sequence
Linker
SEQ ID NO: 94
Gly Gly Gly Gly Ser

Pharmaceutical Compositions According to the Invention

The AAV vector according to the invention can be administered to the human or animal body by conventional methods, which require the formulation of said vectors in a pharmaceutical composition. In one embodiment, the invention relates to a pharmaceutical composition (hereinafter referred to as “pharmaceutical composition according to the invention”) comprising an AAV vector comprises a recombinant viral genome wherein said recombinant viral genome comprises an expression cassette comprising a transcriptional regulatory region operatively linked to a nucleotide sequence encoding ENPP1 or ENPP3 or a functionally equivalent variant thereof.

All the embodiments disclosed in the context of the adeno-associated viral vectors, Herpes simplex vectors, Adenoviral vectors, Alphaviral vectors and Lentiviral vectors according to the invention are also applicable to the pharmaceutical compositions according to the invention.

In some embodiments the pharmaceutical composition may include a therapeutically effective quantity of the AAV vector according to the invention and a pharmaceutically acceptable carrier. In some embodiments the pharmaceutical composition may include a therapeutically effective quantity of the adenoviral vector according to the invention and a pharmaceutically acceptable carrier.

In some embodiments the pharmaceutical composition may include a therapeutically effective quantity of the lentiviral vector according to the invention and a pharmaceutically acceptable carrier.

In some embodiments the pharmaceutical composition may include a therapeutically effective quantity of the alphaviral vector according to the invention and a pharmaceutically acceptable carrier.

In some embodiments the pharmaceutical composition may include a therapeutically effective quantity of the Herpes simplex viral vector according to the invention and a pharmaceutically acceptable carrier.

The term “therapeutically effective quantity” refers to the quantity of the AAV vector according to the invention calculated to produce the desired effect and will generally be determined, among other reasons, by the own features of the viral vector according to the invention and the therapeutic effect to be obtained. The quantity of the viral vector according to the invention that will be effective in the treatment of a disease can be determined by standard clinical techniques described herein or otherwise known in the art. Furthermore, in vitro tests can also be optionally used to help identify optimum dosage ranges. The precise dose to use in the formulation will depend on the administration route, and the severity of the condition, and it should be decided at the doctor's judgment and depending on each patient's circumstances.

Promoters

Vectors used in gene therapy require an expression cassette. The expression cassette consists of three important components: promoter, therapeutic gene and polyadenylation signal. The promoter is essential to control expression of the therapeutic gene. A tissue-specific promoter is a promoter that has activity in only certain cell types. Use of a tissue-specific promoter in the expression cassette can restrict unwanted transgene expression as well as facilitate persistent transgene expression. Commonly used promoters for gene therapy include cytomegalovirus immediate early (CMV-IE) promoter, Rous sarcoma virus long terminal repeat (RSV-LTR), Moloney murine leukaemia virus (MoMLV) LTR, and other retroviral LTR promoters. Eukaryotic promoters can be used for gene therapy, common examples for Eukaryotic promoters include human al-antitrypsin (hAAT) and murine RNA polymerase II (large subunit) promoters. Non Tissue specific promoters such as small nuclear RNA U1b promoter, EF1α promoter, and PGK1 promoter are also available for use in gene therapy. Tissue specific promoters such as Apo A-I, ApoE and a1-antitrypsin (hAAT) enable tissue specific expression of protein of interest in gene therapy. Table I of Papadakis et al. (Promoters and Control Elements: Designing Expression Cassettes for Gene Therapy, Current Gene Therapy, 2004, 4, 89-113) lists examples of transcriptional targeting using eukaryotic promoters in gene therapy, all of which are incorporated by reference in their entirety herein.

Dosage and Mode of Administration

AAV titers are given as a “physical” titer in vector or viral genomes per ml (vg/ml) or (vg/kg) vector or viral genomes per kilogram dosage. QPCR of purified vector particles can be used to determine the titer. One method for performing AAV VG number titration is as follows: purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are quantitated by real-time PCR using primer/probe sets targeting specific region of the viral genome.

A viral composition can be formulated in a dosage unit to contain an amount of a viral vector that is in the range of about 1.0×10⁹ vg/kg to about 1.0×10¹⁵ vg/kg and preferably 1.0×10¹² vg/kg to 1.0×10¹⁴ vg/kg for a human patient. Preferably, the dose of virus in the formulation is 1.0×10⁹ vg/kg, 5.0×10⁹ vg/kg, 1.0x 10¹⁰ vg/kg, 5.0×10¹⁰ vg/kg, 1.0×10¹¹ vg/kg, 5.0×10¹¹ vg/kg, 1.0×10¹² vg/kg, 5.0×10¹²vg/kg, or 1.0×10¹³ vg/kg, 5.0×10¹³ vg/kg, 1.0×10¹⁴ vg/kg, 5.0×10¹⁴ vg/kg, or 1.0×10¹⁵ vg/kg or 5.0×10¹⁵ vg/kg

In some embodiments, the dose administered to a mammal, particularly a human, in the context according to the invention varies with the particular viral vector, the composition containing the vector and the carrier therefor (as discussed above), and the mode of administration. The dose is sufficient to effect a desirable response, e.g., therapeutic or prophylactic response, within a desirable time frame. In terms of viral vector, the dose can be up to a maximum of 1×10¹⁵vg/kg.

The vectors of the present invention permit long term gene expression, resulting in long term effects of a therapeutic protein. The phrases “long term expression”, “sustained expression” and “persistent expression” are used interchangeably. Long term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably, long term expression means expression for at least 90 days, at least 120 days, at least 180 days, at least 250 days, at least 360 days, at least 450 days, at least 720 days or more, more preferably, at least 360 days, at least 450 days, at least 720 days or more. This long-term expression may be achieved by repeated doses (if possible) or by a single dose

Repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

A pharmaceutical composition according to the invention may be administered locally or systemically, intramuscularly, intravenously and parenterally. Delivery of therapeutic compositions according to the invention can be directed to central nervous system, cardiac system, and pulmonary system. A common delivery strategy is direct intramuscular injections. As a non-limiting example, Skeletal muscle has been shown to be a target tissue type that is efficiently transduced. Once transduced, the muscle cells serve as a production site for protein products that can act locally or systemically by many AAV variants.

In an embodiment, the pharmaceutical composition is administered near the tissue or organ whose cells are to be transduced. In a particular embodiment, the pharmaceutical composition according to the invention is administered locally in liver by injection into the liver parenchyma. In another embodiment, the pharmaceutical composition according to the invention is administered systemically.

As a non-limiting example, Systemic administration includes a systemic injection of the AAV vectors according to the invention, such as intramuscular (im), intravascular (ie), intra-arterial (ia), intravenous (iv), intraperitoneal (ip), or sub-cutaneous injections. Preferably, the systemic administration is via im, ip, is or iv injection. In some embodiments, the AAV vectors according to the invention are administered via intravenous injection.

In another embodiment the pharmaceutical compositions according to the invention are delivered to the liver of the subject. Administration to the liver is achieved using methods known in the art, including, but not limited to intravenous administration, intraportal administration, intrabiliary administration, intra-arterial administration, and direct injection into the liver parenchyma. In another embodiment, the pharmaceutical composition is administered intravenously.

A pharmaceutical composition according to the invention may be administered in a single dose or, in particular embodiments according to the invention, multiple doses (e.g. two, three, four, or more administrations) may be employed to achieve a therapeutic effect. Preferably, the AAV vector comprised in the pharmaceutical composition according to the invention are from different serotypes when multiple doses are required to obviate the effects of neutralizing antibodies.

Formulations

The preparations may also contain buffer salts. Alternatively, the compositions may be in powder form for constitution with a suitable vehicle (e.g. sterile pyrogen-free water) before use. When necessary, the composition may also include a local anaesthetic such as lidocaine to relieve pain at the injection site. When the composition is going to be administered by infiltration, it can be dispensed with an infiltration bottle which contains water or saline solution of pharmaceutical quality. When the composition is administered by injection, a water vial can be provided for injection or sterile saline solution, so that the ingredients can be mixed before administration. Preferably, the pharmaceutically acceptable carrier is saline solution and a detergent such as Pluronic®.

Compositions according to the invention may be formulated for delivery to animals for veterinary purposes (e.g. livestock (cattle, pigs, others)), and other non-human mammalian subjects, as well as to human subjects. The AAV vector can be formulated with a physiologically acceptable carrier for use in gene transfer and gene therapy applications. As a non-limiting example, also encompassed is the use of adjuvants in combination with or in admixture with the AAV vector according to the invention. Adjuvants contemplated include, but are not limited to, mineral salt adjuvants or mineral salt gel adjuvants, particulate adjuvants, microparticulate adjuvants, mucosal adjuvants. Adjuvants can be administered to a subject as a mixture with the AAV vector according to the invention or used in combination said AAV vector.

The terms “pharmaceutically acceptable carrier,” “pharmaceutically acceptable diluent,” “pharmaceutically acceptable excipient”, or “pharmaceutically acceptable vehicle”, used interchangeably herein, refer to a non-toxic solid, semisolid, or liquid filler, diluent, encapsulating material, or formulation auxiliary of any conventional type. A pharmaceutically acceptable carrier is essentially non-toxic to recipients at the employed dosages and concentrations and is compatible with other ingredients of the formulation. The number and the nature of the pharmaceutically acceptable carriers depend on the desired administration form. The pharmaceutically acceptable carriers are known and may be prepared by methods well known in the art (Faith i Trillo C., “Tratado de Farmacia Galénica”. Ed. Luzán 5, S. A., Madrid, E S, 1993; Gennaro A, Ed., “Remington: The Science and Practice of Pharmacy” 20th ed. Lippincott Williams & Wilkins, Philadelphia, Pa., US, 2003).

As a non-limiting example, the AAV vector may be formulated for parenteral administration by injection (e.g. by bolus injection or continuous infusion). Formulations for injection may be presented in unit dosage form (e.g. in ampoules or in multi-dose containers) with an added preservative. The viral compositions may take such forms as suspensions, solutions, or emulsions in oily or aqueous vehicles, and may contain formulatory agents such as suspending, stabilizing, or dispersing agents. Liquid preparations of the AAV formulations may be prepared by conventional means with pharmaceutically acceptable additives such as suspending agents (e.g. sorbitol syrup, cellulose derivatives or hydrogenated edible fats), emulsifying agents (e.g. lecithin or acacia), non-aqueous vehicles (e.g. almond oil, oily esters, ethyl alcohol or fractionated vegetable oils), and preservatives (e.g. methyl or propyl-p-hydroxybenzoates or sorbic acid).

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described.

In addition, the composition can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the vector and physiological distress. Immune system suppressors can be administered with the composition method to reduce any immune response to the vector itself or associated with a disorder. Administration of immunosuppressive medications or immunosuppressants is the main method of deliberately induced immunosuppression, in optimal circumstances, immunosuppressive drugs are targeted only at any hyperactive component of the immune system.

Immunosuppressive drugs or immunosuppressive agents or antirejection medications are drugs that inhibit or prevent activity of the immune system. Such drugs include glucocorticoids, cytostatics, antibodies, drugs acting on immunophilins. In pharmacologic (supraphysiologic) doses, glucocorticoids, such as prednisone, dexamethasone, and hydrocortisone are used to suppress various allergic and inflammatory responses. Cytostatics, such as purine analogs, alkylating agents, such as nitrogen mustards (cyclophosphamide), nitrosoureas, platinum compounds, and others. Cyclophosphamide (Baxter's Cytoxan) is probably the most potent immunosuppressive compound. Antimetabolites, for example, folic acid analogues, such as methotrexate, purine analogues, such as azathioprine and mercaptopurine, pyrimidine analogues, such as fluorouracil, and protein synthesis inhibitors. Cytotoxic antibiotics Among these, dactinomycin is the most important. It is used in kidney transplantations. Other cytotoxic antibiotics are anthracyclines, mitomycin C, bleomycin, mithramycin. Antibodies are sometimes used as a quick and potent immunosuppressive therapy to prevent the acute rejection reactions (e.g., anti-CD20 monoclonals).

Alternatively, immune enhancers can be included in the composition to upregulate the body's natural defenses against disease.

Antibiotics, i.e., microbicides and fungicides, can be present to reduce the risk of infection associated with gene transfer procedures and other disorders.

The pharmaceutical composition can be formulated in accordance with routine procedures as a pharmaceutical composition adapted for intravenous, subcutaneous, or intramuscular administration to human beings.

Therapeutic Methods According to the Invention

As a non-limiting example, a viral vector encoding human ENPP1 or ENPP3 is administered to a mammal, resulting in delivery of DNA encoding ENPP1 or ENPP3 and expression of the protein in the mammal, thereby restoring a level of ENPP1 or ENPP3 required to reduce calcification or ossification in soft tissues.

In one aspect, the invention relates to an adeno-associated viral vector comprising a recombinant viral genome wherein said recombinant viral genome comprises an expression cassette comprising a transcriptional regulatory region operatively linked to a nucleotide sequence encoding ENPP1 or ENPP3 or a functionally equivalent variant thereof or a pharmaceutical composition comprising said viral vector for use in the treatment and/or prevention of a disease of pathological calcification or ossification.

In another aspect, the invention relates to the use of an adeno-associated viral vector comprising a recombinant viral genome wherein said recombinant viral genome comprises an expression cassette comprising a transcriptional regulatory region operatively linked to a nucleotide sequence encoding ENPP 1 or ENPP3 or a functionally equivalent variant thereof or a pharmaceutical composition comprising said viral vector for the manufacture of a medicament for the treatment and/or prevention of a disease a disease of pathological calcification or ossification.

In another aspect, the invention provides a method for the treatment and/or prevention of a disease of pathological calcification or ossification in a subject in need thereof which comprises the administration to said subject of an adeno-associated viral vector comprising a recombinant viral genome wherein said recombinant viral genome comprises an expression cassette comprising a transcriptional regulatory region operatively linked to a nucleotide sequence encoding ENPP1 or ENPP3 or a functionally equivalent variant thereof or a pharmaceutical composition comprising said viral vector.

In another aspect, the disease of pathological calcification or ossification being treated by the compositions and methods of this invention, are selected from the group consisting of X-linked hypophosphatemia (XLH), Chronic kidney disease (CKD), Mineral bone disorders (MBD), vascular calcification, pathological calcification of soft tissue, pathological ossification of soft tissue, Generalized arterial calcification of infants (GACI), Ossification of posterior longitudinal ligament (OPLL).

Polynucleotides, Vectors and Plasmids according to the Invention

The invention also relates to polynucleotides which are useful for producing the viral vectors, for example, AAV vectors according to the invention. In one embodiment, the invention relates to a polynucleotide (“polynucleotide according to the invention”) comprising an expression cassette flanked by adeno-associated virus ITRs wherein said expression cassette comprises a transcriptional regulatory region operatively linked to a nucleotide sequence encoding ENPP1 or ENPP3 or a functionally equivalent variant thereof.

In one embodiment the polynucleotide according to the invention comprises a transcriptional regulatory region that comprises a promoter; preferably a constitutive promoter; more preferably a liver-specific promoter; more preferably a liver-specific promoter selected from the group consisting of albumin promoter, phosphoenol pyruvate carboxykinase (PEPCK) promoter and alpha 1-antitrypsin promoter; the most preferred being the human alpha 1-antitrypsin promoter. In another embodiment, the transcriptional regulatory region of the polynucleotide according to the invention further comprises an enhancer operatively linked to the promoter, preferably a liver-specific enhancer, more preferably a hepatic control region enhancer (HCR).

In another embodiment, the expression cassette of the polynucleotide according to the invention further comprises a polyadenylation signal, more preferably the SV40polyA. In another embodiment the ENPP1 encoded by the polynucleotide according to the invention is selected from the group consisting of human ENPP1 and human ENPP3.

The polynucleotide according to the invention could be incorporated into a vector such as, for example, a plasmid. Thus, in another aspect, the invention relates to a vector or plasmid comprising the polynucleotide according to the invention. In a particular embodiment, the polynucleotide according to the invention is incorporated into an adeno-associated viral vector or plasmid.

Preferably, all other structural and non-structural coding sequences necessary for the production of adeno-associated virus are not present in the viral vector since they can be provided in trans by another vector, such as a plasmid, or by stably integrating the sequences into a packaging cell line.

Methods for Obtaining AAV According to the Invention

The invention also relates to a method for obtaining the viral vectors according to the invention, as a non-limiting example, AAV vector. Said AAV vectors can be obtained by introducing the polynucleotides according to the invention into cells that express the Rep and Cap proteins constitutively or wherein the Rep and Cap coding sequences are provided in plasmids or vectors. Thus, in another aspect, the invention relates to a method for obtaining an adeno-associated viral vector comprising the steps of:
(i) providing a cell comprising a polynucleotide according to the invention, AAV Cap proteins, AAV Rep proteins and, optionally, viral proteins upon which AAV is dependent for replication,
(ii) maintaining the cell under conditions adequate for assembly of the AAV and
(iii) purifying the adeno-associated viral vector produced by the cell.

The production of recombinant AAV (rAAV) for vectorizing transgenes have been described previously (Ayuso E, et al., Curr. Gene Ther. 2010, 10:423-436; Okada T, et al., Hum. Gene Ther. 2009, 20:1013-1021; Zhang H, et al., Hum. Gene Ther. 2009, 20:922-929; and Virag T, et al., Hum. Gene Ther. 2009, 20:807-817). These protocols can be used or adapted to generate the AAV according to the invention. Any cell capable of producing adeno-associated viral vectors can be used in the present invention including mammalian and insect cells.

In one embodiment, the producer cell line is transfected transiently with the polynucleotide according to the invention (comprising the expression cassette flanked by ITRs) and with construct(s) that encodes Rep and Cap proteins and provides helper functions. In another embodiment, the cell line supplies stably the helper functions and is transfected transiently with the polynucleotide according to the invention (comprising the expression cassette flanked by ITRs) and with construct(s) that encodes Rep and Cap proteins.

In another embodiment, the cell line supplies stably the Rep and Cap proteins and the helper functions and is transiently transfected with the polynucleotide according to the invention. In another embodiment, the cell line supplies stably the Rep and Cap proteins and is transfected transiently with the polynucleotide according to the invention and a polynucleotide encoding the helper functions. In yet another embodiment, the cell line supplies stably the polynucleotide according to the invention, the Rep and Cap proteins and the helper functions. Methods of making and using these and other AAV production systems have been described in the art.

In another embodiment, the producer cell line is an insect cell line (typically Sf9 cells) that is infected with baculovirus expression vectors that provide Rep and Cap proteins. This system does not require adenovirus helper genes (Ayuso E, et al., Curr. Gene Ther. 2010, 10:423-436).

In another embodiment, the transgene delivery capacity of AAV can be increased by providing AAV ITRs of two genomes that can anneal to form head to tail concatamers. Generally, upon entry of the AAV into the host cell, the single-stranded DNA containing the transgene is converted by the host cell DNA polymerase complexes into double-stranded DNA, after which the ITRs aid in concatamer formation in the nucleus. As an alternative, the AAV may be engineered to be a self-complementary (sc) AAV, which enables the viral vector to bypass the step of second-strand synthesis upon entry into a target cell, providing an scAAV viral vector with faster and, potentially, higher (e.g. up to 100-fold) transgene expression.

For example, the AAV may be engineered to have a genome comprising two connected single-stranded DNAs that encode, respectively, a transgene unit and its complement, which can snap together following delivery into a target cell, yielding a double-stranded DNA encoding the transgene unit of interest. Self-complementary AAV have been described in the art (Carter B, U.S. Pat. No. 6,596,535, Carter B, U.S. Pat. No. 7,125,717, and Takano H, et al., U.S. Pat. No. 7,456,683).

Preferably, all the structural and non-structural coding sequences (Cap proteins and Rep proteins) are not present in the AAV vector since they can be provided in trans by a vector, such as a plasmid. Cap proteins have been reported to have effects on host tropism, cell, tissue, or organ specificity, receptor use, infection efficiency, and immunogenicity of AAV viruses. Accordingly, an AAV Cap for use in an rAAV may be selected taking into consideration, for example, the subject's species (e.g. human or non-human), the subject's immunological state, the subject's suitability for long or short-term treatment, or a particular therapeutic application (e.g. treatment of a particular disease or disorder, or delivery to particular cells, tissues, or organs).

In another embodiment, the Cap protein is derived from the AAV of the group consisting of AAV2, AAV5, AAV7, AAV8, AAV9, AAV10 and AAVrh10 serotypes. In another embodiment, the Cap protein is derived from AAV8.

In some embodiments, an AAV Cap for use in the method according to the invention can be generated by mutagenesis (i.e. by insertions, deletions, or substitutions) of one of the aforementioned AAV Caps or its encoding nucleic acid. In some embodiments, the AAV Cap is at least 70%, 75%, 80%, 85%, 90%, 95%, 98%, or 99% or more similar to one or more of the aforementioned AAV Caps.

In some embodiments, the AAV Cap is chimeric, comprising domains from two, three, four, or more of the aforementioned AAV Caps. In some embodiments, the AAV Cap is a mosaic of VP1, VP2, and VP3 monomers originating from two or three different AAV or a recombinant AAV. In some embodiments, a rAAV composition comprises more than one of the aforementioned Caps.

In some embodiments, an AAV Cap for use in a rAAV composition is engineered to contain a heterologous sequence or other modification. For example, a peptide or protein sequence that confers selective targeting or immune evasion may be engineered into a Cap protein. Alternatively, or in addition, the Cap may be chemically modified so that the surface of the rAAV is polyethylene glycolated (i.e. pegylated), which may facilitate immune evasion. The Cap protein may also be mutagenized (e.g. to remove its natural receptor binding, or to mask an immunogenic epitope).

In some embodiments, an AAV Rep protein for use in the method according to the invention can be generated by mutagenesis (i.e. by insertions, deletions, or substitutions) of one of the aforementioned AAV Reps or its encoding nucleic acid. In some embodiments, the AAV Rep is at least 70%, 75%, 80%, 85%, 90%, 95%, 98%, or 99% or more similar to one or more of the aforementioned AAV Reps.

In another embodiment, the AAV Rep and Cap proteins derive from an AAV serotype selected from the group consisting of AAV2, AAV5, AAV7, AAV8, AAV9, AAV10 and AAVrh10.

In some embodiments, a viral protein upon which AAV is dependent for replication for use in the method according to the invention can be generated by mutagenesis (i.e. by insertions, deletions, or substitutions) of one of the aforementioned viral proteins or its encoding nucleic acid. In some embodiments, the viral protein is at least 70%, 75%, 80%, 85%, 90%, 95%, 98%, or 99% or more similar to one or more of the aforementioned viral proteins.

Methods for assaying the functions of Cap proteins, Rep proteins and viral proteins upon which AAV is dependent for replication are well known in the art. The genes AAV rep, AAV cap and genes providing helper functions can be introduced into the cell by incorporating said genes into a vector such as, for example, a plasmid, and introducing said vector into the cell. The genes can be incorporated into the same plasmid or into different plasmids. In another embodiment, the AAV rep and cap genes are incorporated into one plasmid and the genes providing helper functions are incorporated into another plasmid. Examples of plasmids comprising the AAV rep and cap genes suitable for use with the methods according to the invention include the pHLP19 and pRep6cap6 vectors (Colisi P, U.S. Pat. No. 6,001,650 and Russell D, et al., U.S. Pat. No. 6,156,303).

The polynucleotide according to the invention and the polynucleotides comprising AAV rep and cap genes or genes providing helper functions can be introduced into the cell by using any suitable method well known in the art. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. In a particular embodiment, the transfection is carried out by means of co-precipitation with calcium phosphate. When the cell lacks the expression of any of the AAV rep and cap genes and genes providing adenoviral helper functions, said genes can be introduced into the cell simultaneously with the polynucleotide according to the invention.

Alternatively, said genes can be introduced in the cell before or after the introduction of the polynucleotide according to the invention. In a particular embodiment, the cells are transfected simultaneously with three plasmids:

1) a plasmid comprising the polynucleotide according to the invention

2) a plasmid comprising the AAV rep and cap genes

3) a plasmid comprising the genes providing the helper functions.

Alternatively, the AAV rep and cap genes and genes providing helper functions may be carried by the packaging cell, either episomally and/or integrated into the genome of the packaging cell.

The invention encompasses methods that involve maintaining the cell under conditions adequate for assembly of the AAV. Methods of culturing packaging cells and exemplary conditions which promote the release of AAV vector particles, such as the producing of a cell lysate, may be carried out as described in examples herein. Producer cells are grown for a suitable period of time in order to promote the assembly of the AAV and the release of viral vectors into the media. Generally, cells may be grown for about 24 hours, about 36 hours, about 48 hours, about 72 hours, about 4 days, about 5 days, about 6 days, about 7 days, about 8 days, about 9 days, up to about 10 days. After about 10 days (or sooner, depending on the culture conditions and the particular producer cell used), the level of production generally decreases significantly. Generally, time of culture is measured from the point of viral production. For example, in the case of AAV, viral production generally begins upon supplying helper virus function in an appropriate producer cell as described herein. Generally, cells are harvested about 48 to about 100, preferably about 48 to about 96, preferably about 72 to about 96, preferably about 68 to about 72 hours after helper virus infection (or after viral production begins).

The invention encompasses methods of purifying the adeno-associated viral vector produced by the cell. The AAV according to the invention can be obtained from both: i) the cells transfected with the polynucleotides according to the invention and ii) the culture medium of said cells after a period of time post-transfection, preferably 72 hours. Any method for the purification of the AAV from said cells or said culture medium can be used for obtaining the AAV according to the invention. In a particular embodiment, the AAV according to the invention are purified following an optimized method based on a polyethylene glycol precipitation step and two consecutive cesium chloride (CsCl) gradients. Purified AAV according to the invention can be dialyzed against PBS, filtered and stored at −80° C. Titers of viral genomes can be determined by quantitative PCR following the protocol described for the AAV2 reference standard material using linearized plasmid DNA as standard curve (Lock M, et al., Hum. Gene Ther. 2010; 21:1273-1285).

In another embodiment, the purification is further carried out by a polyethylene glycol precipitation step or a cesium chloride gradient fractionation. In some embodiments, the methods further comprise purification steps, such as treatment of the cell lysate with benzonase, purification of the cell lysate over a CsCl gradient, or purification of the cell lysate with the use of heparin sulphate chromatography (Halbert C, et al., Methods Mol. Biol. 2004; 246:201-212).

Various naturally occurring and recombinant AAV, their encoding nucleic acids, AAV Cap and Rep proteins and their sequences, as well as methods for isolating or generating, propagating, and purifying such AAV, and in particular, their capsids, suitable for use in producing AAV are known in the art.

Animal Models

The following are non-limiting animal models that can be used to test the efficacy of administering ENPP1 or ENPP3 to prevent or reduce the progression of pathological ossification or calcification.
1. Enpp1^{asj/asj} model of Generalized Arterial Calcification of Infancy (GACI); Li, et al., 2013, Disease Models & Mech. 6(5): 1227-35.
2. Enpp1^{asj/asj} model of Generalized Arterial Calcification of Infancy (GACI); Li, et al, 2014, PloS one 9(12): el 13542.
3. ABCC6^{-/-} mouse model of Pseudoxanthoma Elasticum (PXE); Jiang, et al., 2007, J. Invest. Derm. 127(6): 1392-4102.
4. HYP mouse model of X-linked hypophosphatasia (XLH); Liang, et al., 2009, Calcif Tissue Int. 85(3):235-46.
5. LmnaG609G/+ mouse model of Hutchison-Gilford Progeria Syndrome; Villa-Bellosta, etal, 2013, Circulation 127(24):2442-51.
6. Tip toe walking (ttw) mouse model of Ossification of the Posterior Longitudinal Ligament (OPLL) (Okawa, et al, 1998, Nature Genetics 19(3):271-3; Nakamura, et al, 1999, Human Genetics 104(6):492-7) and osteoarthritis (Bertrand, et al, 2012, Annals Rheum. Diseases 71(7): 1249-53).
7. Rat model of chronic kidney disease (CKD) on the adenine diet; Schibler, et al. , 1968, Clin. Sci. 35(2):363-72; O'Neill, etal, 2011, Kidney Int. 79(5):512-7.
8. Mouse model of chronic kidney disease (CKD) on the adenine diet; Jia, et al., 2013, BMC Nephrol. 14:116.
9. 5/6^{th} nephrectomy rat model of CKD; Morrison, 1962, Lab Invest. 11:321-32; Shimamura & Morrison, 1975, Am. J. Pathol. 79(1):95-106.
10. ENPP1 knockout mouse model of GACI and osteopenia; Mackenzie, et al, 2012, PloS one 7(2):e32177.

Animal models, such as the above, are used to test for changes in soft tissue calcification and ossification upon administration of a vector encoding ENPP1 or ENPP3, according to the invention. For example, the following mouse models: (a) Npt2a^{-/-} (b) the double mutant Npt2a^{-/-}/Enpp1^{asj/asj}, and (c) a C57BL/6 mouse (Jackson Labs) that has been subject to diet-induced formation of renal stones, the diet being a high calcium, low magnesium diet (such as Teklad Labs diet TD. 00042, Harlan Labs, Madison, Wis.).

Npt2a^{-/-} mice show kidney stone formation when fed using normal chow starting at weaning age and persist at least until 10 weeks of age. Conversely double mutant Npt2a^{-/-}/Enpp1^{asj/asj} mice present twice the levels of kidney stone formation when compared with Npt2a−/− mice when fed a normal chow. Npt2a^{-/-} mice, and Npt2a^{-/-}/Enpp1^{asj/asj} mice are commercially obtained from Jackson laboratory, ME. Double mutant mice (Npt2a^{-/-}/Enpp1^{asj/asj}) are created by cross breeding Npt2a^{-/-} mice and Enpp1^{asj/asj} mice following standard protocols known in the art (Jackson Laboratory Recourse Manual, (2007, 1-29)). The Npt2a^{-/-} or Npt2a^{-/-}Enpp1^{asj/asj} double mutant mouse models for renal stone related disease can be used to test the efficacy of treatment according to the invention (Khan & Canales, 2011, J. Urol. 186(3):1107-13; Wu, 2015, Urolithiasis 43(Suppl 1):65-76). Oxalate stone-forming rodent models, i.e., ethylene glycol, hydroxyl purine-fed mice or rats, or intraperitoneal injection of sodium oxalate of mice and rats (Khan & Glenton, J. Urology 184:1189-1196), urate stone forming (Wu, et al., 1994, Proc. Natl. Acad. Sci. USA 91(2): 742-6) and cystinuria mouse models (Zee, et al., 2017, Nat. Med. 23(3):288-290; Sahota, et al., 2014, Urology 84(5):1249 e9-15) can also be tested.

In certain embodiments, there is no rodent model that recapitulates the adult form of the human disease GACI, also referred to in the literature as Autosomal Recessive Hypohposphatemic Rickets type 2 (ARHR2) (Levy-Litan, et al, 2010, Am. J. Human Gen. 86(2): 273-8.)

Experimental details on enzymatic activity of ENPP1, enzymatic activity of ENPP3, quantification of plasma PPi, micro-CT scans, quantification of plasma PPi uptake, are described in detail in the patent application and publications of PCT/US2016/33236—Braddock et al., WO 2014/126965—Braddock et al., WO 2017/087936—Braddock et al., and US 2015/0359858—Braddock et al., all of which are herein incorporated in their entirety.

The present invention is further illustrated by the following examples which in no way should be construed as being further limiting. The contents of all cited references (including literature references, issued patents, published patent applications, and co-pending patent applications) cited throughout this application are hereby expressly incorporated by reference.

### EXAMPLES

### Example: 1—Cloning of NPP1 Sequences into AAV System, Generating Constructs for AAV Infection, AAV Production and Purification

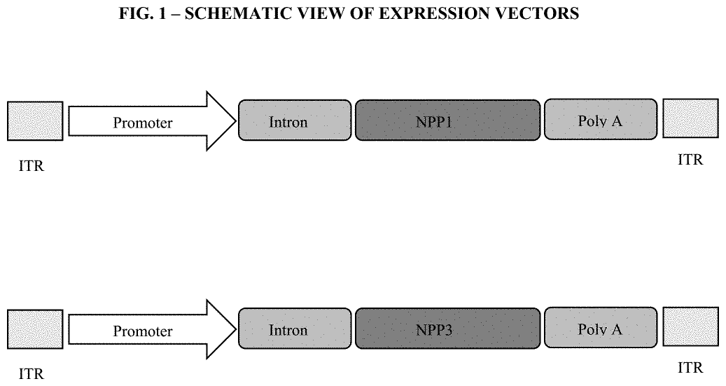

An AAV plasmid used in this example contains an expression cassette flanked by two ITRs from AAV2. The genome of AAV2 may be pseudo typed with AAV8. An expression cassette may have the following elements in the 5′ to 3′ direction: a liver-specific enhancer hepatic control region (HCR), a liver-specific promoter human alpha anti-trypsin (hAAT), an intron, a polynucleotide comprising N terminal Azurocidin signal sequence, the NPP1 cDNA, C terminal Fc sequence, and an SV40 polyadenylation signal. The expression cassette is flanked by the 5′ ITR and the 3′ ITR from AAV2. The construct generated is shown in the schematic of FIG. 1.

ENPP1 protein is a transmembrane protein localized to the cell surface with distinct intramembrane domains. ENPP1 protein was made soluble by omitting the transmembrane domain. Human NPP1 (NCBI accession NP_006199) was modified to express a soluble, recombinant protein by replacing its transmembrane region (e.g., residues 77-98 of ENPP1, NCBI accession NP_006199) with a suitable signal peptide sequence selected from the group consisting of (a). residues 12-30 of human NPP2 (NCBI accession NP_001 124335) or (b). residues 1-22 of ENPP7 or (c), residues 1-24 of ENPPS or (d), human serum albumin or (e), human Azurocidin

SEQ IDS (1-4, 6-15, 17-31 and 42-56) indicate several ENPP1-Fc and ENPP3-Fc constructs, all of which can be used for Cloning of ENPP1 or ENPP3 sequences into AAV system, generating constructs for AAV infection.

The modified NPP1 sequence was cloned using standard molecular biology protocols into a plasmid. A non-coding plasmid carrying the same components of the construct, but without the NPP1 cDNA and having a multi-cloning site was used to produce null particles as a control.

Infectious AAV vector particles are generated in HEK293 cells cultured in roller bottles, by co-transfecting each roller bottle with 125 μg of vector plasmid (containing the ITRs and the expression cassette) together with 125 μg of the rep/cap plasmid (expressing capsid proteins of the AAV particle and proteins necessary for virus replication), and 150 μg of the helper plasmid expressing adenovirus helper functions by calcium phosphate co-precipitation. A total of 10 roller bottles are used for each vector preparation. Approximately three days after transfection, cells are harvested and centrifuged at 2500 g for 10 min. Cell pellet and medium are then processed separately. Cell pellet is thoroughly reconstituted in TBS (50 mM TrisHCl, 150 mM NaCl, 2 mM MgCl2, pH 8.0).

After 3 freeze/thaw cycles the lysate is centrifuged at 2500 g for 30 min. Supernatant from this centrifugation is added to the medium and vector particles are precipitated by incubation with 8% of PEG 8000 (Sigma) for 15 h and pelleted at 2500 g for 30 min. The pellet, containing vectors from cells and medium, is thoroughly reconstituted in TBS, treated with benzonase (Merck) for 30 min at 37° C. and centrifuged at 10,000 g for 10 min. The supernatant is loaded into 37.5 ml ultra-clear tubes (Beckman) containing 1.3-1.5 g/ml CsCl density step gradient and centrifuged for 17 hours at 28,000 rpm in a SW28 rotor (Beckman). Viral bands are collected using a 10 ml syringe and 18-gauge needle and transferred to a new 12.5 ml ultra-clear tube, which is filled up with 1.379 g/ml CsCl solution to generate a continuous gradient. Tubes are centrifuged at 38,000 rpm in SW40Ti rotor (Beckman) for 48 hours. Finally, the band of full particles is collected and dialyzed in PBS using 10 KDa membrane (Slide-A-Lyzer Dialysis Products, Pierce) and filtered with 0.45 μm Millipore filters. This PEG and CsCl-based purification protocol dramatically reduces empty AAV capsids and DNA and protein impurities from the viral stock thus increasing AAV purity, which ultimately results in higher transduction in vivo. The same protocol is used for generating infectious AAV particles carrying the “null” vector which does not encode any ENPP protein.

### Example-2—Expression of ENPP1 Using Different Signal Sequences

ENPP1 is produced by establishing stable transfections in either CHO or HEK293 mammalian cells. To establish stable cell lines, a nucleic acid sequence encoding ENPP1 fusion proteins (such as sequences disclosed elsewhere herein) is placed in an appropriate vector for large scale protein production. There are a variety of such vectors available from commercial sources.

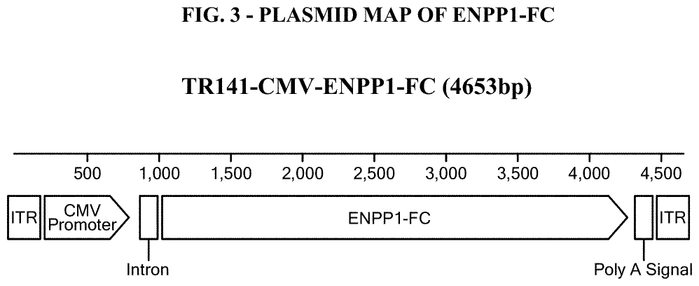

For example, FIG. 3 shows plasmid maps of NPP2^{signal}-NPP1-Fc cloned into the pcDNA3 plasmid, NPP7^{signal}-NPP1-Fc cloned into the pcDNA3 plasmid and Azurocidin^{signal}-NPP1-Fc cloned into the pcDNA3 plasmid with appropriate endonuclease restriction sites. The pcDNA3 plasmids containing the desired protein constructs are stably transfected into expression plasmid using established techniques such as electroporation or lipofectamine, and the cells are grown under antibiotic selection to enhance for stably transfected cells.

Clones of single, stably transfected cells are then established and screened for high expressing clones of the desired fusion protein. Screening of the single cell clones for ENPP1 protein expression are accomplished in a high-throughput manner in 96 well plates using the synthetic enzymatic substrate pNP-TMP as previously described for ENPP1 (Saunders, et al., 2008, Mol. Cancer Ther. 7(10):3352-62; Albright, et al., 2015, Nat Commun. 6:10006).

Upon identification of high expressing clones through screening, protein production is accomplished in shaking flasks or using bio-reactors as previously described for ENPP1 (Albright, et al., 2015, Nat Commun. 6:10006). Purification of ENPP1 is accomplished using a combination of standard purification techniques known in the art.

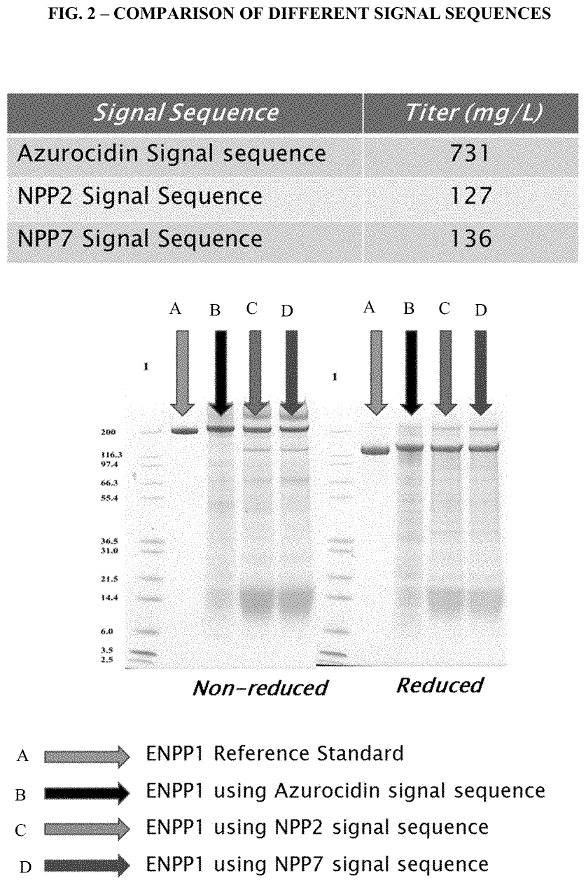

As demonstrated in FIG. 2, the construct comprising Azurocidin signal sequence produces the highest amount of NPP1 protein. The amount ENPP1 protein produced using Azurocidin signal sequence (731 mg/Liter) is surprisingly five-fold higher than when compared to the ENPP1 protein produced using NPP2 (127 mg/Liter) or using NPP7 (136 mg/Liter) signal sequence. The ENPP1 protein thus produced is further purified using additional techniques and/or chromatographic steps as described above, to reach substantially higher purity such as ˜99% purity.

Enzymatic activity of the ENPP1 thus produced is measured by determining the steady state hydrolysis of ATP by human NPP1 using HPLC. Briefly, enzyme reactions are started by addition of 10 nM ENPP1 to varying concentrations of ATP in the reaction buffer containing 20 mM Tris, pH 7.4, 150 mM NaCl, 4.5 nM KCl, 14 μM ZnCl₂, 1 mM MgCl₂ and 1 mM CaCl₂. At various time points, 50 μl reaction solution is removed and quenched with an equal volume of 3M formic acid. The quenched reaction solution is loaded on a C-18 (5 μm, 250×4.6 mm) column (Higgins Analytical) equilibrated in 5 mM ammonium acetate (pH 6.0) solution and eluted with a 0% to 20% methanol gradient. Substrate and products were monitored by UV absorbance at 259 nm and quantified according to the integration of their correspondent peaks and standard curves. The ENPP1 protein is thus characterized following the protocols discussed herein and elsewhere in PCT/2014/015945—Braddock et al.; PCT/2016/033236—Braddock et al. and PCT/2016/063034—Braddock et al.

### Example-3—Injection of AAV Viral Particles Encoding ENPP1-Fc to Mice and Measuring Weight Gain, Bone Density, Bone Strength and Bone Volume

The efficacy of delivery of a vector encoding and capable of expressing NPP1 or NPP3 is tested using a mouse model such as Enpp1^{asj/asj} mouse model, ABCC6^{-/-} mouse model, HYP mouse model, ttw mouse model, mouse model of chronic kidney disease (CKD) or 5/6th nephrectomy rat model of CKD. As a non-limiting example, the following experiment uses Enpp1^{asj/asj} mouse as the mouse model, Azurocidin-NPP1-Fc construct as the polynucleotide being delivered to the mouse model, and the delivery is accomplished by using AAV particles (prepared as shown in Example 1) which encodes ENPP1-Fc protein in vivo.

A person of ordinary skill would recognize the same experiment can be repeated by using alternate mouse models, alternate polynucleotide constructs comprising alternate signal sequences (NPP2, NPP5, NPP7. Albumin or Azurocidin etc.) encoding different ENPP1 fusions proteins (ENPP1-Albumin or ENPP1-Fc or ENPP1 functional equivalents or ENPP1 lacking Fc or Albumin domains etc.) or different ENPP3 fusion proteins (ENPP3-Fc or ENPP3-Albumin or ENPP3-lacking Fc or Albumin domain or ENPP3 functional equivalents etc.) disclosed in the invention for testing the efficacy of gene therapy for treating diseases of pathological calcification or ossification. The Azurocidin-NPP1-Fc construct utilized in the experiment encodes human ENPP1-Fc protein as a proof of concept and the same experiment can be repeated with an Azurocidin-NPP3-Fc construct that encodes human ENPP3-Fc.

Four sets of mice are used in this experiment, each set has at least five mice (6-8 weeks old), before injection of AAV particles, all sets of mice are tolerized by intraperitoneal injection of Titer GK1.5CD4 antibody at a concentration of 1000 μg/ml (final dose of 25-40 μg/animal) to reduce immune responses in mouse to human proteins produced by AAV constructs, a first cohort of ENPP1^{wt} mice that serve as control group are injected with AAV particles that comprise a null vector, a second cohort of Enpp1^{asj/asj} mice that serve as a control group are injected with AAV particles that comprise a null vector, a third cohort of ENPP1^{wt} mice that serve as study group are injected with AAV particles comprising polynucleotide that encodes ENPP1-Fc protein, and a fourth cohort of Enpp1^{asj/asj} that serve as test group are injected with AAV particles comprising polynucleotide that encodes ENPP1-Fc protein. Tolerization injections are repeated weekly (i.e. at Days 7, 14, 21, 28, 35, 42, 49, 56, 63, 70, 77, 84, 91, 98 and 105 days post AAV administration) after the AAV injection to each cohort.

The mice of the experiment are fed with either an acceleration diet ((Harlan Teklad, Rodent diet TD. 00442, Madison, Wis.), which is enriched in phosphorus and has reduced magnesium content) or regular chow (Laboratory Autoclavable Rodent Diet 5010; PMI Nutritional International, Brentwood, Mo.) and after 6-8 weeks of age, all mice receive a retro-orbital injection or tail vein injection of approx. 1×10¹² to 1×10^{15 vg/kg}, preferably 1×10¹³ to 1×10^{14 vg/kg} in PBS pH 7.4. The injected vectors are either empty “null” (control group) or carry the NPP1 gene (study group). Weight measurements are made daily to record any increases or decreases in body weight post AAV injection. Blood, urine , bone and tissue samples from the mice are collected and analyzed as follows. The experimental protocols are listed in detail in Albright et al., Nat Commun. 2015 Dec. 1; 6: 10006, and Caballero et al., PLoS One. 2017; 12(7): e0180098, the contents of all of which are hereby incorporated by reference in their entirety. At the end of the study (at 7, 28 and 112 days, all mice are euthanized following orbital exsanguination in deep anesthesia with isoflurane and vital organs are removed as described in art. (Impaired urinary osteopontin excretion in Npt2a−/− mice., Caballero et al., Am J Physiol Renal Physiol. 2017 Jan. 1; 312(1):F77-F83; Response of Npt2a knockout mice to dietary calcium and phosphorus, Li Yet al., PLoS One. 2017; 12(4):e0176232.).

Quantification of Plasma PPi

Animals are bled retro-orbitally using heparinized, micropipets, and the blood is dispensed into heparin-treated eppendorf tubes and placed on wet ice. The samples are spun in a 4° C. pre-cooled microcentrifuge at 4,000 r.p.m. for 5 min, and plasma is collected and diluted in one volume of 50 mM Tris-Acetate pH=8.0. The collected plasma is filtered through a 300 KDa membrane via ultracentrifugation (NanoSep 300 K, Pall Corp., Ann Arbour, Mich.) and frozen at −80° C. Pyrophosphate is quantitated using standard three-step enzymatic assays using uridine 5′ diphospho[¹⁴C] glucose to record the reaction product, uridine 5′ diphospho[¹⁴C]gluconic acid. (Analysis of inorganic pyrophosphate at the picomole level. Cheung C P, Suhadolnik R I, Anal Biochem. 1977 November; 83(1):61-3). Briefly, a reaction mixture (100 μl) containing 5 mM MgCl2, 90 mM KCL, 63 mM Tris-HCL (pH 7.6), 1 nmol NADP+, 2 nmol glucose 1,6-diphosphate, 400 pmol uridine 5′-diphosphoglucose, 0.02 μCi uridine 5′ diphospho[¹⁴C]glucose, 0.25 units of uridine 5′-diphosphoglucose pyrophosphorylase, 0.25 units of phosphoglucose mutase, 0.5 units of glucose 6-phosphate dehydrogenase, and inorganic pyrophosphate (50-200 pmol) is incubated for 30 min at 37° C. The reaction is terminated by the addition of 200 μl of 2% charcoal well suspended in water. An aliquote of 200 μl of supernatant is then counted in scintillation solution.

In Vivo⁹⁹mPYP Imaging

If desired, bone imaging may be performed. The bone imaging agent 99mTc-pyrophosphate (Pharmalucence, Inc) is evaluated in cohorts of animals using a preclinical microSPECT/CT hybrid imaging system with dual 1 mm pinhole collimators (X-SPECT, Gamma Medica-Ideas)38. Each animal is injected intraperitoneally with 2-5 mCi of the radiolabelled tracer and imaged 1-1.5 h after injection. A CT scan (512 projections at 50 kVp, 800 uA and a magnification factor of 1.25) is acquired for anatomical co-localization with the SPECT image. The SPECT imaging is acquired with 180° per collimator head in a counter-clockwise rotation, 32 projections, 60 s per projection with an ROR of 7.0 cm, FOV of 8.95 cm and an energy window of 140 keV±20. CT images shall be reconstructed with the FLEX X-O CT software (Gamma Medica-Ideas) using a filtered back-projection algorithm. SPECT images shall be reconstructed using the FLEX SPECT software (5 iterations, 4 subsets) and subsequently fused with the CT images and will be analyzed using the AMIRA software.

Quantification of ⁹⁹mPYP Uptake

For the ⁹⁹mPYP murine scans, the animals are imaged within 7 days of injection. The resulting SPECT scans is imported into NIH's ImageJ image processing software and regions of interest are drawn around each animal's head (target organ) and whole body. Per cent injected activity (PIA), often referred to as ‘per cent injected dose’ is calculated by comparing the ratio of counts in the head to the counts in the whole body and expressed as per cent injected dose to give a measure as of the affinity with which the radiotracer is taken up by the region of interest (head). The total counts in each scan is taken as the whole-body measure of injected dose.

Blood and Urine Parameters

Biochemical analyses also may be performed using blood samples (taken by orbital exsanguination) and spot urines collected following an overnight fast at the same time of day between 10 AM and 2 PM. Following deproteinization of heparinized plasma by filtration (NanoSep 300 K, Pall Corp., Ann Arbor, Mich.), plasma and urinary total pyrophosphate (PPi) concentrations are determined using a fluorometric probe (AB112155, ABCAM, Cambridge, Mass.). Urine PPi is corrected for urine creatinine, which is measured by LC-MS/MS or by ELISA using appropriate controls to adjust for inter-assay variability.

Kidney Histology

Left kidneys are fixed in 4% formalin/PBS at 4° C. for 12 hrs and then dehydrated with increasing concentration of ethanol and xylene, followed by paraffin embedding. Mineral deposits are determined on 10 um von Kossa stained sections counterstained with 1% methyl green. Hematoxyline/eosin is used as counterstain for morphological evaluation. Histomorphometric evaluation of sagittal kidney sections that includes cortex, medulla and pelvis are performed blinded by two independent observers using an Osteomeasure System (Osteometrics, Atlanta, Ga.). Percent calcified area is determined by using the formula: % calc. area=100*calcified area/total area (including cortex, medulla and pelvic lumen), and is dependent on number of observed areas per section. Mineralization size is determined by using the formula: calc. size=calcified area/number of observed calcified areas per section.

For transmission electron microscopy, a 1 mm³ block of the left kidney is fixed in 2.5% glutaraldehyde and 2% paraformaldehyde in phosphate buffered saline for 2 hrs., followed by post-fixation in 1% osmium liquid for 2 hours. Dehydration will be carried out using a series of ethanol concentrations (50% to 100%). Renal tissue will be embedded in epoxy resin, and polymerization will be carried out overnight at 60° C. After preparing a thin section (50 nm), the tissues will be double stained with uranium and lead and observed using a Tecnai Biotwin (LaB6, 80 kV) (FEI, Thermo Fisher, Hillsboro, Oreg.).

Histology, Histomorphometry, and Micro-CT

Tibiae and femora of mice are stripped of soft tissue, fixed in 70% ethanol, dehydrated, and embedded in methyl methacrylate before being sectioned and stained with toluidine blue (C. B. Ware et al., Targeted disruption of the low-affinity leukemia inhibitory factor receptor gene causes placental, skeletal, neural and metabolic defects and results in perinatal death. Development 121, 1283-1299 (1995)). Histomorphometric measurements are performed on a fixed region just below the growth plate corresponding to the primary spongiosa (A. M. Parfitt et al., Bone histomorphometry: standardization of nomenclature, symbols, and units. Report of the ASBMR Histomorphometry Nomenclature Committee. J Bone Miner Res 2, 595-610 (1987)) and analyzed by Osteomeasure software (Osteometrics, Atlanta, Ga.). The bones are scanned using a Scanco μCT-35 (Scanco, Brutissellen, Switzerland) and analyzed for numerous structural parameters at both the proximal tibia and distal femur just below the growth plate (trabecular bone) and at the tibial or femoral midshaft (cortical bone).

Bone Biomechanical Testing

Femurs from mice on the acceleration diet are loaded to failure with three-point bending; femurs from mice on regular chow are loaded to failure with four-point bending. All whole bone tests are conducted by loading the femur in the posterior to anterior direction, such that the anterior quadrant is subjected to tensile loads. The widths of the lower and upper supports of the four-point bending apparatus are 7 mm and 3 mm, respectively. Tests are conducted with a deflection rate of 0.05 mm/sec using a servohydraulic testing machine (Instron model 8874; Instron Corp., Norwood, Mass., USA). The load and mid-span deflection is acquired directly at a sampling frequency of 200 Hz. Load-deflection curves are analyzed for stiffness, maximum load, and work to fracture. Yield is defined as a 10% reduction in the secant stiffness (load range normalized for deflection range) relative to the initial tangent stiffness. Femurs are tested at room temperature and kept moist with phosphate-buffered saline (PBS). Post-yield deflection, which is defined as the deflection at failure minus the deflection at yield are measured as well.

### Example 4—Treatment of Chronic Kidney Disease using Viral Vectors Expressing ENPP1 or ENPP3

The following example provides AAV expressing ENPP1 or ENPP3 which are expected to be effective in treating vascular calcification and symptoms associated with CKD. ENPP1-Fc and ENPP3-Fc are used in the examples for illustrative purposes and similar results can be obtained by using other ENPP1 or ENPP3 fusions of the invention.

AAV virions expressing ENPP1-Fc and ENPP3-Fc protein are made according to example 1 and administered to a CKD mouse (which is a model of chronic kidney disease (CKD) (BMC Nephrology, 2013, 14:116). Six sets of mice are used for treatment with ENPP1 and ENPP3.

Control cohorts: in this experiment, a first cohort of ENPP1 wt mice that serve as control group are injected with AAV particles that comprise a null vector and, a second cohort of CKD mice that serve as a control group are injected with AAV particles that comprise a null vector.

ENPP1-treated mice cohorts: a third cohort of ENPP1^{wt} mice are injected with AAV particles engineered to express ENPP1-Fc protein, and a fourth cohort of CKD mice are injected with AAV particles engineered to express ENPP1-Fc protein.

ENPP3-treated mice cohorts: a fifth cohort of ENPP1^{wt} mice are injected with AAV particles engineered to express ENPP3-Fc protein, and a sixth cohort of CKD mice are injected with AAV particles engineered to express ENPP3-Fc protein.

Adenine Diet: The CKD mice are maintained on adenine diet and whereas wildtype mice are maintained on regular chow (Laboratory Autoclavable Rodent Diet 5010; PMI Nutritional International, Brentwood, Mo.). To provide an adenine-containing chow consumed by the CKD mice, adenine is mixed with a casein-based diet that blunted the smell and taste. Adenine is purchased from Sigma Aldrich (MO, USA) and the powdered casein-based diet is purchased from Special Diets Services (SDS, UK) (reference number 824522). Other ingredients of the diet are maize starch (39.3%), casein (20.0%), maltodextrin (14.0%), sucrose (9.2%), maize/corn oil (5%), cellulose (5%), vitamin mix (1.0%), DL-methionine (0.3%) and choline bitartrate (0.2%).

Vector Injection: After two weeks of age, all mice receive a retro-orbital injection or tail vein injection of approx. 1×10¹² to 1×10^{15 vg/kg} preferably. 1×10¹³ to 1×10^{14 vg/kg} in PBS pH 7.4 per mouse. The injected vectors are either empty “null” (control group) or carried the NPP1or NPP3 gene (study group).

Assays: Kidney histology, PPi levels, and blood urine parameters such as FGF-23 levels, vitamin D, Parathyroid hormone (PTH) levels, serum/blood urea levels, blood urea nitrogen (BUN) levels, serum/blood creatine levels and plasma pyrophosphate (PPi) are analyzed for each cohort as described in Example 3. Urine is collected as spot urine samples after spontaneous urination. Serum and urine calcium, phosphorous, creatinine and urea levels are measured on a Konelab 20XTi (Thermo Scientific, Finland). Creatinine concentrations are validated with a colorimetric assay (BioChain, CA USA). PTH is measured by a mouse intact PTH ELISA kit (Immutopics, CA, USA), FGF23 levels are measured with an intact FGF23 ELISA (Kainos, Japan) and Vitamin D is measured with EIA kits (Immunodiagnostic Systems, UK). Experimental details are listed in BMC Nephrology, 2013, 14:116, and PLoS One. 2017 Jul. 13; 12(7).

Results: Untreated CKD mice generally exhibit reduced body weight and signs of declining kidney function such as decreased ratios between urine urea/serum urea and urine creatinine/serum creatinine. In contrast, CKD mice treated with AAV expressing ENPP1 or ENPP3 proteins are expected to show an increase in body weight approaching the body weight ranges of normal WT mice. Generally, serum urea levels ranging from 80-100 mg/dL is considered optimal. Urea levels of above 100 mg/dL are associated with increased morbidity along with weight loss and reduced physical activity. Treated (AAV with ENPP1 or ENPP3) CKD mice are expected to exhibit improved kidney functions manifested by a decrease in serum urea levels and increase in urine urea levels leading to higher urine urea/serum urea ratios.

Renal histology analysis of kidney tissues of CKD mice are expected to show deposition of crystalline structures in regions such as tubular lumen, micro abscesses and dilated tubules, Periodic acid-Schiff (PAS) staining showing dilated Bowman's space, presence of atrophic tubules with protein casts (“thyroidization”) and tubular atrophy with thickening of the tubular basement membrane, presence of mild interstitial fibrosis seen through Ladewig staining and occurrence of extensive calcification of tubular structures seen through von Kossa staining. In contrast, CKD mice treated according to the invention with ENPP1 or ENPP3 are expected to show a reduction or lack of renal mineral deposits in the tubular lumen and soft tissue vasculature with histology similar to that of healthy wildtype mice.

Untreated CKD mice are expected to show a significant increase in serum inorganic phosphorous (pi), increase in PTH and FGF23 levels but a decrease in 1,25(OH)₂-Vitamin D levels and lower PPi levels (˜0.5 μM) when compared with that of healthy wild type mice (Normal levels of PPi are about 2-4 μM; about 10-65 ng/L for PTH; median FGF23 level is 13 RU/ml and normal FGF23 level ranges from 5 to 210 RU/ml; normal Vitamin D levels are 20 ng/mL to 50 ng/mL). In contrast, treated CKD mice are expected to show elevated levels of PPi (˜4-5 μM) which are expected to be higher than the PPi levels found in untreated CKD mice (˜0.5 μM). Thus a person of ordinary skill can determine the therapeutic efficacy of vector based ENPP1 or ENPP3 in treating chronic kidney diseases by observing one or more factors like reduction (25%, or 50%, or 70%, or 90% or 100% reduction) of calcification of soft tissues in kidneys and coronary arteries visualized through histological analysis , increase in serum PPi levels, normalization of vitamin D levels, reduction in FGF23 levels to normal ranges, normalization of PTH levels from blood analysis, increased survival, improved kidney function observed by increase in urine urea and creatine along with increased weight gain.

Treatment of Human Subjects:

A human patient suffering from CKD is treated by providing an intravenal injection containing approximately 5×10¹¹-5×10^{15 vg/kg} in 1× PBS at pH 7.4, in some embodiments approximately 1×10¹²-1×10^{15 vg/kg} in 1× PBS at pH 7.4 per subject capable of delivering and expressing ENPP1 or ENPP3. Successful treatment of CKD is observed by monitoring the one or more aforesaid parameters through periodic blood and urine tests as discussed for mouse models. Instead of histological analysis which requires staining of kidney slices or arterial tissues which is not feasible to perform in living patients, instead one uses noninvasive visualization techniques commonly known in art such as CT scan, ultrasound, or intravenous pyelography to visualize the presence of calcifications and the reduction of calcifications in response to vector-based delivery and expression of ENPP1 or ENPP3 in patients suffering from CKD. Intravenous pyelography is an X-ray exam that uses a contrast medium, which functions as a dye, to help visualize the urinary tract and detect the presence of renal calcifications. Computed tomography is a noninvasive imaging technique that uses X-ray technology to depict internal structures of the body such as the urinary tract. Renal calcifications are visible on CT scans. CT scans collect X-ray images from different angles around the body to generate detailed cross-sectional images as well as three-dimensional images of the body's internal structures and organs. CT scan can also be used in arteries to detect the presence and subsequent reduction of calcification following treatment. A computer analyzes the radiation transmitted through the body to reconstruct the images of the internal structures and organs.

A medical doctor having skill in visualizing soft tissue calcification, cardiac calcification, myocardial infarction undertakes treatment of a subject afflicted with CKD by administering AAV virions expressing human ENPP1 or human ENPP3. The physician administers viral particles that deliver constructs of hENPP1 or hENPP3 and express the corresponding proteins under the control of an inducible promoter. The physician thus has the option to control the dosage (amount of hENPP1 or hENPP3 expressed) based on the rate and extent of improvement of symptoms. Successful treatment is observed by a medical professional of skill in art by observing one or more positive symptoms such as improved kidney function, improved urine creatine levels (normal creatine levels in urine for men are 40-278 mg/dL and 29-226 mg/dL for women), and improved urine-urea levels (normal urea levels in urine for adults are 26-43 g/24 h), normal serum-creatine levels (normal serum creatinine range is 0.6-1.1 mg/dL in women and 0.7-1.3 mg/dL in men), normal vitamin D levels (20 ng/ml to 50 ng/mL is considered adequate for healthy people. A level less than 12 ng/mL indicates vitamin D deficiency), normal blood urea nitrogen levels (BUN level for healthy adults is 7-20 mg/dL), weight gain, increase in serum PPi levels (at least about 4-5 μm), reduction in calcification (25%, or 50%, or 70%, or 90% or 100% reduction) of arterial tissues and or reduction of calcification in kidney tubules visualized by noninvasive techniques such as CT or ultrasound scans.

### Example 5—Treatment of GACI using Viral Vectors Expressing ENPP1 or ENPP3

The following example provides AAV expressing ENPP1 or ENPP3 which are expected to be effective in treating vascular calcification and symptoms associated with GACI. ENPP1-Fc and ENPP3-Fc are used in the examples for illustrative purposes and similar results can be obtained by using other ENPP1 or ENPP3 fusions of the invention.

AAV virions expressing ENPP1-Fc and ENPP3-Fc protein are made according to example 1, and administered to a Enpp1^{asj/asj} mouse (which is a model for Generalized Arterial Calcification of Infancy (Li, et al., 2013, Disease Models & Mech. 6(5): 1227-35). Six sets of mice are used for treatment with ENPP1 and ENPP3.

Control cohorts: in this experiment, a first cohort of ENPP1 wt mice that serve as control group are injected with AAV particles that comprise a null vector and, a second cohort of Enpp1^{asj/asj} mice that serve as a control group are injected with AAV particles that comprise a null vector.

ENPP1-treated mice cohorts: a third cohort of ENPP1^{wt} mice are injected with AAV particles engineered to express ENPP1-Fc protein, and a fourth cohort of Enpp1^{asj/asj} mice are injected with AAV particles engineered to express ENPP1-Fc protein.

ENPP3-treated mice cohorts: a fifth cohort of ENPP1^{wt} mice are injected with AAV particles engineered to express ENPP3-Fc protein, and a sixth cohort of Enpp1^{asj/asj} mice are injected with AAV particles engineered to express ENPP3-Fc protein. The wildtype mice are maintained on regular chow diet and the Enpp1^{as/asj} mice are fed high phosphate Teklad diet.

Vector Injection: After two weeks of age, all mice receive a retro-orbital injection or tail vein injection of approx. 1×10¹² to 1×10^{15 vg/kg} preferably 1×10¹³ to 1×10^{14 vg/kg} in PBS pH 7.4 per mouse. The injected vectors are either empty “null” (control group) or carried the NPP1 or NPP3 gene (study group).

Assay: Kidney histology, PPi levels, and blood urine parameters such as FGF-23 levels, vitamin D, Parathyroid hormone (PTH) levels, serum/blood urea levels, blood urea nitrogen (BUN) levels, serum/blood creatine levels and plasma pyrophosphate (PPi) are analyzed for each cohort as described in Example 3 and 4.

Results: Untreated Enpp1^{asj/asj} mice generally exhibit reduced body weight and increased mortality. In contrast, Enpp1^{asj/asj} mice treated with AAV expressing ENPP1 proteins or ENPP3 proteins are expected to show an increase in body weight approaching the body weight ranges of normal WT mice.

Enpp1^{asj/asj} mice treated with null vector are expected to display calcifications in their hearts, aortas and coronary arteries, and histologic evidence of myocardial infarctions in the free wall of right ventricle, calcifications of coronary arteries, heart, ascending and descending aorta, myocardial cell necrosis, and myocardial fibrosis in the myocardial tissue adjacent to regions of coronary artery calcification. In contrast, Enpp1^{asj/asj} animals treated with AAV expressing ENPP1-Fc or ENPP3-Fc are expected to display an absence of cardiac, arterial, or aortic calcification on histology or post-mortem micro-CT. Enpp1^{asj/asj} mice treated with null vector also show calcifications centered in the renal medulla along with heavy, extensive calcifications, centered in the outer medulla, with extension into the renal cortex. In contrast, Enpp1^{asj/asj} mice treated with according to the invention with ENPP1 or ENPP3 are expected to show a reduction or lack of renal mineral deposits in the tubular lumen and soft tissue vasculature with histology similar to that of healthy wildtype mice.

In addition to survival, daily animal weights, and terminal histology, treatment response is assessed via post-mortem high-resolution micro-CT scans to image vascular calcifications, plasma PPi concentrations, and 99mTc PPi (99mPYP) uptake. None of the WT or treated (vector expressing ENPP1 or ENPP3) Enpp1^{asj/asj} are expected to possess any vascular calcifications via micro-CT, in contrast to the dramatic calcifications are expected in the aortas, coronary arteries, and hearts of the untreated (null vector) Enpp1^{asj/asj} cohort. In addition, serum PPi concentrations of treated (vector expressing ENPP1 or ENPP3) Enpp1^{asj/asj} animals (5.2 μM) are expected to be elevated to WT levels (4.4 μM) and significantly above untreated enpp1asj/asj levels (0.5 μM).

99mPYP is an imaging agent typically employed in cardiac imaging and bone remodeling. It is sensitive to areas of unusually high-bone rebuilding activity since it localizes to the surface of hydroxyapatite and then may be taken up by osteoclasts. Weekly serial imaging of untreated Enpp1^{asj/asj} animals are expected to show greater uptake of 99mPYP in the heads compared with that of treated Enpp1^{asj/asj} animals. Measurements are made on days 30-35 and at days 50-65 post administration of viral particles containing null vector or vector expressing ENPP1. Comparison of these experimental groups are expected to show that ENPP1-Fc or ENPP3-Fc treatment returned 99mPYP uptake in GACI mice to WT levels suggesting that ENPP1-Fc or ENPP3-Fc treatment is able to abrogate unregulated tissue, vibrissae and skull mineralization in Enpp1^{asj/asj} mice by raising the extracellular PPi concentrations. These observations are expected to show that the Enpp1^{asj/asj} mice dosed viral particles containing vector expressing ENPP1-Fc or ENPP3-Fc are free of vascular calcifications and have normal plasma PPi concentrations.

Untreated Enpp1^{asj/asj} mice are also expected to show a significant increase in serum inorganic phosphorous (pi), increase in PTH and FGF23 levels but a decrease in 1,25(OH)2-Vitamin D levels and lower PPi levels (˜0.5 μM) when compared with that of healthy wild type mice (Normal levels of PP are about 2-4 μM; about 10-65 ng/L for PTH; median FGF23 level is 13 RU/ml and normal FGF23 level ranges from 5 to 210 RU/ml; normal Vitamin D levels are 20 ng/mL to 50 ng/mL). In contrast, treated Enpp1^{asj/asj} mice are expected to show elevated levels of PPi (˜4-5 μM) which are expected to be higher than the PPi levels found in untreated CKD mice (˜0.5 μM). Thus a person of ordinary skill can determine the therapeutic efficacy of vector based ENPP1 or ENPP3 in treating GACI by observing one or more factors like reduction (25%, or 50%, or 70%, or 90% or 100% reduction) of calcification of soft tissues in kidneys and coronary arteries visualized through histological analysis , increase in serum PPi levels, normalization of vitamin D levels, reduction in FGF23 levels to normal ranges and normalization of PTH levels from blood analysis, increased survival, improved kidney function observed by increase in urine urea and creatine along with increased weight gain.

Treatment of Human Subjects

A human patient suffering from GACI is treated by providing an injection containing approximately. 5×10¹¹-5×10^{15 vg/kg} in 1× PBS at ₚH 7.4, in some embodiments approximately 1×10¹²-1×10^{15 vg/kg} in 1× PBS at pH 7.4 per subject capable of delivering and expressing hENPP1 or hENPP3. Successful treatment of GACI is observed by monitoring one or more aforesaid parameters through periodic blood and urine tests as discussed for mouse models. Instead of histological analysis which requires staining of kidney slices or arterial tissues which is not feasible to perform in living patients, one instead uses noninvasive visualization techniques as discussed in example 4.

A medical doctor having skill in visualizing soft tissue calcification, cardiac calcification, myocardial infarction undertakes treatment of a subject afflicted with GACI by administering AAV virions expressing hENPP1 or hENPP3. The physician administers viral particles that deliver a construct encoding hENPP1 or hENPP3, the vector expresses the ENPP protein under the control of an inducible promoter. The physician can control the dosage (amount of hENPP1 or hENPP3 expressed) based on the rate and extent of improvement of symptoms. A successful treatment is observed by a medical professional of skill in art by observing one or more positive symptoms such as normal vitamin D levels (20 ng/ml to 50 ng/mL is considered adequate for healthy people. A level less than 12 ng/mL indicates vitamin D deficiency), normal blood urea nitrogen levels (BUN level for healthy adults is 7-20 mg/dL), weight gain, increase in serum PPi levels (at least about 4-5 μm), reduction in calcification (25%, or 50%, or 70%, or 90% or 100% reduction) of arterial tissues and/or reduction of calcification in kidney tubules visualized by noninvasive techniques such as CT or ultrasound scans.

### Example 6—Treatment of PXE Using Viral Vectors Expressing ENPP1 or ENPP3

The following example provides AAV expressing ENPP1 or ENPP3 which are expected to be effective in treating vascular calcification and symptoms associated with PXE. ENPP1-Fc and ENPP3-Fc are used in the examples for illustrative purposes and similar results can be obtained by using other ENPP1 or ENPP3 fusions of the invention.

AAV virions expressing ENPP1-Fc protein and ENPP3-Fc protein are made according to example 1, and administered to a ABCC6^{-/-} mouse (which is a model for Pseudoxanthoma Elasticum; Jiang, et al., 2007, J. Invest. Derm. 127(6): 1392-4102). Six sets of mice are used for treatment with ENPP1 and ENPP3.

Control cohorts: in this experiment, a first cohort of ENPP1 wt mice that serve as control group are injected with AAV particles that comprise a null vector and, a second cohort of ABCC6^{-/-} mice that serve as a control group are injected with AAV particles that comprise a null vector.

ENPP1-treated mice cohorts: a third cohort of ENPP1^{wt} mice are injected with AAV particles engineered to express ENPP1-Fc protein, and a fourth cohort of ABCC6^{-/-} mice are injected with AAV particles engineered to express ENPP1-Fc protein.

ENPP3-treated mice cohorts: a fifth cohort of ENPP1^{wt} mice are injected with AAV particles engineered to express ENPP3-Fc protein, and a sixth cohort of ABCC6^{-/-} mice are injected with AAV particles engineered to express ENPP3-Fc protein. The wildtype mice are maintained on regular chow diet and the ABCC6^{-/-} mice are fed high phosphate Teklad diet.

Vector Injection: After two weeks of age, all mice receive a retro-orbital injection or tail vein injection of approx. 1×10¹² to 1×10^{15 vg/kg} preferably 1×10¹³ to 1×10^{14 vg/kg} in PBS pH 7.4 per mouse. The injected vectors are either empty “null” (control group) or carried the NPP1 or NPP3 gene (study group).

Assays: Kidney histology, PPi levels, and blood urine parameters such as FGF-23 levels, vitamin D, Parathyroid hormone (PTH) levels, serum/blood urea levels, blood urea nitrogen (BUN) levels, serum/blood creatine levels and plasma pyrophosphate (PPi) are analyzed for each cohort as described in Example 3 and 4.

Results: Untreated ABCC6−/− mice generally exhibit reduced body weight and increased mortality. In contrast, ABCC6−/− mice treated with AAV expressing ENPP1 or ENPP3 proteins are expected to show an increase in body weight approaching the body weight ranges of normal WT mice. ABCC6−/− mice treated with null vector are expected to display calcifications in their hearts, aortas and coronary arteries, and histologic evidence of myocardial infarctions in the free wall of right ventricle, calcifications of coronary arteries, heart, ascending and descending aorta, myocardial cell necrosis, and myocardial fibrosis in the myocardial tissue adjacent to regions of coronary artery calcification. In contrast, ABCC6−/− animals treated with vector expressing ENPP1-Fc or ENPP3-Fc are expected to display an absence of cardiac, arterial, or aortic calcification on histology or post-mortem micro-CT. Enpp1^{asj/asj} mice treated with null vector also show calcifications centered in the renal medulla along with heavy, extensive calcifications, centered in the outer medulla, with extension into the renal cortex. In contrast, Enpp1^{asj/asj} mice treated with viral vector-based expression of ENPP1 or ENPP3 are expected to show a reduction or a lack of renal mineral deposits in the tubular lumen and soft tissue vasculature with histology similar to that of healthy wildtype mice.

In addition to survival, daily animal weights, and terminal histology, treatment response is assessed via post-mortem high-resolution micro-CT scans to image vascular calcifications, and plasma PPi concentrations. None of the WT or treated (vector expressing ENPP1) ABCC6^{-/-} are expected to possess any vascular calcifications via micro-CT, in contrast to the dramatic calcifications that are expected to be seen in the aortas, coronary arteries, and hearts of the untreated (null vector) ABCC6^{-/-} cohort. In addition, serum PPi concentrations of treated (vector expressing ENPP1) ABCC6^{-/-} animals (5.2 μM) are expected to be elevated to WT levels (4.4 μM) and significantly above untreated ABCC6^{-/-} levels (0.5 μM).

Untreated ABCC6^{-/-} mice are also expected to show a significant increase in serum inorganic phosphorous (pi), increase in PTH and FGF23 levels but a decrease in 1,25(OH)₂-Vitamin D levels and lower PPi levels (·0.5 μM) when compared with that of healthy wild type mice (Normal levels of PP are about 2-4 μM; about 10-65 ng/L for PTH; median FGF23 level is 13 RU/ml and normal FGF23 level ranges from 5 to 210 RU/ml; normal Vitamin D levels are 20 ng/mL to 50 ng/mL). In contrast, treated ABCC6 mice are expected to show elevated levels of PPi (˜4-5 μM) which are expected to be higher than the PPi levels found in untreated ABCC6^{-/-} mice (0.5 μM). Thus a person of ordinary skill can determine the therapeutic efficacy of vector based ENPP1 or ENPP3 in treating PXE by observing one or more factors like reduction (25%, or 50%, or 70%, or 90% or 100% reduction) of calcification of soft tissues in kidneys and coronary arteries visualized through histological analysis, increase in serum PPi levels, normalization of vitamin D levels, reduction in FGF23 levels to normal ranges and normalization of PTH levels from blood analysis, increased survival and improved kidney function observed by increase in urine urea and creatine along with increased weight gain.

Treatment of Human Subjects:

A human patient suffering from PXE is treated by providing an intravenal injection containing approximately. 5×10¹¹-5×10^{15 vg/kg} in 1× PBS at pH 7.4, in some embodiments approximately 1×10¹²-1×10^{15 vg/kg} in 1× PBS at pH 7.4 per subject capable of delivering and expressing ENPP1 or ENPP3. Successful treatment of PXE is observed by monitoring one or more aforesaid parameters through periodic blood and urine tests as discussed for mouse models. Instead of histological analysis which requires staining of kidney slices or arterial tissues which is not feasible to perform in living patients, one instead uses noninvasive visualization techniques as discussed in example 4.

A medical doctor having skill in visualizing soft tissue calcification, cardiac calcification, myocardial infarction can undertake the treatment of a subject afflicted with PXE by administering AAV virions expressing ENPP1 or ENPP3. The physician can also use viral particles that deliver constructs of ENPP1 or ENPP3 and express the corresponding proteins under the control of an inducible promoter. The physician thus has the option to control the dosage (amount of ENPP1 or ENPP3 expressed) based on the rate and extent of improvement of symptoms. A successful treatment and suitable dosage is readily inferred by a medical professional of skill in art by observing one or more positive symptoms such as normal vitamin D levels (20 ng/ml to 50 ng/mL is considered adequate for healthy people. A level less than 12 ng/mL indicates vitamin D deficiency), disappearance or reduction of size and or number of angioid streaks, reduction or lack of retinal bleeding, normal blood urea nitrogen levels (BUN level for healthy adults is 7-20 mg/dL), weight gain, increase in serum PPi levels (at least about 4-5 μm), reduction in calcification (25%, or 50%, or 70%, or 90% or 100% reduction) of arterial tissues, connective tissues and or reduction of calcification in kidney tubules visualized by noninvasive techniques such as CT or ultrasound scans.

### Example 7—Treatment of OPLL Using Viral Vectors Expressing Human ENPP1 or ENPP3

The following example provides AAV expressing human ENPP1 or ENPP3 which are expected to be effective in treating vascular calcification and symptoms associated with PXE. ENPP1-Fc and ENPP3-Fc fusions are used in the examples for illustrative purposes and similar results can be obtained by using other ENPP1 or ENPP3 fusions of the invention.

AAV virions expressing ENPP1-Fc protein or ENPP3-Fc protein are made according to example 1, and administered to a Tip toe walking (ttw) mouse (which is a model for Ossification of the Posterior Longitudinal Ligament; (Okawa, et al, 1998, Nature Genetics 19(3):271-3; Nakamura, et al, 1999, Human Genetics 104(6):492-7). Six sets of mice are used for treatment with ENPP1 and ENPP3.

Control cohorts: in this experiment, a first cohort of ENPP1 wt mice that serve as control group are injected with AAV particles that comprise a null vector and, a second cohort of ttw mice that serve as a control group are injected with AAV particles that comprise a null vector.

ENPP1-treated mice cohorts: a third cohort of ENPP1^{wt} mice are injected with AAV particles engineered to express ENPP1-Fc protein, and a fourth cohort of ttw mice are injected with AAV particles engineered to express ENPP1-Fc protein.

ENPP3-treated mice cohorts: a fifth cohort of ENPP1^{wt} mice are injected with AAV particles engineered to express ENPP3-Fc protein, and a sixth cohort of ttw mice are injected with AAV particles engineered to express ENPP3-Fc protein. The wildtype mice are maintained on regular chow diet and the ttw mice are fed high phosphate Teklad diet.

Vector injection: After two weeks of age, all mice receive a retro-orbital injection or tail vein injection of approx. 1×10¹² to 1×10^{15 vg/kg} preferably 1×10¹³ to 1×10^{14 vg/kg} in PBS pH 7.4 per mouse. The injected vectors are either empty “null” (control group) or carried the NPP1 or NPP3 gene (study group).

Assays: Kidney histology, PPi levels, and blood urine parameters such as FGF-23 levels, vitamin D, Parathyroid hormone (PTH) levels, serum/blood urea levels, blood urea nitrogen (BUN) levels, serum/blood creatine levels and plasma pyrophosphate (PPi) are analyzed for each cohort as described in Example 3 and 4.

Results: Untreated ttw mice generally exhibit reduced body weight, thickening of spine, lethargy and increased mortality. In contrast, ttw mice treated with AAV expressing ENPP1 proteins or ENPP3 proteins are expected to show an increase in body weight approaching the body weight ranges of normal WT mice, normal alertness, and reduction in spine thickness approaching the thickness of wild type mouse. ttw mice treated with null vector are expected to display calcifications in their hearts, aortas and coronary arteries, and histologic evidence of myocardial infarctions in the free wall of right ventricle, calcifications of coronary arteries, heart, ascending and descending aorta, myocardial cell necrosis, and myocardial fibrosis in the myocardial tissue adjacent to regions of coronary artery calcification. In contrast, ttw animals treated with vector expressing ENPP1-Fc or ENPP3-Fc are expected to display an absence of cardiac, arterial, or aortic calcification on histology or post-mortem micro-CT. ttw mice treated with null vector also show calcifications centered in the renal medulla along with heavy, extensive calcifications, centered in the outer medulla, with extension into the renal cortex. In contrast, ttw mice treated with viral vector-based expression of ENPP1 or ENPP3 are expected to show a reduction or lack of renal mineral deposits in the tubular lumen, reduction of calcification of spine, and soft tissue vasculature with histology similar to that of healthy wildtype mice.

In addition to survival, daily animal weights, and terminal histology, treatment response is assessed via post-mortem high-resolution micro-CT scans to image vascular calcifications, and plasma PPi concentrations. None of the WT or treated (vector expressing ENPP1) ttw are expected to possess any vascular calcifications via micro-CT, in contrast to the dramatic calcifications that are expected to be seen in the aortas, coronary arteries, and hearts of the untreated (null vector) ttw cohort. In addition, serum PPi concentrations of treated (vector expressing ENPP1) ttw⁻ animals (5.2 μM) are expected to be elevated to WT levels (4.4 μM) and significantly above untreated ttw levels (0.5 μM).

Untreated ttw mice are also expected to show a significant increase in serum inorganic phosphorous (pi), increase in PTH and FGF23 levels but a decrease in 1,25(OH)₂-Vitamin D levels and lower PPi levels (˜0.5 μM) when compared with that of healthy wild type mice (Normal levels of PP are about 2-4 μM; about 10-65 ng/L for PTH; median FGF23 level is 13 RU/ml and normal FGF23 level ranges from 5 to 210 RU/ml; normal Vitamin D levels are 20 ng/mL to 50 ng/mL). In contrast, treated ttw mice are expected to show elevated levels of PPi (˜4-5 μM) which are expected to be higher than the PPi levels found in untreated ttw mice (˜0.5 μM). Thus a person of ordinary skill can determine the therapeutic efficacy of vector based ENPP1 or ENPP3 in treating OPLL by observing one or more factors like reduction (25%, or 50%, or 70%, or 90% or 100% reduction) of calcification of soft tissues in kidneys and coronary arteries visualized through histological analysis, increase in serum PPi levels, normalization of vitamin D levels, reduction in FGF23 levels to normal ranges and normalization of PTH levels from blood analysis, increased survival and improved kidney function observed by increase in urine urea and creatine along with increased weight gain.

Treatment of Human Subjects:

A human patient suffering from OPLL is treated by providing an intravenal injection containing approximately. 5×10¹¹-5×10^{15 vg/kg} in 1× PBS at pH 7.4, in some embodiments approximately 1×10¹²-1×10^{15 vg/kg} in 1× PBS at pH 7.4 per subject capable of delivering and expressing hENPP1 or hENPP3. Successful treatment of OPLL is observed by monitoring one or more aforesaid parameters through periodic blood and urine tests as discussed for mouse models. Instead of histological analysis which requires staining of kidney slices or arterial tissues which is not feasible to perform in living patients, one instead uses noninvasive visualization techniques as discussed in example 4.

A medical doctor having skill in visualizing soft tissue calcification, cardiac calcification, myocardial infarction can undertake the treatment of a subject afflicted with OPLL upon administration of AAV virions expressing hENPP1 or hENPP3. In some embodiments, the physician uses viral particles that deliver constructs of hENPP1 or hENPP3 and express the corresponding proteins under the control of an inducible promoter. The physician thus has the option to control the dosage (amount of hENPP1 or hENPP3 expressed) based on the rate and extent of improvement of symptoms. A successful treatment and suitable dosage is readily inferred by a medical professional of skill in art by observing one or more positive symptoms such as normal vitamin D levels (20 ng/ml to 50 ng/mL is considered adequate for healthy people. A level less than 12 ng/mL indicates vitamin D deficiency), normal blood urea nitrogen levels (BUN level for healthy adults is 7-20 mg/dL), weight gain, increase in serum PPi levels (at least about 4-5 μm), reduction in calcification (25%, or 50%, or 70%, or 90% or 100% reduction) of arterial tissues, reduction in thickness of spine and pain senstation, reduction of spinal stenosis visualized by noninvasive techniques such as CT, magnetic resonance imaging (Mill) or ultrasound scans.

### Example 8—Treatment of Osteopenia and or Osteomalacia using Viral Vectors Expressing ENPP1 or ENPP3

The following example provides AAV expressing ENPP1 or ENPP3 which are expected to be effective in treating symptoms associated with Osteopenia and/or Osteomalacia. ENPP1-Fc and ENPP3-Fc are used in the examples for illustrative purposes and similar results can be obtained by using other ENPP1 or ENPP3 fusions of the invention.

AAV virions expressing ENPP1-Fc protein or ENPP3-Fc protein are made according to example 1 and administered to a Tip toe walking (ttw) mouse (which is a mouse model for osteoarthritis (Bertrand, et al, 2012, Annals Rheum. Diseases 71(7): 1249-53)). Six sets of mice are used for treatment with ENPP1 and ENPP3. Similar experiment is repeated using ENPP1 knockout mice (ENPP1^{KO}) which also serves as a model for osteopenia. (Mackenzie, et al, 2012, PloS one 7(2):e32177) in addition to GACI.

Control cohorts: in this experiment, a first cohort of ENPP1 wt mice that serve as control group are injected with AAV particles that comprise a null vector and, a second cohort of ttw (or ENPP1^{KO}) mice that serve as a control group are injected with AAV particles that comprise a null vector.

ENPP1-treated mice cohorts: a third cohort of ENPP1^{wt} mice are injected with AAV particles engineered to express ENPP1-Fc protein, and a fourth cohort of ttw mice (or ENPP1^{KO}) are injected with AAV particles engineered to express ENPP1-Fc protein.

ENPP3-treated mice cohorts: a fifth cohort of ENPP1^{wt} mice are injected with AAV particles engineered to express ENPP3-Fc protein, and a sixth cohort of ttw (or ENPP1^{KO}) mice are injected with AAV particles engineered to express ENPP3-Fc protein. The wildtype mice are maintained on regular chow diet and the ttw mice (or ENPP1^{KO}) are fed high phosphate Teklad diet.

Vector injection: After two weeks of age, all mice receive a retro-orbital injection or tail vein injection of approx. 1×10¹² to 1×10^{15 vg/kg} preferably. 1×10¹³ to 1×10^{14 vg/kg} in PBS pH 7.4 per mouse. The injected vectors are either empty “null” (control group) or carried the NPP1or NPP3 gene (study group).

Assays: Kidney histology, PPi levels, and blood urine parameters such as FGF-23 levels, vitamin D, Parathyroid hormone (PTH) levels, serum/blood urea levels, blood urea nitrogen (BUN) levels, serum/blood creatine levels and plasma pyrophosphate (PPi) are analyzed for each cohort as described in Example 3 and 4.

Histology, Histomorphometry, and Micro-CT: Bone analysis is conducted following the protocols as described in Example 3.

Bone biomechanical testing: Bone analysis is conducted following the protocols as described in Example 3.

Results: Untreated ttw (or ENPP1^{KO}) mice generally exhibit reduced body weight, lethargy, diminished cortical bone thickness and trabecular bone volume, calcification of cartilage and ligaments, reduced bone density in the long bones such as Femur and Tibia, and increased mortality compared to wild type. In contrast, ttw (or ENPP1^{KO}) mice treated with AAV expressing ENPP1 proteins or ENPP3 proteins are expected to show an increase in body weight approaching the body weight ranges of normal WT mice, normal alertness, increases bone mineral density, improved cortical bone thickness and trabecular bone volume, increased bone strength and bone ductility. The ttw (or ENPP1^{KO} mice treated with null vector are expected to display calcifications in their hearts, aortas and coronary arteries, and histologic evidence of myocardial infarctions in the free wall of right ventricle, calcifications of coronary arteries, heart, ascending and descending aorta, myocardial cell necrosis, and myocardial fibrosis in the myocardial tissue adjacent to regions of coronary artery calcification. In contrast, ttw (or ENPP1^{KO}) animals treated with vector expressing ENPP1-Fc or ENPP3-Fc are expected to display an absence of cardiac, arterial, or aortic calcification on histology or post-mortem micro-CT. The ttw (or ENPP1^{KO}) mice treated with null vector also show calcifications centered in the renal medulla along with heavy, extensive calcifications, centered in the outer medulla, with extension into the renal cortex. In contrast, ttw (or ENPP1^{KO} ) mice treated with viral vector based expression of ENPP1 or ENPP3 are expected to show a reduction or lack of renal mineral deposits in the tubular lumen, reduction of calcification of spine, and soft tissue vasculature with histology similar to that of healthy wildtype mice.

In addition to survival, daily animal weights, and terminal histology, treatment response is assessed via post-mortem high-resolution micro-CT scans to image vascular calcifications, and plasma PPi concentrations. None of the WT or treated (vector expressing ENPP1) ttw (or ENPP1^{KO}) are expected to possess any vascular calcifications via micro-CT, in contrast to the dramatic calcifications that are expected to be seen in the aortas, coronary arteries, and hearts of the untreated (null vector) ttw (or ENPP1^{KO}) cohort. In addition, serum PPi concentrations of treated (vector expressing ENPP1) ttw (or ENPP1^{KO}) animals (5.2 μM) are expected to be elevated to WT levels (4.4 μM) and significantly above untreated ttw (or ENPP1^{KO}) levels (0.5 μM).

Untreated ttw (or ENPP1^{KO}) mice are also expected to show a significant increase in serum inorganic phosphorous (pi), increase in PTH and FGF23 levels but a decrease in 1,25(OH)₂-Vitamin D levels and lower PPi levels (˜0.5 μM) when compared with that of healthy wild type mice (Normal levels of PP are about 2-4 μM; about 10-65 ng/L for PTH; median FGF23 level is 13 RU/ml and normal FGF23 level ranges from 5 to 210 RU/ml; normal Vitamin D levels are 20 ng/mL to 50 ng/mL). In contrast, treated ttw (or ENPP1^{KO}) mice are expected to show elevated levels of PPi (˜4-5 μM) which are expected to be higher than the PPi levels found in untreated ttw (or ENPP1^{KO}) mice (˜0.5 μM). Thus a person of ordinary skill can determine the therapeutic efficacy of vector based ENPP1 or ENPP3 in treating Osteopenia or Osteomalcia or Osteoarthritis by observing one or more factors like reduction (25%, or 50%, or 70%, or 90% or 100% reduction) of calcification of soft tissues in kidneys and coronary arteries visualized through histological analysis , increase in serum PPi levels, normalization of vitamin D levels, reduction in FGF23 levels to normal ranges and normalization of PTH levels from blood analysis, improved long bone strength, increased bone density, improved corticular bone thickness and trabecular bone volume, increased survival and improved kidney function observed by increase in urine urea and creatine along with increased weight gain.

Treatment of Human Subjects:

A human patient suffering from Osteopenia or Osteomalacia or Osteoarthritis is treated by providing an intravenal injection containing approximately. 5×10¹¹-5×10^{15 vg/kg} in 1× PBS at pH 7.4, in some embodiments approximately 1×10¹²-1×10^{15 vg/kg} in 1× PBS at pH 7.4 per subject capable of delivering and expressing hENPP1 or hENPP3. Successful treatment of Osteopenia or Osteomalacia or Osteoarthritis is observed by monitoring one or more aforesaid parameters through periodic bone strength, bone density blood and urine tests as discussed for mouse models. Instead of histological analysis which requires staining of kidney slices or arterial tissues which is not feasible to perform in living patients, one instead uses noninvasive visualization techniques as discussed in example 4.

Similarly, patients are subjected to periodic bone density measurements using dual energy x-ray absorptiometry (DXA) or peripheral dual energy x-ray absorptiometry (pDXA) or quantitative ultrasound (QUS) or peripheral quantitative computed tomography (pQCT). Bone density scores obtained from one of these methods provides indication of the condition and progress obtained after the treatment. A T-score of −1.0 or above is considered as normal bone density, a T-score between −1.0 and −2.5 indicates the presence of Osteopenia and whereas a T-score of −2.5 or below indicates the presence of Osteoporosis. A gradual improvement of T-score is expected in patients treated with ENPP1 or ENPP3 of the invention.

A medical doctor having skill in visualizing soft tissue calcification, cardiac calcification, bone density visualization undertakes the treatment of a subject afflicted with Osteopenia or Osteoarthiritis by administration of AAV virions expressing hENPP1 or hENPP3. In some embodiments, the physician uses viral particles that deliver constructs of hENPP1 or hENPP3 and express the corresponding proteins under the control of an inducible promoter. The physician thus has the option to control the dosage (amount of hENPP1 or hENPP3 expressed) based on the rate and extent of improvement of symptoms. A successful treatment and suitable dosage is readily inferred by a medical professional of skill in art by observing one or more positive symptoms such as normal vitamin D levels (20 ng/ml to 50 ng/mL is considered adequate for healthy people. A level less than 12 ng/mL indicates vitamin D deficiency), normal bone density (T score of ≥−1) normal blood urea nitrogen levels (BUN level for healthy adults is 7-20 mg/dL), weight gain, increase in serum PPi levels (at least about 4-5 μm), reduction in calcification (25%, or 50%, or 70%, or 90% or 100% reduction) of arterial tissues, improved bone strength visualized by noninvasive techniques such as CT, magnetic resonance imaging (MRI) or ultrasound scans.

### Example 9—Treatment of ADHR-2 or ARHR-2 and or XLH Using Viral Vectors Expressing ENPP1 or ENPP3

The following example provides AAV expressing ENPP1 or ENPP3 which are expected to be effective in treating symptoms associated with ADHR-2 orARHR-2 or XLH. ENPP1-Fc and ENPP3-Fc are used in the examples for illustrative purposes and similar results can be obtained by using other ENPP1 or ENPP3 fusions of the invention.

AAV virions expressing ENPP1-Fc protein or ENPP3-Fc protein are made according to example 1, and administered to a HYP mouse model of X-linked hypophosphatasia (XLH); (Liang, et al. , 2009, Calcif. Tissue Int. 85(3):235-46). Six sets of mice are used for treatment with ENPP1 and ENPP3. Similar experiment is repeated using ENPP1 age stiffened joint mouse (ENPP1^{asj/asj}) which also serves as a model for ARHR-2. (Am J Hum Genet. 2010 Feb. 12; 86(2): 273-278.) in addition to GACI.

Control cohorts: In this experiment, a first cohort of ENPP1 wt mice that serve as control group are injected with AAV particles that comprise a null vector and, a second cohort of HYP (or ENPP1^{asj/asj}) mice that serve as a control group are injected with AAV particles that comprise a null vector.

ENPP1-treated mice cohorts: a third cohort of ENPP1^{wt} mice are injected with AAV particles engineered to express ENPP1-Fc protein, and a fourth cohort of HYP (or ENPP1^{asj/asj}) mice are injected with AAV particles engineered to express ENPP1-Fc protein.

ENPP3-treated mice cohorts: a fifth cohort of ENPP1^{wt} mice are injected with AAV particles engineered to express ENPP3-Fc protein, and a sixth cohort of HYP (or ENPP1^{asj/asj}) mice are injected with AAV particles engineered to express ENPP3-Fc protein. The wildtype mice are maintained on regular chow diet and the HYP (or ENPP1^{asj/asj}) mice are fed high phosphate Teklad diet.

Vector injection: After two weeks of age, all mice receive a retro-orbital injection or tail vein injection of approx. 1×10¹² to 1×10^{15 vg/kg} preferably 1×10¹³ to 1×10^{14 vg/kg} in PBS pH 7.4 per mouse. The injected vectors are either empty “null” (control group) or carried the NPP1or NPP3 gene (study group).

Assays: Kidney histology, PPi levels, and blood urine parameters such as FGF-23 levels, vitamin D, Parathyroid hormone (PTH) levels, serum/blood urea levels, blood urea nitrogen (BUN) levels, serum/blood creatine levels and plasma pyrophosphate (PPi) are analyzed for each cohort as described in Example 3 and 4.

Histology, Histomorphometry, and Micro-CT: Bone analysis is conducted following the protocols as described in Example 3.

Bone biomechanical testing: Bone analysis is conducted following the protocols as described in Example 3.

Results: Untreated HYP (or ENPP1^{asj/asj}) mice generally exhibit reduced body weight, lethargy, diminished cortical bone thickness and trabecular bone volume , calcification of cartilage and ligaments, reduced bone density in the long bones such as Femur and Tibia, and increased mortality compared to wild type. In contrast, HYP (or ENPP1^{asj/asj}) mice treated with AAV expressing ENPP1 proteins or ENPP3 proteins are expected to show an increase in body weight approaching the body weight ranges of normal WT mice, normal alertness, increases bone mineral density, improved cortical bone thickness and trabecular bone volume, increased bone strength and bone ductility. The HYP (or ENPP1^{asj/asj}) mice treated with null vector are expected to display calcifications in their hearts, aortas and coronary arteries, and histologic evidence of myocardial infarctions in the free wall of right ventricle, calcifications of coronary arteries, heart, ascending and descending aorta, myocardial cell necrosis, and myocardial fibrosis in the myocardial tissue adjacent to regions of coronary artery calcification. In contrast, HYP (or ENPP1^{asj/asj}) mice treated with vector expressing ENPP1-Fc or ENPP3-Fc are expected to display an absence of cardiac, arterial, or aortic calcification on histology or post-mortem micro-CT. The HYP (or ENPP1^{asj/asj}) mice treated with null vector also show calcifications centered in the renal medulla along with heavy, extensive calcifications, centered in the outer medulla, with extension into the renal cortex. In contrast HYP (or ENPP1^{asj/asj}) mice treated with viral vector based expression of ENPP1 or ENPP3 are expected to show a reduction or lack of renal mineral deposits in the tubular lumen, reduction of calcification of spine, and soft tissue vasculature with histology similar to that of healthy wildtype mice.

In addition to survival, daily animal weights, and terminal histology, treatment response is assessed via post-mortem high-resolution micro-CT scans to image vascular calcifications, and plasma PPi concentrations. None of the WT or treated (vector expressing ENPP1) HYP (or ENPP1^{asj/asj}) mice are expected to possess any vascular calcifications via micro-CT, in contrast to the dramatic calcifications that are expected to be seen in the aortas, coronary arteries, and hearts of the untreated (null vector) HYP (or ENPP1^{asj/asj}) cohort. In addition, serum PPi concentrations of treated (vector expressing ENPP1) HYP (or ENPP1^{asj/asj}) mice (5.2 μM) are expected to be elevated to WT levels (4.4 μM) and significantly above untreated HYP (or ENPP1^{asj/asj}) levels (0.5 μM).

Untreated HYP (or ENPP1^{asj/asj}) mice are also expected to show a significant increase in serum inorganic phosphorous (pi), increase in PTH and FGF23 levels but a decrease in 1,25(OH)₂-Vitamin D levels and lower PPi levels (˜0.5 μM) when compared with that of healthy wild type mice (Normal levels of PP are about 2-4 μM; about 10-65 ng/L for PTH; median FGF23 level is 13 RU/ml and normal FGF23 level ranges from 5 to 210 RU/ml; normal Vitamin D levels are 20 ng/mL to 50 ng/mL). In contrast, treated HYP (or ENPP1^{asj/asj}) mice are expected to show elevated levels of PPi (˜4-5 μM) which are expected to be higher than the PPi levels found in untreated HYP (or ENPP) mice (0.5 μM). Thus a person of ordinary skill can determine the therapeutic efficacy of vector based ENPP1 or ENPP3 in treating ADHR-2 or ARHR-2 or XLH by observing one or more factors like reduction (25%, or 50%, or 70%, or 90% or 100% reduction) of calcification of soft tissues in kidneys and coronary arteries visualized through histological analysis , increase in serum PPi levels, normalization of vitamin D levels, reduction in FGF23 levels to normal ranges and normalization of PTH levels from blood analysis, improved long bone strength, increased bone density, improved corticular bone thickness and trabecular bone volume, increased survival and improved kidney function observed by increase in urine urea and creatine along with increased weight gain.

Treatment of Human Subjects:

A human patient suffering from ADHR-2 or ARHR-2 or XLH is treated by providing an intravenal injection containing approximately. 5×10¹¹-5×10^{15 vg/kg} in 1× PBS at pH 7.4, in some embodiments approximately 1×10¹²-1×10^{15 vg/kg} in 1× PBS at pH 7.4 ₚer subject capable of delivering and expressing hENPP1 or hENPP3. Successful treatment of ADHR-2 orARHR-2 or XLH is observed by monitoring one or more aforesaid parameters through periodic bone strength, bone density blood and urine tests as discussed for mouse models. Instead of histological analysis which requires staining of kidney slices or arterial tissues which is not feasible to perform in living patients, one instead uses noninvasive visualization techniques as discussed in example 4.

Similarly, patients are subjected to periodic bone density measurements using dual energy x-ray absorptiometry (DXA) or peripheral dual energy x-ray absorptiometry (pDXA) or quantitative ultrasound (QUS) or peripheral quantitative computed tomography (pQCT). Bone density scores obtained from one of these methods provides indication of the condition and progress obtained after the treatment. A T-score of −1.0 or above is considered as normal bone density, a T-score between −1.0 and −2.5 indicates the presence of Osteopenia and whereas a T-score of −2.5 or below indicates the presence of Osteoporosis. A gradual improvement of T-score is expected in patients treated with ENPP1 or ENPP3 of the invention.

A medical doctor having skill in visualizing soft tissue calcification, cardiac calcification, bone density visualization undertakes the treatment of a subject afflicted with ADHR-2 orARHR-2 or XLH by administering AAV virions expressing hENPP1 or hENPP3. In some embodiments, the physician uses viral particles that deliver constructs of hENPP1 or hENPP3 and express the corresponding proteins under the control of an inducible promoter. The physician thus has the option to control the dosage (amount of hENPP1 or hENPP3 expressed) based on the rate and extent of improvement of symptoms. A successful treatment and suitable dosage is readily inferred by a medical professional of skill in art by observing one or more positive symptoms such as normal vitamin D levels (20 ng/ml to 50 ng/mL is considered adequate for healthy people. A level less than 12 ng/mL indicates vitamin D deficiency), normal bone density (T score of ≥−1) normal blood urea nitrogen levels (BUN level for healthy adults is 7-20 mg/dL), weight gain, increase in serum PPi levels (at least about 4-5 μm), reduction in calcification (25%, or 50%, or 70%, or 90% or 100% reduction) of arterial tissues, improved bone strength visualized by noninvasive techniques such as CT, magnetic resonance imaging (MRI) or ultrasound scans.

### Example 10—Analysis of Plasma PPi levels, ENPP1 Concentration and Activity Levels in Model Mice Post Viral Adminstration

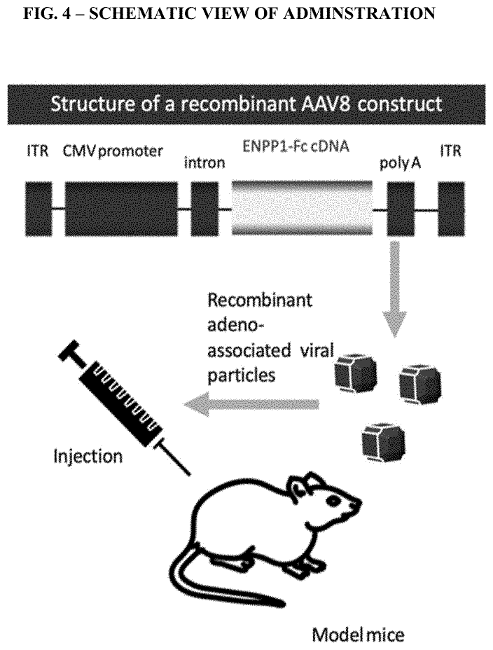

Three cohorts of Normal mice were used for this experiment. Each cohort contains five adult mice. The first cohort was used as a “Control group” and saline solution was injected to the control group. The second cohort was used as the “Low dose group” and AAV vector at 1 e¹³ vg/kg concentration was injected to the low dose group. The Third cohort was used a “High dose group” and AAV vector at 1 e¹⁴ vg/kg concentration was injected to the high dose group. The process of generating viral particles from AAVconstruct and injecting the recombinant AAV viral particles comprising ENPP1 fusion proteins into normal mice is schematically shown in FIG. 4. Mice from all cohorts were bled at 7^{th}, 28^{th} and 56^{th} day post injection to collect blood plasma and serum.

Blood was collected into heparin-treated tubes. Plasma was isolated, and platelets were removed by filtering through a Nanosep 30 kDa Omega centrifugal filter (Pall, OD030C35). The samples were centrifuged at top speed (˜20 kg) at 4° C. for 20 min. The flow-through was collected and placed on dry ice to flash freeze the samples. The samples were stored at −80° C. for later use in assay.

The samples collected were first assayed to determine the activity levels of ENPP1 using the colorimetric substrate, p-nitrophenyl thymidine 5′-monophosphate (Sigma). Plasma samples were incubated with 1 mg/ml p-nitrophenyl thymidine 5′-monophosphate for 1 hr in 1% Triton, 200 mM Tris, pH 8.0 buffer. 100 mM NaOH was added after 1 hr to stop the reaction, and absorbance was measured at 405 nm. Specific activity was determined by following assay protocols disclosed by R& D Systems for recombinant human ENPP-1; Catalog No: 6136-WN.

$$Specific Activity ( pmol / min / µ g ) = ( \frac{\begin{matrix}Adjusted {{V}_{max}}^{*} ( OD / min ) \times \\ Conversion {Factor}^{**} ( pmol / OD )\end{matrix}}{amount of enzyme ( µg )} )$$

$${}^{*} A djusted for Substrate Blank$$

$${}^{**} D erived using calibration standard 4 - Nitrophenol ( Sigma - Aldrich , Catalog £ 241326 ) .$$

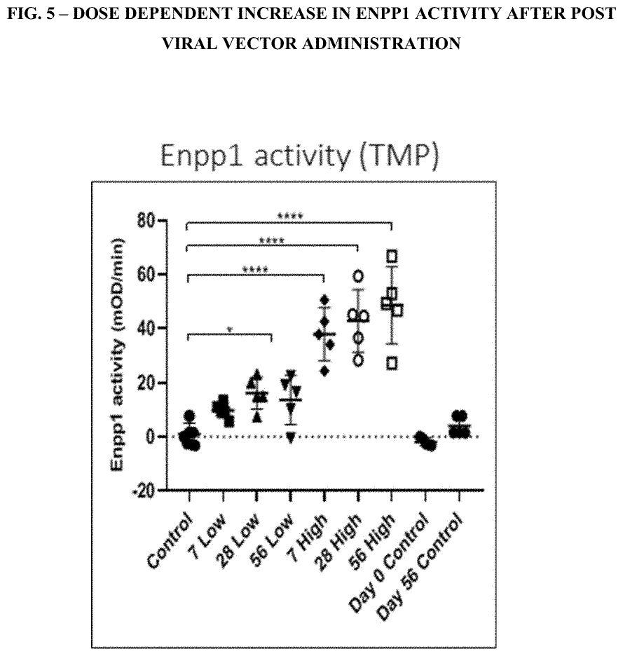

The results of the ENPP1 activity assay are in FIG. 5 and they show that there is a dose dependent increase in ENPP1 activity post injection. Normal mouse plasma was used as a reference standard to normalize the ENPP1 activity levels and One-way ANOVA was used for statistical analysis. FIG. 5 shows that the ENPP1 activity levels were higher in the low dose group when compared with that of the control group. Similarly, the ENPP1 activity levels were higher in the high dose group when compared with that of the low dose group and the control group. Amongst the low dose and high dose cohorts, ENPP1 activity was stable in the plasma samples from day 7 to day 56 in the high-dose group, but there was a slight decrease in the ENPP1 activity from day 28 to day 56 in the low-dose group.

The samples were then assayed to determine the concentration of ENPP1 using sandwich ELISA assay with ENPP1 polyclonal antibody derived from Sigma (SAB1400199). 96 Well Clear Flat Bottom Polystyrene High Bind Microplate (Corning Cat #9018), BSA (Sigma #7906), 10× Dulbecco's Phosphate Buffered Saline (DPBS) (Quality Biological Cat #119-068-101) , Tween-20 (Sigma Cat #P2287), Anti-ENPP1, Antibody Produced in Mouse (Sigma-Aldrich Cat #SAB1400199), Sure Blue TMB Microwell Peroxidase Substrate (1-component) (KPL Prod #52-00-01), 2N Sulphuric acid (BDH Product #BDH7500-1), MilliQ Water, C57BL/6 Mouse Plasma NaHep Pooled Gender (BioIVT cat #MSEO1PLNHPNN), Mouse Serum (BIO IVT elevating Science cat #MSE01SRMPNN) were used for the ELISA assay.

A standard curve for ENPP1-Fc protein is generated by following standard procedures known in art. Briefly serial dilutions of ENPP1-Fc protein ranging from 2 mg/ml to 30 ng·ml were made. The 96 well plate was first coated with 1 μg/1 mL of overnight coat solution comprising the ENPP1 capture antibody in 1× PBS. The wells were then incubated with 5% BSA in PBS for 1 hr and were then washed with post block solution. The ENPP1 dilution samples were added to the coated 96 well plates and incubated for 1.5 hrs. After incubation, the wells were washed four times with 300 μl of 0.05 T % PBST. The washed wells were then treated with 100 μL/well of the detection HRP antibody conjugate and were incubated for 1 hour. After incubation with HRP antibody conjugate, the wells were washed four times with 300 μl of 0.05 T % PBST. The washed wells were then treated with 100 μl of TMB Microwell Peroxidase Substrate per well and incubated in dark for 30 minutes. The wells were then washed four times with 300 μl of 0.05 T % PBST and the reaction was stopped using 2N Sulphuric Acid. The absorbance of the well was read using Microplate Reader at a wavelength of 450 nm. A standard curve was generated using the absorbance read and the corresponding concentration of the ENPP1 serial dilution samples.

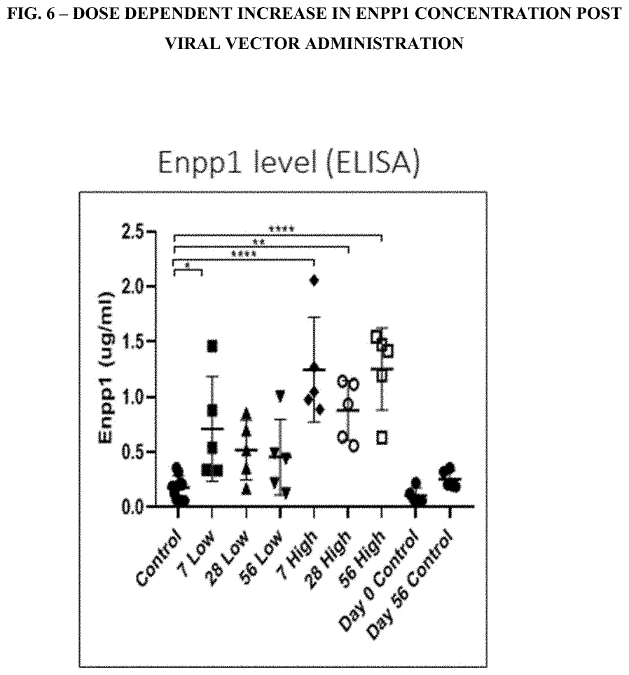

The assay was then repeated using plasma samples obtained from control, low dose and high dose cohorts on 7, 28 and 56 days post viral injection. The absorbance generated in each plasma sample was correlated with the standard curve of ENPP1-Fc to determine concentration of ENPP1-Fc in the plasma samples. The results of ENPP1 concentration assay are shown in FIG. 6 and they show a dose dependent increase in ENPP1 concentration post viral vector injection. Normal mouse plasma was used as a reference standard to normalize the ENPP1 concentration levels and One-way ANOVA was used for statistical analysis. FIG. 6 shows that the ENPP1 concentration was higher in the low dose group when compared with that of the control group. Similarly, the ENPP1 activity levels were higher in the high dose group when compared with that of the low dose group and the control group. Amongst the low dose and high dose cohorts, ENPP1 level was stable in the samples from day 7 to day 56 in the high-dose group, but there was a slight decrease in the ENPP1 level from day 28 to day 56 in the low-dose group

The samples were also assayed to determine the concentration of Plasma PPi using Sulfurylase assay. ATP sulfurylase (NEB-M0394L, Lot#:10028529), Adenosine 5′-phosphosulfate (APS; Santa Cruz, sc-214506)), PPi: 100 uM stock, HEPES pH 7.4 buffer (Boston Bioproducts BB2076), Magnesium sulfate (MgSO4) solution at 1M, Calcium chloride (CaCl2) solution at 1M, BactiterGlo (Promega G8231), Plates (Costar 3915, black flat bottom) and Plate reader (Molecular Devices Spectramax I3x) were used for the PPi-Sulfurylase assay. PPi standards (0.125-4 μM) were prepared in water using serial dilution. PPi standards and PPi in filtered plasma samples were converted into ATP by ATP sulfurylase in the presence of excess adenosine 5′ phosphosulfate (APS). The sample (15 μl) was treated with 5 μl of a mixture containing 8 mM CaCl₂, 2 mM MgSO4, 40 mM HEPES pH7.4, 80 uM APS (Santa Cruz, sc-214506), and 0.1 U/ml ATP sulfurylase (NEB-M0394L). The mixture was incubated for 40 min at 37° C., after which ATP sulfurylase was inactivated by incubation at 90° C. for 10 min. The generated ATP was determined using BactiterGlo (Promega G8231) by mixing 20 μl of treated sample or standard with 20 μl of BactiterGlo reagent. Bioluminescence was subsequently determined in a microplate reader and from the standard curve, the amount of PPi generated in each sample was subsequently determined.

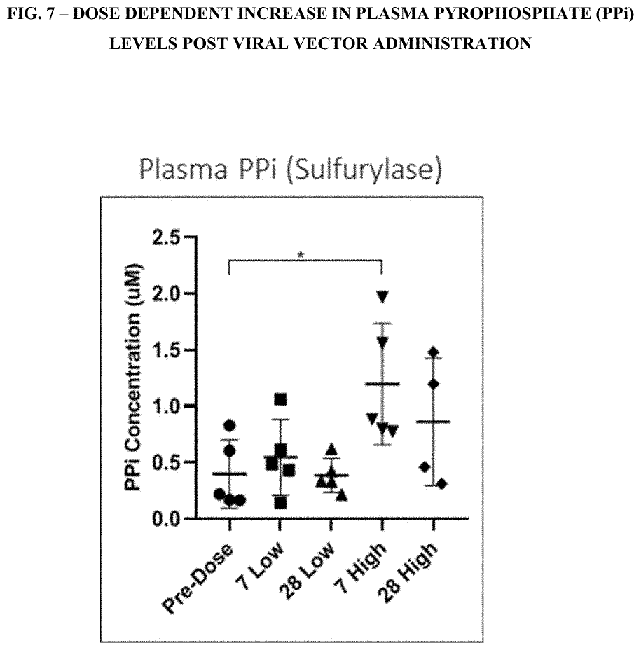

The results of Plasma PPi assay are shown in FIG. 7. Results show a dose dependent increase in Plasma PPi post viral vector injection. Normal mouse plasma was used as a reference standard to normalize the Plasma PPi concentraion levels and One-way ANOVA was used for statistical analysis. FIG. 7 shows that the Plasma PPi concentration was slightly higher in the low dose group when compared with that of the control group. Similarly, the Plasma PPi concentration were higher in the high dose group when compared with that of the low dose group and the control group. Amongst the low dose and high dose cohorts, ENPP1 level was stable in the plasma samples from day 7 to day 56 in the high-dose group, but a slight decrease in the ENPP1 level from day 28 to day 56 in the low-dose group was observed.

In a related experiment, C57/B1 male mice 5-6 weeks old were administered intravenously a single dose of an AAV viral vector at 1 e14 vg/kg, or a vehicle control (containing no AAV vector). Animals were administered GK1.5 (40 μg/mouse one day prior to administration of the viral vector or vehicle, and then 25 μg/mouse every seven days thereafter until completion of the study). The AAV viral vector was engineered to express a fusion protein of ENPP1 and an IgG Fc similar to the polypeptide described in Example 10 except the ENPP1 portion and the IgG Fc portion of the fusion protein were joined by the following linker amino acid sequence: GGGGS. Mice administered the AAV viral vector demonstrated a higher level of ENPP1 enzyme activity than the vehicle only control as measured over an approximately 40 day period.

### Example 11—Analysis of ENPP1 Concentration and Activity Levels in Model Mice 112 Days Post Viral Adminstration

Three cohorts of Normal mice were used for this experiment. Each cohort contains five adult mice. The first cohort was used as a “Control group” and saline solution was injected to the control group. The second cohort was used as the “Low dose group” and AAV vector at 1 e¹³ vg/kg concentration was injected to the low dose group. The Third cohort was used a “High dose group” and AAV vector at 1 e¹⁴ vg/kg concentration was injected to the high dose group. The process of generating viral particles from AAVconstruct and injecting the recombinant AAV viral paritcles comprising ENPP1 fusion proteins into normal mice is schematically shown in FIG. 4. Mice from all cohorts were bled at 7^{th}, 28^{th}, 56^{th} and 112^{th} day post injection to collect blood plasma and serum.

Blood was collected into heparin-treated tubes. The samples were centrifuged at top speed (˜20 kg) at 4° C. for 20 min. The flow-through was collected and placed on dry ice to flash freeze the samples. The samples were stored at −80° C. for later use in assay.

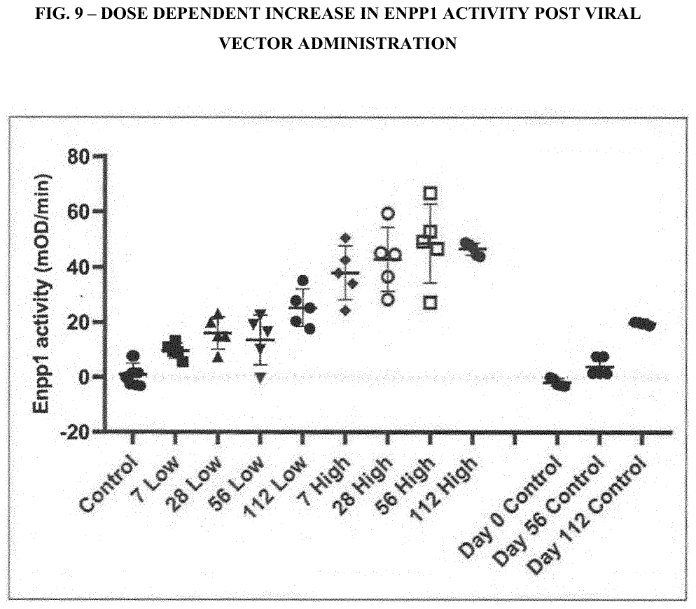

The samples collected were first assayed to determine the activity levels of ENPP1 using the colorimetric substrate, p-nitrophenyl thymidine 5′-monophosphate (Sigma) as described in Example 10. The results of the ENPP1 activity assay are in FIG. 9 and they show that there is a dose dependent increase in ENPP1 activity post injection. Normal mouse plasma was used as a reference standard to normalize the ENPP1 activity levels and One-way ANOVA was used for statistical analysis. FIG. 9 shows that the ENPP1 activity levels were higher in the low dose group when compared with that of the control group. Similarly, the ENPP1 activity levels were higher in the high dose group when compared with that of the low dose group and the control group.

The samples were then assayed to determine the concentration of ENPP1 using sandwich ELISA assay with ENPP1 polyclonal antibody derived from Sigma (SAB1400199) following the protocols taught in Example 10. The assay was then repeated using plasma samples obtained from control, low dose and high dose cohorts on 7, 28, 56 and 112 days post viral injection. The absorbance generated in each plasma sample was correlated with the standard curve of ENPP1-Fc to determine concentration of ENPP1-Fc in the plasma samples.

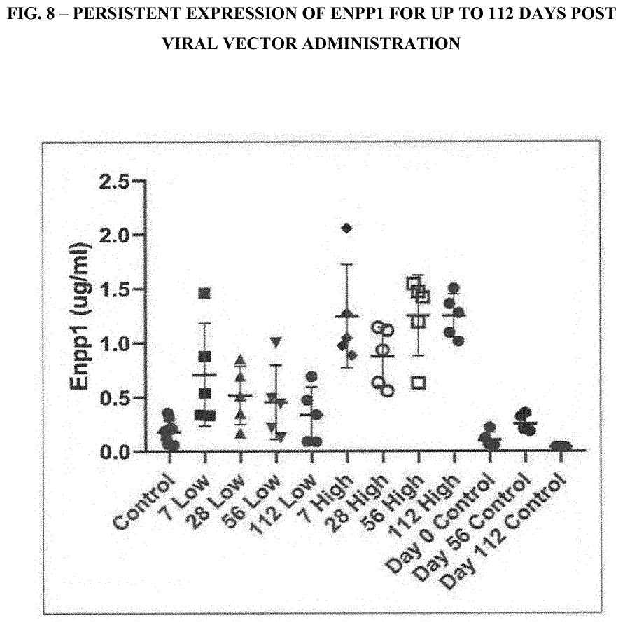

The results of ENPP1 concentration assay are shown in FIG. 8 and they show a dose dependent increase in ENPP1 concentration post viral vector injection. Normal mouse plasma was used as a reference standard to normalize the ENPP1 concentration levels and One-way ANOVA was used for statistical analysis. FIG. 8 shows that the ENPP1 concentration was higher in the low dose group when compared with that of the control group. Similarly, the ENPP1 levels were higher in the high dose group when compared with that of the low dose group and the control group.

### Other Embodiments

From the foregoing description, it will be apparent that variations and modifications may be made to the invention described herein to adopt it to various usages and conditions, including the use of different signal sequences to express functional variants of ENPP1 or ENPP3 or combinations thereof in different viral vectors having different promoters or enhancers or different cell types known in art to treat any diseases characterized by the presence of pathological calcification or ossification are within the scope according to the invention. Other embodiments according to the invention are within the following claims.

Recitation of a listing of elements in any definition of a variable herein includes definitions of that variable as any single element or combination (or sub combination) of listed elements. Recitation of an embodiment herein includes that embodiment as any single embodiment or in combination with any other embodiments or portions thereof.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Other embodiments are within the following claims.

1. A method of treating a subject having an ENPP1 protein deficiency, the method comprising administering to the subject a therapeutically effective amount of a viral vector encoding a polypeptide comprising the catalytic domain of an ENPP1 protein, wherein said administration provides for expression of said polypeptide in said subject, thereby treating said subject, wherein said viral vector is an adeno-associated (AAV) viral vector.

2. The method of claim 1, wherein administration of said viral vector to said subject increases plasma pyrophosphate (PPi) or plasma ENPP1 concentration in said subject.

3. The method of claim 1, wherein said polypeptide sequence comprises the extracellular domain of an ENPP1.

4. The method of claim 1, wherein said polypeptide comprises the transmembrane domain of an ENPP1 protein.

5. The method of claim 1, wherein said polypeptide comprises residues 99-925 (Pro Ser Cys to Gin Glu Asp) of SEQ ID NO: 1.

6. The method of claim 1, wherein said polypeptide comprises residues 1-833 (Phe Thr Ala to Gin Glu Asp) of SEQ ID NO: 89 or residues 1-830 (Gly Leu Lys to Gin Glu Asp) of SEQ ID NO: 91.

7. The method of claim 1, wherein said viral vector comprises a polynucleotide sequence encoding said polypeptide and a promoter sequence that directs transcription of said polynucleotide.

8. The method of claim 7, wherein said polynucleotide encodes a signal peptide which is amino-terminal to the polynucleotide sequence encoding the catalytic domain of said ENPP1 protein.

9. The method of claim 8, wherein said signal peptide is an Azurocidin signal peptide.

10. The method of claim 1, wherein said AAA vector has a serotype selected from the group consisting of: AAV1, AAV2, AAV3, AAV4, AAV5, AAV6, AAV7, AAV8, AAV9, and AAV-rh74.

11. The method of claim 1, wherein said polypeptide is a fusion protein comprising: (i) an ENPP1 protein and (ii) a half-life extending domain.

12. The method of claim 11, wherein said half-life extending domain is an IgG Fc domain or a functional fragment of said IgG Fc domain.

13. The method of claim 11, wherein said half-life extending domain is an albumin domain or a functional fragment of said albumin domain.

14. The method of claim 11, wherein said half-life extending domain is carboxy terminal to said ENPP1 protein in the fusion protein.

15. The method of claim 8, wherein said polynucleotide encodes a linker sequence that joins said ENPPI protein and said half-life extending domain of said fusion protein.

16. The method of claim 1, wherein said polypeptide comprises the amino acid sequence of SEQ ID NO: 89, 91, 92, or 93.

17. A method of treating a subject having an ENPPI protein deficiency, the method comprising administering to the subject a therapeutically effective amount of a vector encoding a polypeptide comprising the catalytic domain of an ENPP1 protein, thereby treating said subject, wherein the vector is an AAV8 serotype viral vector.

18. The method of claim 17, wherein said polypeptide comprises residues 99-925 (Pro Ser Cys to Gln Glu Asp) of SEQ ID NO: 1.

19. The method of claim 17, wherein said polypeptide comprises residues 1-833 (Phe Thr Ala to Gin Glu Asp) of SEQ ID NO: 89 or residues 1-830 (Gly Leu Lys to Gin Glu Asp) of SEQ ID NO: 91.

20. The method of claim 17, wherein said polypeptide is a fusion protein comprising: (i) an ENPP1 protein and (ii) a half-life extending domain.

21. The method of claim 20, wherein said half-life extending domain is an IgG Fc domain or a functional fragment of said IgG Fe domain.

22. The method of claim 17, wherein said polypeptide comprises the amino acid sequence of SEQ ID NO: 89, 91, 92, or 93.

23. A method of treating a subject having an ENPP1 protein deficiency, the method comprising administering to the subject a therapeutically effective amount of an adeno-associated viral vector encoding a fusion polypeptide comprising: (i) an ENPPT protein and (ii) a half-life extending domain, thereby treating said subject.

24. The method of claim 23, wherein said half-life extending domain is an IgG Fc domain or a functional fragment of said IgG Fe domain.

25. The method of claim 1, wherein said polypeptide comprises the amino acid sequence of SEQ ID NO: 89, 91, 92, or 93.

26. A method of treating a subject having an ENPP1 protein deficiency, the method comprising administering to the subject a therapeutically effective amount of an adeno-associated viral (AAV) vector encoding a fusion polypeptide comprising: (i) an ENPPI protein and (ii) a half-life extending domain, thereby treating said subject, wherein the AAV vector is an AAV8 serotype viral vector.

27. The method of claim 26, wherein said polypeptide comprises the amino acid sequence of SEQ ID NO: 89.

28. The method of claim 26, wherein said polypeptide comprises the amino acid sequence of SEQ ID NO: 91.

29. The method of claim 26, wherein said polypeptide comprises the amino acid sequence of SEQ NO: 92.

30. The method of claim 26, wherein said polypeptide comprises the amino acid sequence of SEQ ID NO: 93.
